(19) United States
(12) Patent Application Publication
(10) Pub. No.: US 2026/0366039 A1
(43) Pub. Date: Nov. 19, 2026

(54) NOVEL INSECT INHIBITORY PROTEINS

(71) Applicant: Monsanto Technology LLC, St. Louis, MO (US)

(72) Inventors: David J. Bowen, Wildwood, MO (US); Catherine A. Chay, Ballwin, MO (US); Todd A. Ciche, San Diego, CA (US); Stanislaw Flasinski, Ballwin, MO (US); Arlene R. Howe, Clarkson Valley, MO (US)

(21) Appl. No.: 19/748,807

(22) Filed: Jul. 21, 2026

Related U.S. Application Data
(62) Division of application No. 18/635,866, filed on Apr. 15, 2024.
Application No. 18/635,866 is a division of application No. 17/934,287, filed on Sep. 22, 2022, now Pat. No. 11,987,800.
Application No. 17/934,287 is a division of application No. 16/580,583, filed on Sep. 24, 2019, now Pat. No. 11,512,324.
(60) Provisional application No. 62/736,236, filed on Sep. 25, 2018.

(51) Int. Cl. C12N 15/82; A01N 63/50; C07K 14/24

(52) U.S. Cl. CPC C12N 15/8286; A01N 63/50; C07K 14/24

(57) ABSTRACT

A pesticidal protein class of PirA, PirB, and PirAB fusion proteins exhibiting toxic activity against Coleopteran, Lepidopteran, and Hemipteran pest species is disclosed. DNA constructs are provided which contain a recombinant nucleic acid sequence encoding the PirA, PirB, and PirAB fusion proteins. Transgenic plants, plant cells, seed, and plant parts resistant to Coleopteran, Lepidopteran, and Hemipteran infestation are provided which contain recombinant nucleic acid sequences encoding the PirA, PirB, and PirAB fusion proteins. Methods for detecting the presence of the recombinant nucleic acid sequences or the proteins of the present invention in a biological sample, and methods of controlling Coleopteran, Lepidopteran, and Hemipteran species pests using the PirA, PirB, and PirAB fusion proteins are also provided.

### REFERENCE TO RELATED APPLICATION

This application is a divisional of U.S. patent application Ser. No. 18/635,866, filed Apr. 15, 2024, which is a divisional of U.S. patent application Ser. No. 17/934,287, filed Sep. 22, 2022, now U.S. Pat. No. 11,987,800, which is a divisional of U.S. patent application Ser. No. 16/580,583, filed Sep. 24, 2019, now U.S. Pat. No. 11,512,324, which claims the benefit of U.S. Provisional Patent Application No. 62/736,236, filed Sep. 25, 2018, each of which is herein incorporated by reference in its entirety.

### INCORPORATION OF SEQUENCE LISTING

The file named “MONS465USD3_ST26.xml” containing a computer-readable form of the Sequence Listing was created on Jul. 17, 2026. This file is 359,579 bytes (measured in MS-Windows®) is contemporaneously filed by electronic submission (using the United States Patent Office EFS-Web filing system), and is incorporated into this application by reference in its entirety.

### FIELD OF THE INVENTION

The invention generally relates to the field of insect inhibitory proteins. A novel class of proteins exhibiting insect inhibitory activity against agriculturally-relevant pests of crop plants and seeds is disclosed. In particular, the disclosed proteins are insecticidally active against agriculturally-relevant pests of crop plants and seeds, particularly Coleopteran and Lepidopteran species of insect pests. Plants, plant parts, and seeds containing a recombinant polynucleotide construct encoding one or more of the disclosed toxin proteins are provided.

### BACKGROUND OF THE INVENTION

Improving crop yield from agriculturally significant plants including, among others, corn, soybean, sugarcane, rice, wheat, vegetables, and cotton, has become increasingly important. In addition to the growing need for agricultural products to feed, clothe and provide energy for a growing human population, climate-related effects and pressure from the growing population to use land other than for agricultural practices are predicted to reduce the amount of arable land available for farming. These factors have led to grim forecasts of food security, particularly in the absence of major improvements in plant biotechnology and agronomic practices. In light of these pressures, environmentally sustainable improvements in technology, agricultural techniques, and pest management are vital tools to expand crop production on the limited amount of arable land available for farming.

Insects, particularly insects within the Lepidoptera, Coleoptera, and Hemipteran orders, are considered a major cause of damage to field crops, thereby decreasing crop yields over infested areas. Historically, the intensive application of synthetic chemical insecticides was relied upon as the pest control agent in agriculture. Concerns for the environment and human health, in addition to emerging resistance issues, stimulated the research and development of biological pesticides. This research effort led to the progressive discovery and use of various entomopathogenic microbial species, including bacteria.

The biological control paradigm shifted when the potential of entomopathogenic bacteria, especially bacteria belonging to the genus Bacillus, was discovered and developed as a biological pest control agent. Strains of the bacterium Bacillus thuringiensis (Bt) have been used as a source for pesticidal proteins since it was discovered that Bt strains show a high toxicity against specific insects. Bt strains are known to produce delta-endotoxins that are localized within parasporal crystalline inclusion bodies at the onset of sporulation and during the stationary growth phase (e.g., Cry proteins), and are also known to produce secreted insecticidal protein. Upon ingestion by a susceptible insect, delta-endotoxins as well as secreted toxins exert their effects at the surface of the midgut epithelium, disrupting the cell membrane, leading to cell disruption and death. Genes encoding insecticidal proteins have also been identified in bacterial species other than Bt, including other Bacillus and a diversity of additional bacterial species, such as Brevibacillus laterosporus, Lysinibacillus sphaericus (“Ls” formerly known as Bacillus sphaericus), Paenibacillus popilliae, Photorhabdus and Xenorhabdus.

Crystalline and secreted soluble insecticidal toxins are highly specific for their hosts and have gained worldwide acceptance as alternatives to chemical insecticides. For example, insecticidal toxin proteins have been employed in various agricultural applications to protect agriculturally important plants from insect infestations, decrease the need for chemical pesticide applications, and increase yields. Insecticidal toxin proteins are used to control agriculturally-relevant pests of crop plants by mechanical methods, such as spraying to disperse microbial formulations containing various bacteria strains onto plant surfaces, and by using genetic transformation techniques to produce transgenic plants and seeds expressing insecticidal toxin protein.

The use of transgenic plants expressing insecticidal toxin proteins has been globally adapted. For example, in 2012, 26.1 million hectares were planted with transgenic crops expressing Bt toxins (James, C., Global Status of Commercialized Biotech/GM Crops: 2012. ISAAA Brief No. 44). The global use of transgenic insect-protected crops and the limited number of insecticidal toxin proteins used in these crops has created a selection pressure for existing insect alleles that impart resistance to the currently-utilized insecticidal proteins.

The development of resistance in target pests to insecticidal toxin proteins creates the continuing need for discovery and development of new forms of insecticidal toxin proteins that are useful for managing the increase in insect resistance to transgenic crops expressing insecticidal toxin proteins. New protein toxins with improved efficacy and which exhibit control over a broader spectrum of susceptible insect species will reduce the number of surviving insects which can develop resistance alleles. In addition, the use in one plant of two or more transgenic insecticidal toxin proteins toxic to the same insect pest and displaying different modes of action reduces the probability of resistance in any single target insect species.

Thus, the inventors herein disclose a protein toxin family from Xenorhabdus and Photorhabdus along with similar toxin proteins, variant proteins, and exemplary recombinant proteins that exhibit insecticidal activity against target Lepidopteran, Coleopteran, and Hemipteran pest species.

### SUMMARY OF THE INVENTION

Disclosed herein is a group of pesticidal proteins with insect inhibitory activity (toxin proteins), referred to herein as PirAB (Photorhabdus insect related) protein toxins, which are shown to exhibit inhibitory activity against one or more pests of crop plants. The proteins in the PirAB protein toxin class can be used alone, or as fusions of a PirA protein and a PirB protein, or in combination with other insecticidal proteins and toxic agents in formulations and in planta, thus providing alternatives to insecticidal proteins and insecticide chemistries currently in use in agricultural systems.

In one embodiment, disclosed in this application is a recombinant nucleic acid molecule comprising a heterologous promoter operably linked to a polynucleotide segment encoding a pesticidal protein or fragment thereof, wherein: (a) said pesticidal protein comprises the amino acid sequence of SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157; or (b) said pesticidal protein comprises an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143; or (c) said polynucleotide segment hybridizes to a polynucleotide having the nucleotide sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 57, 59, 61, 63, 65, 67, 69, 71, 73, 75, 77, 79, 81, 83, 85, 87, 89, 91, 93, 95, 97, 99, 101, 103, 104, 106, 108, 110, 112, 114, 116, 118, 120, 122, 124, 126, 128, 130, 132, 134, 136, 138, 140, 142, 144, 146, 148, 150, 152, 154, 156, or 158; or (d) said recombinant nucleic acid molecule is in operable linkage with a vector, and said vector is selected from the group consisting of a plasmid, phagemid, bacmid, cosmid, and a bacterial or yeast artificial chromosome. The recombinant nucleic acid molecule can comprise a sequence that functions to express the pesticidal protein in a plant; or is expressed in a plant cell to produce a pesticidally effective amount of pesticidal protein.

In another embodiment of this application are host cells comprising a recombinant nucleic acid molecule of the application, wherein the host cell is selected from the group consisting of a bacterial and a plant cell. Contemplated host cells include Agrobacterium, Rhizobium, Bacillus, Brevibacillus, Escherichia, Pseudomonas, Klebsiella, Pantoea, and Erwinia. In certain embodiments said Bacillus species is Bacillus cereus or Bacillus thuringiensis, said Brevibacillus is Brevibacillus laterosperus, or said Escherichia is Escherichia coli. Contemplated plant host cells include a dicotyledonous cell and a monocotyledonous cell. Further contemplated plant host cells include an alfalfa, banana, barley, bean, broccoli, cabbage, Brassica, carrot, cassava, castor, cauliflower, celery, chickpea, Chinese cabbage, citrus, coconut, coffee, corn, clover, cotton (Gossypium sp.), a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell.

In yet another embodiment, the pesticidal protein exhibits activity against Coleopteran insect, including Western Corn Rootworm, Southern Corn Rootworm, Northern Corn Rootworm, Mexican Corn Rootworm, Brazilian Corn Rootworm, Colorado Potato Beetle, Brazilian Corn Rootworm complex consisting of Diabrotica viridula and Diabrotica speciosa, Crucifer Flea Beetle, Striped Flea Beetle, and Western Black Flea Beetle.

In another embodiment, the pesticidal protein exhibits activity against a Lepidopteran insect, including Black Cutworm, Corn Earworm, Diamondback Moth, European Corn Borer, Fall Armyworm, Southern Armyworm, Soybean Looper, Southwestern Corn Borer, Tobacco Budworm, Velvetbean Caterpillar, Sugarcane Borer, Lesser Cornstalk Borer, Black Armyworm, Beet Armyworm, Old World Bollworm, Oriental leaf Worm, or Pink Bollworm.

In yet another embodiment, the pesticidal protein exhibits activity against an insect species of the order of Hemiptera, including Southern Green Stinkbug, Neotropical Brown Stinkbug, Southern Green Stink Bug, Neotropical Brown Stink Bug, Redbanded Stink Bug, Black-Spined Green-Belly Stink Bug species, Brown-Winged Stink Bug, Brown Stink Bug, Green Stink Bug, Brown Marmorated Stink Bug, Western Tarnished Plant Bug, or Tarnished Plant Bug.

Also contemplated in this application are plants comprising a recombinant nucleic acid molecule comprising a heterologous promoter operably linked to a polynucleotide segment encoding a pesticidal protein or fragment thereof, wherein: (a said pesticidal protein comprises the amino acid sequence of SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157; or (b) said pesticidal protein comprises an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143; or (c) said polynucleotide segment hybridizes under stringent hybridization conditions to the compliment of the nucleotide sequence of to SEQ ID NOs: 49, 51, 52, 53, 54, 55, 56, 146, 148, 150, 152, 154, 156, or 158; or (d) said plant exhibits a detectable amount of said pesticidal protein. In certain embodiments the pesticidal protein comprises SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157. In one embodiment, the plant is either a monocot or a dicot. In another embodiment, the plant is selected from the group consisting of an alfalfa, banana, barley, bean, broccoli, cabbage, Brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat.

In further embodiments, seeds comprising the recombinant nucleic acid molecules are disclosed.

In another embodiment, an insect inhibitory composition comprising the recombinant nucleic acid molecules disclosed in this application are contemplated. The insect inhibitory composition can further comprise a nucleotide sequence encoding at least one other pesticidal agent that is different from said pesticidal protein. The at least one other pesticidal agent is selected from the group consisting of an insect inhibitory protein, an insect inhibitory dsRNA molecule, and an ancillary protein. The at least one other pesticidal agent in the insect inhibitory composition exhibits activity against one or more pest species of the orders Lepidoptera, Coleoptera, or Hemiptera. The at least one other pesticidal agent in the insect inhibitory composition is in one embodiment selected from the group consisting of: a Cry1A, Cry1Ab, Cry1Ac, Cry1A.105, Cry1Ae, Cry1B, Cry1C, Cry1C variants, Cry1D, Cry1E, Cry1F, Cry1A/F chimeras, Cry1G, Cry1H, Cry1I, Cry1J, Cry1K, Cry1L, Cry2A, Cry2Ab, Cry2Ae, Cry3, Cry3A variants, Cry3B, Cry4B, Cry6, Cry7, Cry8, Cry9, Cry15, Cry34, Cry35, Cry43A, Cry43B, Cry51Aa1, ET29, ET33, ET34, ET35, ET66, ET70, TIC400, TIC407, TIC417, TIC431, TIC800, TIC807, TIC834, TIC853, TIC900, TIC901, TIC1201, TIC1415, TIC2160, TIC3131, TIC836, TIC860, TIC867, TIC869, TIC1100, VIP3A, VIP3B, VIP3Ab, AXMI-AXMI-, AXMI-88, AXMI-97, AXMI-102, AXMI-112, AXMI-117, AXMI-100, AXMI-115, AXMI-113, and AXMI-005, AXMI134, AXMI-150, AXMI-171, AXMI-184, AXMI-196, AXMI-204, AXMI-207, AXMI-209, AXMI-205, AXMI-218, AXMI-220, AXMI-221z, AXMI-222z, AXMI-223z, AXMI-224z and AXMI-225z, AXMI-238, AXMI-270, AXMI-279, AXMI-345, AXMI-335, AXMI-R1 and variants thereof, IP3 and variants thereof, DIG-3, DIG-5, DIG-10, DIG-657 DIG-11, Cry71Aa1, Cry72Aa1, PHI-4 variants, PIP-72 variants, PIP-45 variants, PIP-64 variants, PIP-74 variants, PIP-75 variants, PIP-77 variants, Axmi422, Dig-305, Axmi440, PIP-47 variants, Axmi281, BT-009, BT-0012, BT-0013, BT-0023, BT0067, BT-0044, BT-0051, BT-0068, BT-0128, DIG-17, DIG-90, DIG-79, Cry1JP578V, Cry1JPS1, and Cry1 JPS1P578V.

Commodity products comprising a detectable amount of the recombinant nucleic acid molecules disclosed in this application are contemplated. Such commodity products include commodity corn bagged by a grain handler, corn flakes, corn cakes, corn flour, corn meal, corn syrup, corn oil, corn silage, corn starch, corn cereal, and the like, and corresponding cotton commodity products such as whole or processed cotton seed, cotton oil, lint, seeds and plant parts processed for feed or food, fiber, paper, biomasses, and fuel products such as fuel derived from cotton oil or pellets derived from cotton gin waste, and corresponding soybean commodity products such as whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts, and corresponding rice, wheat, sorghum, pigeon pea, peanut, fruit, melon, and vegetable commodity products including where applicable, juices, concentrates, jams, jellies, marmalades, and other edible forms of such commodity products containing a detectable amount of such polynucleotides and or polypeptides of this application.

Also contemplated in this application is a method of producing seed comprising the recombinant nucleic acid molecules disclosed in this application. The method comprises planting at least one of the seed comprising the recombinant nucleic acid molecules disclosed in this application; growing plant from the seed; and harvesting seed from the plants, wherein the harvested seed comprises the recombinant nucleic acid molecules in this application.

In another illustrative embodiment, a plant resistant to insect infestation is provided, wherein the cells of said plant comprise: (a) a recombinant nucleic acid molecule encoding an insecticidally effective amount of a pesticidal protein as set forth in SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157; or (b) said pesticidal protein comprises an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143.

Also disclosed in this application are methods for controlling a Coleopteran or Lepidopteran species pest, and controlling a Coleopteran or Lepidopteran species pest infestation of a plant, particularly a crop plant. The method comprises, in one embodiment, (a) contacting the pest with an insecticidally effective amount of one or more pesticidal proteins as set forth in SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157; or (b) said pesticidal protein comprises an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143.

Further provided herein is a method of detecting the presence of a recombinant nucleic acid molecule comprising a polynucleotide segment encoding a pesticidal protein or fragment thereof, wherein: (a) said pesticidal protein comprises the amino acid sequence of SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157; or (b) said pesticidal protein comprises an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143; or (c) said polynucleotide segment hybridizes to a polynucleotide having the nucleotide sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 57, 59, 61, 63, 65, 67, 69, 71, 73, 75, 77, 79, 81, 83, 85, 87, 89, 91, 93, 95, 97, 99, 101, 103, 104, 106, 108, 110, 112, 114, 116, 118, 120, 122, 124, 126, 128, 130, 132, 134, 136, 138, 140, 142, 144, 146, 148, 150, 152, 154, 156, or 158. In one embodiment of the invention, the method comprises contacting a sample of nucleic acids with a nucleic acid probe that hybridizes under stringent hybridization conditions with genomic DNA from a plant comprising a polynucleotide segment encoding a pesticidal protein or fragment thereof provided herein, and does not hybridize under such hybridization conditions with genomic DNA from an otherwise isogenic plant that does not comprise the segment, wherein the probe is homologous or complementary to SEQ ID NOs: 49, 51, 52, 53, 54, 55, 56, 146, 148, 150, 152, 154, 156, or 158, or a sequence that encodes a pesticidal protein comprising an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143. The method may further comprise (a) subjecting the sample and probe to stringent hybridization conditions; and (b) detecting hybridization of the probe with DNA of the sample.

Also provided by the invention are methods of detecting the presence of a pesticidal protein or fragment thereof in a sample comprising protein, wherein said pesticidal protein comprises the amino acid sequence of SEQ ID NOs:2, 4, 6, 8, 10, 12, 14, 16, 18, 20, 22, 24, 26, 28, 30, 32, 34, 36, 38, 40, 42, 44, 46, 48, 50, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133, 135, 137, 139, 141, 143, 145, 147, 149, 151, 153, 155, or 157; or said pesticidal protein comprises an amino acid sequence having: (i) at least 65% identity to SEQ ID NOs:44, 46, 48, 123, 127, 129, 131, 133, and 145; or (ii) at least 70% identity to SEQ ID NOs:109, 121, and 125; or (iii) at least 80% identity to SEQ ID NOs: 12, 18, 24, 36, 42, 62, 68, 74, 80, 86, 98, 113, 117, 119, 147, 149, 153, 155, and 157; or (iv) at least 82% identity to SEQ ID NOs:30, 92, 111, 115, and 151; or (v) at least 86% identity to SEQ ID NOs:6 and 50; or (vi) at least 94% identity to SEQ ID NOs:137 and 141; or (vii) at least 97% identity to SEQ ID NOs:4, 26, and 32; or (viii) at least 98% identity to SEQ ID NOs:2, 28, 34, 102, and 102; or (ix) at least 99% identity to SEQ ID NO:135; or (x) 100% identity to SEQ ID NOs:8, 10, 14, 16, 20, 22, 38, 40, 58, 60, 64, 66, 70, 72, 76, 78, 82, 84, 88, 90, 94, 96, 100, 105, 107, 139, and 143. In one embodiment, the method comprises: (a) contacting a sample with an immunoreactive antibody; and (b) detecting binding of the antibody with the pesticidal protein or fragment thereof, wherein binding indicates the presence of the protein. In some embodiments, the step of detecting comprises an ELISA, or a Western blot.

### BRIEF DESCRIPTION OF THE SEQUENCES

SEQ ID NO:1 is a nucleic acid sequence obtained from Xenorhabdus nematophila strain ISB000002 encoding a TIC4771 PirA pesticidal protein sequence.

SEQ ID NO:2 is the amino acid sequence of the TIC4771 PirA protein.

SEQ ID NO:3 is a nucleic acid sequence obtained from Xenorhabdus nematophila strain ISB000002 encoding a TIC4772 PirB pesticidal protein sequence.

SEQ ID NO:4 is the amino acid sequence of the TIC4472 PirB protein.

SEQ ID NO:5 is a nucleic acid sequence encoding a PirAB fusion protein, TIC6880 comprised of the TIC4771 and TIC4772 coding sequences in operable linkage and in frame.

SEQ ID NO:6 is the amino acid sequence of the TIC6880 PirAB fusion protein.

SEQ ID NO:7 is a nucleic acid sequence obtained from Xenorhabdus ehlersii strain 85823 encoding a TIC7575 PirA pesticidal protein sequence.

SEQ ID NO:8 is the amino acid sequence of the TIC7575 PirA protein.

SEQ ID NO:9 is a nucleic acid sequence obtained from Xenorhabdus ehlersii strain 85823 encoding a TIC7576 PirB pesticidal protein sequence.

SEQ ID NO:10 is the amino acid sequence of the TIC7576 PirB protein.

SEQ ID NO:11 is a nucleic acid sequence encoding a PirAB fusion protein, TIC9316 comprised of the TIC7575 and TIC7576 coding sequences in operable linkage and in frame.

SEQ ID NO:12 is the amino acid sequence of the TIC9316 PirAB fusion protein.

SEQ ID NO:13 is a nucleic acid sequence obtained from Xenorhabdus cabanillasii strain 85908 encoding a TIC7660 PirA pesticidal protein sequence.

SEQ ID NO:14 is the amino acid sequence of the TIC7660 PirA protein.

SEQ ID NO:15 is a nucleic acid sequence obtained from Xenorhabdus cabanillasii strain 85908 encoding a TIC7661 PirB pesticidal protein sequence.

SEQ ID NO:16 is the amino acid sequence of the TIC7661 PirB protein.

SEQ ID NO:17 is a nucleic acid sequence encoding a PirAB fusion protein, TIC9317 comprised of the TIC7660 and TIC7661 coding sequences in operable linkage and in frame.

SEQ ID NO:18 is the amino acid sequence of the TIC9317 PirAB fusion protein.

SEQ ID NO:19 is a nucleic acid sequence obtained from Xenorhabdus ehlersii strain 85887 encoding a TIC7662 PirA pesticidal protein sequence.

SEQ ID NO:20 is the amino acid sequence of the TIC7662 PirA protein.

SEQ ID NO:21 is a nucleic acid sequence obtained from Xenorhabdus ehlersii strain 85887 encoding a TIC7663 PirB pesticidal protein sequence.

SEQ ID NO:22 is the amino acid sequence of the TIC7663 PirB protein.

SEQ ID NO:23 is a nucleic acid sequence encoding a PirAB fusion protein, TIC9318 comprised of the TIC7662 and TIC7663 coding sequences in operable linkage and in frame.

SEQ ID NO:24 is the amino acid sequence of the TIC9318 PirAB fusion protein.

SEQ ID NO:25 is a nucleic acid sequence obtained from Xenorhabdus poinarii strain 86198 encoding a TIC7664 PirA pesticidal protein sequence.

SEQ ID NO:26 is the amino acid sequence of the TIC7664 PirA protein.

SEQ ID NO:27 is a nucleic acid sequence obtained from Xenorhabdus poinarii strain 86198 encoding a TIC7665 PirB pesticidal protein sequence.

SEQ ID NO:28 is the amino acid sequence of the TIC7665 PirB protein.

SEQ ID NO:29 is a nucleic acid sequence encoding a PirAB fusion protein, TIC9319 comprised of the TIC7664 and TIC7665 coding sequences in operable linkage and in frame.

SEQ ID NO:30 is the amino acid sequence of the TIC9319 PirAB fusion protein.

SEQ ID NO:31 is a nucleic acid sequence obtained from Photorhabdus luminescens strain 86197 encoding a TIC7666 PirA pesticidal protein sequence.

SEQ ID NO:32 is the amino acid sequence of the TIC7666 PirA protein.

SEQ ID NO:33 is a nucleic acid sequence obtained from Photorhabdus luminescens strain 86197 encoding a TIC7667 pesticidal PirB protein sequence.

SEQ ID NO:34 is the amino acid sequence of the TIC7667 PirB protein.

SEQ ID NO:35 is a nucleic acid sequence encoding a PirAB fusion protein, TIC9322 comprised of the TIC7666 and TIC7667 coding sequences in operable linkage and in frame.

SEQ ID NO:36 is the amino acid sequence of the TIC9322 PirAB fusion protein.

SEQ ID NO:37 is a nucleic acid sequence obtained from Photorhabdus luminescens strain 86194 encoding a TIC7668 PirA pesticidal protein sequence.

SEQ ID NO:38 is the amino acid sequence of the TIC7668 PirA protein.

SEQ ID NO:39 is a nucleic acid sequence obtained from Photorhabdus luminescens strain 86194 encoding a TIC7669 PirB pesticidal protein sequence.

SEQ ID NO:40 is the amino acid sequence of the TIC7669 PirB protein.

SEQ ID NO:41 is a nucleic acid sequence encoding a PirAB fusion protein, TIC9320 comprised of the TIC7668 and TIC7669 coding sequences in operable linkage and in frame.

SEQ ID NO:42 is the amino acid sequence of the TIC9320 PirAB fusion protein.

SEQ ID NO:43 is a nucleic acid sequence obtained from an unknown bacterial strain comprised within a microbiome encoding a TIC7939 pesticidal PirA protein sequence.

SEQ ID NO: 44 is the amino acid sequence of the TIC7939 PirA protein.

SEQ ID NO:45 is a nucleic acid sequence obtained from an unknown bacterial strain comprised within a microbiome encoding a TIC7940 PirB pesticidal protein sequence.

SEQ ID NO:46 is the amino acid sequence of the TIC7940 PirB protein.

SEQ ID NO:47 is a nucleic acid sequence encoding a PirAB fusion protein, TIC9321 comprised of the TIC7939 and TIC7940 coding sequences in operable linkage and in frame.

SEQ ID NO:48 is the amino acid sequence of the TIC9321 PirAB fusion protein.

SEQ ID NO:49 is a synthetic coding sequence used for expression in plant cells encoding a TIC6880PL PirAB fusion protein wherein an additional alanine codon is inserted immediately following the initiating methionine codon of the TIC4771 protein encoding fragment.

SEQ ID NO:50 is the amino acid sequence of the TIC6880PL PirAB fusion protein.

SEQ ID NO:51 is a synthetic coding sequence used for expression in plant cells encoding a TIC9316 PirAB fusion protein.

SEQ ID NO:52 is a synthetic coding sequence used for expression in plant cells encoding a TIC9317 PirAB fusion protein.

SEQ ID NO:53 is a synthetic coding sequence used for expression in plant cells encoding a TIC9318 PirAB fusion protein.

SEQ ID NO:54 is a synthetic coding sequence used for expression in plant cells encoding a TIC9319 PirAB fusion protein.

SEQ ID NO:55 is a synthetic coding sequence used for expression in plant cells encoding a TIC9320 PirAB fusion protein.

SEQ ID NO:56 is a synthetic coding sequence used for expression in plant cells encoding a TIC9322 PirAB fusion protein.

SEQ ID NO:57 is a nucleic acid sequence obtained from Shewanella violacea strain DSS12 encoding a TIC10357 pesticidal PirA protein sequence.

SEQ ID NO:58 is the amino acid sequence of the TIC10357 PirA protein.

SEQ ID NO:59 is a nucleic acid sequence obtained from Shewanella violacea strain DSS12 encoding a TIC10366 pesticidal PirB protein sequence.

SEQ ID NO:60 is the amino acid sequence of the TIC10366 PirB protein.

SEQ ID NO:61 is a nucleic acid sequence encoding a PirAB fusion protein, TIC10375 comprised of the TIC10357 and TIC10366 coding sequences in operable linkage and in frame.

SEQ ID NO:62 is the amino acid sequence of the TIC10375 PirAB fusion protein.

SEQ ID NO:63 is a nucleic acid sequence obtained from Photorhabdus luminescens strain laumondii TTO1 encoding a TIC10358 pesticidal PirA protein sequence.

SEQ ID NO:64 is the amino acid sequence of the TIC10358 PirA protein.

SEQ ID NO:65 is a nucleic acid sequence obtained from Photorhabdus luminescens strain laumondii TTO1 encoding a TIC10367 pesticidal PirB protein sequence.

SEQ ID NO:66 is the amino acid sequence of the TIC10367 PirB protein.

SEQ ID NO:67 is a nucleic acid sequence encoding a PirAB fusion protein, TIC10376 comprised of the TIC10358 and TIC10367 coding sequences in operable linkage and in frame.

SEQ ID NO:68 is the amino acid sequence of the TIC10376 PirAB fusion protein.

SEQ ID NO:69 is a nucleic acid sequence obtained from Photorhabdus asymbiotica encoding a TIC10360 pesticidal PirA protein sequence.

SEQ ID NO:70 is the amino acid sequence of the TIC10360 PirA protein.

SEQ ID NO:71 is a nucleic acid sequence obtained from Photorhabdus asymbiotica encoding a TIC10369 pesticidal PirB protein sequence.

SEQ ID NO:72 is the amino acid sequence of the TIC10369 PirB protein.

SEQ ID NO:73 is a nucleic acid sequence encoding a PirAB fusion protein, TIC10377 comprised of the TIC10360 and TIC10369 coding sequences in operable linkage and in frame.

SEQ ID NO:74 is the amino acid sequence of the TIC10377 PirAB fusion protein.

SEQ ID NO:75 is a nucleic acid sequence obtained from Xenorhabdus sp. strain NBAII XenSa04 encoding a TIC10361 pesticidal PirA protein sequence.

SEQ ID NO:76 is the amino acid sequence of the TIC10361 PirA protein.

SEQ ID NO:77 is a nucleic acid sequence obtained from Xenorhabdus sp. strain NBAII XenSa04 encoding a TIC10370 pesticidal PirB protein sequence.

SEQ ID NO:78 is the amino acid sequence of the TIC10370 PirB protein.

SEQ ID NO:79 is a nucleic acid sequence encoding a PirAB fusion protein, TIC10378 comprised of the TIC10361 and TIC10370 coding sequences in operable linkage and in frame.

SEQ ID NO:80 is the amino acid sequence of the TIC10378 PirAB fusion protein.

SEQ ID NO:81 is a nucleic acid sequence obtained from Yersinia aldovae strain 670-83 encoding a TIC10362 pesticidal PirA protein sequence.

SEQ ID NO:82 is the amino acid sequence of the TIC10362 PirA protein.

SEQ ID NO:83 is a nucleic acid sequence obtained from Yersinia aldovae strain 670-83 encoding a TIC10371 pesticidal PirB protein sequence.

SEQ ID NO:84 is the amino acid sequence of the TIC10371 PirB protein.

SEQ ID NO:85 is a nucleic acid sequence encoding a PirAB fusion protein, TIC10379 comprised of the TIC10362 and TIC10371 coding sequences in operable linkage and in frame.

SEQ ID NO:86 is the amino acid sequence of the TIC10379 PirAB fusion protein.

SEQ ID NO:87 is a nucleic acid sequence obtained from Xenorhabdus doucetiae strain FRM16 encoding a TIC10363 pesticidal PirA protein sequence.

SEQ ID NO:88 is the amino acid sequence of the TIC10363 PirA protein.

SEQ ID NO:89 is a nucleic acid sequence obtained from Xenorhabdus doucetiae strain FRM16 encoding a TIC10372 pesticidal PirB protein sequence.

SEQ ID NO:90 is the amino acid sequence of the TIC10372 PirB protein.

SEQ ID NO:91 is a nucleic acid sequence encoding a PirAB fusion protein, TIC10380 comprised of the TIC10363 and TIC10372 coding sequences in operable linkage and in frame.

SEQ ID NO:92 is the amino acid sequence of the TIC10380 PirAB fusion protein.

SEQ ID NO:93 is a nucleic acid sequence obtained from Xenorhabdus griffiniae strain BMMCB encoding a TIC10364 pesticidal PirA protein sequence.

SEQ ID NO:94 is the amino acid sequence of the TIC10364 PirA protein.

SEQ ID NO:95 is a nucleic acid sequence obtained from Xenorhabdus griffiniae strain BMMCB encoding a TIC10373 pesticidal PirB protein sequence.

SEQ ID NO:96 is the amino acid sequence of the TIC10373 PirB protein.

SEQ ID NO:97 is a nucleic acid sequence encoding a PirAB fusion protein, TIC10381 comprised of the TIC10364 and TIC10364 coding sequences in operable linkage and in frame.

SEQ ID NO:98 is the amino acid sequence of the TIC10381 PirAB fusion protein.

SEQ ID NO:99 is a nucleic acid sequence obtained from Xenorhabdus nematophila encoding a TIC10359 pesticidal PirA protein sequence.

SEQ ID NO:100 is the amino acid sequence of the TIC10359 PirA protein.

SEQ ID NO:101 is a nucleic acid sequence obtained from Xenorhabdus nematophila encoding a TIC10368 pesticidal PirB protein sequence.

SEQ ID NO:102 is the amino acid sequence of the TIC10368 PirB protein.

SEQ ID NO:103 is a nucleic acid sequence encoding an operon comprised of the coding sequences TIC10359 and TIC10368.

SEQ ID NO:104 is a nucleic acid sequence obtained from Photorhabdus luminescens strain Hm encoding a PirA_ABE68878 pesticidal PirA protein sequence.

SEQ ID NO:105 is the amino acid sequence of the PirA_ABE68878 PirA protein.

SEQ ID NO:106 is a nucleic acid sequence obtained from Photorhabdus luminescens strain Hm encoding a PirB_ABE68879 pesticidal PirB protein sequence.

SEQ ID NO:107 is the amino acid sequence of the PirB_ABE68879 PirB protein.

SEQ ID NO:108 is a nucleic acid sequence encoding a PirAB fusion protein, TIC10434 comprised of the PirA_ABE68878 and PirB_ABE68879 coding sequences in operable linkage and in frame.

SEQ ID NO:109 is the amino acid sequence of the TIC10434 PirAB fusion protein.

SEQ ID NO:110 is a nucleic acid sequence encoding a PirAB fusion protein, TIC11210 comprised of the TIC7575 and TIC7665 coding sequences in operable linkage and in frame.

SEQ ID NO:111 is the amino acid sequence of the TIC11210 PirAB fusion protein.

SEQ ID NO:112 is a nucleic acid sequence encoding a PirAB fusion protein, TIC11211 comprised of the TIC7575 and TIC7667 coding sequences in operable linkage and in frame.

SEQ ID NO:113 is the amino acid sequence of the TIC11211 PirAB fusion protein.

SEQ ID NO:114 is a nucleic acid sequence encoding a PirAB fusion protein, TIC11212 comprised of the TIC7662 and TIC7665 coding sequences in operable linkage and in frame.

SEQ ID NO:115 is the amino acid sequence of the TIC11212 PirAB fusion protein.

SEQ ID NO:116 is a nucleic acid sequence encoding a PirAB fusion protein, TIC11301 comprised of the TIC7575 and TIC7661 coding sequences in operable linkage and in frame.

SEQ ID NO:117 is the amino acid sequence of the TIC11301 PirAB fusion protein.

SEQ ID NO:118 is a nucleic acid sequence encoding a f PirAB fusion protein, TIC11302 comprised of the TIC7660 and TIC7576 coding sequences in operable linkage and in frame.

SEQ ID NO:119 is the amino acid sequence of the TIC11302 f PirAB fusion protein.

SEQ ID NO:120 is a nucleic acid sequence encoding a PirAB fusion protein, TIC11440 comprised of the TIC4771, TIC4771, and TIC4472 coding sequences in operable linkage and in frame.

SEQ ID NO:121 is the amino acid sequence of the TIC11440 PirAB fusion protein.

SEQ ID NO:122 is a nucleic acid sequence encoding a PirAB fusion protein, TIC11441 comprised of the TIC7575, TIC7575, and TIC7576 coding sequences in operable linkage and in frame.

SEQ ID NO:123 is the amino acid sequence of the TIC11441 f PirAB fusion protein.

SEQ ID NO:124 is a nucleic acid sequence encoding a PirAB fusion protein, TIC11442 comprised of the TIC7575, TIC4771, and TIC4472 coding sequences in operable linkage and in frame.

SEQ ID NO:125 is the amino acid sequence of the TIC11442 PirAB fusion protein.

SEQ ID NO:126 is a nucleic acid sequence encoding a PirAB fusion protein, TIC11443 comprised of the TIC7660, TIC7575, and TIC7576 coding sequences in operable linkage and in frame.

SEQ ID NO:127 is the amino acid sequence of the TIC11443 PirAB fusion protein.

SEQ ID NO:128 is a nucleic acid sequence encoding a PirAB fusion protein, TIC11444 comprised of the TIC7660 and TIC7576 coding sequences in operable linkage and in frame.

SEQ ID NO:129 is the amino acid sequence of the TIC11444 PirAB fusion protein.

SEQ ID NO:130 is a nucleic acid sequence encoding a PirAB fusion protein, TIC11445 comprised of the TIC7660, TIC7662, and TIC7663 coding sequences in operable linkage and in frame.

SEQ ID NO:131 is the amino acid sequence of the TIC11445 PirAB fusion protein.

SEQ ID NO:132 is a nucleic acid sequence encoding a fusion protein, TIC11446 comprised of the TIC7662, TIC7660, and TIC7661 coding sequences in operable linkage and in frame.

SEQ ID NO:133 is the amino acid sequence of the TIC11446 PirAB fusion protein.

SEQ ID NO:134 is a nucleic acid sequence obtained from Xenorhabdus nematophila strain MDI-0035777 encoding a TIC11505 pesticidal PirB protein sequence.

SEQ ID NO:135 is the amino acid sequence of the TIC11505 PirB protein.

SEQ ID NO:136 is a nucleic acid sequence encoding a PirAB fusion protein, TIC11506 comprised of the TIC10364 and TIC11505 coding sequences in operable linkage and in frame.

SEQ ID NO:137 is the amino acid sequence of the TIC11506 PirAB fusion protein.

SEQ ID NO:138 is a nucleic acid sequence obtained from Xenorhabdus bovienii strain MI-0035808 encoding a TIC11510 pesticidal PirB protein sequence.

SEQ ID NO:139 is the amino acid sequence of the TIC11510 PirB protein.

SEQ ID NO:140 is a nucleic acid sequence encoding a PirAB fusion protein, TIC11512 comprised of the TIC10364 and TIC11510 coding sequences in operable linkage and in frame.

SEQ ID NO:141 is the amino acid sequence of the TIC11512 PirAB fusion protein.

SEQ ID NO:142 is a nucleic acid sequence obtained from Xenorhabdus nematophila strain AN6/1 encoding a TIC11511 pesticidal PirB protein sequence.

SEQ ID NO:143 is the amino acid sequence of the TIC11511 PirB protein.

SEQ ID NO:144 is a nucleic acid sequence encoding a PirAB fusion protein, TIC11513 comprised of the TIC10364 and TIC11511 coding sequences in operable linkage and in frame.

SEQ ID NO:145 is the amino acid sequence of the TIC11513 PirAB fusion protein.

SEQ ID NO:146 is a synthetic coding sequence used for expression in plant cells encoding a TIC10376PL PirAB fusion protein wherein an additional alanine codon is inserted immediately following the initiating methionine codon of the TIC10358 protein encoding fragment.

SEQ ID NO:147 is the amino acid sequence of the TIC10376PL PirAB fusion protein.

SEQ ID NO:148 is a synthetic coding sequence used for expression in plant cells encoding a TIC10378PL PirAB fusion protein wherein an additional alanine codon is inserted immediately following the initiating methionine codon of the TIC10361 protein encoding fragment.

SEQ ID NO:149 is the amino acid sequence of the TIC10378PL PirAB fusion protein.

SEQ ID NO:150 is a synthetic coding sequence used for expression in plant cells encoding a TIC10380PL PirAB fusion protein wherein an additional alanine codon is inserted immediately following the initiating methionine codon of the TIC10363 protein encoding fragment.

SEQ ID NO:151 is the amino acid sequence of the TIC10380PL PirAB fusion protein.

SEQ ID NO:152 is a synthetic coding sequence used for expression in plant cells encoding a TIC10381PL PirAB fusion protein wherein an additional alanine codon is inserted immediately following the initiating methionine codon of the TIC10364 protein encoding fragment.

SEQ ID NO:153 is the amino acid sequence of the TIC10381PL PirAB fusion protein.

SEQ ID NO:154 is a synthetic coding sequence used for expression in plant cells encoding a TIC11103 PirAB fusion protein comprised of the TIC7661 and TIC7660 coding sequences operably linked.

SEQ ID NO:155 is the amino acid sequence of the TIC11103 PirAB fusion protein.

SEQ ID NO:156 is a synthetic coding sequence used for expression in plant cells encoding a TIC11104 PirAB fusion protein comprised of the TIC7663 and TIC7662 coding sequences operably linked.

SEQ ID NO:157 is the amino acid sequence of the TIC11104 PirAB fusion protein.

SEQ ID NO:158 is a synthetic coding sequence used for expression in plant cells encoding a TIC11302 PirAB fusion protein.

SEQ ID NO:159 is a synthetic coding sequence encoding a Histidine tag that is operably linked to coding sequences expressed in Escherichia coli and used for protein purification.

SEQ ID NO:160 is the amino acid sequence of the Histidine tag.

### DETAILED DESCRIPTION OF THE INVENTION

A problem in the art of agricultural pest control can be characterized as a need for new toxin proteins that are efficacious against target pests, exhibit broad spectrum toxicity against target pest species, are capable of being expressed in plants without causing undesirable agronomic issues, and provide an alternative mode of action compared to current toxins that are used commercially in plants.

Disclosed herein are novel PirAB pesticidal protein classes, exemplified by the PirA proteins TIC4771, TIC7575, TIC7660, TIC7662, TIC7664, TIC7666, TIC7668, TIC7939, TIC10357, TIC10358, TIC10360, TIC10361, TIC10362, TIC10363, TIC10364, TIC10359, and PirA_ABE68878 (collectively, “The PirA Proteins”); the PirB proteins TIC4772, TIC7576, TIC7661, TIC7663, TIC7665, TIC7667, TIC7669, TIC7940, TIC10366, TIC10367, TIC10369, TIC10370, TIC10371, TIC10372, TIC10373, TIC10368, PirB_ABE68879, TIC11505, TIC11510, and TIC11511 (collectively, “The PirB Proteins”); and the PirAB fusion proteins, TIC6880, TIC9316, TIC9317, TIC9318, TIC9319, TIC9322, TIC9320, TIC9321, TIC6880PL, TIC10375, TIC10376, TIC10377, TIC10378, TIC10379, TIC10380, TIC10381, TIC10434, TIC11210, TIC11211, TIC11212, TIC11301, TIC11302, TIC11440, TIC11441, TIC11442, TIC11443, TIC11444, TIC11445, TIC11446, TIC11506, TIC11512, TIC11513, TIC10376PL, TIC10378PL, TIC10380PL, TIC10381PL, TIC11103, and TIC11104 (collectively, “The PirAB Fusion Protein”) which provide resistance against Coleopteran, Hemipteran, and Lepidopteran insect pests.

Also disclosed are synthetic coding sequences designed for expression in a plant cell that encode the PirAB fusion proteins, TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302. Further disclosed are recombinant nucleic acid molecules comprising a promoter in operable linkage to a coding sequence encoding one or more of The PirA Proteins, The PirB Proteins, or The PirAB Fusion Proteins.

Reference in this application to “PirA proteins”, “PirA protein toxin”, “PirA toxin protein”, “PirA pesticidal protein”, “PirA-related toxins”, or “PirA-related toxin protein”, and the like, refer to any novel pesticidal protein or insect inhibitory protein, that comprises, that consists of, that is substantially homologous to, that is similar to, or that is derived from any pesticidal protein or insect inhibitory protein sequence of TIC4771 (SEQ ID NO:2), TIC7575 (SEQ ID NO:8), TIC7660 (SEQ ID NO:14), TIC7662 (SEQ ID NO:20), TIC7664 (SEQ ID NO:26), TIC7666 (SEQ ID NO:32), TIC7668 (SEQ ID NO:38), TIC7939 (SEQ ID NO:44), TIC10357 (SEQ ID NO:58), TIC10358 (SEQ ID NO:64), TIC10360 (SEQ ID NO:70), TIC10361 (SEQ ID NO:76), TIC10362 (SEQ ID NO:82), TIC10363 (SEQ ID NO:88), TIC10364 (SEQ ID NO:94), TIC10359 (SEQ ID NO:100), and PirA_ABE68878 (SEQ ID NO:105) and pesticidal or insect inhibitory segments thereof, or combinations thereof, that confer activity against Coleopteran pests, Hemipteran pests, and Lepidopteran pests, including any protein exhibiting pesticidal or insect inhibitory activity if alignment of such protein with any of the PirA proteins results in amino acid sequence identity of any fraction percentage from about 20 to about 100 percent.

Reference in this application to “PirB proteins”, “PirB protein toxin”, “PirB toxin protein”, “PirB pesticidal protein”, “PirB-related toxins”, or “PirB-related toxin protein”, and the like, refer to any novel pesticidal protein or insect inhibitory protein, that comprises, that consists of, that is substantially homologous to, that is similar to, or that is derived from any pesticidal protein or insect inhibitory protein sequence of TIC4772 (SEQ ID NO:4), TIC7576 (SEQ ID NO:10), TIC7661 (SEQ ID NO:16), TIC7663 (SEQ ID NO:22), TIC7665 (SEQ ID NO:28), TIC7667 (SEQ ID NO:34), TIC7669 (SEQ ID NO:40), TIC7940 (SEQ ID NO:46), TIC10366 (SEQ ID NO:60), TIC10367 (SEQ ID NO:66), TIC10369 (SEQ ID NO:72), TIC10370 (SEQ ID NO:78), TIC10371 (SEQ ID NO:84), TIC10372 (SEQ ID NO:90), TIC10373 (SEQ ID NO:96), TIC10368 (SEQ ID NO:102), PirB_ABE68879 (SEQ ID NO:107), TIC11505 (SEQ ID NO:135), TIC11510 (SEQ ID NO:139), and TIC11511 (SEQ ID NO:143) and pesticidal or insect inhibitory segments thereof, or combinations thereof, that confer activity against Coleopteran pests, Hemipteran pests, and Lepidopteran pests, including any protein exhibiting pesticidal or insect inhibitory activity if alignment of such protein with and of the PirB proteins results in amino acid sequence identity of any fraction percentage from about 24 to about 100 percent.

The term “PirAB fusion protein” is used in this application to describe a protein that comprises both a PirA protein contiguous with a PirB protein. The DNA sequence encoding the PirAB fusion protein can comprise a coding sequence encoding a PirA protein operably linked and in frame with a coding sequence encoding a PirB protein such that when it is expressed in a cell produces a fusion protein comprising both a PirA protein and PirB protein. The PirA protein can be comprised of a PirA protein and a PirB protein derived from the same bacterial operon, or alternatively, can be comprised of a PirA protein and a PirB protein derived from different bacterial operons. Exemplary PirAB fusion proteins wherein the PirA protein is contiguous with a PirB protein are provided in Table 1.

TABLE 1
Exemplary PirAB fusion proteins comprised of a PirA protein contiguous with
a PirB protein and the corresponding PirA and PirB proteins comprised within.

| PirA Protein | | PirB Protein | | PirAB Fusion Protein | |
|---|---|---|---|---|---|
| | Protein | | Protein | | Protein |
| | SEQ ID | | SEQ ID | | SEQ ID |
| Toxin | NO: | Toxin | NO: | Toxin | NO: |
| TIC4771 | 2 | TIC4772 | 4 | TIC6880 | 6 |
| TIC7575 | 8 | TIC7576 | 10 | TIC9316 | 12 |
| TIC7660 | 14 | TIC7661 | 16 | TIC9317 | 18 |
| TIC7662 | 20 | TIC7663 | 22 | TIC9318 | 24 |
| TIC7664 | 26 | TIC7665 | 28 | TIC9319 | 30 |
| TIC7666 | 32 | TIC7667 | 34 | TIC9322 | 36 |
| TIC7668 | 38 | TIC7669 | 40 | TIC9320 | 42 |
| TIC7939 | 44 | TIC7940 | 46 | TIC9321 | 48 |
| TIC4771* | 2 | TIC4772 | 4 | TIC6880PL* | 6 |
| TIC10357 | 58 | TIC10366 | 60 | TIC10375 | 62 |
| TIC10358 | 64 | TIC10367 | 66 | TIC10376 | 68 |
| TIC10360 | 70 | TIC10369 | 72 | TIC10377 | 74 |
| TIC10361 | 76 | TIC10370 | 78 | TIC10378 | 80 |
| TIC10362 | 82 | TIC10371 | 84 | TIC10379 | 86 |
| TIC10363 | 88 | TIC10372 | 90 | TIC10380 | 92 |
| TIC10364 | 94 | TIC10373 | 96 | TIC10381 | 98 |
| PirA_ABE68878 | 105 | PirB_ABE68879 | 107 | TIC10434 | 109 |
| TIC10358* | 64 | TIC10367 | 66 | TIC10376PL* | 147 |
| TIC10361* | 76 | TIC10370 | 78 | TIC10378PL* | 149 |
| TIC10363* | 88 | TIC10372 | 90 | TIC10380PL* | 151 |
| TIC10364* | 94 | TIC10373 | 96 | TIC10381PL* | 153 |
| TIC7575 | 8 | TIC7665 | 28 | TIC11210 | 111 |
| TIC7575 | 8 | TIC7667 | 34 | TIC11211 | 113 |
| TIC7662 | 20 | TIC7665 | 28 | TIC11212 | 115 |
| TIC7575 | 8 | TIC7661 | 16 | TIC11301 | 117 |
| TIC7660 | 14 | TIC7576 | 10 | TIC11302 | 119 |
| TIC10364 | 94 | TIC11505 | 135 | TIC11506 | 137 |
| TIC10364 | 94 | TIC11510 | 139 | TIC11512 | 141 |
| TIC10364 | 94 | TIC11511 | 143 | TIC11513 | 145 |

*comprises an additional alanine residue immediately following the initiating methionine residue.

The term “PirAB fusion protein” is also used in this application to describe a protein that comprises a PirB protein contiguous with a PirA protein. The DNA sequence encoding the PirAB fusion protein of this type can comprise a coding sequence encoding a PirB protein operably linked and in frame with a coding sequence encoding a PirA protein such that when it is expressed in a cell it produces a fusion protein comprising both a PirB protein and PirA protein. The PirB protein can be comprised of a PirB protein and a PirA protein derived from the same bacterial operon, or alternatively, can be comprised of a PirB protein and a PirA protein derived from different operons. Exemplary proteins wherein a PirB protein is contiguous with a PirA protein are provided in Table 2.

TABLE 2
Exemplary PirAB fusion proteins comprised of a
PirB protein contiguous with a PirA
protein and the corresponding
PirB and PirA proteins comprised within.

| | | | | PirAB Fusion | |
|---|---|---|---|---|---|
| PirB Protein | | PirA Protein | | Protein | |
| | Protein | | Protein | | Protein |
| | SEQ ID | | SEQ ID | | SEQ ID |
| Toxin | NO: | Toxin | NO: | Toxin | NO: |
| TIC7661 | 16 | TIC7660 | 14 | TIC11103 | 155 |
| TIC7663 | 22 | TIC7662 | 20 | TIC11104 | 157 |

The term “PirAB fusion protein” is also used in this application to describe a protein that comprises two PirA proteins contiguous with a PirB protein. The DNA sequence encoding the PirAB fusion protein of this type can comprise a coding sequence encoding a PirA protein, operably linked to a coding sequence encoding the same PirA protein or a different PirA protein, operably linked to a coding sequence encoding a PirB protein such that when it is expressed in a cell it produces a fusion protein comprising a PirA protein, another PirA protein, and a PirB protein. Exemplary PirAB fusion proteins comprising two PirA proteins contiguous with a PirB protein are provided in Table 3.

TABLE 3
Exemplary PirAB fusion proteins comprised of a PirA protein contiguous with another PirA protein,
and contiguous with a PirB protein, and the corresponding PirA and PirB proteins comprised within.

| PirA Protein | | PirA Protein | | PirB Protein | | PirAB Fusion Protein | |
|---|---|---|---|---|---|---|---|
| | Protein | | Protein | | Protein | | Protein |
| Toxin | SEQ ID NO: | Toxin | SEQ ID NO: | Toxin | SEQ ID NO: | Toxin | SEQ ID NO: |
| TIC4771 | 2 | TIC4771 | 2 | TIC4772 | 4 | TIC11440 | 121 |
| TIC7575 | 8 | TIC7575 | 8 | TIC7576 | 10 | TIC11441 | 123 |
| TIC7575 | 8 | TIC4771 | 2 | TIC4772 | 4 | TIC11442 | 125 |
| TIC7660 | 14 | TIC7575 | 8 | TIC7576 | 10 | TIC11443 | 127 |
| TIC7575 | 8 | TIC7660 | 14 | TIC7661 | 16 | TIC11444 | 129 |
| TIC7660 | 14 | TIC7662 | 20 | TIC7663 | 22 | TIC11445 | 131 |
| TIC7662 | 20 | TIC7660 | 14 | TIC7661 | 16 | TIC11446 | 133 |

The term “PirAB fusion protein” is also used in this application to describe a protein that comprises multiple PirA and/or multiple PirB proteins contiguous with one another. The multiple PirA and/or PirB proteins can be duplicated PirA or PirB proteins, or can be different PirA or PirB proteins. The combination of multiple PirA and/or PirB proteins as a fusion protein can increase activity against a specific target pest species or may increase the range of pest species in which activity is present.

The term “segment” or “fragment” is used in this application to describe consecutive amino acid or nucleic acid sequences that are shorter than the complete amino acid or nucleic acid sequence describing one of The PirA Proteins, The PirB Proteins, or The PirAB Proteins. A segment or fragment exhibiting insect inhibitory activity is also disclosed in this application if alignment of such segment or fragment, with the corresponding section of the PirA proteins set forth in SEQ ID NOs:2, 8, 14, 20, 26, 32, 38, 44, 58, 64, 70, 76, 82, 88, 94, 100, or 105; the PirB proteins set forth in SEQ ID NOs:4, 10, 16, 22, 28, 34, 40, 46, 60, 66, 72, 78, 84, 90, 96, 102, 107, 135, 139, or 143; or the PirAB fusion proteins set forth in SEQ ID NOs:6, 12, 18, 24, 30, 36, 42, 48, 50, 62, 68, 74, 80, 86, 92, 98, 109, 111, 113, 115, 117, 119, 121, 123, 125, 127, 129, 131, 133, 137, 141, 145, 147, 149, 151, 153, 155, or 157, or related family member insecticidal protein results in an alignment from about 65 to about 100 percent identity between the segment or fragment and the corresponding section of the aligned protein. A segment or fragments of one of The PirA Proteins, The PirB Proteins, or The PirAB Proteins described herein may comprise at least about 50 contiguous amino acids, at least about 75 contiguous amino acids, at least about 100 contiguous amino acids, at least about 125 contiguous amino acids, at least about 150 contiguous amino acids, at least about 200 contiguous amino acids, at least about 250 contiguous amino acids, at least about 300 contiguous amino acids, at least about 350 contiguous amino acids, at least about 400 contiguous amino acids, at least about 450 contiguous amino acids, at least about 500 contiguous amino acids, at least about 550 contiguous amino acids, or at least about 600 contiguous amino acids of one of The PirA Proteins, The PirB Proteins, or The PirAB Proteins. A segment or fragment of one of The PirA Proteins, The PirB Proteins, or The PirAB Proteins described herein may exhibit the activity of the base sequence.

Reference in this application to the terms “active” or “activity”, “pesticidal activity” or “pesticidal” or “insecticidal activity”, “insect inhibitory” or “insecticidal” refer to efficacy of a toxic agent, such as a protein toxin, in inhibiting (inhibiting growth, feeding, fecundity, or viability), suppressing (suppressing growth, feeding, fecundity, or viability), controlling (controlling the pest infestation, controlling the pest feeding activities on a particular crop containing an effective amount of one or more of The PirA Proteins, The PirB Proteins or The PirAB Fusion Proteins. These terms are intended to include the result of providing a pesticidally effective amount of a toxic protein to a pest where the exposure of the pest to the toxic protein results in morbidity, mortality, reduced fecundity, or stunting. These terms also include repulsion of the pest from the plant, a tissue of the plant, a plant part, seed, plant cells, or from the particular geographic location where the plant may be growing, as a result of providing a pesticidally effective amount of the toxic protein in or on the plant. In general, pesticidal activity refers to the ability of a toxic protein to be effective in inhibiting the growth, development, viability, feeding behavior, mating behavior, fecundity, or any measurable decrease in the adverse effects caused by an insect feeding on this protein, protein fragment, protein segment or polynucleotide of a particular target pest, including but not limited to insects of the order Lepidoptera, Coleoptera or Hemiptera. The toxic protein can be produced by the plant or can be applied to the plant or to the environment within the location where the plant is located. The terms “bioactivity”, “effective”, “efficacious” or variations thereof are also terms interchangeably utilized in this application to describe the effects of proteins of the present invention on target insect pests.

A pesticidally effective amount of a toxic agent, when provided in the diet of a target pest, exhibits pesticidal activity when the toxic agent contacts the pest. A toxic agent can be a pesticidal protein or one or more chemical agents known in the art. Pesticidal or insecticidal chemical agents and pesticidal or insecticidal protein agents can be used alone or in combinations with each other. Chemical agents include, but are not limited to, dsRNA molecules targeting specific genes for suppression in a target pest, organochlorides, organophosphates, carbamates, pyrethroids, neonicotinoids, and ryanoids. Pesticidal or insecticidal protein agents include the protein toxins set forth in this application, as well as other proteinaceous toxic agents including those that target Coleopteran, Lepidopteran, Hemipteran, Thysanopteran, or Dipteran pest species.

The “Photorhabdus insect-related” proteins, or PirAB proteins, are binary toxins with pesticidal activity against some insects. Some PirAB proteins have been shown to have Lepidopteran activity when injected into the insect hemocoel. However, when presented in the insect diet, the oral application of the PirAB proteins have shown little to no activity (see, for example, Yang et al. (2017) PirAB protein from Xenorhabdus nematophila HB310 exhibits a binary toxin with insecticidal activity and cytotoxicity in Galleria mellonella. J. Invertebr Pathol, 148: 43-50; Li et al. (2014) Photorhabdus luminescens PirAB-fusion protein exhibits both cytotoxicity and insecticidal activity. FEMS Microbial Lett, 356: 23-31; Wu and Yunhong (2016) Scientific Reports 6, Article number: 34996; doi:10.1038/srep34996; and Zhang et al. (2013) XaxAB-like binary toxin from Photorhabdus luminescens exhibits both insecticidal activity and cytotoxicity. FEMS Microbiol Lett 350: 48-56). Oral activity of the PirAB proteins against Lepidotera have been reported but those studies have relied on the insect ingesting a diet comprising E. coli bacteria expressing the PirAB proteins and not purified toxin provided in the insect diet (see, for example, Waterfield et al. (2005) The Photorhabdus Pir toxins are similar to a developmentally regulated insect protein but show no juvenile hormone esterase activity. FEMS Microbiol Lett, 245: 47-52 and Blackburn et al. (2006) Remarkable susceptibility of the diamondback moth (Plutella xylostella) to ingestion of Pir toxins from Photorhabdus luminescens. Entomologia Experimentalis et Applicata, 121: 31-37). In stark contrast, herein, as described in the Examples, protein preparations of The PirA Proteins, The PirB Proteins, and The PirAB Fusion Proteins were provided in the insect diet bioassays. Oral activity against Lepidopteran, Coleopteran, and Hemipteran insect pests was observed and is presented in the Examples. In addition, leaf discs derived from plants expressing the PirAB fusion proteins, TIC9316, TIC9317, and TIC9318 were used in oral insect feeding studies which demonstrated activity against the Lepidopteran insect pest species European corn borer and Southwestern corn borer (SWCB). Further, leaf discs derived from plants expressing TIC10376, TIC10378, TIC10380, and TIC10381 demonstrated activity against SWCB. Also, as described in the Examples, TIC9315 and TIC11302 demonstrated activity against Western Corn Rootworm pests in stably transformed plants.

It is intended that reference to a pest, particularly a pest of a crop plant, means insect pests of crop plants, particularly those that are controlled by at least one of The PirA Proteins, The PirB Protein, and The PirAB Proteins, a related family member insecticidal protein, or a segment or fragment thereof.

As described in the Examples, one or more of The PirA Proteins, The PirB Proteins, or The PirAB Proteins exhibits insecticidal activity towards insect pests from the Coleopteran, Hemipteran, and Lepidopteran insect pest species, including adults, pupae, larvae, and neonates.

The insects of the order Lepidoptera include, but are not limited to, armyworms, cutworms, loopers, and heliothines in the Family Noctuidae, e.g., fall armyworm (Spodoptera frugiperda), Beet armyworm (Spodoptera exigua), Black armyworm (Spodoptera exempta), Bertha armyworm (Mamestra configurata), Southern armyworm (Spodoptera eridania), Black cutworm (Agrotis ipsilon), Cabbage looper (Trichoplusia ni), Soybean looper (Pseudoplusia includens), Velvetbean caterpillar (Anticarsia gemmatalis), Green cloverworm (Hypena scabra), Tobacco budworm (Heliothis virescens), Granulate cutworm (Agrotis subterranea), Armyworm (Pseudaletia unipuncta), Western cutworm (Agrotis orthogonia); borers, casebearers, webworms, coneworms, cabbageworms and skeletonizers from the Family Pyralidae, e.g., European corn borer (Ostrinia nubilalis), Navel orangeworm (Amyelois transitella), Corn root webworm (Crambus caliginosellus), Sod webworm (Herpetogramma licarsisalis), Sunflower moth (Homoeosoma electellum), Lesser cornstalk borer (Elasmopalpus lignosellus); leafrollers, budworms, seed worms, and fruit worms in the Family Tortricidae, e.g., Codling moth (Cydia pomonella), Grape berry moth (Endopiza viteana), Oriental fruit moth (Grapholita molesta), Sunflower bud moth (Suleima helianthana); and many other economically important Lepidoptera, e.g., Diamondback moth (Plutella xylostella), Pink bollworm (Pectinophora gossypiella) and Gypsy moth (Lymantria dispar). Other insect pests of order Lepidoptera include, e.g., Cotton leaf worm (Alabama argillacea), Fruit tree leaf roller (Archips argyrospila), European leafroller (Archips rosana) and other Archips species, (Chilo suppressalis, Asiatic rice borer, or rice stem borer), Rice leaf roller (Cnaphalocrocis medinalis), Corn root webworm (Crambus caliginosellus), Bluegrass webworm (Crambus teterrellus), Southwestern corn borer (Diatraea grandiosella), Surgarcane borer (Diatraea saccharalis), Spiny bollworm (Earias insulana), Spotted bollworm (Earias vittella), Old World bollworm (Helicoverpa armigera), Corn earworm (Helicoverpa zea, also known as soybean podworm and cotton bollworm), Tobacco budworm (Heliothis virescens), Sod webworm (Herpetogramma licarsisalis), Western bean cutworm (Striacosta albicosta), European grape vine moth (Lobesia botrana), Citrus leafminer (Phyllocnistis citrella), large white butterfly (Pieris brassicae), small white butterfly (Pieris rapae, also known as imported cabbageworm), Tobacco cutworm (Spodoptera litura, also known as cluster caterpillar), and Tomato leafminer (Tuta absoluta).

The insects of the order Coleoptera include, but are not limited to, Agriotes spp., Anthonomus spp., Atomaria linearis, Chaetocnema tibialis, Cosmopolites spp., Curculio spp., Dermestes spp., Diabrotica spp., Epilachna spp., Eremnus spp., Leptinotarsa decemlineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp. and Trogoderma spp, particularly when the pest is Western Corn Rootworm (Diabrotica virgifera, WCR), Northern Corn Rootworm (Diabrotica barberi, NCR), Mexican Corn Rootworm (Diabrotica virgifera zeae, MCR), Brazilian Corn Rootworm (Diabrotica balteata, BZR), Southern Corn Rootworm (Diabrotica undecimpunctata howardii, SCR), Colorado potato beetle (Leptinotarsa decemlineata, CPB), a Brazilian Corn Rootworm complex (BCR, consisting of Diabrotica viridula and Diabrotica speciosa), Crucifer Flea Beetle (Phyllotreta cruciferae), Striped Flea Beetle (Phyllotreta striolata), and Western Black Flea Beetle (Phyllotreta pusilla).

The insects of the order Hemiptera include, but are not limited to, Stink Bugs of the family Pentatomidae: Green Stink Bugs from the genus Chinavia (Chinavia hilaris, Chinavia marginata, and Chinavia pensylvanica), Stink bugs of the genus Chlorochroa (Chlorochroa granulose, Chlorochroa kanei, Chlorochroa ligata, Chlorochroa lineate, Chlorochroa opuntiae, Chlorochroa persimilis, Chlorochroa rossiana, Chlorochroa sayi, Chlorochroa uhleri, Chlorochroa belfragii, Chlorochroa faceta, Chlorochroa osborni, Chlorochroa saucia, and Chlorochroa senilis), Southern Green Stink Bug (Nezara viridula), Stink Bugs from the genus Edessa (Edessa meditabunda, Edessa bifida, and Edessa florida), the Neotropical Brown Stink Bug (Euschistus heros), stink bugs from the genus Euschistus (Euschistus acuminatus, Euschistus biformis, Euschistus conspersus, Euschistus crenator, Euschistus egglestoni, Euschistus ictericus, Euschistus inflatus, Euschistus latimarginatus, Euschistus obscures, Euschistus politus, Euschistus quadrator, Euschistus sevus, Euschistus strenuous, Euschistus tristigmus, and Euschistus variolarius), Brown Marmorated Stink Bug (Halyomorpha halys), Red-Shouldered Stink Bug (Thyanta accerra), stink bugs of the genus Thyanta (Thyanta calceata, Thyanta custator, Thyanta pallidovirens, Thyanta perditor, Thyanta maculate, and Thyanta pseudocasta), the Green Belly Stink Bug (Dichelops melacanthus) and other stink bugs of the genus Dichelops (Dichelops avilapiresi, Dichelops bicolor, Dichelops dimidatus, Dichelops furcatus, Dichelops furcifrons, Dichelops lobatus, Dichelops miriamae, Dichelops nigrum, Dichelops peruanus, Dichelops phoenix, and Dichelops saltensis), the Red Banded Stink Bug (Piezodorus guildinni) as well as Piezodorus lituratus; and insects of the family of Plataspidae such as Kudzu Bug (Megacopta cribraria), Western tarnished plant bug (Lygus hesperus), and Tarnished plant bug (Lygus lineolaris).

Reference in this application to an “isolated DNA molecule”, or an equivalent term or phrase, is intended to mean that the DNA molecule is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding an insecticidal protein or any naturally occurring insecticidal variant of that protein would be an isolated nucleotide sequence so long as the nucleotide sequence was not within the DNA of the bacterium from which the sequence encoding the protein is naturally found. A synthetic nucleotide sequence encoding the amino acid sequence of the naturally occurring insecticidal protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As described further herein, an operon containing two open reading frames (ORFs) encoding the PirA protein, TIC4771 (SEQ ID NO:1) and the PirB protein, TIC4772 (SEQ ID NO:3) was discovered in DNA obtained from Xenorhabdus nematophila strain ISB000002 which encodes the protein toxins presented as SEQ ID NO:2 and SEQ ID NO:4, respectively. The two ORFs were used to make a PirAB fusion protein encoding DNA sequence, TIC6880 (SEQ ID NO:5) wherein the two coding sequences were operably linked and in frame to produce the TIC6880 PirAB fusion protein presented as SEQ ID NO:6. Bioassay using microbial host cell-derived TIC4771 demonstrated activity against the Lepidopteran species Corn earworm (Helicoverpa zea, CEW), Diamondback Moth (Plutella xylostella, DEM), European corn borer (Ostrinia nubilalis, ECB), Velvet Bean Caterpillar (Anticarsia gemmatalis, VBC), and Southern Army Worm (Spodoptera eridania, SAW); the Coleopteran species Colorado potato beetle (Leptinotarsa decemlineata, CPB); and the Hemipteran species Tarnished plant bug (Lygus lineolaris, TPB). Bioassay using microbial host cell-derived TIC4772 demonstrated activity against the Lepidopteran species CEW, DBM, and VBC and the Hemipteran species TPB. Bioassay using microbial host cell-derived PirAB fusion protein, TIC6880 comprised of TIC4771 and TIC4772, demonstrated activity against the Lepidopteran species Fall armyworm (Spodoptera frugiperda, FAW), CEW, Southwestern Corn Borer (Diatraea grandiosella, SWCB), DBM, ECB, and VBC, the Coleopteran species CPB and Western Corn Rootworm (Diabrotica virgifera, WCR); the Hemipteran species Tarnished plant bug (Lygus lineolaris, TPB), Western tarnished plant bug (Lygus Hesperus, WTP), Southern Green Stink Bug (Nezara viridula, SGB), and Neotropical Brown Stink Bug (Euschistus heros, NBSB), and the Dipteran species Yellow fever mosquito (Aedes aegypti, YFM).

An operon containing two ORFs encoding the PirA protein, TIC7575 (SEQ ID NO:7) and the PirB protein, TIC7576 (SEQ ID NO:9) was discovered in DNA obtained from Xenorhabdus ehlersii strain 85823 which encodes the protein toxins presented as SEQ ID NO:8 and SEQ ID NO:10, respectively. The two ORFs were used to make a PirAB fusion protein encoding DNA sequence, TIC9316 (SEQ ID NO:11) wherein the two coding sequences were operably linked and in frame to produce the TIC9316 PirAB fusion protein presented as SEQ ID NO:12. Bioassay using microbial host cell-derived TIC7575 and TIC7576 did not demonstrate activity against the insects tested in assay. However, bioassay using the PirAB fusion protein TIC9316—comprised of TIC7575 and TIC7576—demonstrated activity against the Lepidopteran species SWCB, Black cutworm (Agrotis ipsilon, BCW), SAW, Tobacco budworm (Heliothis virescens, TBW), ECB, and VBC, the Coleopteran species CPB, and the Hemipteran species TPB, WTP, SGB, and NBSB.

An operon containing two ORFs encoding the PirA protein, TIC7660 (SEQ ID NO:13) and the PirB protein, TIC7661 (SEQ ID NO:15) was discovered in DNA obtained from Xenorhabdus cabanillasii strain 85908 which encodes the protein toxins presented as SEQ ID NO:14 and SEQ ID NO:16, respectively. The two ORFs were used to make a PirAB fusion protein encoding DNA sequence, TIC9317 (SEQ ID NO:17) wherein the two coding sequences were operably linked and in frame to produce the TIC9317 PirAB fusion protein presented as SEQ ID NO:18. Bioassay using microbial host cell-derived TIC7660 and TIC7661 did not demonstrate activity against the insects used in assay. However, bioassay using the PirAB fusion protein TIC9317—comprised of TIC7660 and TIC7661—demonstrated activity against the Lepidopteran species SWCB, ECB, and VBC, the Coleopteran species CPB and WCR, and the Hemipteran species TPB, WTP, and SGB.

An operon containing two ORFs encoding the PirA protein, TIC7662 (SEQ ID NO:19) and the PirB protein, TIC7663 (SEQ ID NO:21) was discovered in DNA obtained from Xenorhabdus ehlersii strain 85887 which encodes the protein toxins presented as SEQ ID NO:20 and SEQ ID NO:22, respectively. The two ORFs were used to make a PirAB fusion protein encoding DNA sequence, TIC9318 (SEQ ID NO:23) wherein the two coding sequences were operably linked and in frame to produce the TIC9318 PirAB fusion protein presented as SEQ ID NO:24. Bioassay using microbial host cell-derived TIC7662 and TIC7663 did not demonstrate activity against the insects used in assay. However, bioassay using the PirAB fusion protein TIC9318—comprised of TIC7662 and TIC7663—demonstrated activity against the Lepidopteran species SWCB, BCW, TBW, ECB, and VBC, the Coleopteran species CPB and WCR, and the Hemipteran species TPB, WTP, SGB, and NBSB.

An operon containing two ORFs encoding the PirA protein, TIC7664 (SEQ ID NO:25) and the PirB protein, TIC7665 (SEQ ID NO:27) was discovered in DNA obtained from Xenorhabdus poinarii strain 86198 which encodes the protein toxins presented as SEQ ID NO:26 and SEQ ID NO:28, respectively. The two ORFs were used to make a PirAB fusion protein encoding DNA sequence, TIC9319 (SEQ ID NO:29) wherein the two coding sequences were operably linked and in frame to produce the TIC9319 PirAB fusion protein presented as SEQ ID NO:30. Bioassay using microbial host cell-derived TIC7664 demonstrated activity against the Coleopteran species CPB. Bioassay using microbial host cell-derived TIC7665 demonstrated activity against the Lepidopteran species TBW. Bioassay using the PirAB fusion protein, TIC9319—comprised of TIC7664 and TIC76653—demonstrated activity against the Lepidopteran species SWCB, BCW, ECB, and VBC, the Coleopteran species CPB, and the Hemipteran species TPB, WTP, and SGB.

An operon containing two ORFs encoding the PirA protein TIC7666 (SEQ ID NO:31) and the PirB protein TIC7667 (SEQ ID NO:33) was discovered in DNA obtained from Photorhabdus luminescens strain 86197 which encodes the protein toxins presented as SEQ ID NO:32 and SEQ ID NO:34, respectively. The two ORFs were used to make a PirAB fusion protein encoding DNA sequence, TIC9322 (SEQ ID NO:35) wherein the two coding sequences were operably linked and in frame to produce the TIC9322 PirAB fusion protein presented as SEQ ID NO:36. Bioassay using microbial host cell-derived TIC7666 did not demonstrate activity against the insects used in assay. Bioassay using microbial host cell-derived TIC7667 demonstrated activity against the Lepidopteran species SWCB. Bioassay using the PirAB fusion protein TIC9322—comprised of TIC7666 and TIC7667—demonstrated activity against the Lepidopteran species SWCB and VBC, the Coleopteran species CPB, and the Hemipteran species TPB.

An operon containing two ORFs encoding the PirA protein, TIC7668 (SEQ ID NO:37) and the PirB protein, TIC7669 (SEQ ID NO:39) was discovered in DNA obtained from Photorhabdus luminescens strain 86194 which encodes the protein toxins presented as SEQ ID NO:38 and SEQ ID NO:40, respectively. The two ORFs were used to make a PirAB fusion protein encoding DNA sequence, TIC9320 (SEQ ID NO:41) wherein the two coding sequences were operably linked and in frame to produce the TIC9320 PirAB fusion protein presented as SEQ ID NO:42. Bioassay using microbial host cell-derived TIC7668 and TIC7669 did not demonstrate activity against the insects used in assay. However, bioassay using the PirAB fusion protein TIC9320—comprised of TIC7668 and TIC7669—demonstrated activity against the Lepidopteran species SWCB, ECB, and VBC, the Coleopteran species CPB and WCR, and the Hemipteran species TPB, SGB, and NBSB.

An operon containing two ORFs encoding the PirA protein, TIC7939 (SEQ ID NO:43) and the PirB protein, TIC7940 (SEQ ID NO:45) was discovered in DNA obtained from an unknown bacterial species comprised within a microbiome which encodes the protein toxins presented as SEQ ID NO:44 and SEQ ID NO:46, respectively. The two ORFs were used to make a PirAB fusion protein encoding DNA sequence, TIC9321 (SEQ ID NO:47), wherein the two coding sequences were operably linked and in frame to produce the TIC9321 PirAB fusion protein presented as SEQ ID NO:48.

An operon containing two ORFs encoding the PirA protein, TIC10357 (SEQ ID NO:57) and the PirB protein, TIC10366 (SEQ ID NO:59) was discovered in DNA obtained from Shewanella violacea which encodes the protein toxins presented as SEQ ID NO:58 and SEQ ID NO:60, respectively. The two open reading frames were used to make a PirAB fusion protein encoding DNA sequence, TIC10375 (SEQ ID NO:61), wherein the two coding sequences were operably linked and in frame to produce the TIC10375 PirAB fusion protein presented as SEQ ID NO:62.

An operon containing two ORFs encoding the PirA protein, TIC10358 (SEQ ID NO:63) and the PirB protein, TIC10367 (SEQ ID NO:65) was discovered in DNA obtained from Photorhabdus luminescens strain laumondii TTO1 which encodes the protein toxins presented as SEQ ID NO:64 and SEQ ID NO:66, respectively. The two ORFs were used to make a PirAB fusion protein encoding DNA sequence TIC10376 (SEQ ID NO:67) wherein the two coding sequences were operably linked and in frame to produce the TIC10376 PirAB fusion protein presented as SEQ ID NO:68. Bioassay using microbial host cell-derived TIC10358 and TIC10367 did not demonstrate activity against the insects used in assay. However, bioassay using the PirAB fusion protein TIC10376—comprised of TIC10358 and TIC10367—demonstrated activity against the Lepidopteran species SWCB and the Coleopteran species Northern Corn Rootworm (Diabrotica barberi, NCR) and WCR.

An operon containing two ORFs encoding the PirA protein, TIC10360 (SEQ ID NO:69) and the PirB protein, TIC10369 (SEQ ID NO:71) was discovered in DNA obtained from Photorhabdus asymbiotica which encodes the protein toxins presented as SEQ ID NO:70 and SEQ ID NO:72, respectively. The two ORFs were used to make a PirAB fusion protein encoding DNA sequence TIC10377 (SEQ ID NO:73) wherein the two coding sequences were operably linked and in frame to produce the TIC10377 PirAB fusion protein presented as SEQ ID NO:74.

An operon containing two ORFs encoding the PirA protein TIC10361 (SEQ ID NO:75) and the PirB protein TIC10370 (SEQ ID NO:77) was discovered in DNA obtained from Xenorhabdus sp. strain NBAII XenSa04 which encodes the protein toxins presented as SEQ ID NO:76 and SEQ ID NO:78, respectively. The two ORFs were used to make a PirAB fusion protein encoding DNA sequence TIC10378 (SEQ ID NO:79) wherein the two coding sequences were operably linked and in frame to produce the TIC10378 PirAB fusion protein presented as SEQ ID NO:80.

An operon containing two ORFs encoding the PirA protein TIC10362 (SEQ ID NO:81) and the PirB protein TIC10371 (SEQ ID NO:83) was discovered in DNA obtained from Yersinia aldovae strain 670-83 which encodes the protein toxins presented as SEQ ID NO:82 and SEQ ID NO:84, respectively. The two ORFs were used to make a PirAB fusion protein encoding DNA sequence TIC10379 (SEQ ID NO:85) wherein the two coding sequences were operably linked and in frame to produce the TIC10379 PirAB fusion protein presented as SEQ ID NO:86.

An operon containing two ORFs encoding the PirA protein TIC10363 (SEQ ID NO:87) and the PirB protein TIC10372 (SEQ ID NO:89) was discovered in DNA obtained from Xenorhabdus doucetiae strain FRM16 which encodes the protein toxins presented as SEQ ID NO:88 and SEQ ID NO:90, respectively. The two ORFs were used to make a PirAB fusion protein encoding DNA sequence TIC10380 (SEQ ID NO:91) wherein the two coding sequences were operably linked and in frame to produce the TIC10380 PirAB fusion protein presented as SEQ ID NO:92. Bioassay using microbial host cell-derived TIC10363 and TIC10372 did not demonstrate activity against the insects used in assay. However, bioassay using the PirAB fusion protein TIC10380—comprised of TIC10363 and TIC10372—demonstrated activity against the Lepidopteran species FAW, the Coleopteran species NCR and WCR and the Hemipteran species NBSB.

An operon containing two ORFs encoding the PirA protein TIC10364 (SEQ ID NO:93) and the PirB protein TIC10373 (SEQ ID NO:95) was discovered in DNA obtained from Xenorhabdus griffiniae strain BMMCB which encodes the protein toxins presented as SEQ ID NO:94 and SEQ ID NO:96, respectively. The two ORFs were used to make a PirAB fusion protein encoding DNA sequence TIC10381 (SEQ ID NO:97) wherein the two coding sequences were operably linked and in frame to produce the TIC10381 PirAB fusion protein presented as SEQ ID NO:98. Bioassay using microbial host cell-derived TIC10364 and TIC10373 did not demonstrate activity against the insects used in assay. However, bioassay using the PirAB fusion protein TIC10378—comprised of TIC10364 and TIC10373—demonstrated activity against the Coleopteran species NCR and WCR and the Hemipteran species NBSB.

An operon containing two ORFs encoding the PirA protein TIC10359 (SEQ ID NO:99) and the PirB protein TIC10368 (SEQ ID NO:101) was discovered in DNA obtained from Xenorhabdus nematophila which encodes the protein toxins presented as SEQ ID NO:100 and SEQ ID NO:102, respectively. An operon sequence comprising both TIC10359 and TIC10368 is presented as SEQ ID NO: 103.

An operon containing two ORFs encoding the PirA protein PirA_ABE68878 (SEQ ID NO:104) and the PirB protein, PirB_ABE68879 (SEQ ID NO:106) was discovered in DNA obtained from Photorhabdus luminescens strain Hm which encodes the protein toxins presented as SEQ ID NO:105 and SEQ ID NO:107, respectively. The two ORFs were used to make a PirAB fusion protein encoding DNA sequence TIC10434 (SEQ ID NO:108) wherein the two coding sequences were operably linked and in frame to produce the TIC10378 PirAB fusion protein presented as SEQ ID NO:109. Bioassay using microbial host cell-derived PirAB fusion protein TIC10434—comprised of PirA_ABE68878 and PirB_ABE68879—demonstrated activity against the Coleopteran species NCR and WCR.

An operon containing the PirB protein ORF encoding TIC11505 (SEQ ID NO:134) was discovered from Xenorhabdus nematophila strain MDI-0035777 which encodes the protein toxin presented as SEQ ID NO:135. The PirAB fusion protein TIC11056 encoding sequence (SEQ ID NO:136) comprised the TIC10364 coding sequence operably linked in frame with the TIC11505 coding sequence to produce the TIC11506 PirAB fusion protein presented as SEQ ID NO:137.

An operon containing the PirB protein ORF encoding TIC11510 (SEQ ID NO:138) was discovered from Xenorhabdus nematophila strain MDI-0035777 which encodes the protein toxin presented as SEQ ID NO:139. The PirAB fusion protein TIC11512 encoding sequence (SEQ ID NO:140) comprised the TIC10364 coding sequence operably linked in frame with the TIC11505 coding sequence to produce the TIC11056 PirAB fusion protein presented as SEQ ID NO:141.

An operon containing the PirB protein ORF encoding TIC11511 (SEQ ID NO:142) was discovered from Xenorhabdus nematophila strain MDI-0035777 which encodes the protein toxin presented as SEQ ID NO:143. The PirAB fusion protein TIC11513 encoding sequence (SEQ ID NO:144) comprised the TIC10364 coding sequence operably linked in frame with the TIC11513 coding sequence to produce the TIC11056 PirAB fusion protein presented as SEQ ID NO:145.

The PirAB fusion proteins, TIC11210, TIC11211, and TIC11301 comprised the PirA protein TIC7575 and the PirB proteins, TIC7665, TIC7667, and TIC7661, respectively. The PirAB fusion protein, TIC11212 comprises the PirA protein TIC7662 and the PirB protein TIC7665. The PirAB fusion protein, TIC11302 comprises the PirA protein, TIC7660 and the PirB protein, TIC7576. The PirAB fusion proteins TIC11210 and TIC11211 demonstrated activity against the Lepidopteran species SWCB and the Hemipteran species NBSB. The PirAB fusion proteins TIC11301 and TIC11302 demonstrated activity against the Lepidopteran species SWCB, ECB, and VBC, the Coleopteran species WCR, and the Hemipteran species NBSB and WTP.

The PirAB fusion proteins, TIC11103 and TIC11104 comprised a PirB protein congruent with a PirA Protein. The PirAB fusion protein, TIC11103 is comprised of the PirB protein, TIC7661 and the PirA protein, TIC7660. The PirAB fusion protein, TIC11104 is comprised of the PirB protein TIC7663 and the PirA protein TIC7662.

The PirAB fusion protein TIC11140 is comprised of a duplication of the PirA protein TIC4771 and the PirB protein TIC4472. The PirAB fusion protein TIC11141 is comprised of a duplication of the PirA protein TIC7575 and the PirB protein TIC7576. The PirAB fusion protein TIC11142 is comprised of the PirA proteins, TIC7575 and TIC4771, and the PirB protein TIC4772. The PirAB fusion protein TIC11443 is comprised of the PirA proteins TIC7660 and TIC7575 and the PirB protein, TIC7576. The PirAB fusion protein TIC11444 is comprised of the PirA proteins TIC7575 and TIC7660 and the PirB protein TIC7661. The PirAB fusion protein TIC11445 is comprised of the PirA proteins TIC7660 and TIC7662 and the PirB protein TIC7663. The PirAB fusion protein TIC11446 is comprised of the PirA proteins TIC7662 and TIC7660 and the PirB protein TIC7661. Bacterial host cell derived TIC11442 demonstrated activity against the Hemipteran pest species NBSB. Bacterial host cell derived TIC11444 demonstrated activity against the Lepidopteran species SWCB and the Hemipteran species NBSB.

As described in the Examples, synthetic DNA sequences encoding the PirAB fusion proteins TIC6880PL (SEQ ID NO:49), TIC9316 (SEQ ID NO:51), TIC9317 (SEQ ID NO:53), TIC9318 (SEQ ID NO:55), TIC9320 (SEQ ID NO:57), TIC9322 (SEQ ID NO:59), TIC10376PL (SEQ ID NO:146), TIC10378PL (SEQ ID NO:148), TIC10380PL (SEQ ID NO:150), TIC10381PL (SEQ ID NO:152), TIC11103 (SEQ ID NO:154), TIC11104 (SEQ ID NO:156), and TIC11302 (SEQ ID NO:158) were designed for expression in a plant cell. Corn plants transformed with binary transformation plasmid constructs expressing TIC9316, TIC9317, and TIC9318 demonstrated activity against the insect pest species European corn borer and Southwestern corn borer.

For expression in plant cells, The PirAB Fusion Proteins can be expressed to reside in the cytosol or targeted to various organelles of the plant cell. For example, targeting a protein to the chloroplast may result in increased levels of expressed protein in a transgenic plant while preventing off-phenotypes from occurring. Targeting may also result in an increase in pest resistance efficacy in the transgenic event. A target peptide or transit peptide is a short (3-70 amino acids long) peptide chain that directs the transport of a protein to a specific region in the cell, including the nucleus, mitochondria, endoplasmic reticulum (ER), chloroplast, apoplast, peroxisome and plasma membrane. Some target peptides are cleaved from the protein by signal peptidases after the proteins are transported. For targeting to the choloroplast, proteins contain transit peptides which are around 40-50 amino acids. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (See, Klee et al., Mol. Gen. Genet. 210:437-442, 1987) or the Petunia hybrida EPSPS CTP (CTP4) (See, della-Cioppa et al., Proc. Natl. Acad. Sci. USA 83:6873-6877, 1986) has been shown to target heterologous EPSPS protein sequences to chloroplasts in transgenic plants (See, U.S. Pat. Nos. 5,627,061; 5,633,435; and 5,312,910; and EP 0218571; EP 189707; EP 508909; and EP 924299). For targeting one of The PirAB Fusion Proteins to the chloroplast, a sequence encoding a chloroplast transit peptide is placed 5′ in operable linkage and in frame to a synthetic coding sequence encoding one of The PirAB Fusion Proteins that has been designed for optimal expression in plant cells.

It is contemplated that additional toxin protein sequences related to The PirA Proteins, The PirB Proteins, and The PirAB Fusion Proteins can be created by using the naturally occurring amino acid sequence of The PirA Proteins, The PirB Proteins, and The PirAB Fusion Proteins to combine differences at the amino acid sequence level into novel amino acid sequence variants and making appropriate changes to the recombinant nucleic acid sequence encoding the variants.

This disclosure further contemplates that improved variants of The PirA Proteins, The PirB Proteins, and The PirAB Fusion Proteins can be engineered in planta by using various gene editing methods known in the art. Such technologies used for genome editing include, but are not limited to, ZFN (zinc-finger nuclease), meganucleases, TALEN (Transcription activator-like effector nucleases), and CRISPR (Clustered Regularly Interspaced Short Palindromic Repeats)/Cas (CRISPR-associated) systems. These genome editing methods can be used to alter the toxin protein coding sequence transformed within a plant cell to a different toxin coding sequence. Specifically, through these methods, one or more codons within the toxin coding sequence is altered to engineer a new protein amino acid sequence. Alternatively, a fragment within the coding sequence is replaced or deleted, or additional DNA fragments are inserted into the coding sequence, to engineer a new toxin coding sequence. The new coding sequence can encode a toxin protein with new properties such as increased activity or spectrum against insect pests, as well as provide activity against an insect pest species wherein resistance has developed against the original insect toxin protein. The plant cell comprising the gene edited toxin coding sequence can be used by methods known in the art to generate whole plants expressing the new toxin protein.

Proteins that resemble The PirA Proteins, The PirB Proteins, and The PirAB Fusion Proteins can be identified by comparison to each other using various computer-based algorithms known in the art. For example, amino acid sequence identities of proteins related to The PirA Proteins, The PirB Proteins, and The PirAB Fusion Proteins can be analyzed using a Clustal W alignment using these default parameters: Weight matrix: blosum, Gap opening penalty: 10.0, Gap extension penalty: 0.05, Hydrophilic gaps: On, Hydrophilic residues: GPSNDQERK, Residue-specific gap penalties: On (Thompson, et al (1994) Nucleic Acids Research, 22:4673-4680). Percent amino acid identity is further calculated by the product of 100% multiplied by (amino acid identities/length of subject protein). Other alignment algorithms are also available in the art and provide results similar to those obtained using a Clustal W alignment.

It is intended that a protein exhibiting insect inhibitory activity against a Lepidopteran, or Coleopteran, or Hemipteran insect species is related to The PirA Proteins if alignment of such query protein with TIC7939 exhibits at least 65% to about 100% amino acid identity along the length of the query protein that is about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity (or any fraction of a percentage in this range) between query and subject protein; or if alignment of such query protein with TIC7664 or TIC7666 exhibits at least 97% to about 100% amino acid identity along the length of the query protein that is about 97%, 98%, 99%, 100% amino acid sequence identity (or any fraction of a percentage in this range) between query and subject protein; or if alignment of such query protein with TIC4771 exhibits at least 98% to about 100% amino acid identity along the length of the query protein that is about 98%, 99%, 100% amino acid sequence identity (or any fraction of a percentage in this range) between query and subject protein; or if alignment of such query protein with TIC7575, TIC7660, TIC7662, TIC7668, TIC10357, TIC10358, TIC10360, TIC10361, TIC10362, TIC10364, TIC10359, or PirA_ABE68878 exhibits 100% identity between query and subject protein.

It is also intended that a protein exhibiting insect inhibitory activity against a Lepidopteran, or Coleopteran, or Hemipteran insect species is related to The PirB Proteins if alignment of such query protein with TIC7940 exhibits at least 65% to about 100% amino acid identity along the length of the query protein that is about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity (or any fraction of a percentage in this range) between query and subject protein; or if alignment of such query protein with TIC4772 exhibits at least 97% to about 100% amino acid identity along the length of the query protein that is about 97%, 98%, 99%, 100% amino acid sequence identity (or any fraction of a percentage in this range) between query and subject protein; or if alignment of such query protein with TIC4772 exhibits at least 97% to about 100% amino acid identity along the length of the query protein that is about 97%, 98%, 99%, 100% amino acid sequence identity (or any fraction of a percentage in this range) between query and subject protein; or if alignment of such query protein with TIC7665, TIC7667, or TIC10368 exhibits at least 98% to about 100% amino acid identity along the length of the query protein that is about 98%, 99%, 100% amino acid sequence identity (or any fraction of a percentage in this range) between query and subject protein; or if alignment of such query protein with TIC7576, TIC7661, TIC7663, TIC7669, TIC10366, TIC10367, TIC10369, TIC10370, TIC10371, TIC10372, TIC10373, PirB_ABE68879, TIC11510, or TIC11511 exhibits 100% amino acid sequence identity between query and subject protein.

It is also intended that a protein exhibiting insect inhibitory activity against a Lepidopteran, or Coleopteran, or Hemipteran insect species is related to The PirAB Fusion Proteins if alignment of such query protein with TIC9321, TIC11411, TIC11443, TIC11444, TIC11445, TIC11446, TIC11513 exhibits at least 65% to about 100% amino acid identity along the length of the query protein that is about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity (or any fraction of a percentage in this range) between query and subject protein; or if alignment of such query protein with TIC10434, TIC11440, or TIC11442 exhibits at least 70% to about 100% amino acid identity along the length of the query protein that is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity (or any fraction of a percentage in this range) between query and subject protein; or if alignment of such query protein with TIC9316, TIC9317, TIC9318, TIC9322, TIC9320, TIC10375, TIC10376, TIC10377, TIC10378, TIC10379, TIC10381, TIC11211, TIC11301, TIC11302, TIC10376PL, TIC10378PL, TIC10381PL, TIC11103, or TIC11104 exhibits at least 80% to about 100% amino acid identity along the length of the query protein that is about 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity (or any fraction of a percentage in this range) between query and subject protein; or if alignment of such query protein with TIC9319, TIC10380, TIC11210, TIC11212, or TIC10380PL exhibits at least 82% to about 100% amino acid identity along the length of the query protein that is about 82%, 83%, 84%, 85%, 86%, 87%, 88%, 89%, 90%, 91%, 92%, 93%, 94%, 95%, 96%, 97%, 98%, 99%, 100% amino acid sequence identity (or any fraction of a percentage in this range) between query and subject protein; or if alignment of such query protein with TIC6880 or TIC6880PL exhibits at least 86% to about 100% amino acid identity along the length of the query protein that is about 86%, 87%, 88%, 89%, 90%, 91%, 92%, 93%, 94%, 95%, 96%, 97%, 98%, 99% 100% amino acid sequence identity (or any fraction of a percentage in this range) between query and subject protein; or if alignment of such query protein with TIC11506 or TIC11512 exhibits at least 94% to about 100% amino acid identity along the length of the query protein that is about 94%, 95%, 96%, 97%, 98%, 99%, 100% amino acid sequence identity (or any fraction of a percentage in this range) between query and subject protein.

Exemplary PirA proteins TIC4771, TIC7575, TIC7660, TIC7662, TIC7664, TIC7666, TIC7668, TIC7939, TIC10357, TIC10358, TIC10360, TIC10361, TIC10362, TIC10363, TIC10364, TIC10359, and PirA_ABE68878 were aligned with each other using a Clustal W algorithm. A pair-wise matrix of percent amino acid sequence identities for each of the full-length proteins was created, as reported in Tables 4 and 5.

TABLE 4
Pair-wise matrix display of PirA proteins.

| Sequence | TIC7666 | TIC7668 | PirA_ABE68878 | TIC10360 | TIC7660 | TIC10361 | TIC7662 | TIC10364 |
|---|---|---|---|---|---|---|---|---|
| TIC7666 | — | 94.7 | 92.5 | 85.7 | 51.1 | 48.1 | 48.1 | 49.6 |
| TIC7668 | 94.7 | — | 94 | 88.7 | 51.1 | 49.6 | 48.9 | 50.4 |
| PirA_ABE68878 | 89.1 | 90.6 | — | 81.2 | 47.8 | 46.4 | 47.1 | 48.6 |
| TIC10360 | 85.7 | 88.7 | 84.2 | — | 50.4 | 50.4 | 52.6 | 52.6 |
| TIC7660 | 48.2 | 48.2 | 46.8 | 47.5 | — | 93.6 | 75.2 | 71.6 |
| TIC10361 | 44.8 | 46.2 | 44.8 | 46.9 | 92.3 | — | 75.5 | 73.4 |
| TIC7662 | 45.4 | 46.1 | 46.1 | 49.6 | 75.2 | 76.6 | — | 85.1 |
| TIC10364 | 46.5 | 47.2 | 47.2 | 49.3 | 71.1 | 73.9 | 84.5 | — |

TABLE 5
Pair-wise matrix display of PirA proteins.

| Sequence | TIC7666 | TIC7668 | PirA_ABE68878 | TIC10360 | TIC7660 | TIC10361 | TIC7662 | TIC10364 |
|---|---|---|---|---|---|---|---|---|
| TIC7575 | 43.3 | 45.4 | 44 | 47.5 | 72.3 | 73.8 | 87.2 | 83.7 |
| TIC4771 | 45.2 | 45.9 | 45.9 | 48.1 | 72.6 | 75.6 | 75.6 | 76.3 |
| TIC7664 | 45.2 | 45.9 | 45.9 | 48.9 | 70.4 | 71.9 | 74.1 | 74.1 |
| TIC10363 | 45.3 | 46 | 45.3 | 48.2 | 71.5 | 74.5 | 80.3 | 78.8 |
| TIC10359 | 47.4 | 48.9 | 47.4 | 51.1 | 71.9 | 71.9 | 74.1 | 72.6 |
| TIC7939 | 35.3 | 36.7 | 36 | 36.7 | 41.7 | 42.4 | 41.7 | 41.7 |
| TIC10358 | 40.3 | 41 | 41 | 40.3 | 36.8 | 38.2 | 39.6 | 37.5 |
| TIC10362 | 42.6 | 44.9 | 44.1 | 44.9 | 36.8 | 37.5 | 43.4 | 40.4 |
| TIC10357 | 23.7 | 23.7 | 22.8 | 24.6 | 21.9 | 22.8 | 26.3 | 25.4 |

Exemplary PirB proteins TIC4772, TIC7576, TIC7661, TIC7663, TIC7665, TIC7667, TIC7669, and TIC7940 were aligned with each other using a Clustal W algorithm. A pair-wise matrix of percent amino acid sequence identities for each of the full-length proteins was created, as reported in Tables 6 and 7.

TABLE 6
Pair-wise matrix display of PirB proteins.

| Sequence | TIC7667 | TIC7669 | PirB_ABE68879 | TIC10369 | TIC7576 | TIC10373 | TIC7663 | TIC7661 | TIC10370 |
|---|---|---|---|---|---|---|---|---|---|
| TIC7667 | — | 95.5 | 93.6 | 93.3 | 49.6 | 48.7 | 49.6 | 48.9 | 49.4 |
| TIC7669 | 95.5 | — | 95.7 | 94.5 | 50.4 | 49.6 | 50.4 | 49.4 | 49.9 |
| PirB_ABE68879 | 93.6 | 95.7 | — | 94 | 50.1 | 49.2 | 50.8 | 49.2 | 49.9 |
| TIC10369 | 93.3 | 94.5 | 94 | — | 49.4 | 48.9 | 50.4 | 49.2 | 49.4 |
| TIC7576 | 48.9 | 49.6 | 49.4 | 48.7 | — | 94.8 | 90.6 | 87.5 | 89.9 |
| TIC10373 | 48 | 48.9 | 48.5 | 48.2 | 94.8 | — | 92.7 | 86.4 | 87.8 |
| TIC7663 | 48.9 | 49.6 | 50.1 | 49.6 | 90.6 | 92.7 | — | 84.2 | 86.1 |
| TIC7661 | 48.2 | 48.7 | 48.5 | 48.5 | 87.5 | 86.4 | 84.2 | — | 95.3 |
| TIC10370 | 48.7 | 49.2 | 49.2 | 48.7 | 89.9 | 87.8 | 86.1 | 95.3 | — |
| TIC10372 | 48.4 | 49.1 | 48.6 | 47.9 | 84.4 | 82.1 | 80.9 | 79.5 | 80.9 |
| TIC11510 | 47.1 | 47.1 | 47.6 | 47.6 | 79.5 | 79 | 79.7 | 77.2 | 78.3 |
| TIC11511 | 47.3 | 47.3 | 47.8 | 47.8 | 80 | 79.5 | 80.2 | 77.6 | 78.8 |
| TIC10368 | 47.6 | 47.6 | 48 | 48 | 80.2 | 79.7 | 80.4 | 77.9 | 79 |
| TIC11505 | 47 | 47 | 47.5 | 47.5 | 79.3 | 78.8 | 79.5 | 77 | 78.1 |
| TIC4772 | 47.2 | 47.9 | 47.9 | 47.2 | 79.7 | 78.5 | 79.7 | 76.2 | 78 |
| TIC7665 | 49 | 49.3 | 49.5 | 49.5 | 81.2 | 80.2 | 80.9 | 78.7 | 79.7 |
| TIC10367 | 41.7 | 42.4 | 42 | 42.7 | 43.2 | 42.7 | 43.6 | 42.7 | 43.2 |
| TIC10371 | 45.2 | 44.7 | 44.5 | 45 | 48.8 | 48.6 | 48.1 | 48.3 | 48.3 |
| TIC7940 | 37 | 38.2 | 37.7 | 37 | 41.5 | 42.5 | 43 | 41.8 | 42.2 |
| TIC10366 | 24.8 | 25.1 | 24.8 | 25.8 | 25.1 | 24.8 | 24.4 | 24.6 | 25.1 |

TABLE 7
Pair-wise matrix display of PirB proteins.

| Sequence | TIC10372 | TIC11510 | TIC11511 | TIC10368 | TIC11505 | TIC4772 | TIC7665 | TIC10367 | TIC10371 | TIC7940 | TIC10366 |
|---|---|---|---|---|---|---|---|---|---|---|---|
| TIC7667 | 49.6 | 48.2 | 48.4 | 48.7 | 48.7 | 48.2 | 48.4 | 41.5 | 44.9 | 37 | 25.3 |
| TIC7669 | 50.4 | 48.2 | 48.4 | 48.7 | 48.7 | 48.9 | 48.7 | 42.2 | 44.4 | 38.2 | 25.5 |
| PirB_ABE68879 | 49.9 | 48.7 | 48.9 | 49.2 | 49.2 | 48.9 | 48.9 | 41.8 | 44.2 | 37.7 | 25.3 |
| TIC10369 | 49.2 | 48.7 7 | 48.9 | 49.2 | 49.2 | 48.2 | 48.9 | 42.5 | 44.6 | 37 | 26.3 |
| TIC7576 | 85.4 | 80.2 | 80.7 | 80.9 | 80.9 | 80.2 | 79.1 | 42.4 | 47.8 | 40.9 | 25.2 |
| TIC10373 | 83.1 | 79.8 | 80.2 | 80.5 | 80.5 | 79.1 | 78.1 | 41.9 | 47.5 | 41.9 | 24.9 |
| TIC7663 | 81.9 | 80.5 | 80.9 | 81.2 | 81.2 | 80.2 | 78.8 | 42.8 | 47.1 | 42.4 | 24.5 |
| TIC7661 | 80.5 | 77.9 | 78.4 | 78.6 | 78.6 | 76.7 | 76.7 | 41.9 | 47.3 | 41.2 | 24.7 |
| TIC10370 | 81.9 | 79.1 | 79.5 | 79.8 | 79.8 | 78.6 | 77.6 | 42.4 | 47.3 | 41.6 | 25.2 |
| TIC10372 | — | 79.1 | 79.5 | 79.8 | 79.8 | 78.8 | 76.5 | 40.5 | 47.2 | 39.8 | 24.2 |
| TIC11510 | 79.3 | — | 99.5 | 99.3 | 99.3 | 86.5 | 81.6 | 42.2 | 46.4 | 41 | 24.5 |
| TIC11511 | 79.7 | 99.5 | — | 99.8 | 99.8 | 86.9 | 81.6 | 42.2 | 46.9 | 41 | 24.2 |
| TIC10368 | 80 | 99.3 | 99.8 | — | 100 | 87.2 | 81.8 | 42 | 46.6 | 41 | 24 |
| TIC11505 | 79 | 98.2 | 98.6 | 98.8 | — | 86.2 | 80.9 | 41.5 | 46.1 | 40.6 | 24 |
| TIC4772 | 79.2 | 86.7 | 87.1 | 87.4 | 87.4 | — | 80.8 | 42.5 | 47 | 41.8 | 24.5 |
| TIC7665 | 79.5 | 84.5 | 84.5 | 84.8 | 84.8 | 83.6 | — | 43 | 47.3 | 41.5 | 25.8 |
| TIC10367 | 41.7 | 43.4 | 43.4 | 43.2 | 43.2 | 43.6 | 42.7 | — | 57.6 | 35 | 22.1 |
| TIC10371 | 48.8 | 47.8 | 48.3 | 48.1 | 48.1 | 48.3 | 47.1 | 57.7 | — | 38.2 | 25.5 |
| TIC7940 | 40.8 | 42 | 42 | 42 | 42 | 42.7 | 41.1 | 34.8 | 37.9 | — | 24.6 |
| TIC10366 | 24.4 | 24.6 | 24.4 | 24.1 | 24.4 | 24.6 | 25.1 | 21.5 | 24.8 | 24.1 | — |

Exemplary PirAB fusion proteins TIC6880, TIC9316, TIC9317, TIC9318, TIC9319, TIC9322, TIC9320, TIC9321, TIC6880PL, TIC10375, TIC10376, TIC10377, TIC10378, TIC10379, TIC10380, TIC10381, TIC10434, TIC11210, TIC11211, TIC11212, TIC11301, TIC11302, TIC11440, TIC11441, TIC11442, TIC11443, TIC11444, TIC11445, TIC11446, TIC11506, TIC11512, TIC11513, TIC10376PL, TIC10378PL, TIC10380PL, TIC10381PL, TIC11103, and TIC11104 were aligned with each other using a Clustal W algorithm. A pair-wise matrix of percent amino acid sequence identities for each of the full-length proteins was created, as reported in Tables 8, 9, 10, and 11.

TABLE 8
Pair-wise matrix display of PirAB fusion proteins.

| Sequence | TIC9322 | TIC9320 | TIC10434 | TIC10377 | TIC11211 | TIC6880 | TIC11440 | TIC6880PL | TIC11442 | TIC11506 |
|---|---|---|---|---|---|---|---|---|---|---|
| TIC9322 | — | 95.3 | 93.3 | 91.5 | 87.3 | 47.8 | 47.8 | 47.8 | 47.8 | 48.7 |
| TIC9320 | 95.3 | — | 95.3 | 93.1 | 84.4 | 48.6 | 48.6 | 48.6 | 48.6 | 48.9 |
| TIC10434 | 92.5 | 94.4 | — | 90.8 | 82 | 48.1 | 48.1 | 48.1 | 48.1 | 48.8 |
| TIC10377 | 91.5 | 93.1 | 91.7 | — | 82.6 | 48 | 48 | 48 | 48 | 49.6 |
| TIC11211 | 86.1 | 83.2 | 81.6 | 81.4 | — | 52.7 | 52.7 | 52.9 | 52.7 | 53.4 |
| TIC6880 | 46.9 | 47.6 | 47.6 | 47.1 | 52.4 | — | 99.6 | 99.8 | 99.6 | 85.3 |
| TIC11440 | 37.8 | 38.4 | 38.4 | 38 | 42.3 | 80.4 | — | 80.2 | 95.6 | 68.8 |
| TIC6880PL | 46.8 | 47.5 | 47.5 | 47 | 52.5 | 99.6 | 99.3 | — | 99.3 | 85.1 1 |
| TIC11442 | 37.5 | 38.1 | 38.1 | 37.6 | 41.9 | 79.7 | 94.7 | 79.5 | — | 68.2 |
| TIC11506 | 46.7 | 46.9 | 47.2 | 47.6 | 51.9 | 83.3 | 83.3 | 83.3 | 83.3 | — |
| TIC11513 | 46.9 | 47.1 | 47.5 | 47.8 | 52.2 | 83.9 | 83.9 | 83.9 | 83.9 | 99.8 |
| TIC11512 | 46.8 | 46.9 | 47.3 | 47.6 | 52 | 83.5 | 83.5 | 83.5 | 83.5 | 99.5 |
| TIC11210 | 47.2 | 47.7 | 47.6 | 48.3 | 55.5 | 81.1 | 81.1 | 81.1 | 81.1 | 84.7 |
| TIC11212 | 47.2 | 47.6 | 47.7 | 48.5 | 54.2 | 80.9 | 80.9 | 80.9 | 80.9 | 84.7 |
| TIC9319 | 47.7 | 48.1 | 48.3 | 48.8 | 54.3 | 83.2 | 83.2 | 83.1 | 83.2 | 82.5 |
| TIC10380 | 47.8 | 48.5 | 48 | 48.1 | 53.8 | 79.2 | 79.2 | 79 | 79.2 | 79.9 |
| TIC10380PL | 47.7 | 48.4 | 47.9 | 48.1 | 53.9 | 78.9 | 78.9 | 79.2 | 78.9 | 79.8 |
| TIC10381 | 47.4 | 48.3 | 48 | 48.1 | 52.7 | 78 | 78 | 78 | 78 | 84.8 |
| TIC10381PL | 47.4 | 48.2 | 47.9 | 48.1 | 52.6 | 77.8 | 77.8 | 77.8 | 77.8 | 84.5 |
| TIC9318 | 47.5 | 48.2 | 48.6 | 48.9 | 54.4 | 78.4 | 78.4 | 78.4 | 78.4 | 81.8 |
| TIC11445 | 38 | 38.6 | 38.9 | 39.2 | 43.6 | 62.8 | 76.5 | 62.8 | 77.2 | 65.5 |
| TIC9316 | 47.5 | 48.4 | 47.9 | 48.1 | 56 | 79 | 79 | 79 | 79 | 81.3 |
| TIC11441 | 38 | 38.8 | 38.3 | 38.5 | 44.8 | 63.2 | 77.8 | 63.2 | 82.9 | 65.1 |
| TIC11302 | 48.1 | 48.8 | 48.1 | 48.1 | 52.3 | 77.7 | 77.7 | 77.7 | 77.7 | 78.3 |
| TIC11443 | 38.5 | 39 | 38.5 | 38.5 | 41.9 | 62.2 | 62.2 | 62.2 | 62.2 | 62.7 |
| TIC9317 | 47.5 | 48.1 | 47.3 | 47.9 | 51.6 | 75.4 | 75.4 | 75.4 | 75.4 | 76.9 |
| TIC11446 | 38 | 38.5 | 37.9 | 38.3 | 41.3 | 60.4 | 74.8 | 60.4 | 77.5 | 61.5 |
| TIC11444 | 38 | 38.5 | 37.9 | 38.3 | 41.3 | 60.4 | 75 | 60.4 | 80.1 | 61.5 |
| TIC11301 | 46.8 | 47.5 | 47 | 47.7 | 54.9 | 76.5 | 76.5 | 76.5 | 76.5 | 80 |
| TIC10378 | 47.7 | 48.6 | 48.1 | 48.4 | 51.8 | 76.9 | 76.9 | 76.9 | 76.9 | 78 |
| TIC10378PL | 47.6 | 48.5 | 48 | 48.3 | 51.7 | 76.8 | 76.8 | 76.8 | 76.8 | 77.7 |
| TIC11103 | 36.2 | 36.6 | 36.4 | 36.4 | 36.2 | 58.1 | 58.1 | 58.1 | 58.1 | 59 |
| TIC11104 | 36.7 | 37.3 | 37.6 | 37.3 | 36.7 | 60.1 | 60.1 | 60.1 | 60.1 | 60.6 |
| TIC10376 | 41.2 | 41.9 | 41.5 | 41.9 | 40.8 | 41.9 | 41.9 | 41.9 | 41.9 | 41.5 |
| TIC10376PL | 41.1 | 41.8 | 41.5 | 41.8 | 40.7 | 41.8 | 41.8 | 41.8 | 41.8 | 41.5 |
| TIC10379 | 43.7 | 43.7 | 43.7 | 43.5 | 43.1 | 46.2 | 46.2 | 46.2 | 46.2 | 46.6 |
| TIC9321 | 36.4 | 37.5 | 37.3 | 36.2 | 37.6 | 42.1 | 42.1 | 42.1 | 42.1 | 41.2 |
| TIC10375 | 24.4 | 24.6 | 24.2 | 25.3 | 24.2 | 23.7 | 23.7 | 23.7 | 23.7 | 23.3 |

TABLE 9
Pair-wise matrix display of PirAB fusion proteins.

| Sequence | TIC11513 | TIC11512 | TIC11210 | TIC11212 | TIC9319 | TIC10380 | TIC10380PL | TIC10381 | TIC10381PL | TIC9318 |
|---|---|---|---|---|---|---|---|---|---|---|
| TIC9322 | 48.6 | 48.4 | 47.5 | 47.5 | 47.5 | 49.1 | 49.1 | 48.7 | 48.7 | 48.7 |
| TIC9320 | 48.7 | 48.6 | 48 | 47.8 | 47.8 | 49.8 | 49.8 | 49.6 | 49.6 | 49.5 |
| TIC10434 | 48.7 | 48.5 | 47.4 17 | 47.6 | 47.6 | 48.8 | 48.8 | 48.8 | 48.8 | 49.4 |
| TIC10377 | 49.5 | 49.3 | 48.6 | 48.7 | 48.6 | 49.5 | 49.5 | 49.5 | 49.5 | 50.2 |
| TIC11211 | 53.2 | 53 | 55 | 53.8 | 53.2 | 54.5 | 54.6 | 53.4 | 53.4 | 55 |
| TIC6880 | 85.1 | 84.7 | 79.9 | 79.8 | 81.2 | 79.8 | 79.6 | 78.5 | 78.5 | 78.9 |
| TIC11440 | 68.6 | 68.3 | 64.5 | 64.3 | 65.5 | 64.3 | 64.2 | 63.3 | 63.3 | 63.6 |
| TIC6880PL | 84.9 | 84.6 | 79.8 | 79.6 | 80.9 | 79.4 | 79.8 | 78.4 | 78.4 | 78.7 |
| TIC11442 | 68 | 67.8 | 63.9 | 63.8 | 64.9 | 63.8 | 63.6 | 62.8 | 62.8 | 63.1 |
| TIC11506 | 99 | 98.6 | 81.6 | 81.6 | 78.6 | 78.6 | 78.6 | 83.5 | 83.3 | 80.4 |
| TIC11513 | — | 99.6 | 82.1 | 82.1 | 79.2 | 79.2 | 79.2 | 84.1 | 83.9 | 80.9 |
| TIC11512 | 99.6 | — | 82.1 | 82.1 | 79.2 | 78.8 | 78.8 | 83.7 | 83.5 | 80.6 |
| TIC11210 | 84.5 | 84.5 | — | 96.8 | 93.2 | 79.1 | 79.1 | 80.9 | 80.7 | 82.2 |
| TIC11212 | 84.5 | 84.5 | 96.8 | — | 92.8 | 79.3 | 79.3 | 80.9 | 80.7 | 85.2 |
| TIC9319 | 82.3 | 82.3 | 94.2 | 93.8 | — | 79.4 | 79.2 | 78.7 | 78.7 | 79.2 |
| TIC10380 | 79.7 7 | 79.4 | 77.4 | 77.6 | 76.9 | — | 99.8 | 81.7 | 81.7 | 81.1 |
| TIC10380PL | 79.6 | 79.2 | 77.3 | 77.5 | 76.6 | 99.6 | — | 81.5 | 81.5 | 81 |
| TIC10381 | 84.7 | 84.3 | 79.2 | 79.2 | 76.2 | 81.7 | 81.7 | — | 99.8 | 90.7 |
| TIC10381PL | 84.3 | 84 | 78.9 | 78.9 | 76.1 | 81.5 | 81.5 | 99.6 | — | 90.3 |
| TIC9318 | 81.6 | 81.3 | 80.6 | 83.6 | 76.9 | 81.3 | 81.3 | 90.8 | 90.6 | — |
| TIC11445 | 65.3 | 65.1 | 64.5 | 66.9 | 61.5 | 65.1 | 65.1 | 72.7 | 72.6 | 80.1 |
| TIC9316 | 81.1 | 80.7 | 83.4 | 80.4 | 77.2 | 83.7 | 83.7 | 92 | 91.9 | 89.8 |
| TIC11441 | 64.9 | 64.6 | 66.8 | 64.4 | 61.8 | 67 | 67 | 73.7 | 73.6 | 71.9 |
| TIC11302 | 78.1 | 77.7 | 77.2 | 77.7 | 76.1 | 81.6 | 81.6 | 89 | 88.9 | 86.7 |
| TIC11443 | 62.5 | 62.2 | 61.8 | 62.2 | 61 | 65.3 | 65.3 | 71.3 | 71.1 | 69.4 |
| TIC9317 | 76.7 | 76.3 | 75.8 | 76.1 | 74.4 | 77.9 | 77.9 | 82.7 | 82.5 | 82 |
| TIC11446 | 61.4 | 61.1 | 60.7 | 61 | 59.5 | 62.4 | 62.4 | 66.2 | 66.1 | 65.6 |
| TIC11444 | 61.4 | 61.1 | 60.7 | 61 | 59.5 | 62.4 | 62.4 | 66.2 | 66.1 | 65.6 |
| TIC11301 | 79.9 | 79.5 | 82.3 | 79.2 | 75.6 | 79.9 | 79.9 | 85.9 | 85.7 | 84.8 |
| TIC10378 | 77.8 | 77.5 | 76.6 | 76.9 | 75.4 | 79.4 | 79.4 | 84.3 | 84.2 | 83.5 |
| TIC10378PL | 77.5 | 77.2 | 76.3 | 76.6 | 75.2 | 79.3 | 79.3 | 84 | 84.4 | 83.1 |
| TIC11103 | 58.8 | 58.5 | 57.4 | 57.4 | 57.4 | 60.4 | 60.4 | 64.8 | 64.8 | 63.3 |
| TIC11104 | 60.4 | 60.1 | 59 | 59 | 59 | 61.5 | 61.5 | 69.6 | 69.6 | 75.1 |
| TIC10376 | 41.7 | 41.7 | 41.4 | 41.7 | 40.8 | 40.6 | 40.6 | 41.5 | 41.5 | 42.8 |
| TIC10376PL | 41.6 | 41.6 | 41.3 | 41.6 | 40.7 | 40.6 | 40.6 | 41.5 | 41.5 | 42.7 |
| TIC10379 | 46.7 | 46.4 | 45.5 | 46.6 | 45.3 | 47.1 | 47.1 | 46.9 | 46.7 | 47.3 |
| TIC9321 | 41.2 | 41.2 | 40.3 | 40.9 | 40.9 | 41 | 41 | 41.9 | 41.9 | 42.5 |
| TIC10375 | 23.5 | 23.7 | 24.6 | 24.6 | 25 | 24.2 | 24.2 | 24.2 | 24.2 | 24 |

TABLE 10
Pair-wise matrix display of PirAB fusion proteins.

| Sequence | TIC11445 | TIC9316 | TIC11441 | TIC11302 | TIC11443 | TIC9317 | TIC11446 | TIC11444 | TIC11301 |
|---|---|---|---|---|---|---|---|---|---|
| TIC9322 | 48.7 | 48.7 | 48.7 | 49.3 | 49.3 | 48.7 | 48.7 | 48.7 | 48 |
| TIC9320 | 49.5 | 49.6 | 49.6 | 50 | 50 | 49.3 | 49.3 | 49.3 | 48.7 |
| TIC10434 | 49.4 | 48.7 | 48.7 | 48.8 | 48.8 | 48.1 | 48.1 | 48.1 | 47.8 |
| TIC10377 | 50.2 | 49.3 | 49.3 | 49.3 | 49.3 | 49.1 | 49.1 | 49.1 | 48.9 |
| TIC11211 | 55 | 56.6 | 56.6 | 52.9 | 52.9 | 52.1 | 52.1 | 52.1 | 55.5 |
| TIC6880 | 78.9 | 79.4 | 79.4 | 78.2 | 78.2 | 75.8 | 75.8 | 75.8 | 76.9 |
| TIC11440 | 77.5 | 64 | 78.8 | 63 | 63 | 61.2 | 75.8 15 | 75.9 | 62 |
| TIC6880PL | 78.7 | 79.3 | 79.3 | 78 | 78 | 75.7 | 75.7 | 75.7 | 76.8 |
| TIC11442 | 77.6 | 63.5 | 83.2 | 62.5 | 62.5 | 60.7 | 77.8 | 80.4 | 61.5 |
| TIC11506 | 80.4 | 79.9 | 79.9 | 76.9 | 76.9 | 75.5 | 75.5 | 75.5 | 78.6 |
| TIC11513 | 80.9 | 80.4 | 80.4 | 77.4 | 77.4 | 76 | 76 | 76 | 79.2 |
| TIC11512 | 80.6 | 80 | 80 | 77.1 | 77.1 | 75.7 | 75.7 | 75.7 | 78.8 |
| TIC11210 | 82.2 | 85 | 85 | 78.7 | 78.7 | 77.3 | 77.3 | 77.3 | 84 |
| TIC11212 | 85.2 | 82 | 82 | 79.3 | 79.3 | 77.7 | 77.7 | 77.7 | 80.7 |
| TIC9319 | 79.2 | 79.6 | 79.6 | 78.5 | 78.5 | 76.7 | 76.7 | 76.7 | 78 |
| TIC10380 | 81.1 | 83.6 | 83.6 | 81.5 | 81.5 | 77.8 | 77.8 | 77.8 | 79.7 |
| TIC10380PL | 81 | 83.5 | 83.5 | 81.3 | 81.3 | 77.6 | 77.6 | 77.6 | 79.6 |
| TIC10381 | 90.7 | 91.9 | 91.9 | 88.9 | 88.9 | 82.5 | 82.5 | 82.5 | 85.7 |
| TIC10381PL | 90.3 | 91.5 | 91.5 | 88.6 | 88.6 | 82.2 | 82.2 | 82.2 | 85.4 |
| TIC9318 | 100 | 89.8 | 89.8 | 86.7 | 86.7 | 82 | 82 | 82 | 84.8 |
| TIC11445 | — | 71.9 | 86.6 | 69.4 | 69.4 | 65.6 | 80.6 | 80.3 | 67.9 |
| TIC9316 | 89.8 | — | 100 | 93.5 | 93.5 | 83.9 | 83.9 | 83.9 | 90.1 |
| TIC11441 | 86.6 | 80.1 | — | 74.8 | 74.8 | 67.2 | 84.6 | 87.1 | 72.1 |
| TIC11302 | 86.7 | 93.5 | 93.5 | — | 100 | 90.1 | 90.1 | 90.1 | 83.9 |
| TIC11443 | 69.4 | 74.8 | 74.8 | 80.1 | — | 72.1 | 72.1 | 72.1 | 67.2 |
| TIC9317 | 82 | 83.9 | 83.9 | 90.1 | 90.1 | — | 100 | 100 | 93.5 |
| TIC11446 | 80.6 | 67.2 | 84.6 | 72.1 | 72.1 | 80.1 | — | 97.5 | 74.8 |
| TIC11444 | 80.3 | 67.2 | 87.1 | 72.1 | 72.1 | 80.1 | 97.5 | — | 74.8 |
| TIC11301 | 84.8 | 90.1 | 90.1 | 83.9 | 83.9 | 93.5 | 93.5 | 93.5 | — |
| TIC10378 | 83.5 | 85.7 | 85.7 | 90.3 | 90.3 | 94.4 | 94.4 | 94.4 | 90 |
| TIC10378PL | 83.1 | 85.4 | 85.4 | 90 | 90 | 94 | 94 | 94 | 89.6 |
| TIC11103 | 63.3 | 65.7 | 65.7 | 65.7 | 65.7 | 75.1 | 75.1 | 75.1 | 75.1 |
| TIC11104 | 75.1 | 68 | 68 | 68 | 68 | 63.3 | 63.3 | 63.3 | 63.3 |
| TIC10376 | 42.8 | 42.1 | 42.1 | 41.7 | 41.7 | 41.4 | 41.4 | 41.4 | 41.7 |
| TIC10376PL | 42.7 | 42 | 42 | 41.6 | 41.6 | 41.3 | 41.3 | 41.3 | 41.6 |
| TIC10379 | 47.3 | 46.9 16 | 46.9 | 46 | 46 | 45.7 | 45.7 | 45.7 | 46.4 |
| TIC9321 | 42.5 | 40.9 | 40.9 | 41.2 | 41.2 | 41.4 | 41.4 | 41.4 | 41 |
| TIC10375 | 24 | 24.6 | 24.6 | 24 | 24 | 23.7 | 23.7 | 23.7 | 24.2 |

TABLE 11
Pair-wise matrix display of PirAB fusion proteins.

| Sequence | TIC10378 | TIC10378PL | TIC11103 | TIC11104 | TIC10376 | TIC10376PL | TIC10379 | TIC9321 | TIC10375 |
|---|---|---|---|---|---|---|---|---|---|
| TIC9322 | 49.1 | 49.1 | 37.1 | 37.7 | 41.8 | 41.8 | 43.7 | 36.8 | 23.9 |
| TIC9320 | 50 | 50 | 37.5 | 38.2 | 42.6 | 42.6 | 43.7 | 37.9 | 24.1 |
| TIC10434 | 49 | 49 | 37 | 38.2 | 41.8 | 41.8 | 43.3 | 37.3 | 23.5 |
| TIC10377 | 49.8 | 49.8 | 37.3 | 38.2 | 42.6 | 42.6 | 43.5 | 36.6 | 24.8 |
| TIC11211 | 52.5 | 52.5 | 36.6 | 37.1 | 40.9 | 40.9 | 42.5 | 37.5 | 23.4 |
| TIC6880 | 77.6 | 77.6 | 58.4 | 60.4 | 41.7 | 41.7 | 45.3 | 41.7 | 22.7 |
| TIC11440 | 62.6 | 62.6 | 47.1 | 48.7 | 33.7 | 33.7 | 36.5 | 33.7 | 18.3 |
| TIC6880PL | 77.5 | 77.5 | 58.3 | 60.3 | 41.7 | 41.7 | 45.2 | 41.7 | 22.7 |
| TIC11 42 | 62.1 | 62.1 | 46.7 | 48.3 | 33.4 | 33.4 | 36.2 | 33.4 | 18.2 |
| TIC11506 | 76.9 | 76.7 | 58 | 59.5 | 40.5 | 40.5 | 44.6 | 39.9 | 21.9 |
| TIC11513 | 77.4 | 77.2 | 58.3 | 59.9 | 41 | 41 | 45.2 | 40.3 | 22.2 |
| TIC11512 | 77.1 | 76.9 | 58 | 59.5 | 41 | 41 | 44.8 | 40.3 | 22.4 |
| TIC11210 | 78.4 | 78.2 | 58.6 | 60.2 | 41.8 | 41.8 | 45.2 | 40.5 | 24 |
| TIC11212 | 78.7 | 78.6 | 58.6 | 60.2 | 42.2 | 42.2 | 46.3 | 41.1 | 24 |
| TIC9319 | 78 | 78 | 59.2 | 60.8 | 41.7 | 41.7 | 45.5 | 41.5 | 24.6 |
| TIC10380 | 79.5 | 79.5 | 60.3 | 61.4 | 40.2 | 40.2 | 45.9 | 40.4 | 23.1 |
| TIC10380PL | 79.4 | 79.4 | 60.2 | 61.3 | 40.1 | 40.1 | 45.8 | 40.3 | 23.1 |
| TIC10381 | 84.5 | 84.3 | 64.7 | 69.5 | 41.1 | 41.1 | 45.7 | 41.3 | 23.1 |
| TIC10381PL | 84.2 | 84.5 | 64.6 | 69.4 | 41 | 41 | 45.4 | 41.2 | 23.1 |
| TIC9318 | 83.7 | 83.6 | 63.3 | 75.1 | 42.4 | 42.4 | 46.1 | 41.9 | 23 |
| TIC11445 | 67 | 66.9 | 50.6 | 60.1 | 33.9 | 33.9 | 36.9 | 33.5 | 18.4 |
| TIC9316 | 86 | 85.9 | 65.7 | 68 | 41.7 7 | 41.7 | 45.8 | 40.3 | 23.5 |
| TIC11441 | 68.9 | 68.7 | 52.6 | 54.5 | 33.4 | 33.4 | 36.6 | 32.2 | 18.8 |
| TIC11302 | 90.6 | 90.5 | 65.7 | 68 | 41.3 | 41.3 | 44.9 | 40.6 | 23 |
| TIC11443 | 72.6 | 72.4 | 52.6 | 54.5 | 33.1 | 33.1 | 35.9 | 32.5 | 18.4 |
| TIC9317 | 94.7 | 94.5 | 75.1 | 63.3 | 41 | 41 | 44.5 | 40.8 | 22.6 |
| TIC11446 | 75.8 | 75.7 | 60.1 | 50.6 | 32.8 | 32.8 | 35.6 | 32.7 | 18.1 |
| TIC11444 | 75.8 | 75.7 | 60.1 | 50.6 | 32.8 | 32.8 | 35.6 | 32.7 | 18.1 |
| TIC11301 | 90.3 | 90.1 | 75.1 | 63.3 | 41.3 | 41.3 | 45.2 | 40.5 | 23.1 |
| TIC10378 | — | 99.8 | 71.3 | 64.4 | 41.5 | 41.5 | 44.5 | 41.4 | 23.1 |
| TIC10378PL | 99.6 | — | 71.2 | 64.3 | 41.5 | 41.5 | 44.3 | 41.3 | 23 |
| TIC11103 | 71.6 | 71.6 | — | 82 | 31.6 | 31.6 | 35.7 | 30.9 | 18.4 |
| TIC11104 | 64.7 | 64.7 | 82 | — | 32.3 | 32.3 | 35.5 | 31.8 | 18.2 |
| TIC10376 | 42.1 | 42.1 | 31.9 | 32.6 | — | 99.8 | 54.9 | 34.8 | 21.6 |
| TIC10376PL | 42 | 42 | 31.9 | 32.6 | 99.6 | — | 54.8 | 34.7 | 21.5 |
| TIC10379 | 45.8 | 45.7 | 36.6 | 36.4 | 55.8 | 55.8 | — | 37.1 | 24.5 |
| TIC9321 | 42.1 | 42.1 | 31.4 | 32.3 | 34.9 | 34.9 | 36.7 | — | 22 |
| TIC10375 | 24.2 | 24.2 | 19.2 | 19 | 22.4 | 22.4 | 25 | 22.7 | — |

In addition to percent identity, The PirA Proteins, The PirB Proteins, and the PirAB Fusion Proteins can also be related by primary structure (conserved amino acid motifs), by length (about 133 to about 141 amino acids for PirA; about 414 to about 428 amino acids for PirB; about 549 to about 566 amino acids for the PirAB fusion proteins), and by other characteristics. Characteristics of The PirA Proteins, The PirB Proteins, and The PirAB Fusion Proteins are reported in Table 12.

TABLE 12
Selected characteristics of PirA proteins, PirB proteins, and PirAB proteins.

| | | | | | No. of | No. of | | |
|---|---|---|---|---|---|---|---|---|
| | | | | | Strongly | Strongly | | No. of |
| | Molecular | Amino | | Charge | Basic (—) | Acidic | No. of | Polar |
| | Weight (in | Acid | Isoelectric | at PH | Amino | Amino | Hydrophobic | Amino |
| Protein | Daltons) | Length | Point | 7.0 | Acids | Acids | Amino Acids | Acids |
| TIC4771 | 14963.57 | 135 | 5.6164 | -1.5 | 14 | 14 | 65 | 70 |
| TIC4772 | 48146.05 | 428 | 4.5643 | −16.0 | 41 | 53 | 216 | 212 |
| TIC6880 | 63092.59 | 563 | 4.6836 | −17.5 | 55 | 67 | 281 | 282 |
| TIC6880PL | 63163.67 | 564 | 4.6836 | −17.5 | 55 | 67 | 282 | 282 |
| TIC7575 | 15655.22 | 141 | 5.0636 | −2.0 | 12 | 13 | 68 | 73 |
| TIC7576 | 47775.12 | 425 | 4.7039 | −12.5 | 44 | 53 | 221 | 204 |
| TIC9316 | 63412.32 | 566 | 4.7572 | −14.5 | 56 | 66 | 289 | 277 |
| TIC7660 | 15352.83 | 141 | 4.5839 | −4.0 | 11 | 14 | 72 | 69 |
| TIC7661 | 47774.39 | 425 | 4.7572 | −5.5 | 50 | 51 | 222 | 203 |
| TIC9317 | 63109.20 | 566 | 5.1542 | −9.5 | 61 | 65 | 294 | 272 |
| TIC7662 | 15761.42 | 141 | 4.6130 | −3.5 | 11 | 14 | 68 | 73 |
| TIC7663 | 47895.35 | 425 | 5.0745 | −7.5 | 48 | 52 | 221 | 204 |
| TIC9318 | 63638.76 | 566 | 4.9378 | −11.0 | 59 | 66 | 289 | 277 |
| TIC7664 | 14950.65 | 135 | 5.0636 | −2.0 | 12 | 13 | 68 | 67 |
| TIC7665 | 46819.71 | 414 | 4.6887 | −12.5 | 43 | 52 | 214 | 200 |
| TIC9319 | 61752.34 | 549 | 4.7452 | −14.5 | 55 | 65 | 282 | 267 |
| TIC7666 | 14751.43 | 133 | 4.6137 | −4.0 | 12 | 15 | 68 | 65 |
| TIC7667 | 46246.08 | 419 | 5.4603 | −5.5 | 50 | 51 | 220 | 199 |
| TIC9322 | 60979.49 | 552 | 5.1485 | −9.5 | 62 | 66 | 288 | 264 |
| TIC7668 | 14785.54 | 133 | 5.1215 | −2.5 | 14 | 15 | 68 | 65 |
| TIC7669 | 46249.04 | 419 | 5.3001 | −6.5 | 50 | 52 | 222 | 197 |
| TIC9320 | 61016.56 | 552 | 5.2518 | −9.0 | 64 | 67 | 290 | 262 |
| TIC7939 | 15470.32 | 139 | 6.2480 | −0.5 | 15 | 14 | 70 | 69 |
| TIC7940 | 47493.26 | 419 | 4.8783 | −11.0 | 53 | 60 | 218 | 201 |
| TIC9321 | 62945.57 | 558 | 5.0432 | −11.5 | 68 | 74 | 288 | 270 |
| TIC10357 | 12838.48 | 114 | 4.7910 | −3.0 | 11 | 13 | 68 | 46 |
| TIC10366 | 47691.61 | 427 | 4.3657 | −22.0 | 43 | 62 | 221 | 206 |
| TIC10375 | 60512.07 | 541 | 4.4263 | −25.0 | 54 | 75 | 289 | 252 |
| TIC10358 | 16198.29 | 144 | 7.7512 | 1.5 | 20 | 17 | 68 | 76 |
| TIC10367 | 47147.69 | 417 | 7.7679 | 5.5 | 60 | 49 | 222 | 195 |
| TIC10376 | 63327.96 | 561 | 7.8092 | 7.0 | 80 | 66 | 290 | 271 |
| TIC10376PL | 63399.04 | 562 | 7.8092 | 7.0 | 80 | 66 | 291 | 271 |
| TIC10360 | 14976.76 | 133 | 4.8490 | −2.5 | 14 | 16 | 64 | 69 |
| TIC10369 | 46322.33 | 419 | 5.6804 | −4.0 | 50 | 50 | 224 | 195 |
| TIC10377 | 61281.07 | 552 | 5.3889 | −6.5 | 64 | 66 | 288 | 264 |
| TIC10361 | 15629.20 | 143 | 4.7632 | −3.0 | 11 | 13 | 71 | 72 |
| TIC10370 | 47710.32 | 425 | 5.0742 | −8.0 | 48 | 52 | 225 | 200 |
| TIC10378 | 63321.51 | 568 | 4.9947 | −11.0 | 59 | 65 | 296 | 272 |
| TIC10378PL | 63392.59 | 569 | 4.9947 | −11.0 | 59 | 65 | 297 | 272 |
| TIC10362 | 15173.94 | 136 | 5.1440 | −1.5 | 13 | 14 | 62 | 74 |
| TIC10371 | 46947.97 | 416 | 5.8572 | −3.5 | 47 | 46 | 218 | 198 |
| TIC10379 | 62103.90 | 552 | 5.6801 | −5.0 | 60 | 60 | 280 | 272 |
| TIC10363 | 15195.80 | 137 | 4.7774 | −3.0 | 11 | 13 | 67 | 70 |
| TIC10372 | 48400.97 | 430 | 4.7717 | −11.0 | 45 | 53 | 229 | 201 |
| TIC10380 | 63578.76 | 567 | 4.7697 | −14.0 | 56 | 66 | 296 | 271 |
| TIC10380PL | 63649.84 | 568 | 4.7697 | −14.0 | 56 | 66 | 297 | 271 |
| TIC10364 | 15833.53 | 142 | 4.4792 | −5.0 | 11 | 15 | 72 | 70 |
| TIC10373 | 47791.02 | 425 | 4.7003 | −12.5 | 44 | 53 | 220 | 205 |
| TIC10381 | 63606.54 | 567 | 4.6406 | −17.5 | 55 | 68 | 292 | 275 |
| TIC10381PL | 63677.62 | 568 | 4.6406 | −17.5 | 55 | 68 | 293 | 275 |
| TIC10359 | 14949.54 | 135 | 4.7873 | −3.5 | 12 | 14 | 68 | 67 |
| TIC10368 | 48194.25 | 429 | 4.7481 | −11.5 | 43 | 51 | 219 | 210 |
| PirA_ABE68878 | 15303.18 | 138 | 6.2470 | −5.0 | 15 | 14 | 71 | 67 |
| PirB_ABE68879 | 46424.34 | 419 | 5.2938 | −6.5 | 50 | 52 | 224 | 195 |
| TIC10434 | 61709.50 | 557 | 5.4906 | −7.0 | 65 | 66 | 295 | 262 |
| TIC11103 | 63109.20 | 566 | 5.1542 | −9.5 | 61 | 65 | 294 | 272 |
| TIC11104 | 63638.76 | 566 | 4.9378 | −11.0 | 59 | 66 | 289 | 277 |
| TIC11210 | 62456.91 | 555 | 4.7452 | −14.5 | 55 | 65 | 282 | 273 |
| TIC11211 | 61883.28 | 560 | 5.3591 | −7.5 | 62 | 64 | 288 | 272 |
| TIC11212 | 62563.12 | 555 | 4.6679 | −16.0 | 54 | 66 | 282 | 273 |
| TIC11301 | 63411.60 | 566 | 5.3685 | −7.5 | 62 | 64 | 290 | 276 |
| TIC11302 | 63109.93 | 566 | 4.6735 | −16.5 | 55 | 67 | 293 | 273 |
| TIC11440 | 78037.15 | 698 | 4.7751 | −19.0 | 69 | 81 | 346 | 352 |
| TIC11441 | 79049.52 | 707 | 4.7960 | −16.5 | 68 | 79 | 357 | 350 |
| TIC11442 | 78728.81 | 704 | 4.7287 | −19.5 | 67 | 80 | 349 | 355 |
| TIC11443 | 78747.13 | 707 | 4.7204 | −18.5 | 67 | 80 | 361 | 346 |
| TIC11444 | 78746.41 | 707 | 5.1362 | −11.5 | 73 | 78 | 362 | 345 |
| TIC11445 | 78973.57 | 707 | 4.8575 | −15.0 | 70 | 80 | 361 | 346 |
| TIC11446 | 78852.61 | 707 | 5.0123 | −13.0 | 72 | 79 | 362 | 345 |
| TIC10364 | 15833.53 | 142 | 4.4792 | −5.0 | 11 | 15 | 72 | 70 |
| TIC11505 | 48750.86 | 434 | 4.6998 | −12.5 | 43 | 52 | 221 | 213 |
| TIC11506 | 64566.38 | 576 | 4.6400 | −17.5 | 54 | 67 | 293 | 283 |
| TIC11510 | 48184.21 | 429 | 4.7481 | −11.5 | 43 | 51 | 218 | 211 |
| TIC11512 | 63999.72 | 571 | 4.6730 | −16.5 | 54 | 66 | 290 | 281 |
| TIC11511 | 48208.27 | 429 | 4.7481 | −11.5 | 43 | 51 | 219 | 210 |
| TIC11513 | 64023.79 | 571 | 4.6730 | −16.5 | 54 | 66 | 291 | 280 |

As described further in the Examples of this application, recombinant nucleic acid molecule sequences encoding The PirAB Fusion Proteins were designed for use in plants. Exemplary plant-optimized recombinant nucleic acid molecule sequences that were designed for use in plants are presented as SEQ ID NOs:49, 51, 52, 53, 54, 55, 56, 146, 148, 150, 152, 154, 156, and 158.

Expression cassettes and vectors containing these recombinant nucleic acid molecule sequences can be constructed and introduced into corn, soybean, cotton or other plant cells in accordance with transformation methods and techniques known in the art. For example, Agrobacterium-mediated transformation is described in U.S. Patent Application Publications 2009/0138985A1 (soybean), 2008/0280361A1 (soybean), 2009/0142837A1 (corn), 2008/0282432 (cotton), 2008/0256667 (cotton), 2003/0110531 (wheat), 2001/0042257 A1 (sugar beet), U.S. Pat. No. 5,750,871 (canola), 7,026,528 (wheat), and 6,365,807 (rice), and in Arencibia et al. (1998) Transgenic Res. 7:213-222 (sugarcane) all of which are incorporated herein by reference in their entirety. Transformed cells can be regenerated into transformed plants that express the PirAB fusion proteins TIC6880PL, TIC9316, TIC9317, TIC9318, TIC9319, TIC9320, or TIC9322. To test pesticidal activity, bioassays are performed in the presence of Lepidoptera pest larvae using plant leaf disks obtained from transformed plants as described in the Examples. To test pesticidal activity against Coleopteran pests, transformed plants of Rₒ and F₁ generation are used in root worm assay as described in the example below. To test pesticidal activity against Hemipteran pests, pods, corn ears or leaves of transformed plants are used in assay, either from tissue removed from the plant or remaining on the plant as described in the Examples.

As an alternative to traditional transformation methods, a DNA sequence, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. As understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

Recombinant nucleic acid molecule compositions that encode The PirA Proteins, The PirB Proteins, or the PirAB Fusion Proteins, or related insecticidal proteins are contemplated. For example, The PirA Proteins, The PirB Proteins, or The PirAB Fusion Proteins, or related insecticidal proteins can be expressed with recombinant DNA constructs in which a polynucleotide molecule with an ORF encoding the protein is operably linked to genetic expression elements such as a promoter and any other regulatory element necessary for expression in the system for which the construct is intended. Non-limiting examples include a plant-functional promoter operably linked to the PirAB fusion proteins, TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 or related family member insecticidal protein encoding sequences for expression of the protein in plants or a Bt-functional promoter operably linked to a PirA proteins such as PirA_ABE68878, or the PirB proteins TIC4772, TIC7576, TIC7661, TIC7663, TIC7665, TIC7667, TIC7669, TIC7940, TIC10366, TIC10367, TIC10369, TIC10370, TIC10371, TIC10372, TIC10373, TIC10368, PirB_ABE68879, TIC11505, TIC11510, and TIC11511; or the PirAB fusion proteins, TIC6880, TIC9316, TIC9317, TIC9318, TIC9319, TIC9322, TIC9320, TIC9321, TIC6880PL, TIC10375, TIC10376, TIC10377, TIC10378, TIC10379, TIC10380, TIC10381, TIC10434, TIC11210, TIC11211, TIC11212, TIC11301, TIC11302, TIC11440, TIC11441, TIC11442, TIC11443, TIC11444, TIC11445, TIC11446, TIC11506, TIC11512, TIC11513, TIC10376PL, TIC10378PL, TIC10380PL, TIC10381PL, TIC11103, and TIC11104; or related insecticidal protein encoding sequences for expression of the protein in a Bt bacterium or other Bacillus species. Other elements can be operably linked to the PirA protein PirA_ABE68878, the PirB proteins TIC4772, TIC7576, TIC7661, TIC7663, TIC7665, TIC7667, TIC7669, TIC7940, TIC10366, TIC10367, TIC10369, TIC10370, TIC10371, TIC10372, TIC10373, TIC10368, PirB_ABE68879, TIC11505, TIC11510, and TIC11511, or the PirAB fusion proteins, TIC6880, TIC9316, TIC9317, TIC9318, TIC9319, TIC9322, TIC9320, TIC9321, TIC6880PL, TIC10375, TIC10376, TIC10377, TIC10378, TIC10379, TIC10380, TIC10381, TIC10434, TIC11210, TIC11211, TIC11212, TIC11301, TIC11302, TIC11440, TIC11441, TIC11442, TIC11443, TIC11444, TIC11445, TIC11446, TIC11506, TIC11512, TIC11513, TIC10376PL, TIC10378PL, TIC10380PL, TIC10381PL, TIC11103, and TIC11104, or related insecticidal protein encoding sequences including, but not limited to, enhancers, introns, untranslated leaders, encoded protein immobilization tags (HIS-tag), translocation peptides (i.e., plastid transit peptides, signal peptides), polypeptide sequences for post-translational modifying enzymes, ribosomal binding sites, and RNAi target sites.

Exemplary recombinant polynucleotide molecules provided herewith include, but are not limited to, a heterologous promoter operably linked to a polynucleotide such as SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO 49, SEQ ID NO:51, SEQ ID NO:52, SEQ ID NO:53, SEQ ID NO:54, SEQ ID NO:55, SEQ ID NO:56, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:104, SEQ ID NO:106, SEQ ID NO:108, SEQ ID NO:110, SEQ ID NO:112, SEQ ID NO:114, SEQ ID NO: 116, SEQ ID NO: 118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, and SEQ ID NO:158 that encodes a polypeptide or protein having the amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO 48, SEQ ID NO:50, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO: 78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, and SEQ ID NO:157. A heterologous promoter can also be operably linked to synthetic DNA coding sequences encoding a plastid targeted TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 or untargeted TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 or related insecticidal proteins. The codons of a recombinant nucleic acid molecule encoding for proteins disclosed herein can be substituted by synonymous codons (known in the art as a silent substitution).

As used herein, the term “recombinant” refers to a non-natural DNA, protein, or organism that would not normally be found in nature and was created by human intervention. A “recombinant DNA molecule” is a DNA molecule comprising a combination of DNA molecules that would not naturally occur together and is the result of human intervention. For example, a DNA molecule that is comprised of a combination of at least two DNA molecules heterologous to each other, such as a DNA molecule that comprises a transgene and the plant genomic DNA adjacent to the transgene, is a recombinant DNA molecule.

As used herein, the term “heterologous” refers to the combination of two or more DNA molecules when such a combination is not normally found in nature. For example, the two DNA molecules may be derived from different species and/or the two DNA molecules may be derived from different genes, e.g., different genes from the same species or the same genes from different species. A regulatory element is thus heterologous with respect to an operably linked transcribable DNA molecule if such a combination is not normally found in nature, i.e., the transcribable DNA molecule does not naturally occur operably linked to the regulatory element.

A recombinant DNA construct comprising an encoding sequence for The PirA Proteins, The PirB Proteins, or The PirAB Fusion Proteins, or related insecticidal protein can further comprise a region of DNA that encodes for one or more insect inhibitory agents which can be configured to concomitantly express or co-express with a DNA sequence encoding The PirA Protein, The PirB Protein, or The PirAB Fusion Protein, or a related insecticidal protein, an insect inhibitory dsRNA molecule, or an ancillary protein. Ancillary proteins include, but are not limited to, co-factors, enzymes, binding-partners, or other agents that function to aid in the effectiveness of an insect inhibitory agent, for example, by aiding its expression, influencing its stability in plants, optimizing free energy for oligomerization, augmenting its toxicity, and increasing its spectrum of activity. An ancillary protein may facilitate the uptake of one or more insect inhibitory agents, for example, or potentiate the toxic effects of the toxic agent.

A recombinant DNA construct can be assembled so that all proteins or dsRNA molecules are expressed from one promoter or each protein or dsRNA molecules is under separate promoter control or some combination thereof. The PirA Proteins, The PirB Proteins, or the PirAB Fusion Proteins, and related proteins of this invention can be expressed from a multi-gene expression system in which one or more of The PirA Proteins, The PirB Proteins, or The PirAB Fusion Proteins, or related proteins is expressed from a common nucleotide segment which also contains other open reading frames and promoters, depending on the type of expression system selected. For example, a bacterial multi-gene expression system can utilize a single promoter to drive expression of multiply-linked/tandem open reading frames from within a single operon (i.e., polycistronic expression). In another example, a plant multi-gene expression system can utilize multiply-unlinked expression cassettes each expressing a different protein or other agent such as one or more dsRNA molecules.

Recombinant nucleic acid molecules or recombinant DNA constructs comprising The PirA Proteins, The PirB Proteins, or the PirAB Fusion Proteins, or related family member protein encoding sequence can be delivered to host cells by vectors, e.g., a plasmid, baculovirus, synthetic chromosome, virion, cosmid, phagemid, phage, or viral vector. Such vectors can be used to achieve stable or transient expression of The PirA Proteins, The PirB Proteins, or the PirAB Fusion Proteins, or related protein encoding sequences in a host cell, or subsequent expression of the encoded polypeptide. An exogenous recombinant polynucleotide or recombinant DNA construct that comprises a protein encoding sequence and that is introduced into a host cell is referred herein as a “transgene.”

Transgenic bacteria, transgenic plant cells, transgenic plants, and transgenic plant parts that contain a recombinant polynucleotide that expresses any one or more of The PirA Proteins, The PirB Proteins, or the PirAB Fusion Proteins, or related protein encoding sequences. The term “bacterial cell” or “bacterium” can include, but is not limited to, an Agrobacterium, a Bacillus, an Escherichia, a Salmonella, a Pseudomonas, or a Rhizobium cell. The term “plant cell” or “plant” can include but is not limited to a dicotyledonous cell or a monocotyledonous cell. Contemplated plants and plant cells include, but are not limited to, alfalfa, banana, barley, bean, broccoli, cabbage, brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell or plant. In other embodiments, transgenic plants and transgenic plant parts regenerated from a transgenic plant cell are provided. In certain embodiments, the transgenic plants can be obtained from a transgenic seed, by cutting, snapping, grinding or otherwise disassociating the part from the plant. In certain embodiments, the plant part can be a seed, a boll, a leaf, a flower, a stem, a root, or any portion thereof, or a non-regenerable portion of a transgenic plant part. As used in this context, a “non-regenerable” portion of a transgenic plant part is a portion that cannot be induced to form a whole plant or that cannot be induced to form a whole plant that is capable of sexual and/or asexual reproduction. In certain embodiments, a non-regenerable portion of a plant part is a portion of a transgenic seed, boll, leaf, flower, stem, or root.

Methods of making transgenic plants that comprise insect, Coleoptera- or Lepidoptera- or Hemiptera-inhibitory amounts of a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302; or related protein are provided. Such plants can be made by introducing a recombinant polynucleotide that encodes any TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302, or related protein provided in this application into a plant cell, and selecting a plant derived from said plant cell that expresses an insect, Coleoptera-, Lepidoptera-, or Hempitera-inhibitory amount of the proteins. Plants can be derived from the plant cells by regeneration, seed, pollen, or meristem transformation techniques. Methods for transforming plants are known in the art.

Processed plant products, wherein the processed product comprises a detectable amount of a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302, or related protein, an insect inhibitory segment or fragment thereof, or any distinguishing portion thereof, are also disclosed in this application. In certain embodiments, the processed product is selected from the group consisting of plant parts, plant biomass, oil, meal, sugar, animal feed, flour, flakes, bran, lint, hulls, processed seed, and seed. In certain embodiments, the processed product is non-regenerable. The plant product can comprise commodity or other products of commerce derived from a transgenic plant or transgenic plant part, where the commodity or other products can be tracked through commerce by detecting nucleotide segments or expressed RNA or proteins that encode or comprise distinguishing portions of a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302; or related protein.

Plants expressing a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302, or related protein can be crossed by breeding with transgenic events expressing other toxin proteins and/or expressing other transgenic traits such as herbicide tolerance genes, genes conferring yield or stress tolerance traits, and the like, or such traits can be combined in a single vector so that the traits are all linked.

As described further in the Examples, sequences encoding TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 were designed for use in plants. Expression cassettes and vectors containing these synthetic or artificial nucleotide sequences can be constructed and introduced into corn, cotton, and soybean plant cells in accordance with transformation methods and techniques which are known in the art. Transformed cells are regenerated into transformed plants that are observed to be expressing TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302. To test pesticidal activity, bioassays are performed in the presence of Lepidopteran, Coleopteran and Hemipteran pests.

As further described in the Examples, sequences encoding a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302, or related protein and sequences having a substantial percentage identity to these proteins can be identified using methods known to those of ordinary skill in the art such as polymerase chain reaction (PCR), thermal amplification and hybridization. For example, a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein or related proteins can be used to produce antibodies that bind specifically to related proteins, and can be used to screen for and to find other protein members that are closely related.

Furthermore, nucleotide sequences encoding a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein, or related protein can be used as probes and primers for screening to identify other members of the class using thermal-cycle or isothermal amplification and hybridization methods. For example, oligonucleotides derived from sequences as set forth as SEQ ID NO:49, SEQ ID NO:51, SEQ ID NO:52, SEQ ID NO:53, SEQ ID NO:54, SEQ ID NO:55, SEQ ID NO:56, SEQ ID NO:146, SEQ ID NO:148, SEQ ID NO:150, SEQ ID NO:152, SEQ ID NO:154, SEQ ID NO:156, and SEQ ID NO:158 can be used to determine the presence or absence of a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein, or related protein transgene in a deoxyribonucleic acid sample derived from a commodity product. Given the sensitivity of certain nucleic acid detection methods that employ oligonucleotides, it is anticipated that oligonucleotides derived from the sequences as set forth as SEQ ID NO:49, SEQ ID NO:51, SEQ ID NO:52, SEQ ID NO:53, SEQ ID NO:54, SEQ ID NO:55, SEQ ID NO:56, SEQ ID NO:146, SEQ ID NO:148, SEQ ID NO:150, SEQ ID NO:152, SEQ ID NO:154, SEQ ID NO:156, and SEQ ID NO:158 can be used to detect a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 transgene in commodity products derived from pooled sources where only a fraction of the commodity product is derived from a transgenic plant containing any of SEQ ID NO:49, SEQ ID NO:51, SEQ ID NO:52, SEQ ID NO:53, SEQ ID NO:54, SEQ ID NO:55, SEQ ID NO:56, SEQ ID NO:146, SEQ ID NO:148, SEQ ID NO:150, SEQ ID NO:152, SEQ ID NO:154, SEQ ID NO:156, or SEQ ID NO:158. It is further recognized that such oligonucleotides can be used to introduce nucleotide sequence variation in SEQ ID NO:49, SEQ ID NO:51, SEQ ID NO:52, SEQ ID NO:53, SEQ ID NO:54, SEQ ID NO:55, SEQ ID NO:56, SEQ ID NO:146, SEQ ID NO:148, SEQ ID NO:150, SEQ ID NO:152, SEQ ID NO:154, SEQ ID NO:156, or SEQ ID NO:158. Such “mutagenesis” oligonucleotides are useful for identification of TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302, or related amino acid sequence variants exhibiting a range of insect inhibitory activity or varied expression in transgenic plant host cells.

Nucleotide sequence homologs, e.g., insecticidal proteins encoded by nucleotide sequences that hybridize to each or any of the sequences disclosed in this application under hybridization conditions, are also an embodiment of the present invention. The invention also provides a method for detecting a first nucleotide sequence that hybridizes to a second nucleotide sequence, wherein the first nucleotide sequence (or its reverse complement sequence) encodes a pesticidal protein or pesticidal fragment thereof and hybridizes under stringent hybridization conditions to the second nucleotide sequence. In such case, the second nucleotide sequence can be the nucleotide sequence selected from the group consisting of: SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:52, SEQ ID NO:53, SEQ ID NO:54, SEQ ID NO:55, SEQ ID NO:56, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO: 130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, and SEQ ID NO:158 under stringent hybridization conditions. Nucleotide coding sequences hybridize to one another under appropriate hybridization conditions and the proteins encoded by these nucleotide sequences cross react with antiserum raised against any one of the other proteins. Stringent hybridization conditions, as defined herein, comprise at least hybridization at 42 °C followed by two washes for five minutes each at room temperature with 2×SSC, 0.1% SDS, followed by two washes for thirty minutes each at 65 °C in 0.5×SSC, 0.1% SDS. Washes at even higher temperatures constitute even more stringent conditions, e.g., hybridization conditions of 68 °C, followed by washing at 68 °C, in 2×SSC containing 0.1% SDS.

One skilled in the art will recognize that, due to the redundancy of the genetic code, many other sequences are capable of encoding proteins related to The PirA Proteins, The PirB Proteins, or the PirAB Fusion Proteins, and those sequences, to the extent that they function to express pesticidal proteins either in Bacillus strains or in plant cells, are embodiments of the present invention, recognizing of course that many such redundant coding sequences will not hybridize under these conditions to the native Xenorhabdus or Photorhabdus sequences encoding The PirA Proteins, The PirB Proteins, or The PirAB Fusion Proteins. This application contemplates the use of these and other identification methods known to those of ordinary skill in the art, to identify The PirA Proteins, The PirB Proteins, or The PirAB Fusion Proteins, or related protein-encoding sequences and sequences having a substantial percentage identity thereto.

Methods of controlling insects, in particular Lepidoptera, Coleoptera, or Hempiteran infestations of crop plants, with the TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein, or related proteins, are also disclosed in this application. Such methods can comprise growing a plant comprising an insect-, Coleoptera-, or Lepidoptera- or Hemiptera-inhibitory amount of a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302, or related toxin protein. In certain embodiments, such methods can further comprise any one or more of: (i) applying any composition comprising or encoding a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein, or related toxin protein to a plant or a seed that gives rise to a plant; and (ii) transforming a plant or a plant cell that gives rise to a plant with a polynucleotide encoding a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein, or related toxin protein. In general, it is contemplated that a TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 protein, or related toxin protein can be provided in a composition, provided in a microorganism, or provided in a transgenic plant to confer insect inhibitory activity against Lepidopteran, Coleopteran or Hemipteran insects.

In certain embodiments, a recombinant nucleic acid molecule of a The PirA Proteins, The PirB Proteins, or The PirAB Fusion Proteins, or related toxin protein is the insecticidally active ingredient of an insect inhibitory composition prepared by culturing recombinant Bacillus or any other recombinant bacterial cell transformed to express one of The PirA Proteins, The PirB Proteins, or The PirAB Fusion Proteins, or related toxin protein under conditions suitable for expression. Such a composition can be prepared by desiccation, lyophilization, homogenization, extraction, filtration, centrifugation, sedimentation, or concentration of a culture of such recombinant cells expressing/producing said recombinant polypeptide. Such a process can result in a Bacillus or other entomopathogenic bacterial cell extract, cell suspension, cell homogenate, cell lysate, cell supernatant, cell filtrate, or cell pellet. By obtaining the recombinant polypeptides so produced, a composition that includes the recombinant polypeptides can include bacterial cells, bacterial spores, and parasporal inclusion bodies and can be formulated for various uses, including as agricultural insect inhibitory spray products or as insect inhibitory formulations in diet bioassays.

In one embodiment, to reduce the likelihood of resistance development, an insect inhibitory composition comprising one of The PirA Proteins, The PirB Proteins, or The PirAB Fusion Proteins, or a related protein can further comprise at least one additional polypeptide known to those of ordinary skill in the art that exhibits insect inhibitory activity against the same Lepidopteran, Coleopteran, or Hemipteran insect species, but which is different from The PirA Proteins, The PirB Proteins, or The PirAB Fusion Proteins, or a related toxin protein. Possible additional polypeptides for such a composition include an insect inhibitory protein and an insect inhibitory dsRNA molecule. One example for the use of such ribonucleotide sequences to control insect pests is described in Baum, et al. (U.S. Patent Publication 2006/0021087 A1).

Such additional polypeptide for the control of Lepidopteran pests may be selected from the group consisting of an insect inhibitory protein, such as, but not limited to, Cry1A (U.S. Pat. No. 5,880,275), Cry1Ab, Cry1Ac, Cry1A.105, Cry1Ae, Cry1B (U.S. Patent Publication Ser. No. 10/525,318), Cry1C (U.S. Pat. No. 6,033,874), Cry1D, Cry1 Da and variants thereof, Cry1E, Cry1F, and Cry1A/F chimeras (U.S. Pat. Nos. 7,070,982; 6,962,705; and 6,713,063), Cry1G, Cry1H, Cry1I, Cry1J, Cry1K, Cry1L, Cry1-type chimeras such as, but not limited to, TIC836, TIC860, TIC867, TIC869, and TIC1100 (International Application Publication WO2016/061391 (A2)), TIC2160 (International Application Publication WO2016/061392(A2)), Cry2A, Cry2Ab (U.S. Pat. No. 7,064,249), Cry2Ae, Cry4B, Cry6, Cry7, Cry8, Cry9, Cry15, Cry43A, Cry43B, Cry51Aa1, ET66, TIC400, TIC800, TIC834, TIC1415, Vip3A, VIP3Ab, VIP3B, AXMI-001, AXMI-002, AXMI-030, AXMI-035, AND AXMI-045 (U.S. Patent Publication 2013-0117884 A1), AXMI-52, AXMI-58, AXMI-88, AXMI-97, AXMI-102, AXMI-112, AXMI-117, AXMI-100 (U.S. Patent Publication 2013-0310543 A1), AXMI-115, AXMI-113, AXMI-005 (U.S. Patent Publication 2013-0104259 A1), AXMI-134 (U.S. Patent Publication 2013-0167264 A1), AXMI-150 (U.S. Patent Publication 2010-0160231 A1), AXMI-184 (U.S. Patent Publication 2010-0004176 A1), AXMI-196, AXMI-204, AXMI-207, AXMI-209 (U.S. Patent Publication 2011-0030096 A1), AXMI-218, AXMI-220 (U.S. Patent Publication 2014-0245491 A1), AXMI-221z, AXMI-222z, AXMI-223z, AXMI-224z, AXMI-225z (U.S. Patent Publication 2014-0196175 A1), AXMI-238 (U.S. Patent Publication 2014-0033363 A1), AXMI-270 (U.S. Patent Publication 2014-0223598 A1), AXMI-345 (U.S. Patent Publication 2014-0373195 A1), AXMI-335 (International Application Publication WO2013/134523(A2)), DIG-3 (U.S. Patent Publication 2013-0219570 A1), DIG-5 (U.S. Patent Publication 2010-0317569 A1), DIG-11 (U.S. Patent Publication 2010-0319093 A1), AfIP-1A and derivatives thereof (U.S. Patent Publication 2014-0033361 A1), AfIP-1B and derivatives thereof (U.S. Patent Publication 2014-0033361 A1), PIP-1APIP-1B (U.S. Patent Publication 2014-0007292 A1), PSEEN3174 (U.S. Patent Publication 2014-0007292 A1), AECFG-592740 (U.S. Patent Publication 2014-0007292 A1), Pput_1063 (U.S. Patent Publication 2014-0007292 A1), DIG-657 (International Application Publication WO2015/195594(A2)), Pput_1064 (U.S. Patent Publication 2014-0007292 A1), GS-135 and derivatives thereof (U.S. Patent Publication 2012-0233726 A1), GS153 and derivatives thereof (U.S. Patent Publication 2012-0192310 A1), GS154 and derivatives thereof (U.S. Patent Publication 2012-0192310 A1), GS155 and derivatives thereof (U.S. Patent Publication 2012-0192310 A1), SEQ ID NO:2 and derivatives thereof as described in U.S. Patent Publication 2012-0167259 A1, SEQ ID NO:2 and derivatives thereof as described in U.S. Patent Publication 2012-0047606 A1, SEQ ID NO:2 and derivatives thereof as described in U.S. Patent Publication 2011-0154536 A1, SEQ ID NO:2 and derivatives thereof as described in U.S. Patent Publication 2011-0112013 A1, SEQ ID NO:2 and 4 and derivatives thereof as described in U.S. Patent Publication 2010-0192256 A1, SEQ ID NO:2 and derivatives thereof as described in U.S. Patent Publication 2010-0077507 A1, SEQ ID NO:2 and derivatives thereof as described in U.S. Patent Publication 2010-0077508 A1, SEQ ID NO:2 and derivatives thereof as described in U.S. Patent Publication 2009-0313721 A1, SEQ ID NO:2 or 4 and derivatives thereof as described in U.S. Patent Publication 2010-0269221 A1, SEQ ID NO:2 and derivatives thereof as described in U.S. Pat. No. 7,772,465 (B2), CF161_0085 and derivatives thereof as described in WO2014/008054 A2, Lepidopteran toxic proteins and their derivatives as described in US Patent Publications US2008-0172762 A1, US2011-0055968 A1, and US2012-0117690 A1; SEQ ID NO:2 and derivatives thereof as described in U.S. Pat. No. 7,510,878(B2), SEQ ID NO:2 and derivatives thereof as described in U.S. Pat. No. 7,812,129(B1), Cry71Aa1 and Cry72Aa1 (US Patent Publication US2016-0230187 A1), Axmi422 (US Patent Publication US2016-0201082 A1), Axmi440 (US Patent Publication US2016-0185830 A1), Axmi281 (US Patent Publication 2016-0177332 A1), BT-0044, BT-0051, BT-0068, BT-0128 and variants thereof (WO 2016-094159 A1), BT-009, BT-0012, BT-0013, BT-0023, BT0067 and variants thereof (WO 2016-094165 A1), Cry1JP578V, Cry1JPS1, Cry1 JPS1P578V (WO 2016-061208 A1); and the like.

Such additional polypeptide for the control of Coleopteran pests may be selected from the group consisting of an insect inhibitory protein, such as, but not limited to, Cry3Bb (U.S. Pat. No. 6,501,009), Cry1C variants, Cry3A variants, Cry3, Cry3B, Cry34/35, 5307, AXMI134 (U.S. Patent Publication 2013-0167264 A1) AXMI-184 (U.S. Patent Publication 2010-0004176 A1), AXMI-205 (U.S. Patent Publication 2014-0298538 A1), AXMI-207 (U.S. Patent Publication 2013-0303440 A1), AXMI-218, AXMI-220 (U.S. Patent Publication 20140245491A1), AXMI-221z, AXMI-223z (U.S. Patent Publication 2014-0196175 A1), AXMI-279 (U.S. Patent Publication 2014-0223599 A1), AXMI-R1 and variants thereof (U.S. Patent Publication 2010-0197592 A1, TIC407, TIC417, TIC431, TIC807, TIC853, TIC901, TIC1201, TIC3131, DIG-10 (U.S. Patent Publication 2010-0319092 A1), eHIPs (U.S. Patent Application Publication No. 2010/0017914), IP3 and variants thereof (U.S. Patent Publication 2012-0210462 A1), ω-Hexatoxin-Hv1a (U.S. Patent Application Publication 2014-0366227 A1), PHI-4 variants (U.S. Patent Application Publication 2016-0281105 A1), PIP-72 variants (WO 2016-144688 A1), PIP-45 variants, PIP-64 variants, PIP-74 variants, PIP-75 variants, and PIP-77 variants (WO 2016-144686 A1), DIG-305 (WO 2016109214 A1), PIP-47 variants (U.S. Patent Publication 2016-0186204 A1), DIG-17, DIG-90, DIG-79 (WO 2016-057123 A1), DIG-303 (WO 2016-070079 A1); and the like.

Such additional polypeptides for the control of Hemipteran pests may be selected from the group consisting of Hemipteran-active proteins such as, but not limited to, TIC1415 (US Patent Publication 2013-0097735 A1), TIC807 (U.S. Pat. No. 8,609,936), TIC852 and TIC853 (U.S. Patent Publication 2010-0064394 A1), TIC834 and variants thereof (U.S. Patent Publication 2013-0269060 A1), AXMI-036 (U.S. Patent Publication 2010-0137216 A1), and AXMI-171 (U.S. Patent Publication 2013-0055469 A1), Cry64Ba and Cry64Ca (Liu et al., (2018) Cry64Ba and Cry64Ca, Two ETX/MTX2-Type Bacillus thuringiensis Insecticidal Protein Active against Hemipteran Pests. Applied and Environmental Microbiology, 84(3): 1-11).

In other embodiments, such composition/formulation can further comprise at least one additional polypeptide that exhibits insect inhibitory activity to an insect that is not inhibited by an otherwise insect inhibitory protein of the present invention to expand the spectrum of insect inhibition obtained, e.g., an additional polypeptide that exhibits insect inhibitory activity to Thysanopterans.

The possibility for insects to develop resistance to certain insecticides has been documented in the art. One insect resistance management strategy is to employ transgenic crops that express two distinct insect inhibitory agents that operate through different modes of action. Therefore, any insects with resistance to either one of the insect inhibitory agents can be controlled by the other insect inhibitory agent. Another insect resistance management strategy employs the use of plants that are not protected to the targeted Coleopteran or Lepidopteran pest species to provide a refuge for such unprotected plants. One particular example is described in U.S. Pat. No. 6,551,962, which is incorporated by reference in its entirety.

Other embodiments such as topically applied pesticidal chemistries that are designed for controlling pests that are also controlled by the proteins disclosed herein to be used with proteins in seed treatments, spray on, drip on, or wipe on formulations can be applied directly to the soil (a soil drench), applied to growing plants expressing the proteins disclosed herein, or formulated to be applied to seed containing one or more transgenes encoding one or more of the proteins disclosed. Such formulations for use in seed treatments can be applied with various stickers and tackifiers known in the art. Such formulations can contain pesticides that are synergistic in mode of action with the proteins disclosed, so that the formulation pesticides act through a different mode of action to control the same or similar pests that can be controlled by the proteins disclosed, or that such pesticides act to control pests within a broader host range or plant pest species that are not effectively controlled by one of The PirA Proteins, The PirB Proteins, or The PirAB Fusion Proteins, or related pesticidal proteins.

The aforementioned composition/formulation can further comprise an agriculturally-acceptable carrier, such as a bait, a powder, dust, pellet, granule, spray, emulsion, a colloidal suspension, an aqueous solution, a Bacillus spore/crystal preparation, a seed treatment, a recombinant plant cell/plant tissue/seed/plant transformed to express one or more of the proteins, or bacterium transformed to express one or more of the proteins. Depending on the level of insect inhibitory or insecticidal inhibition inherent in the recombinant polypeptide and the level of formulation to be applied to a plant or diet assay, the composition/formulation can include various by weight amounts of the recombinant polypeptide, e.g. from 0.0001% to 0.001% to 0.01% to 1% to 99% by weight of the recombinant polypeptide.

### EXAMPLES

In view of the foregoing, those of skill in the art should appreciate that changes can be made in the specific aspects which are disclosed and still obtain a like or similar result without departing from the spirit and scope of the invention. Thus, specific structural and functional details disclosed herein are not to be interpreted as limiting. It should be understood that the entire disclosure of each reference cited herein is incorporated within the disclosure of this application.

### Example 1

### Discovery of the PirA Proteins and the PirB Proteins and the Construction of the PirAB Fusion Proteins

This Example describes the discovery of the pesticidal PirA proteins TIC4771, TIC7575, TIC7660, TIC7662, TIC7664, TIC7666, TIC7668, TIC7939, TIC10357, TIC10358, TIC10360, TIC10361, TIC10362, TIC10363, TIC10364, TIC10359, and PirA_ABE68878 (collectively, “The PirA Proteins”), PirB proteins TIC4772, TIC7576, TIC7661, TIC7663, TIC7665, TIC7667, TIC7669, TIC7940, TIC10366, TIC10367, TIC10369, TIC10370, TIC10371, TIC10372, TIC10373, TIC10368, PirB_ABE68879, TIC11505, TIC11510, and TIC11511 (collectively “The PirB Proteins”), and the creation of the PirAB fusion proteins, TIC6880, TIC9316, TIC9317, TIC9318, TIC9319, TIC9322, TIC9320, TIC9321, TIC6880PL, TIC10375, TIC10376, TIC10377, TIC10378, TIC10379, TIC10380, TIC10381, TIC10434, TIC11210, TIC11211, TIC11212, TIC11301, TIC11302, TIC11440, TIC11441, TIC11442, TIC11443, TIC11444, TIC11445, TIC11446, TIC11506, TIC11512, TIC11513, TIC10376PL, TIC10378PL, TIC10380PL, TIC10381PL, TIC11103, and TIC11104 (collectively, “The PirAB Fusion Proteins”).

Sequences encoding Photorabdus and Xenorabdus PirAB pesticidal proteins were identified from proprietary collections as well as public sequence information, synthesized, cloned, sequence confirmed, and tested in insect bioassay. Bacterial operons were identified from Photorabdus and Xenorabdus species, each operon comprising a PirA and a PirB coding sequence. The pesticidal PirA proteins TIC4771, TIC7575, TIC7660, TIC7662, TIC7664, TIC7666, TIC7668, TIC7939, TIC10357, TIC10358, TIC10360, TIC10361, TIC10362, TIC10363, TIC10364, TIC10359, and PirA_ABE68878; and PirB proteins TIC4772, TIC7576, TIC7661, TIC7663, TIC7665, TIC7667, TIC7669, TIC7940, TIC10366, TIC10367, TIC10369, TIC10370, TIC10371, TIC10372, TIC10373, TIC10368, PirB_ABE68879, TIC11505, TIC11510, and TIC11511, were isolated from the Photorabdus and Xenorabdus species listed in Table 13. With respect to the proteins TIC7939 and TIC7940, the operon was identified from a microbiome sample and the bacterial species from which it was derived is still unknown.

TABLE 13
Novel PirA and PirB pesticidal toxin proteins and corresponding Photorabdus and
Xenorabdus species.

| | PirA Protein | | | PirB Protein | | |
|---|---|---|---|---|---|---|
| | | | Protein | | | Protein |
| | | Nucleotide | SEQ | | Nucleotide | SEQ |
| | | SEQ ID | ID | | SEQ ID | ID |
| Bacterial Species | Toxin | NO: | NO: | Toxin | NO: | NO: |
| Xenorhabdus nematophila | TIC4771 | 1 | 2 | TIC4772 | 3 | 4 |
| ISB000002 | | | | | | |
| Xenorhabdus ehlersii | TIC7575 | 7 | 8 | TIC7576 | 9 | 10 |
| 85823 | | | | | | |
| Xenorhabdus cabanillasii | TIC7660 | 13 | 14 | TIC7661 | 15 | 16 |
| 85908 | | | | | | |
| Xenorhabdus ehlersii | TIC7662 | 19 | 20 | TIC7663 | 21 | 22 |
| 85887 | | | | | | |
| Xenorhabdus poinarii | TIC7664 | 25 | 26 | TIC7665 | 27 | 28 |
| 86198 | | | | | | |
| Photorhabdus | TIC7666 | 31 | 32 | TIC7667 | 33 | 34 |
| luminescens 86197 | | | | | | |
| Photorhabdus | TIC7668 | 37 | 38 | TIC7669 | 39 | 40 |
| luminescens 86194 | | | | | | |
| Microbiome | TIC7939 | 43 | 44 | TIC7940 | 45 | 46 |
| Shewanella violacea | TIC10357 | 57 | 58 | TIC10366 | 59 | 60 |
| DSS12 | | | | | | |
| Photorhabdus | TIC10358 | 63 | 64 | TIC10367 | 65 | 66 |
| luminescens laumondii | | | | | | |
| TTO1 | | | | | | |
| Photorhabdus | TIC10360 | 69 | 70 | TIC10369 | 71 | 72 |
| asymbiotica | | | | | | |
| Xenorhabdus sp. NBAII | TIC10361 | 75 | 76 | TIC10370 | 77 | 78 |
| XenSa04 | | | | | | |
| Yersinia aldovae 670-83 | TIC10362 | 81 | 82 | TIC10371 | 83 | 84 |
| Xenorhabdus doucetiae | TIC10363 | 87 | 88 | TIC10372 | 89 | 90 |
| FRM16 | | | | | | |
| Xenorhabdus griffiniae | TIC10364 | 93 | 94 | TIC10373 | 95 | 96 |
| BMMCB | | | | | | |
| Xenorhabdus nematophila | TIC10359 | 99 | 100 | TIC10368 | 101 | 102 |
| Photorhabdus | PirA_ABE68878 | 104 | 105 | PirB_ABE68879 | 106 | 107 |
| luminescens Hm | | | | | | |
| Xenorhabdus nematophila | | | | TIC11505 | 134 | 135 |
| MDI-0035777 | | | | | | |
| Xenorhabdus bovienii | | | | TIC11510 | 138 | 139 |
| MDI-0035808 | | | | | | |
| Xenorhabdus nematophila | | | | TIC11511 | 142 | 143 |
| AN6/1 | | | | | | |

Sequences encoding Photorabdus and Xenorabdus PirA and PirB pesticidal proteins were identified from proprietary collections as well as public sequence information, synthesized, cloned, sequence confirmed, and tested in insect bioassay. Bacterial operons were identified and polymerase chain reaction (PCR) primers were designed based upon contigs derived from sequencing of each Photorabdus and Xenorabdus species listed in Table 13. Amplicons of the full-length coding sequence for each protein toxin were produced using total DNA isolated from each species listed in Table 13. Each of the amplicons were cloned using methods known in the art into Bacillus thuringiensis (Bt) expression vectors in operable linkage with a Bt expressible promoter.

Fusion proteins comprising the PirA and PirB proteins were made using methods known in the art. The coding sequences encoding the PirAB fusion proteins comprised PirA and PirB protein coding sequences operably linked, so when expressed in a cell a protein was produced comprising the PirA and PirB proteins contiguous with each other. PirAB fusion proteins comprised of a PirA protein contiguous with a PirB protein are presented in Table 1. The PirAB fusion proteins presented in Table 1 are comprised of a PirA and PirB protein derived from the same bacterial operon, or a PirA and PirB protein derived from different bacterial operons. PirAB fusion proteins comprised of a PirB protein contiguous with a PirA protein are presented in Table 2. The PirAB fusion proteins in Table 2 are comprised of a PirA and PirB protein derived from the same bacterial operon. PirAB fusion proteins comprised of a PirA protein contiguous with another PirA protein which is in turn contiguous with a PirB protein are presented in Table 3. The PirA protein components of the PirAB fusion proteins presented in Table 3 can be duplicated PirA proteins or different PirA proteins.

### Example 2

### PirA Proteins, PirB Proteins, and PirAB Fusion Proteins Demonstrate Lepidopteran, Coleopteran, and Hemipteran Activity in Insect Bioassay

This Example illustrates inhibitory activity exhibited by The PirA Proteins, The PirB Proteins, and The PirAB Fusion Proteins against various species of Lepidoptera, Coleoptera, and Hemiptera.

The PirA Proteins, The PirB proteins, and The PirAB Fusion Proteins were expressed in Bt and E. coli and assayed for toxicity against various species of Lepidoptera, Coleoptera, Hemiptera, and Dipteran. Preparations of each toxin were assayed against the Lepidopteran pest species Fall Armyworm (Spodoptera frugiperda, FAW), Corn Earworm (Helicoverpa zea, (CEW), also known as Soybean Podworm and Cotton Bollworm), Southwestern Corn Borer (Diatraea grandiosella, SWCB), Diamondback Moth (Plutella xylostella, DBM), European Corn Borer (Ostrinia nubilalis, ECB), Velvetbean Caterpillar (Anticarsia gemmatalis, VBC), Black Cutworm (Agrotis ipsilon, BCW), Southern Armyworm (Spodoptera eridania, SAW), Soybean Looper (Pseudoplusia includes, SBL), and Tobacco Budworm (Heliothis virescens, TBW); the Coleopteran pest species Colorado potato beetle (Leptinotarsa decemlineata, CPB), Northern Corn Rootworm (Diabrotica barberi, NCR), Southern Corn Rootworm (Diabrotica undecimpunctata howardii, SCR), and Western Corn Rootworm (Diabrotica virgifera, WCR); the Hemipteran species Southern Green Stink Bug (Nezara viridula, SG), Neotropical Brown Stink Bug (Euschistus heros, NBSB), Tarnished plant bug (Lygus lineolaris, TPB), and Western tarnished plant bug (Lygus Hesperus, WTP); and the Dipteran species Yellow Fever Mosquito (Aedes aegypti, YFM).

Transformed Bt and E. coli expressing The PirA Proteins, The PirB Proteins, and The PirAB Fusion Proteins were grown and spores or solubilized proteins were added to the insect diet for assay. Mortality and stunting were evaluated by comparing the growth and development of insects on a diet containing one or more of The PirA Proteins, The PirB Proteins, and The PirAB Fusion Proteins, to insects on a diet with an untreated control culture. Activity was observed for Lepidopteran, Coleopteran, Hemipteran, and Dipteran insect pests. The bioassay activity observed for each protein is presented in Tables 14 (Lepidopteran) and 15 (Coleopteran, Hemipteran, and Dipteran), wherein “+” indicates activity, an empty cell indicates no activity observed, and “NT” indicates the toxin was not assayed against that specific insect pest.

TABLE 14
Bioassay activity of PirA Proteins, PirB Proteins, and PirAB Fusion proteins,
against Lepidopteran insect pests.

| Type | Toxin | FAW | CEW | SWCB | DBM | ECB | VBC | BCW | SAW | SBL | TBW |
|---|---|---|---|---|---|---|---|---|---|---|---|
| PirA | TIC4771 | | + | | + | + | + | | + | | |
| PirB | TIC4772 | | + | | + | | + | | | | |
| Fusion | TIC6880 | + | + | + | + | + | + | | | | |
| PirA | TIC7575 | NT | NT | | NT | | | | | | |
| PirB | TIC7576 | NT | NT | | NT | | | | | | |
| Fusion | TIC9316 | NT | | + | NT | + | + | + | + | | + |
| PirA | TIC7660 | NT | NT | | NT | | | | | | |
| PirB | TIC7661 | NT | NT | | NT | | | | | | |
| Fusion | TIC9317 | NT | NT | + | NT | + | + | | | | |
| PirA | TIC7662 | NT | NT | | NT | | | | | | |
| PirB | TIC7663 | NT | NT | | NT | | | | | | |
| Fusion | TIC9318 | NT | NT | + | NT | + | + | + | | | + |
| PirA | TIC7664 | NT | NT | | NT | | | | | | |
| PirB | TIC7665 | NT | NT | | NT | | | | | | + |
| Fusion | TIC9319 | NT | NT | + | NT | + | + | + | | | |
| PirA | TIC7666 | NT | NT | | NT | | | | | | |
| PirB | TIC7667 | NT | NT | + | NT | | | | | | |
| Fusion | TIC9322 | + | + | + | NT | | + | | | | |
| PirA | TIC7668 | NT | NT | | NT | | | | | | |
| PirB | TIC7669 | NT | NT | | NT | | | | | | |
| Fusion | TIC9320 | NT | NT | + | NT | + | + | | | | |
| Fusion | TIC9321 | NT | NT | | NT | | | | | | |
| PirA | TIC10357 | NT | NT | | NT | NT | NT | | | | NT |
| PirB | TIC10366 | NT | NT | | NT | NT | NT | | | | NT |
| Fusion | TIC10375 | NT | NT | | NT | NT | NT | | | | NT |
| PirA | TIC10358 | NT | NT | | NT | NT | NT | | | | NT |
| PirB | TIC10367 | NT | NT | | NT | NT | NT | | | | NT |
| Fusion | TIC10376 | NT | NT | + | NT | NT | NT | | | | NT |
| PirA | TIC10360 | NT | NT | | NT | NT | NT | | | | NT |
| PirB | TIC10369 | NT | NT | | NT | NT | NT | | | | NT |
| Fusion | TIC10377 | NT | NT | | NT | NT | NT | | | | NT |
| PirA | TIC10361 | NT | NT | | NT | NT | NT | | | | NT |
| PirB | TIC10370 | NT | NT | | NT | NT | NT | | | | NT |
| Fusion | TIC10378 | NT | NT | + | NT | NT | NT | | | | NT |
| PirA | TIC10362 | NT | NT | | NT | NT | NT | | | | NT |
| PirB | TIC10371 | NT | NT | | NT | NT | NT | | | | NT |
| Fusion | TIC10379 | NT | | + | NT | NT | NT | | | | NT |
| PirA | TIC10363 | NT | NT | | NT | NT | NT | | | | NT |
| PirB | TIC10372 | NT | NT | + | NT | NT | NT | | | | NT |
| Fusion | TIC10380 | + | NT | | NT | NT | NT | | | | NT |
| PirA | TIC10364 | NT | NT | | NT | NT | NT | | | | NT |
| PirB | TIC10373 | NT | NT | | NT | NT | NT | | | | NT |
| Fusion | TIC10381 | | NT | | NT | NT | NT | | | | NT |
| PirB | TIC10368 | NT | NT | | NT | NT | NT | | | | NT |
| Fusion | TIC10434 | | NT | | NT | NT | NT | | | | NT |
| Fusion | TIC11103 | NT | NT | + | NT | NT | NT | NT | NT | NT | NT |
| Fusion | TIC11104 | NT | NT | | NT | NT | NT | NT | NT | NT | NT |
| Fusion | TIC11210 | NT | NT | + | NT | NT | NT | + | | | NT |
| Fusion | TIC11211 | NT | NT | + | NT | NT | NT | NT | NT | NT | NT |
| Fusion | TIC11212 | NT | NT | + | NT | NT | NT | | | | NT |
| Fusion | TIC11301 | | NT | + | NT | + | + | | NT | NT | NT |
| Fusion | TIC11302 | | NT | + | NT | + | + | | NT | NT | NT |

TABLE 15
Bioassay activity of PirA Proteins, PirB Proteins, and PirAB Fusion Proteins
against Coleopteran, Hemipteran, and Dipteran insect pests.

| | | Coleopteran | | | | Hemipteran | | | | Dipteran |
|---|---|---|---|---|---|---|---|---|---|---|
| Type | Toxin | CPB | NCR | SCR | WCR | SGB | NBSB | TPB | WTP | YFM |
| PirA | TIC4771 | + | NT | NT | NT | NT | | + | | NT |
| PirB | TIC4772 | | NT | NT | | NT | | + | | NT |
| Fusion | TIC6880 | + | NT | | + | + | + | + | + | + |
| PirA | TIC7575 | | NT | NT | | NT | NT | | | NT |
| PirB | TIC7576 | | NT | NT | NT | NT | NT | | | |
| Fusion | TIC9316 | + | NT | NT | | + | + | + | + | NT |
| PirA | TIC7660 | | NT | NT | NT | NT | NT | | | NT |
| PirB | TIC7661 | | NT | NT | NT | NT | NT | | | NT |
| Fusion | TIC9317 | + | | | + | + | | + | + | NT |
| PirA | TIC7662 | | NT | NT | | NT | NT | | | NT |
| PirB | TIC7663 | | NT | NT | NT | NT | NT | | | NT |
| Fusion | TIC9318 | + | | | + | + | + | + | + | NT |
| PirA | TIC7664 | + | NT | NT | NT | NT | NT | | | NT |
| PirB | TIC7665 | | NT | NT | NT | NT | NT | | | NT |
| Fusion | TIC9319 | + | NT | NT | | + | | + | + | NT |
| PirA | TIC7666 | | NT | NT | | NT | NT | | | NT |
| PirB | TIC7667 | | NT | NT | NT | NT | NT | | | NT |
| Fusion | TIC9322 | + | NT | NT | | | | + | | NT |
| PirA | TIC7668 | | NT | NT | NT | NT | NT | | | NT |
| PirB | TIC7669 | | NT | NT | NT | NT | NT | | | NT |
| Fusion | TIC9320 | + | NT | NT | | + | + | + | | NT |
| Fusion | TIC9321 | + | NT | NT | | NT | NT | | | NT |
| PirA | TIC10357 | NT | NT | NT | NT | NT | NT | NT | | NT |
| PirB | TIC10366 | NT | NT | NT | NT | NT | NT | NT | | NT |
| Fusion | TIC10375 | NT | NT | NT | NT | NT | NT | NT | | NT |
| PirA | TIC10358 | NT | NT | NT | NT | NT | NT | NT | | NT |
| PirB | TIC10367 | NT | NT | NT | NT | NT | NT | NT | | NT |
| Fusion | TIC10376 | NT | + | NT | + | NT | | NT | | NT |
| PirA | TIC10360 | NT | NT | NT | NT | NT | NT | NT | | NT |
| PirB | TIC10369 | NT | NT | NT | NT | NT | NT | NT | | NT |
| Fusion | TIC10377 | NT | NT | NT | NT | NT | NT | NT | | NT |
| PirA | TIC10361 | NT | NT | NT | NT | NT | NT | NT | | NT |
| PirB | TIC10370 | NT | NT | NT | NT | NT | NT | NT | | NT |
| Fusion | TIC10378 | NT | + | NT | + | NT | + | NT | | NT |
| PirA | TIC10362 | NT | NT | NT | NT | NT | NT | NT | | NT |
| PirB | TIC10371 | NT | NT | NT | NT | NT | NT | NT | | NT |
| Fusion | TIC10379 | NT | + | NT | + | NT | NT | NT | | NT |
| PirA | TIC10363 | NT | NT | NT | NT | NT | NT | NT | | NT |
| PirB | TIC10372 | NT | NT | NT | NT | NT | NT | NT | | NT |
| Fusion | TIC10380 | NT | + | NT | + | NT | + | NT | NT | NT |
| PirA | TIC10364 | NT | NT | NT | NT | NT | NT | NT | | NT |
| PirB | TIC10373 | NT | NT | NT | NT | NT | NT | NT | | NT |
| Fusion | TIC10381 | NT | + | NT | + | NT | + | NT | | NT |
| PirB | TIC10368 | NT | NT | NT | NT | NT | NT | NT | | NT |
| Fusion | TIC10434 | NT | + | NT | + | NT | | NT | NT | NT |
| Fusion | TIC11103 | NT | NT | NT | NT | NT | | NT | | NT |
| Fusion | TIC11104 | NT | NT | NT | NT | NT | + | NT | NT | NT |
| Fusion | TIC11210 | NT | NT | NT | NT | NT | + | NT | | NT |
| Fusion | TIC11211 | NT | NT | NT | NT | NT | + | NT | | NT |
| Fusion | TIC11212 | NT | NT | NT | NT | NT | | NT | NT | NT |
| Fusion | TIC11301 | NT | + | NT | + | NT | + | NT | + | NT |
| Fusion | TIC11302 | NT | NT | NT | + | NT | + | NT | + | NT |

As can be seen in Tables 14 and 15, the PirA and PirB toxin proteins alone in most instances did not demonstrate insecticidal activity. However, the fusion proteins comprising the PirA and PirB protein from each operon showed a wide spectrum of activity against Lepidopteran, Coleopteran, Hemipteran, and Dipteran insect pest species. Some of the PirA and PirB proteins did demonstrate oral activity when tested in their individual capacity. For example the PirA protein TIC4771 demonstrated activity against the Lepidopteran species CEW, DBM, ECB, VBC, SAW, the Coleopteran species CPB, and the Hemipteran species TPB. The PirB Protein TIC4772 demonstrated activity against the Lepidopteran species CEW, DBM, VBC, SAW, and the Hemipteran species TPB. When the TIC4771 and TIC4772 proteins were combined into the PirAB fusion protein TIC6880 most of the activity against the Lepidopteran activity was retained (CEW, DBM, ECB, and VBC). Activity against SAW was lost, but activity against two more insect species FAW and SWCB was seen. TIC6880 also demonstrated additional activity relative to the individual TIC4471 and TIC4772 with respect to Coleopteran and Hemipteran insect pest species. With respect to Coleoptera, TIC6880 retained activity against CPB, and added activity against WCR. With respect to Hemiptera, TIC6880 retained activity against TPB and added observed activity against SGB, NBSB, and WTP. TIC6880 also demonstrated activity against the Dipteran species YFM, which was not seen by TIC4771 or TIC4772.

While the PirA and PirB proteins TIC7575 and TIC7576, respectively, did not demonstrate insect inhibitory activity against the insects assayed, the corresponding PirAB fusion protein, TIC9316 demonstrated activity against the Lepidopteran species SWCB, ECB, VBC, BCW, SAW, and TBW. TIC9316 also demonstrated activity against the Coleopteran species CPB and the Hemipteran species SGB, NBSB, TPB, and WTP.

The PirA and PirB proteins TIC7660 and TIC7661, respectively did not demonstrate activity. However, the corresponding PirAB fusion protein TIC9317 demonstrated activity against the Lepidopteran species SWCB, ECB, and VBC, the Coleopteran species CPB and WCR, and the Hemipteran species SGB, TPB, and WTP.

The PirA and PirB proteins TIC7662 and TIC7663, respectively, did not demonstrate activity. However, the corresponding PirAB fusion protein TIC9318 demonstrated activity against the Lepidopteran species SWCB, ECB, VBC, BCW, and TBW, the Coleopteran species CPB and WCR, and the Hemipteran species SGB, NBSB, TPB, and WTP.

The PirA protein TIC7664 demonstrated activity against the Coleopteran species CPB. The PirB protein TIC7665 demonstrated activity against the Lepidopteran species TBW. The corresponding PirAB fusion protein TIC9319 demonstrated activity against the Lepidopteran species SWCB, ECB, VBC, and BCW, the Coleopteran species CPB and WCR, and the Hemipteran species SGB, TPB, and WTP.

The PirA protein TIC7666 did not demonstrate insect inhibitory activity. The PirB protein TIC7667 demonstrated activity against the Lepidopteran species SWCB. The corresponding PirAB fusion protein TIC9322 demonstrated activity against the Lepidopteran species FAW, CEW, SWCB and VBC, the Coleopteran species CPB, and the Hemipteran species TPB.

The PirA and PirB proteins TIC7668 and TIC7669, respectively, did not demonstrate activity. The corresponding PirAB fusion protein TIC9320 demonstrated activity against the Lepidopteran species SWCB, ECB, and VBC, the Coleopteran species CPB, and the Hemipteran species SGB, NBSB, and TPB.

The PirAB fusion protein TIC9321 demonstrated activity against the Coleopteran pest CPB.

The PirA protein TIC10357, PirB protein TIC10366, and the corresponding PirAB fusion protein TIC10375 did not demonstrate activity against the limited number of Lepidopteran assayed.

The PirA protein TIC10358 and PirB protein TIC10367, respectively, did not show activity against the insect species assayed. However, the corresponding PirAB fusion protein TIC10376 demonstrated activity against the Lepidopteran insect pest species, SWCB and the Coleopteran insect pest species, NCR and WCR.

The PirA protein TIC10360 and the PirB protein TIC10369, respectively, did not demonstrate activity against the limited number of Lepidopteran insect pest species assayed.

The PirA protein TIC10361 and PirB protein TIC10370, respectively, did not demonstrate activity against the limited number of Lepidopteran insect pest species assayed. However, the corresponding fusion protein TIC10378 demonstrated activity against the Lepidopteran insect pest species, SWCB, the Coleopteran pest species, NCR and WCR, and the Hemipteran pest species, NBSB.

The PirA protein TIC10362 and the PirB protein TIC10371 did not demonstrate activity against the limited number of Lepidopteran insect pest species assayed.

The PirA protein TIC10363 did not demonstrate activity against the limited number of insect pest species assayed. The PirB protein TIC10372 demonstrated activity against the Lepidopteran insect species SWCB. The corresponding fusion protein TIC10380 retained activity against the Lepidopteran insect pest species, SWCB and added activity against the Coleopteran pest species NCR and WCR and the Hemipteran pest species NBSB.

The PirA protein TIC10364 and PirB protein TIC10373 did not demonstrate activity against the limited number of insect pest species assayed. The corresponding fusion protein TIC10381 demonstrated activity against the Coleopteran pest species, NCR and WCR, and the Hemipteran pest species NBSB.

The PirAB fusion protein TIC10434 demonstrated activity against the Coleopteran pest species NCR and WCR.

The PirAB fusion protein TIC11103 demonstrated activity against the Lepidopteran pest species SWCB.

The PirAB fusion protein TIC1104 demonstrated activity against the Hemipteran species NBSB.

The PirAB fusion protein TIC11210 demonstrated activity against the Lepidopteran pest species SWCB and BCW and the Hemipteran pest species NBSB.

The PirAB fusion protein TIC11211 demonstrated activity against the Lepidopteran pest species SWCB and the Hemipteran species NBSB.

The PirAB fusion protein TIC11212 demonstrated activity against the Lepidopteran insect pest species SWCB.

The PirAB fusion protein TIC11301 demonstrated activity against the Lepidopteran pest species SWCB, ECB, and VBC, the Coleopteran pest species NCR and WCR, and the Hemipteran pest species NBSB and WTP.

The PirAB fusion protein TIC11302 demonstrated activity against the Lepidopteran pest species SWCB, ECB, and VBC, the Coleopteran pest species WCR, and the Hemipteran pest species NBS and WTP.

### Example 3

### Mixtures of the PirA, PirB, and PirAB Fusion Proteins Demonstrate Lepidopteran, Coleopteran, and Hemipteran Activity in Insect Bioassay

This Example illustrates inhibitory activity exhibited by mixing the PirA proteins TIC7575 and TIC7660 with the PirB proteins TIC7576 and TIC7661, as well as by mixing the PirAB fusion proteins TIC9316 and TIC9317; TIC9316 with TIC11301; and TIC9317 and TIC11302 in various concentrations.

Mixtures of the PirA and PirB proteins and mixtures of the PirAB fusion proteins at various concentrations were presented in insect diet and assayed for activity against the Lepidopteran pest species BCW, SWC, and VBC, the Coleopteran pest species WCR and NCR, and the Hemipteran pest species NBSB. The mixtures comprised different concentrations of the toxin proteins. Table 16 below shows the insect species in which activity was observed for each mixture.

TABLE 16
Bioassay activity of mixtures of the PirA and PirB toxin
proteins and mixtures of the PirAB fusion proteins.

| Mixture | Activity |
|---|---|
| TIC7575 0.0625 mg/mL; TIC7576 0.1875 mg/mL | BCW; VBC; NBSB |
| TIC7575 0.0625 mg/mL; TIC7576 0.1875 mg/mL; | NCR; WCR; NBSB |
| double for .5 mg/mL final | |
| TIC7575 0.0625 mg/mL; TIC7661 0.1875 mg/mL; | NCR; WCR |
| double for .5 mg/mL final | |
| TIC7660 0.0625 mg/mL; TIC7576 0.1875 mg/mL; | NCR; WCR |
| double for .5 mg/mL final | |
| TIC7660 0.0625 mg/mL; TIC7661 0.1875 mg/mL; | NCR; WCR |
| double for .5 mg/mL final | |
| TIC7575 0.0625 mg/mL; TIC7576 0.125 mg/mL; | WCR; VBC |
| TIC7660 0.0625 mg/mL | |
| TIC7575 0.0625 mg/ml; TIC7660 0.0625 mg/mL; | WCR |
| TIC7661 0.125 mg/mL | |
| TIC9316 0.04 mg/mL; TIC9317 0.01 mg/mL | VBC |
| TIC9316 0.025 mg/mL; TIC9317 0.025 mg/mL | VBC |
| TIC9316 0.25 mg/ml; TIC9317 0.25 mg/mL | NBS |
| TIC9316 0.4 mg/mL; TIC9317 0.1 mg/mL | NBS |
| TIC9316 0.04 mg/mL; TIC11301 0.01 mg/mL | VBC; SWC |
| TIC9316 0.025 mg/mL; TIC11301 0.025 mg/mL | VBC |
| TIC9316 0.01 mg/mL; TIC11301 0.04 mg/mL | VBC |
| TIC9316 0.1 mg/mL; TIC11301 0.4 mg/mL | NBS |
| TIC9316 0.4 mg/mL; TIC11301 0.1 mg/mL | VBC; NBSB |
| TIC9317 0.01 mg/mL; TIC11302 0.04 mg/mL | VBC |
| TIC9317 0.25 mg/mL; TIC11302 0.25 mg/mL | NBS |

As can be seen in Table 16, mixtures of the PirA proteins TIC7575 and TIC7660 with the PirB proteins TIC7576 and TIC7661 provided activity similar to corresponding the corresponding fusion proteins, TIC9316 and TIC9317. Mixtures of the PirAB fusion proteins TIC9316 and TIC9317; TIC9316 with TIC11301; and TIC9317 and TIC11302 demonstrated activity similar to one or both of the fusion proteins.

### Example 4

### Design of Synthetic Coding Sequences Encoding the PirAB Fusion Proteins TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 for Expression in Plant Cells

Synthetic or artificial coding sequences were constructed for use in expression of the PirAB fusion proteins TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 in plants. These synthetic coding sequences were cloned into a binary plant transformation vectors and used to transform plant cells. The synthetic nucleic acid sequences were synthesized according to methods generally described in U.S. Pat. No. 5,500,365, avoiding certain inimical problem sequences such as ATTTA and A/T rich plant polyadenylation sequences while preserving the amino acid sequence of the PirAB Fusion protein. The synthetic coding sequence for the PirAB fusion proteins TIC6880PL, TIC9316, TIC9317, TIC9318, TIC9319, TIC9320, TIC9322, TIC10376PL, TIC10378PL, TIC10380PL, TIC10381PL, and TIC11302 are presented in Table 17. The coding sequences encoding TIC6880PL, TIC10376PL, TIC10378PL, TIC10380PL, and TIC10381PL contained an additional alanine codon immediately following the initiating methionine residue of the corresponding PirA coding sequences portion within the PirAB fusion protein coding sequence.

TABLE 17
Synthetic coding sequences used for expression in plant
cells encoding PirAB fusion proteins TIC6880PL, TIC9316,
TIC9317, TIC9318, TIC9319, TIC9320, TIC9322, TIC10376PL,
TIC10378PL, TIC10380PL, TIC10381PL, and TIC11302.

| | Nucleotide | Protein | | |
|---|---|---|---|---|
| PirAB Fusion | SEQ ID | SEQ ID | | |
| Protein | NO: | NO: | PirA Protein | PirB Protein |
| TIC6880PL* | 49 | 50 | TIC4771* | TIC4772 |
| TIC9316 | 51 | 12 | TIC7575 | TIC7576 |
| TIC9317 | 52 | 18 | TIC7660 | TIC7661 |
| TIC9318 | 53 | 24 | TIC7662 | TIC7663 |
| TIC9319 | 54 | 30 | TIC7664 | TIC7665 |
| TIC9320 | 55 | 42 | TIC7668 | TIC7669 |
| TIC9322 | 56 | 36 | TIC7666 | TIC7667 |
| TIC10376PL* | 146 | 147 | TIC10358* | TIC10367 |
| TIC10378PL* | 148 | 149 | TIC10361* | TIC10370 |
| TIC10380PL* | 150 | 151 | TIC10363* | TIC10372 |
| TIC10381PL* | 152 | 153 | TIC10364* | TIC10373 |
| TIC11302 | 158 | 119 | TIC7660 | TIC7576 |

*comprises an additional alanine residue immediately following the initiating methionine residue.

The synthetic coding sequences and corresponding protein sequences for TIC11103 and TIC11104 are presented in Table 18 below.

TABLE 18
Synthetic coding sequences used for expression in plant cells
encoding PirAB fusion proteins TIC11103 and TIC11104.

| | Nucleotide | Protein | | |
|---|---|---|---|---|
| PirAB Fusion | SEQ ID | SEQ ID | | |
| Protein | NO: | NO: | PirB Protein | PirA Protein |
| TIC11103 | 154 | 155 | TIC7661 | TIC7660 |
| TIC11104 | 156 | 157 | TIC7663 | TIC7662 |

### Example 5

### Expression Cassettes for Expression of PirAB Fusion Proteins in Plant Cells

A variety of plant expression cassettes were designed with the sequences as set forth in Table 17. Such expression cassettes are useful for transient expression in plant protoplasts or transformation of plant cells. Typical expression cassettes are designed with respect to the eventual placement of the protein within the plant cell. For a plastid targeted protein, the synthetic TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 pesticidal protein coding sequences are operably linked in frame with a chloroplast targeting signal peptide coding sequence. The resulting plant transformation vectors comprise a first transgene cassette for expression of the pesticidal protein which comprises a constitutive promoter, operably linked 5′ to a leader, operably linked 5′ to an intron (or optionally no intron), operably linked 5′ to a synthetic coding sequence encoding a plastid targeted or untargeted TIC6880PL, TIC9316, TIC9317, TIC9318, TIC9319, TIC9320, TIC9322, TIC10376PL, TIC10378PL, TIC10380PL, TIC10381PL, TIC11103, TIC11104, and TIC11302 protein, which is in turn operably linked 5′ to a 3′ UTR and, a second transgene cassette for the selection of transformed plant cells using glyphosate or antibiotic selection. All of the elements described above are arranged contiguously often with additional sequence provided for the construction of the expression cassette such as restriction endonuclease sites or ligation independent cloning sites.

### Example 6

### Transgenic Corn Plants Expressing TIC9316, TIC9317, TIC9318, TIC10376, TIC10378, TIC10380, and TIC10381 Demonstrate Activity Against Lepidopteran Pest Species

This Example illustrates the inhibitory activity of the PirAB fusion proteins TIC9316, TIC9317, TIC9318, TIC10378, TIC10380, and TIC10381 when expressed in transgenic corn plants and assayed against Lepidopteran insect pest species.

Binary plant transformation vectors comprising transgene cassettes designed to express TIC9316, TIC9317, TIC9318, TIC10376, TIC10378, TIC10380, or TIC10381 were cloned using methods known in the art. The plant transformation vector comprised a first transgene cassette for expression of the TIC9316, TIC9317, TIC9318, TIC10376, TIC10378, TIC10380, or TIC10381 pesticidal protein which comprised a plant expressible promoter, operably linked 5′ to a leader, operably linked 5′ to an intron, operably linked 5′ to a synthetic coding sequence encoding TIC9316, TIC9317, TIC9318, TIC10376, TIC10378, TIC10380, or TIC10381, operably linked 5′ to a 3′ UTR and, a second transgene cassette for the selection of transformed plant cells using glyphosate. The resulting vectors were used to stably transform corn plants using an Agrobacterium-mediated transformation method. The transformed cells were induced to form plants by methods known in the art. Bioassays using plant leaf disks were performed analogous to those described in U.S. Pat. No. 8,344,207. A non-transformed corn plant was used to obtain tissue to be used as a negative control. Multiple transformation events from each binary vector were assessed against the Lepidopteran pest species FAW, CEW, SWCB, ECB, and BCW.

Several transformed events expressing TIC9316 demonstrated good to moderate inhibitory activity against SWCB and ECB. Likewise, transformed events expressing TIC9317 also demonstrated good to moderate inhibitory activity against SWCB and ECB. Transformed events expressing TIC9318 demonstrated good to excellent inhibitory activity against SWCB and ECB. Transformed events expressing TIC10376, TIC10378, TIC10380, and TIC10381 demonstrated good to moderate activity against SWCB.

### Example 7

Assay of Activity of PirAB Fusion Proteins Against Coleopteran Corn Rootworm Pests when Expressed in Stably Transformed Corn Plants

This Example illustrates the inhibitory activity of TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 against different Coleopteran species that feed on corn roots.

Binary plant transformation vectors comprising transgene cassettes designed to express both plastid targeted and untargeted TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 are cloned using methods known in the art and comprise the sequences as shown in Tables 17 and 18. The resulting vectors are used to stably transform corn plants using methods known in the art. Single T-DNA insertion events are selected and grown. Pesticidal activity is assayed against the Coleopteran pests NCR, SCR, and WCR feeding on the roots of the stably transformed corn plants.

R₀ stably transformed plants are used to assay for Coleopteran resistance as well as generating F₁ progeny. Multiple single copy events are selected from each binary vector transformation. A portion of the events arising from each binary vector transformation are used in the R₀ Coleopteran assay, while another portion of events are used to generate F₁ progeny for further testing.

The R₀ assay plants are transplanted to eight inch pots. The plants are inoculated with eggs from WCR, NCR, or SCR. The eggs are incubated for approximately ten (10) days prior to inoculation to allow hatching to occur four (4) days after inoculation to ensure a sufficient number of larvae survive and are able to attack the corn roots. The transformed plants are inoculated at approximately V2 to V3 stage. The plants are grown after infestation for approximately twenty-eight (28) days. The plants are removed from the pots with the roots being carefully washed to remove all soil. The damage to the roots is assessed using a damage rating scale of 1-5, as presented in Table 19. Comparison is also made to the negative controls to assure the assay has been performed properly. Multiple R₀ events for each binary vector transformation are used in the Coleopteran assay. Low root damage scores indicate resistance conferred by the tested PirAB fusion protein to the tested Coleopteran pest.

TABLE 19
R₀ root damage rating scores.

| Root Damage Score | Description |
|---|---|
| 1 | No visible feeding |
| 2 | Some feeding; no pruning |
| 3 | Pruning of at least one root |
| 4 | Entire node pruned |
| 5 | More than one node pruned |

A portion of the R₀ stably transformed events arising from each binary vector transformation are used to produce F₁ progeny. The R₀ stably transformed plants are allowed to self-fertilize, producing F₁ progeny. The F₁ seed is planted. Heterozygous plants are identified through molecular methods known in the art and used for assay against Coleopteran pests, as well as ELISA expression measurements of toxin protein. A portion of the heterozygous F₁ progeny from each event are used for insect assay, while another portion is used to measure toxin protein expression.

Eggs from WCR, NCR, or SCR are incubated for approximately ten (10) days to allow hatching within four (4) days after inoculation. For WCR, each pot is inoculated with about two thousand eggs. For NCR, less eggs may be used due to the lower availability of eggs from this species. The plants are inoculated at approximately V2 to V3 stage. The plants are grown after infestation for approximately twenty-eight (28) days. The plants are removed from the pots with the roots being carefully washed to remove all soil. The damage to the roots are assessed using a damage rating scale of 0-3, as presented in Table 20. Comparison is made to the negative control to assure the assay has been performed properly. Low root damage scores indicate resistance conferred by TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302 to the Coleopteran pest.

TABLE 20
F₁ root damage rating scores.

| Root Damage | |
|---|---|
| Score | Description |
| 0 | No visible feeding |
| 0.01-0.09 | Feeding scars and tracks |
| 0.1-0.9 | Root pruning, but less than a full node |
| 1.0-1.9 | At least a full node (or equivalent) |
| | destroyed to within 1.5 inches of plant |
| 2.0-2.9 | Two or more nodes gone |
| 3 | Three or more nodes gone |

### Example 8

Assay of Activity of the PirAB Fusion Proteins Against Lepidopteran Pests when Expressed in Stably Transformed Corn, Soybean or Cotton Plants.

This Example illustrates the assay of activity against various Lepidopteran pest species fed tissue from stably transformed corn, soybean or cotton plants expressing one of The PirAB Fusion Proteins.

Binary plant transformation vectors comprising transgene cassettes designed to express both plastid targeted and untargeted versions of The PirAB Fusion Proteins are cloned using methods known in the art and comprise the coding sequences as presented in Tables 17 and 18.

Corn, soybean, or cotton is transformed with the binary transformation vectors described above using an Agrobacterium-mediated transformation method. The transformed cells are induced to form plants by methods known in the art. Bioassays using plant leaf disks are performed analogous to those described in U.S. Pat. No. 8,344,207. A non-transformed corn, soybean, or cotton plant is used to obtain tissue to be used as a negative control. Multiple transformation events from each binary vector are assessed against Lepidopteran pests such as, but not limited to, BCW, CEW, DBM, ECB, FAW, SAW, SBL, SWCB, TBW, and VBC. Those insects demonstrating stunting and/or mortality in the insect bioassay are determined to be susceptible to the effects of The PirAB Fusion Proteins tested.

### Example 9

Assay of Activity of the PirAB Fusion Proteins Against Flea Beetle Pests when Expressed in Stably Transformed Canola Plants

This Example illustrates the assay of activity of against various species of Flea Beetle when allowed to feed on whole transgenic canola plants or tissues derived from transgenic canola plants expressing one of The PirAB Fusion Proteins.

Binary plant transformation vectors comprising transgene cassettes designed to express both plastid targeted and untargeted versions of The PirAB Fusion Proteins are cloned using methods known in the art and comprise the coding sequences as presented in Tables 17 and 18.

The resulting binary transformation vectors are used to stably transform canola plant cells using methods known in the art. The transformed cells are induced to form plants. Bioassays using plant leaf disks are performed analogous to those described in U.S. Pat. No. 8,344,207 using field collected Flea Beetles. A non-transformed canola plant is used to obtain tissue to be used as a negative control. Multiple transformation events from each binary vector are assessed against Coleopteran Flea Beetle pests such as, but not limited to, Crucifer Flea Beetle (Phyllotreta cruciferae), Striped Flea Beetle (Phyllotreta striolata), and Western Black Flea Beetle (Phyllotreta pusilla). Flea Beetle mortality is determined each day as the Beetles continue to feed. Leaf discs are changed every two (2) to three (3) days over a twelve (12) day period to assure fresh material is available to the Flea Beetles for feeding, and to reduce any impact of protein degradation in the sample.

Alternatively, transformed canola plants can be planted in a field where Flea Beetle infestations are present. The plants can be housed in a tent to prevent those Flea Beetles that emerge from the soil from escaping the experimental plots. Damage ratings of the canola leaves can be taken to determine which plants experienced lesser damage and demonstrated resistance to the Flea Beetles.

### Example 10

### Assay of the Activity of the PirAB Fusion Proteins Against Hemipteran Pests in Stably Transformed Soybean Plants

This Example describes the assay of activity against Hemipteran insect pests in soybean plants stably transformed to express one of The PirAB Fusion Proteins.

Binary plant transformation vectors comprising transgene cassettes designed to express both plastid targeted and untargeted versions of one of The PirAB Fusion Proteins are cloned using methods known in the art and comprise the coding sequences as presented in Tables 17 and 18. Soybean plants are transformed using the binary plant transformation vectors. The transformed soybean plant cells are induced to form whole plants. Assay for activity against the Hemipteran pests is performed using a variety of techniques which will depend upon the species of Hemipteran pests and the preferred target tissue of that pest. For example, the Hemipteran pest species of Stink Bugs typically feed on the developing seeds and pods of the soybean plant. To assay for activity against Stink Bugs, R5 stage pods are harvested from the transgenic soybean plants expressing one of The PirAB Fusion Proteins and placed in a covered Petri dish or large multi-well plate containing a layer of either agar or wet paper to provide humidity to the feeding environment. Second instar Stink Bug nymphs are placed in the Petri dish or large multi-well plate. A cover providing for the exchange of oxygen while preventing desiccation is placed over the feeding environment. The Stink Bug nymphs are allowed to feed for several days. Measurements of stunting and mortality are taken and compared to Stink Bugs nymphs feeding on pods from untransformed soybean plants.

Alternatively, assay of activity can also be performed on whole stably transformed plants. Transformed plants expressing one of The PirAB Fusion Proteins are grown in a growth chamber or in the greenhouse. At R5 stage, the plants are enclosed in a cage made from breathable plastic “pollination” sheets (Vilutis and Company Inc, Frankfort, IL). The sheet sleeves are secured to the main stem just above the soil surface using a Velcro® tie. Each plant is infested with a specific number of second instar Stink Bug nymphs. The nymphs are released into each individual cage through a small slit on the cage side and then the cage is securely closed ensuring the insects won't escape. The nymphs are allowed to feed on the soybean pods for several days to a week or more. Observations are taken each day to determine measurements of stunting and mortality. At the end of the feeding period, the live and dead nymphs are collected. The plants are cut below the cages and moved to a laboratory where the insects are collected for each plant. Before opening the cage, the plants are vigorously shaken to ensure all of the insects fall off from their feeding sites to the base of the cage. Then the cage base is opened and all plant material is removed and placed on a black sheet. The insects can be collected using an aspirator or some other means. The number of insects and their developmental stage is recorded for each plant. Also, the number and developmental stage of dead nymphs is also recorded. These measurements are compared to the measurements obtained from negative control, un-transformed plants.

Delays in development of the Stink Bug nymphs (stunting) or mortality are interpreted as an indication of toxicity if, when compared to the un-transformed controls, there is a significant difference.

### Example 11

### Assay of the Activity of the PirAB Fusion Proteins Against Hemipteran Pests in Stably Transformed Corn Plants

This Example describes the assay of activity against Hemipteran insect pests in corn plants stably transformed to express one of The PirAB Fusion Proteins.

Binary plant transformation vectors comprising transgene cassettes designed to express both plastid targeted and untargeted versions of one of The PirAB Fusion Proteins are cloned using methods known in the art and comprise the coding sequences as presented in Tables 17 and 18. Corn plants are transformed using the binary plant transformation vectors. The transformed corn plant cells are induced to form whole plants. Assay for activity against the Hemipteran pests is performed using a variety of techniques which will depend upon the species of Hemipteran pests and the preferred target tissue of that pest. For example, the Hemipteran pest species of Stink Bugs typically feed on the young corn plants in late spring or early summer, resulting in holes in the leaf, and if severe, deformed plants. In late summer, Stink Bugs typically feed on the ear itself, directly destroying the kernels.

One method to assay for Stink Bug activity is to expose the Stink Bug nymphs to leaf discs derived from stably transformed corn plants expressing one of The PirAB Fusion Proteins in large multi-well plates. Second stage instar Stink Bug nymphs are placed in large multi-well plates with leaf discs derived from the stably transformed corn plants and allowed to feed for several days. Measurements of stunting and mortality are taken and compared to Stink Bug nymphs who have fed on un-transformed corn leaf discs.

Alternatively, whole transformed plants can be used to assay for Stink Bug activity. Stably transformed corn plants expressing one of The PirAB Fusion Proteins are enclosed in cages in a similar manner as described for soybean plants in Example 4. Second instar nymphs are introduced to V3 stage corn plants and allowed to feed for several days to a week. After the prescribed feeding period, the live and dead nymphs are collected. Measurements of stunting and mortality are compared to un-transformed control plants.

To assay Stink Bug activity using stably transformed corn ears, a similar approach can be taken as that of assaying in V3 stage plants. The developing corn ears of stably transformed corn plants expressing one of The PirAB Fusion Proteins are encapsulated using sheets of material that permit the free exchange of air while preventing escape of the Stink Bug nymphs. The encapsulated ears are infested with second instar stage Stink Bug nymphs and allowed to feed on the developing kernels of the ear for several days to a week. Measurements of stunting and mortality are compared to un-transformed control plant ears.

### Example 12

### Assay of the Activity of the PirAB Fusion Proteins Against Hemipteran Pests in Stably Transformed Cotton Plants

This Example describes the assay of activity against Hemipteran insect pests in cotton plants stably transformed to express one of The PirAB Fusion Proteins.

Binary plant transformation vectors comprising transgene cassettes designed to express both plastid targeted and untargeted versions of one of The PirAB Fusion Proteins are cloned using methods known in the art and comprise the coding sequences as presented in Tables 17 and 18. Cotton plants are transformed using the binary plant transformation vectors. The transformed cotton plant cells are induced to form whole plants. Assay for activity against the Hemipteran pests is performed using a variety of techniques which will depend upon the species of Hemipteran pests and the preferred target tissue of that pest. For example, the Hemipteran pest species of Stink Bugs are typically seed feeders, and thus, injury to cotton bolls is the primary concern. They primarily damage cotton by piercing the bolls and feeding on the seeds. Their feeding activity can result in dark spots about 1/16 of an inch in diameter on the outside of larger bolls where feeding occurred. Seed feeding may result in reduced lint production and stained lint near the feeding site. Because of their size, adults and fourth and fifth instar nymphs have the greatest potential for damaging bolls, and it is therefore important to kill the insect in its earlier nymphal stages. The Hemipteran pest species of Lygus primarily feed on the squares and young bolls. The nymphs are more voracious feeders and tend to cause the most severe damage. When feeding on squares, Lygus target the developing anthers which often results in the square shriveling and falling from the plant. For those squares that develop into bolls, the bolls may have anthers that are incapable of forming pollen, unfertilized seeds, and empty locules. When feeding on bolls, Lygus target the developing seeds, causing small black sunken spots on the outside of the boll.

One method to assay activity of The PirAB Fusion Proteins in stably transformed cotton plants is to use squares in an insect bioassay. The squares are harvested from transformed cotton plants expressing TIC6880PL, TIC9316, TIC9317, TIC9318, TIC9319, TIC9320, TIC9322, TIC10376PL, TIC10378PL, TIC10380PL, TIC10381PL, TIC11103, TIC11104, or TIC11302. The squares can be put into a petri dish or each square into a well of a large well plate. Young neonate Lygus or Stink Bug nymphs are placed into the petri dish or large well plate and allowed to feed for a prescribed time. Measurements of stunting and mortality are taken over the time course of feeding and compared to controls in which squares derived from untransformed cotton plants are used in assay.

Alternatively, assay of activity can be performed on whole transformed cotton plants. For example, to assay against Lygus species, R₁ seeds derived from plants expressing one of The PirAB Fusion Proteins are sown in 10 inch pots. An untransformed cotton plant, preferably from the same variety as the transformed plants, is used as a negative control. Plants are maintained in an environment chamber with a photoperiod of sixteen (16) hours of light at thirty-two (32) degrees Celsius and eight (8) hours of dark at twenty three (23) degrees Celsius, and a light intensity between eight hundred (800) and nine hundred (900) micro-Einsteins. At forty (40) to forty-five (45) days after planting, the individual plants are enclosed in a cage made from breathable plastic “pollination” sheets (Vilutis and Company Inc, Frankfort, IL). The sheet sleeves are secured to the main stem just above the soil surface using a Velcro® tie. Two pairs of sexually mature male and female Lygus lineolaris or Lygus hesperus adults (six days old) from a laboratory culture are collected into a fourteen-milliliter round-bottom plastic tube (Bacton Dickson Labware, Franklin Lakes, NJ) and used for each plant. The adults are released into each individual cage through a small slit on the cage side and then the cage is securely closed ensuring the insects would not escape. The insects are allowed to mate and the plants are kept in the cage for twenty-one (21) days.

After twenty-one (21) days, the plants are then cut below the cages and moved to a laboratory where the insects are collected for each plant and counted. Before opening the cage, the plants are vigorously shaken to ensure all of the insects fall off from their feeding sites to the base of the cage. Then the cage base is opened and all plant material removed and placed on a black sheet. The insects are collected using an aspirator. The plant is then thoroughly inspected to recover any remaining insects. The number of insects collected and their developmental stage are recorded for each plant. The insect counts are divided into several groups based upon maturity of the Lygus: nymphs up to 3^{rd} instar, 4^{th} instar, 5^{th} instar and adults.

To assay against Stink Bug species, R1 seeds derived from plants expressing one of The PirAB Fusion Proteins are sown into pots and grown and caged as described above. Untransformed cotton plants are also used as a negative control. Second instar Stink Bug nymphs are used to infest the plants and allowed to feed on the squares and bolls for several days or weeks. The caged plants are collected as described above and the collected stink bugs are examined and scored for mortality, as well as, maturity of the nymphs recorded. These scores are then compared to the negative control plants.

### Example 13

TIC9318 and TIC11302 Demonstrates Activity Against Western Corn Rootworm Pests when Expressed in Stably Transformed Corn Plants

This Example illustrates the inhibitory activity of TIC9318 and TIC11302 against Western Corn Rootworm (Diabrotica virgifera, WCR) in stably transformed corn plants.

Corn plants were transformed with binary plant transformation constructs comprising an expression cassette for the expression of either TIC9318 or TIC11302. The binary plant transformation vectors comprised transgene cassettes designed to express TIC9318 and TIC11302, and were cloned using methods known in the art. The plant transformation vectors comprised a first transgene cassette for expression of the TIC9318 or TIC11302 pesticidal protein which comprised a plant expressible promoter, operably linked 5′ to a leader, operably linked 5′ to an intron, operably linked 5′ to a synthetic coding sequence encoding TIC9318 or TIC11302, operably linked 5′ to a 3′ UTR and, a second transgene cassette for the selection of transformed plant cells using glyphosate. The resulting vectors were used to stably transform corn plants using an Agrobacterium-mediated transformation method. The transformed cells were induced to form plants by methods known in the art.

R₀ stably transformed plants were used to assay TIC11302 for WCR resistance as well as generating F₁ progeny. Multiple single copy events were selected from each binary vector transformation. A portion of the events arising from each binary vector transformation were used in the R₀ WCR assay.

The R₀ assay plants were transplanted to eight inch pots. The plants were inoculated with approximately two thousand eggs each from WCR. The eggs were incubated for approximately ten (10) days prior to inoculation to allow hatching to occur four (4) days after inoculation to ensure a sufficient number of larvae survive and were able to attack the corn roots. Each pot was inoculated with approximately two thousand WCR eggs. The transformed plants were inoculated at approximately V2 to V3 stage. The plants were grown after infestation for approximately twenty-eight (28) days. The plants were removed from the pots with the roots being carefully washed to remove all soil. The damage to the roots was assessed using a damage rating scale of 1-5, as presented in Table 19 of Example 17. Comparison was also made to the negative controls to assure the assay has been performed properly. Multiple R₀ events for each TIC11302 binary vector transformation were used in the WCR assay.

A portion of the R₀ stably transformed events arising from each binary vector transformation of TIC9318 and TIC11302 were used to produce F₁ progeny. The R₀ stably transformed plants were allowed to self-fertilize, producing F₁ progeny. The F₁ seed was planted in eight inch pots. Heterozygous plants were identified through molecular methods known in the art and were used for assay against WCR. Inoculation with the WCR eggs was as described for the R₀ stably transformed events as described above. The damage to the roots were assessed using a damage rating scale of 0-3, as presented in Table 20 of Example 7. Comparison was made to the negative control to assure the assay has been performed properly. The average Root Damage Rating (RDR) for each construct is presented in Table 21 below, wherein “NT” indicates not tested.

TABLE 21
Average Root Damage Rating (RDR) for corn plants stably transformed with
TIC9318 or TIC11302.

| | PirAB | Nucleotide | Protein | | R₀ Neg. | | F₁ Neg. |
|---|---|---|---|---|---|---|---|
| | Fusion | SEQ ID | SEQ ID | | Control | | Control |
| Construct | Protein | NO: | NO: | R₀ RDR | RDR | F₁ RDR | RDR |
| Construct_1 | TIC9318 | 53 | 24 | NT | NT | 1.4 | 2.8 |
| Construct_2 | TIC9318 | 53 | 24 | NT | NT | 1.5 | 1.8 |
| Construct_3 | TIC9318 | 53 | 24 | NT | NT | 1.2 | 1.8 |
| Construct_4 | TIC11302 | 158 | 119 | 2.9 | 4.1 | 1.5 | 1.8 |
| Construct_5 | TIC11302 | 158 | 119 | 2.8 | 4.1 | 1.4 | 1.8 |
| Construct_6 | TIC11302 | 158 | 119 | 2.9 | 4.1 | 1.4 | 1.8 |

As can be seen in Table 21 above, both TIC9318 and TIC11302 demonstrated resistance to Western Corn Rootworm (Diabrotica virgifera virgifera) when compared to the negative controls.

All of the compositions disclosed and claimed herein can be made and executed without undue experimentation in light of the present disclosure. While the compositions of this invention have been described in terms of the foregoing illustrative embodiments, it will be apparent to those of skill in the art that variations, changes, modifications, and alterations may be applied to the composition described herein, without departing from the true concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

All publications and published patent documents cited in the specification are incorporated herein by reference to the same extent as if each individual publication or patent application was specifically and individually indicated to be incorporated by reference.

1. A recombinant nucleic acid molecule comprising a heterologous promoter operably linked to a polynucleotide segment encoding a pesticidal protein, wherein
(a) the pesticidal protein comprises (i) an amino acid sequence having at least 95% identity to the amino acid sequence of SEQ ID NO:8 and (ii) an amino acid sequence having at least 95% identity to the amino acid sequence of SEQ ID NO:10; or
(b) wherein the pesticidal protein comprises an amino acid sequence having at least 95% identity to the amino acid sequence of SEQ ID NO:12.

2. The recombinant nucleic acid molecule of claim 1, wherein:
the recombinant nucleic acid molecule is in operable linkage with a vector, and said vector is selected from the group consisting of a plasmid, phagemid, bacmid, cosmid, and a bacterial or yeast artificial chromosome.

3. The recombinant nucleic acid molecule of claim 1, defined as present within a host cell, wherein said host cell is selected from the group consisting of a bacterial and a plant cell.

4. The recombinant nucleic acid molecule of claim 3, wherein the bacterial host cell is from a genus of bacteria selected from the group consisting of: Agrobacterium, Rhizobium, Bacillus, Brevibacillus, Escherichia, Pseudomonas, Klebsiella, Pantoea, and Erwinia.

5. The recombinant nucleic acid molecule of claim 4, wherein the Bacillus species is Bacillus cereus or Bacillus thuringiensis, said Brevibacillus is Brevibacillus laterosperus, or said Escherichia is Escherichia coli.

6. The recombinant nucleic acid molecule of claim 3, wherein said plant cell is a dicotyledonous or a monocotyledonous plant cell.

7. The recombinant nucleic acid molecule of claim 6, wherein said plant host cell is selected from the group consisting of an alfalfa, banana, barley, bean, broccoli, cabbage, Brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell.

8. The recombinant nucleic acid molecule of claim 1, wherein said pesticidal protein exhibits activity against a Coleopteran insect.

9. The recombinant nucleic acid molecule of claim 8, wherein said insect is Western Corn Rootworm, Southern Corn Rootworm, Northern Corn Rootworm, Mexican Corn Rootworm, Brazilian Corn Rootworm, Colorada Potato Beetle, Brazilian Corn Rootworm complex consisting of Diabrotica viridula and Diabrotica speciosa, Crucifer Flea Beetle, Striped Flea Beetle, or Western Black Flea Beetle.

10. The recombinant nucleic acid molecule of claim 1, wherein said pesticidal protein exhibits activity against an insect species of the order of Lepidoptera.

11. The recombinant nucleic acid molecule of claim 10, wherein said insect is Black Cutworm, Corn Earworm, Diamondback Moth, European Corn Borer, Fall Armyworm, Southern Armyworm, Soybean Looper, Southwestern Corn Borer, Tobacco Budworm, Velvetbean Caterpillar, Sugarcane Borer, Lesser Cornstalk Borer, Black Armyworm, Beet Armyworm, Old World Bollworm, Oriental leaf Worm, or Pink Bollworm.

12. The recombinant nucleic acid molecule of claim 1, wherein said pesticidal protein exhibits activity against an insect species in the order of Hemiptera.

13. The recombinant nucleic acid molecule of claim 12, wherein said insect is Southern Green Stinkbug, Neotropical Brown Stinkbug, Redbanded Stink Bug, Black-Spined Green-Belly Stink Bug species, Brown-Winged Stink Bug, Brown Stink Bug, Green Stink Bug, Brown Marmorated Stink Bug Western Tarnished Plant Bug, or Tarnished Plant Bug.

14. A plant or part thereof comprising the recombinant nucleic acid molecule of claim 1.

15. The plant or part thereof of claim 14, wherein said plant is a monocot plant or a dicot plant.

16. The plant or part thereof of claim 14, wherein the plant is selected from the group consisting of an alfalfa, banana, barley, bean, broccoli, cabbage, Brassica, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat.

17. A seed of the plant of claim 14, wherein said seed comprises said recombinant nucleic acid molecule.

18. An insect inhibitory composition comprising the recombinant nucleic acid molecule of claim 1.

19. The insect inhibitory composition of claim 18, further comprising a nucleotide sequence encoding at least one other pesticidal agent that is different from said pesticidal protein.

20. The insect inhibitory composition of claim 19, wherein said at least one other pesticidal agent is selected from the group consisting of an insect inhibitory protein, an insect inhibitory dsRNA molecule, and an ancillary protein.

21. The insect inhibitory composition of claim 19, wherein said at least one other pesticidal agent exhibits activity against one or more pest species of the orders Lepidoptera, Coleoptera, or Hemiptera.

22. The insect inhibitory composition of claim 21, wherein said at least one other pesticidal agent is a protein selected from the group consisting of a Cry1A, Cry1Ab, Cry1Ac, Cry1A.105, Cry1Ae, Cry1B, Cry1C, Cry1C variants, Cry1D, Cry1E, Cry1F, Cry1A/F chimeras, Cry1G, Cry1H, Cry1I, Cry1J, Cry1K, Cry1L, Cry2A, Cry2Ab, Cry2Ae, Cry3, Cry3A variants, Cry3B, Cry4B, Cry6, Cry7, Cry8, Cry9, Cry15, Cry34, Cry35, Cry43A, Cry43B, Cry51Aa1, ET29, ET33, ET34, ET35, ET66, ET70, TIC400, TIC407, TIC417, TIC431, TIC800, TIC807, TIC834, TIC853, TIC900, TIC901, TIC1201, TIC1415, TIC2160, TIC3131, TIC836, TIC860, TIC867, TIC869, TIC1100, VIP3A, VIP3B, VIP3Ab, AXMI-AXMI-, AXMI-88, AXMI-97, AXMI-102, AXMI-112, AXMI-117, AXMI-100, AXMI-115, AXMI-113, and AXMI-005, AXMI134, AXMI-150, AXMI-171, AXMI-184, AXMI-196, AXMI-204, AXMI-207, AXMI-209, AXMI-205, AXMI-218, AXMI-220, AXMI-221z, AXMI-222z, AXMI-223z, AXMI-224z and AXMI-225z, AXMI-238, AXMI-270, AXMI-279, AXMI-345, AXMI-335, AXMI-R1 and variants thereof, IP3 and variants thereof, DIG-3, DIG-5, DIG-10, DIG-657 DIG-11, Cry71Aa1, Cry72Aa1, PHI-4 variants, PIP-72 variants, PIP-45 variants, PIP-64 variants, PIP-74 variants, PIP-75 variants, PIP-77 variants, Axmi422, Dig-305, Axmi440, PIP-47 variants, Axmi281, BT-009, BT-0012, BT-0013, BT-0023, BT0067, BT-0044, BT-0051, BT-0068, BT-0128, DIG-17, DIG-90, DIG-79, Cry1JP578V, Cry1JPS1, and Cry1 JPS1P578V.

23. The insect inhibitory composition of claim 18, further defined as comprising a plant cell that expresses said recombinant nucleic acid molecule.

24. A commodity product produced from the plant or part thereof of claim 14, wherein the commodity product comprises a detectable amount of said recombinant nucleic acid molecule or a pesticidal protein encoded thereby.

25. The commodity product of claim 24, selected from the group consisting of commodity corn bagged by a grain handler, corn flakes, corn cakes, corn flour, corn meal, corn syrup, corn oil, corn silage, corn starch, corn cereal, and the like, whole or processed cotton seed, cotton oil, lint, seeds and plant parts processed for feed or food, fiber, paper, biomasses, and fuel products such as fuel derived from cotton oil or pellets derived from cotton gin waste, whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts.

26. A method of producing seed comprising:
a) planting at least a first seed according to claim 17.
b) growing a plant from the seed; and
c) harvesting seed from the plant, wherein said harvested seed comprises said recombinant nucleic acid molecule.

27. A plant resistant to insect infestation, wherein the cells of said plant comprise the recombinant nucleic acid molecule of claim 1.

28. A method for controlling a Coleopteran, Lepidopteran, or Hemipteran species pest or pest infestation, said method comprising:
contacting the pest with an insecticidally effective amount of a pesticidal protein the pesticidal protein comprises an amino acid sequence having at least 95% identity to the amino acid sequence of SEQ ID NO:8 and an amino acid sequence having at least 95% identity to the amino acid sequence of SEQ ID NO:10.

29-31. (canceled)

32. The recombinant nucleic acid molecule of claim 1, wherein the pesticidal protein comprises an amino acid sequence having at least 95% identity to the amino acid sequence of SEQ ID NO:12.

33. The recombinant nucleic acid molecule of claim 1, wherein the polynucleotide segment encoding a pesticidal protein comprises a nucleotide sequence having at least 95% identity to the nucleotide sequence of SEQ ID NO:51 or SEQ ID NO:11.


SEQUENCE LISTING (160 sequences)

SEQ ID NO: 1 - DNA, 408 bp, Xenorhabdus nematophila
  source 1..408: mol_type = other DNA
  misc_feature 1..408: note = A nucleic acid sequence obtained from Xenorhabdus nematophilastrain ISB000002 encoding a TIC4771 PirA pesticidal proteinsequence.
atgattacaataaatataagtggtggtagtataaaaattagtaacaacataggatcagaaactgatatcaaaaatacacctttttcagaacctctttcaattagtaattataaggatatgacaatagagccacattcgtctatccaagcaacaagaactgatacaccaattattcctgaaacacgaccaaattattatgtagctaattccggccctgccgcatcagtgagagctgttttttattggtctcattcttttacatcagaatggttcgaacattcatctatcattgtaaaagcaggagaagatggaatattgaactcacctagcaattctgtatattacagtaaggttgtcatttacaacgatacggataaacgggcctttgtcacaggttatgacaaataa

SEQ ID NO: 2 - protein, 135 aa, Xenorhabdus nematophila
  REGION 1..135: note = The amino acid sequence of the TIC4771 PirA protein.
MITINISGGSIKISNNIGSETDIKNTPFSEPLSISNYKDMTIEPHSSIQATRTDTPIIPETRPNYYVANSGPAASVRAVFYWSHSFTSEWFEHSSIIVKAGEDGILNSPSNSVYYSKVVIYNDTDKRAFVTGYDK

SEQ ID NO: 3 - DNA, 1287 bp, Xenorhabdus nematophila
  source 1..1287: mol_type = other DNA
  misc_feature 1..1287: note = A nucleic acid sequence obtained from Xenorhabdus nematophilastrain ISB000002 encoding a TIC4772 PirB pesticidal proteinsequence.
atgaataacgaacttatgaacacaaatgaatcacaaccttcagagacattatctttaattaatgaatctatattaacagcaccttatgccgtttctacccctaattatgaatgggatatgtcatcaataataaaagatgccattattggaggtataggatttattcccgggccgggttcagcaatatcgtttttgctagggctattttggccgcaacaaacagacaatacctgggagcaaattctccaaaaagtagaacagatgatagaggaagcgaatttaaaaactattcaaggaatactgaacggagatatacaagaaataaaaggaaagatggaacatgtggaatatatgctagaaacctcaccaggcactcaagaaagccatgacgcatatatgttcttagcgagatatctggtaagtatagatgaaaaattcaaatcttttgataataaaacaaattatcaaattcttccaatgtacaccaatacgcttatgttacaggcaccttactggaaaatgggtatagagaagaaaaatgatattttgctaacagatatagaagttaatgaattaaaacagcttatcgaaaatctatatgccaaggccaatagctatattcatgaagtgtatacccgtgaatacgataatgcggtaaatacctcaacagcaacaacgattaccaataatttattgtctgtaagagggtattgtttattacatggattagagtgccttgaagtccttgatcatatacaaaataataatcttgatcagagcttctatccgaaaactatcagttattctactgtatttgatcgctcaacaaacaaaacaagactccaggctcttaccgaagacgagcaaatggaagaaccactcaaaccctcttttattaatggggaatataataaaataaaatcactgattggatatgtacagagaattggaaacgcccctagagttggaggtataaaaattacatttactaatggatcatctcatactctgggtacagtgacctcagaatcaaactcaattgaactaaatgatagtgttataaccagtgtggaagtatggggaaatggtgctgttgatgaggcattctttacattaagtgacggtcgtcaatttaggcttggtcaacgctatgccagtaactacagaaaatatgctgttgatggccactatatttcaggattgtacttagccagtgatgagccttcacttgctggtcaagccgcaggtattgcagtttcatatcatatattggttgataagaaataa

SEQ ID NO: 4 - protein, 428 aa, Xenorhabdus nematophila
  REGION 1..428: note = The amino acid sequence of the TIC4772 PirB protein.
MNNELMNTNESQPSETLSLINESILTAPYAVSTPNYEWDMSSIIKDAIIGGIGFIPGPGSAISFLLGLFWPQQTDNTWEQILQKVEQMIEEANLKTIQGILNGDIQEIKGKMEHVEYMLETSPGTQESHDAYMFLARYLVSIDEKFKSFDNKTNYQILPMYTNTLMLQAPYWKMGIEKKNDILLTDIEVNELKQLIENLYAKANSYIHEVYTREYDNAVNTSTATTITNNLLSVRGYCLLHGLECLEVLDHIQNNNLDQSFYPKTISYSTVFDRSTNKTRLQALTEDEQMEEPLKPSFINGEYNKIKSLIGYVQRIGNAPRVGGIKITFTNGSSHTLGTVTSESNSIELNDSVITSVEVWGNGAVDEAFFTLSDGRQFRLGQRYASNYRKYAVDGHYISGLYLASDEPSLAGQAAGIAVSYHILVDKK

SEQ ID NO: 5 - DNA, 1692 bp, synthetic construct
  source 1..1692: mol_type = other DNA
  misc_feature 1..1692: note = A nucleic acid sequence encoding a PirAB fusion protein, TIC6880comprised of the TIC4771 and TIC4772 coding sequences in operablelinkage and in frame.
atgattacaataaatataagtggtggtagtataaaaattagtaacaacataggatcagaaactgatatcagaaatacacctttttcagaacctctttcaattagtaattataaggatatgacaatagagccacattcgtctatccaagcaacaagaactgatacaccaattattcctgaaacacgaccaaattattatgtagctaattccggccctgccgcatcagtgagagctgttttttattggtctcattcttttacatcagaatggttcgaacattcatctatcattgtaaaagcaggagaagatggaatattgaactcacctagcaattctgtatattacagtaaggttgtcatttacaacgatacggataaacgggcctttgtcacaggttatgacaaaatgaataacgaacttatgaacacaaatgaatcacaaccttcagagacattatctttaattaatgaatctatattaacagcaccttatgccgtttctacccctaattatgaatgggatatgtcatcaataataaaagatgccattattggaggtataggatttattcccgggccgggttcagcaatatcgtttttgctagggctattttggccgcaacaaacagacaatacctgggagcaaattctccaaaaagtagaacagatgatagaggaagcgaatttaaaaactattcaaggaatactgaacggagatatacaagaaataaaaggaaagatggaacatgtggaatatatgctagaaacctcaccaggcactcaagaaagccatgacgcatatatgttcttagcgagatatctggtaagtatagatgaaaaattcaaatcttttgataataaaacaaattatcaaattcttccaatgtacaccaatacgcttatgttacaggcaccttactggaaaatgggtatagagaagaaaaatgatattttgctaacagatatagaagttaatgaattaaaacagcttatcgaaagtctatatgccaaggccaatagctatattcatgaagtgtatacccgtgaatacgataatgcggtaaatacctcaacagcaacaacgattaccaataatttattgtctgtaagagggtattgtttattacatggattagagtgccttgaagtccttgatcatatacaaaataataatcttgatcagagcttctatccgaaaactatcagttattctactgtatttgatcgctcaacaaacaaaacaagactccaggctcttaccgaagacgagcaaatggaagaaccactcaaaccctcttttattaatggggaatataataaaataaaatcactgattggatatgtacagagaattggaaacgcccctagagttggaggtataaaaattacatttactaatggatcatctcatactctgggtacagtgacctcagaatcaaactcaattgaactaaatgatagtgttataaccagtgtggaagtatggggaaatggtgctgttgatgaggcattctttacattaagtgacggtcgtcaatttaggcttggtcaacgctatgccagtaactacagaaaatatgctgttgatggccactatatttcaggattgtacttagccagtgatgagccttcacttgctggtcaagccgcaggtattgcagtttcatatcatatattggttgataagaaataa

SEQ ID NO: 6 - protein, 563 aa, synthetic construct
  REGION 1..563: note = The amino acid sequence of the TIC6880 PirAB fusion protein
MITINISGGSIKISNNIGSETDIRNTPFSEPLSISNYKDMTIEPHSSIQATRTDTPIIPETRPNYYVANSGPAASVRAVFYWSHSFTSEWFEHSSIIVKAGEDGILNSPSNSVYYSKVVIYNDTDKRAFVTGYDKMNNELMNTNESQPSETLSLINESILTAPYAVSTPNYEWDMSSIIKDAIIGGIGFIPGPGSAISFLLGLFWPQQTDNTWEQILQKVEQMIEEANLKTIQGILNGDIQEIKGKMEHVEYMLETSPGTQESHDAYMFLARYLVSIDEKFKSFDNKTNYQILPMYTNTLMLQAPYWKMGIEKKNDILLTDIEVNELKQLIESLYAKANSYIHEVYTREYDNAVNTSTATTITNNLLSVRGYCLLHGLECLEVLDHIQNNNLDQSFYPKTISYSTVFDRSTNKTRLQALTEDEQMEEPLKPSFINGEYNKIKSLIGYVQRIGNAPRVGGIKITFTNGSSHTLGTVTSESNSIELNDSVITSVEVWGNGAVDEAFFTLSDGRQFRLGQRYASNYRKYAVDGHYISGLYLASDEPSLAGQAAGIAVSYHILVDKK

SEQ ID NO: 7 - DNA, 426 bp, Xenorhabdus ehlersii
  source 1..426: mol_type = other DNA
  misc_feature 1..426: note = A nucleic acid sequence obtained from Xenorhabdus ehlersii strain85823 encoding a TIC7575 PirA pesticidal protein sequence.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagtaa

SEQ ID NO: 8 - protein, 141 aa, Xenorhabdus ehlersii
  REGION 1..141: note = The amino acid sequence of the TIC7575 PirA protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNK

SEQ ID NO: 9 - DNA, 1278 bp, Xenorhabdus ehlersii
  source 1..1278: mol_type = other DNA
  misc_feature 1..1278: note = A nucleic acid sequence obtained from Xenorhabdus ehlersii strain85823 encoding a TIC7576 PirB pesticidal protein sequence.
atgaatatctcaccgattaatgtatctgaaaatgaaacattacctgaactcactgatgttatgcttattgtgccttatacaacatctacccctgattatgaatgggatatgtcatcaattataaaggatgcgattattggcggcgtagggtttattccaggagcaggctctgcaatgtccttcctattgggactattttggcctcaacagaaagataatacatgggaacagatcctccaaaaagtagaacagatgatagagaatgccgttctgcaaactattaaaggaatacttaatggagatatacaagaaatcaaggggaaaatggaacatgtgcaatacatgctggaaacctcgcctggcagtcaggaaagtcatgacgcatatatgttcctggctagatacctggtgagtatagatgaaaaattcaagtcttttgataataaaacaaactaccagatcctgccgatgtacactaacacggttatgttacaaatcccttattggaaaatgggaatagagaagaaaaatgatattgggctgacagatattgaagtcaatgagttaaaacagcttatcgataaattggtcgacaaggccaagagttacatccatacgatgtatacgaatgaatataatgatgccataaatacatcaacagcatcgagtgtcactaataatttactctctgtaagaggatattgtttattacacggtttagagtgtattgagttaattgaacatctacaaaacaatagcctcgaaagtggtttttatcctaaaactatcagttattcaactgtatttgatcgtcagactaacaaaatgagaattcaggctcttacagaagacgatcaaatgcaggaaccctttaagccatctttaatcaacggcaaatacaataaaatacaatccttgcttggatatgtacaaagaattggaaatgcacctagagtggggggtattaaaatcacctttgccaacggttcatcctatacacttggcacagtaacatcagaaacgagttcaattgaactcaatgatagtgttatcgaaagattggaagtatggggcaatggcgctgttgatgaggcattatttacgttaagtgatgggcgtcaactcagagtcggtgagcgctacgcgacaaaatatagaaaatatgctgttgatggacactatattgcaggactgtacttagctagcgatgaaccttcacttgctggtcaagccgcaggtattgccgtttcataccatatgttggatgataaaaaataa

SEQ ID NO: 10 - protein, 425 aa, Xenorhabdus ehlersii
  REGION 1..425: note = The amino acid sequence of the TIC7576 PirB protein.
MNISPINVSENETLPELTDVMLIVPYTTSTPDYEWDMSSIIKDAIIGGVGFIPGAGSAMSFLLGLFWPQQKDNTWEQILQKVEQMIENAVLQTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDKLVDKAKSYIHTMYTNEYNDAINTSTASSVTNNLLSVRGYCLLHGLECIELIEHLQNNSLESGFYPKTISYSTVFDRQTNKMRIQALTEDDQMQEPFKPSLINGKYNKIQSLLGYVQRIGNAPRVGGIKITFANGSSYTLGTVTSETSSIELNDSVIERLEVWGNGAVDEALFTLSDGRQLRVGERYATKYRKYAVDGHYIAGLYLASDEPSLAGQAAGIAVSYHMLDDKK

SEQ ID NO: 11 - DNA, 1701 bp, synthetic construct
  source 1..1701: mol_type = other DNA
  misc_feature 1..1701: note = A nucleic acid sequence encoding a PirAB fusion protein, TIC9316comprised of the TIC7575 and TIC7576 coding sequences in operablelinkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgaatatctcaccgattaatgtatctgaaaatgaaacattacctgaactcactgatgttatgcttattgtgccttatacaacatctacccctgattatgaatgggatatgtcatcaattataaaggatgcgattattggcggcgtagggtttattccaggagcaggctctgcaatgtccttcctattgggactattttggcctcaacagaaagataatacatgggaacagatcctccaaaaagtagaacagatgatagagaatgccgttctgcaaactattaaaggaatacttaatggagatatacaagaaatcaaggggaaaatggaacatgtgcaatacatgctggaaacctcgcctggcagtcaggaaagtcatgacgcatatatgttcctggctagatacctggtgagtatagatgaaaaattcaagtcttttgataataaaacaaactaccagatcctgccgatgtacactaacacggttatgttacaaatcccttattggaaaatgggaatagagaagaaaaatgatattgggctgacagatattgaagtcaatgagttaaaacagcttatcgataaattggtcgacaaggccaagagttacatccatacgatgtatacgaatgaatataatgatgccataaatacatcaacagcatcgagtgtcactaataatttactctctgtaagaggatattgtttattacacggtttagagtgtattgagttaattgaacatctacaaaacaatagcctcgaaagtggtttttatcctaaaactatcagttattcaactgtatttgatcgtcagactaacaaaatgagaattcaggctcttacagaagacgatcaaatgcaggaaccctttaagccatctttaatcaacggcaaatacaataaaatacaatccttgcttggatatgtacaaagaattggaaatgcacctagagtggggggtattaaaatcacctttgccaacggttcatcctatacacttggcacagtaacatcagaaacgagttcaattgaactcaatgatagtgttatcgaaagattggaagtatggggcaatggcgctgttgatgaggcattatttacgttaagtgatgggcgtcaactcagagtcggtgagcgctacgcgacaaaatatagaaaatatgctgttgatggacactatattgcaggactgtacttagctagcgatgaaccttcacttgctggtcaagccgcaggtattgccgtttcataccatatgttggatgataaaaaataa

SEQ ID NO: 12 - protein, 566 aa, synthetic construct
  REGION 1..566: note = The amino acid sequence of the TIC9316 PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMNISPINVSENETLPELTDVMLIVPYTTSTPDYEWDMSSIIKDAIIGGVGFIPGAGSAMSFLLGLFWPQQKDNTWEQILQKVEQMIENAVLQTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDKLVDKAKSYIHTMYTNEYNDAINTSTASSVTNNLLSVRGYCLLHGLECIELIEHLQNNSLESGFYPKTISYSTVFDRQTNKMRIQALTEDDQMQEPFKPSLINGKYNKIQSLLGYVQRIGNAPRVGGIKITFANGSSYTLGTVTSETSSIELNDSVIERLEVWGNGAVDEALFTLSDGRQLRVGERYATKYRKYAVDGHYIAGLYLASDEPSLAGQAAGIAVSYHMLDDKK

SEQ ID NO: 13 - DNA, 426 bp, Xenorhabdus cabanillasii
  source 1..426: mol_type = other DNA
  misc_feature 1..426: note = A nucleic acid sequence obtained from Xenorhabdus cabanillasiistrain 85908 encoding a TIC7660 PirA pesticidal protein sequence.
atgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaataa

SEQ ID NO: 14 - protein, 141 aa, Xenorhabdus cabanillasii
  REGION 1..141: note = The amino acid sequence of the TIC7660 PirA protein.
MITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNK

SEQ ID NO: 15 - DNA, 1278 bp, Xenorhabdus cabanillasii
  source 1..1278: mol_type = other DNA
  misc_feature 1..1278: note = A nucleic acid sequence obtained from Xenorhabdus cabanillasiistrain 85908 encoding a TIC7661 PirB pesticidal protein sequence.
atgaatactacacctattactgtatctacaaatgaaacatcgcctttaatgactgacgtaatgcccatggatctttatgcaatatccacacctgattatgaatgggacatgtcgtcaatcataaaggatgctgttattggtggcataggatttattccaggtccgggcccggcaatatccttcctgttagggctattttggcctcagcagaaagacaatacttgggagcaaattctccagaaagtagagcagatgatagagaatgctgttctacaaactattaaaggaatacttaatggagaagttcaagagatcaaagggaaaatggaacatgtagaatccatgctgaaaaactcgcctggcagtcaggaaagtcatgatgcatatatgttcctggcgagatatctggttagtatagatgaaaaattcaaatcttttgacaatagaacaaattaccagcttctcccaatgtatactaacactattatgttacagatcccttattggaaaatgggaatagagaagaaaaaagatattgggctgacagatattgaagttaatgaattaaaagaacttatcgataaattggttgataaggccaaaaactatattcatacgatgtatactaatgaacataataatgctgtaaacacatcaacagcagagagtgtcactaataatttattatctgtaagaggatattgtttattacacggtttagaatgtattgagttaatcgagcatatacagaataatagccttgagagtggtttctatcctaaaattatcagttattcgactgcgtttgatcgtcctactaacaaaatgagaattcaggctcttacagaagatgatgcaatgcaggagcctttcaaaccatctttaatcaatgggaaatataataaaatccaatccttgactggatatgtacaaagaattgggaatgcacctagagttggtggtatcagaatcacatttaccaacggctcatcttatacacttggtacagtgacctcagaaacgcattcaattaagctaaacgatagtgttatcgaaagcttggaagtatgggggaatggtgctgttgatgaggcgttatttaagttaagtgatgggcgtttattgcgtattggtgagcgctacgcgaaaaaatacagaaaatatgctgttgataatcactatattgcggggatttacttagccagcgatgagccttcacttgctggtcaagccgcaggtattgccgtttcatatcatatgatggctgacaaaaaataa

SEQ ID NO: 16 - protein, 425 aa, Xenorhabdus cabanillasii
  REGION 1..425: note = The amino acid sequence of the TIC7661 PirB protein.
MNTTPITVSTNETSPLMTDVMPMDLYAISTPDYEWDMSSIIKDAVIGGIGFIPGPGPAISFLLGLFWPQQKDNTWEQILQKVEQMIENAVLQTIKGILNGEVQEIKGKMEHVESMLKNSPGSQESHDAYMFLARYLVSIDEKFKSFDNRTNYQLLPMYTNTIMLQIPYWKMGIEKKKDIGLTDIEVNELKELIDKLVDKAKNYIHTMYTNEHNNAVNTSTAESVTNNLLSVRGYCLLHGLECIELIEHIQNNSLESGFYPKIISYSTAFDRPTNKMRIQALTEDDAMQEPFKPSLINGKYNKIQSLTGYVQRIGNAPRVGGIRITFTNGSSYTLGTVTSETHSIKLNDSVIESLEVWGNGAVDEALFKLSDGRLLRIGERYAKKYRKYAVDNHYIAGIYLASDEPSLAGQAAGIAVSYHMMADKK

SEQ ID NO: 17 - DNA, 1701 bp, synthetic construct
  source 1..1701: mol_type = other DNA
  misc_feature 1..1701: note = A nucleic acid sequence encoding a PirAB fusion protein, TIC9317comprised of the TIC7660 and TIC7661 coding sequences in operablelinkage and in frame.
atgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaaatgaatactacacctattactgtatctacaaatgaaacatcgcctttaatgactgacgtaatgcccatggatctttatgcaatatccacacctgattatgaatgggacatgtcgtcaatcataaaggatgctgttattggtggcataggatttattccaggtccgggcccggcaatatccttcctgttagggctattttggcctcagcagaaagacaatacttgggagcaaattctccagaaagtagagcagatgatagagaatgctgttctacaaactattaaaggaatacttaatggagaagttcaagagatcaaagggaaaatggaacatgtagaatccatgctgaaaaactcgcctggcagtcaggaaagtcatgatgcatatatgttcctggcgagatatctggttagtatagatgaaaaattcaaatcttttgacaatagaacaaattaccagcttctcccaatgtatactaacactattatgttacagatcccttattggaaaatgggaatagagaagaaaaaagatattgggctgacagatattgaagttaatgaattaaaagaacttatcgataaattggttgataaggccaaaaactatattcatacgatgtatactaatgaacataataatgctgtaaacacatcaacagcagagagtgtcactaataatttattatctgtaagaggatattgtttattacacggtttagaatgtattgagttaatcgagcatatacagaataatagccttgagagtggtttctatcctaaaattatcagttattcgactgcgtttgatcgtcctactaacaaaatgagaattcaggctcttacagaagatgatgcaatgcaggagcctttcaaaccatctttaatcaatgggaaatataataaaatccaatccttgactggatatgtacaaagaattgggaatgcacctagagttggtggtatcagaatcacatttaccaacggctcatcttatacacttggtacagtgacctcagaaacgcattcaattaagctaaacgatagtgttatcgaaagcttggaagtatgggggaatggtgctgttgatgaggcgttatttaagttaagtgatgggcgtttattgcgtattggtgagcgctacgcgaaaaaatacagaaaatatgctgttgataatcactatattgcggggatttacttagccagcgatgagccttcacttgctggtcaagccgcaggtattgccgtttcatatcatatgatggctgacaaaaaataa

SEQ ID NO: 18 - protein, 566 aa, synthetic construct
  REGION 1..566: note = The amino acid sequence of the TIC9317 PirAB fusion protein.
MITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNKMNTTPITVSTNETSPLMTDVMPMDLYAISTPDYEWDMSSIIKDAVIGGIGFIPGPGPAISFLLGLFWPQQKDNTWEQILQKVEQMIENAVLQTIKGILNGEVQEIKGKMEHVESMLKNSPGSQESHDAYMFLARYLVSIDEKFKSFDNRTNYQLLPMYTNTIMLQIPYWKMGIEKKKDIGLTDIEVNELKELIDKLVDKAKNYIHTMYTNEHNNAVNTSTAESVTNNLLSVRGYCLLHGLECIELIEHIQNNSLESGFYPKIISYSTAFDRPTNKMRIQALTEDDAMQEPFKPSLINGKYNKIQSLTGYVQRIGNAPRVGGIRITFTNGSSYTLGTVTSETHSIKLNDSVIESLEVWGNGAVDEALFKLSDGRLLRIGERYAKKYRKYAVDNHYIAGIYLASDEPSLAGQAAGIAVSYHMMADKK

SEQ ID NO: 19 - DNA, 420 bp, Xenorhabdus ehlersii
  source 1..420: mol_type = other DNA
  misc_feature 1..420: note = A nucleic acid sequence obtained from Xenorhabdus ehlersii strain85887 encoding a TIC7662 PirA pesticidal protein sequence.
atgagtacaatcaatatcaatataagtagcagtaccgttaccgtcatcacgaataacggagaaacgccagtcccactcacttacaatacaaatacacctgaatcagaacctcttaccgtcaatccttatagggatatgacaatagagccacgctcttctattgaagcaacaaggattgatacaccgattattcccgaaacacgccctaattattatgtagccaattcaggcccggcttcatcagttagggccgttttttattggtcccattctttcacatcacaatggttcgaatattcctctatcatcgtcaaagccggggaagatggcatattagaatcaccaagcaattctttatattacagcaaagtcgtcatttataatgataccgataaacgcgcctttgtgacgggatataat

SEQ ID NO: 20 - protein, 141 aa, Xenorhabdus ehlersii
  REGION 1..141: note = The amino acid sequence of the TIC7662 PirA protein.
MSTININISSSTVTVITNNGETPVPLTYNTNTPESEPLTVNPYRDMTIEPRSSIEATRIDTPIIPETRPNYYVANSGPASSVRAVFYWSHSFTSQWFEYSSIIVKAGEDGILESPSNSLYYSKVVIYNDTDKRAFVTGYNK

SEQ ID NO: 21 - DNA, 1278 bp, Xenorhabdus ehlersii
  source 1..1278: mol_type = other DNA
  misc_feature 1..1278: note = A nucleic acid sequence obtained from Xenorhabdus ehlersii strain85887 encoding a TIC7663 PirB pesticidal protein sequence.
atgaataccactctgattaatgtatctgaaaaagaaacattgcctgtacaaactgatatcatgcttatcgcgccttattcagtatcgacccccgattatgaatgggatatgtcctcactcatcaaggatgccattattggtggcgtagggtttattcccgtcgtaggttccgcaatgtccttcctgctaggattattttggccccaacagaaagataatacttgggagcaaattctccaaaaagtcgagcagatgatcgagaatgcccagctaaatacgattaaaggaatacttaatggcgatatacaagagatcaaaggaaaaatggagcatgtacaatacatgttggaaacctcgccgggcagtcaagaaagtcatgatgcctatatgttcctggccagatatctggtgagtatcgatgagaaatttaagtcttttgataataaaacaaactatcaaattttgccgatgtatacgaacacggttatgttgcagatcccttattggaaaatggggatcgagaagaaaaatgatattgggctgaccgatattgaagtcaatgagttaaaacagcttatcgacacattggttgacagagccaggaactatattcatacgatgtatgaaagagaatatgataatgccatcaacacctcaaccgcggcgagcgtcactaataatttattgtccgtcagaggatattgcctgttacacggtttagagtgtattgaaaccattgaacatctgcaaaataatagccttaatagtggtttctatcctaaaaccattagttattcaacggtatttgatcgtcccacgaacaaaacgagaattcaggctctgaccgaagatgaccaaatgcaagagcctttcaagccagctttaattggcggtaagtacaataaaataaaatcattgcttggctatgtacgaagaattgggaatgcccccagagtggggggaattaaggtcacctttaccaacggatcatcttatacacttggcacagtcacatcagaaacggactcaattgagctaaatgagagtgttatcgaaagattagaagtatggggcaatggtgctgttgatgaggcattatttacgttaagcgatgggcgccaactcaggatcggcgagcgctacgcgaaaaaatacagaaaatatgctgttgatggacactatatttcagggctgtacttagccagcgatgaaccttcccttgctggtcaggccgcaggtattgccgtttcataccatatgcttgctgataaaaaataa

SEQ ID NO: 22 - protein, 425 aa, Xenorhabdus ehlersii
  REGION 1..425: note = The amino acid sequence of the TIC7663 PirB protein.
MNTTLINVSEKETLPVQTDIMLIAPYSVSTPDYEWDMSSLIKDAIIGGVGFIPVVGSAMSFLLGLFWPQQKDNTWEQILQKVEQMIENAQLNTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDTLVDRARNYIHTMYEREYDNAINTSTAASVTNNLLSVRGYCLLHGLECIETIEHLQNNSLNSGFYPKTISYSTVFDRPTNKTRIQALTEDDQMQEPFKPALIGGKYNKIKSLLGYVRRIGNAPRVGGIKVTFTNGSSYTLGTVTSETDSIELNESVIERLEVWGNGAVDEALFTLSDGRQLRIGERYAKKYRKYAVDGHYISGLYLASDEPSLAGQAAGIAVSYHMLADKK

SEQ ID NO: 23 - DNA, 1701 bp, synthetic construct
  source 1..1701: mol_type = other DNA
  misc_feature 1..1701: note = A nucleic acid sequence encoding a PirAB fusion protein, TIC9318comprised of the TIC7662 and TIC7663 coding sequences in operablelinkage and in frame.
atgagtacaatcaatatcaatataagtagcagtaccgttaccgtcatcacgaataacggagaaacgccagtcccactcacttacaatacaaatacacctgaatcagaacctcttaccgtcaatccttatagggatatgacaatagagccacgctcttctattgaagcaacaaggattgatacaccgattattcccgaaacacgccctaattattatgtagccaattcaggcccggcttcatcagttagggccgttttttattggtcccattctttcacatcacaatggttcgaatattcctctatcatcgtcaaagccggggaagatggcatattagaatcaccaagcaattctttatattacagcaaagtcgtcatttataatgataccgataaacgcgcctttgtgacgggatataataagatgaataccactctgattaatgtatctgaaaaagaaacattgcctgtacaaactgatatcatgcttatcgcgccttattcagtatcgacccccgattatgaatgggatatgtcctcactcatcaaggatgccattattggtggcgtagggtttattcccgtcgtaggttccgcaatgtccttcctgctaggattattttggccccaacagaaagataatacttgggagcaaattctccaaaaagtcgagcagatgatcgagaatgcccagctaaatacgattaaaggaatacttaatggcgatatacaagagatcaaaggaaaaatggagcatgtacaatacatgttggaaacctcgccgggcagtcaagaaagtcatgatgcctatatgttcctggccagatatctggtgagtatcgatgagaaatttaagtcttttgataataaaacaaactatcaaattttgccgatgtatacgaacacggttatgttgcagatcccttattggaaaatggggatcgagaagaaaaatgatattgggctgaccgatattgaagtcaatgagttaaaacagcttatcgacacattggttgacagagccaggaactatattcatacgatgtatgaaagagaatatgataatgccatcaacacctcaaccgcggcgagcgtcactaataatttattgtccgtcagaggatattgcctgttacacggtttagagtgtattgaaaccattgaacatctgcaaaataatagccttaatagtggtttctatcctaaaaccattagttattcaacggtatttgatcgtcccacgaacaaaacgagaattcaggctctgaccgaagatgaccaaatgcaagagcctttcaagccagctttaattggcggtaagtacaataaaataaaatcattgcttggctatgtacgaagaattgggaatgcccccagagtggggggaattaaggtcacctttaccaacggatcatcttatacacttggcacagtcacatcagaaacggactcaattgagctaaatgagagtgttatcgaaagattagaagtatggggcaatggtgctgttgatgaggcattatttacgttaagcgatgggcgccaactcaggatcggcgagcgctacgcgaaaaaatacagaaaatatgctgttgatggacactatatttcagggctgtacttagccagcgatgaaccttcccttgctggtcaggccgcaggtattgccgtttcataccatatgcttgctgataaaaaataa

SEQ ID NO: 24 - protein, 566 aa, synthetic construct
  REGION 1..566: note = The amino acid sequence of the TIC9318 PirAB fusion protein.
MSTININISSSTVTVITNNGETPVPLTYNTNTPESEPLTVNPYRDMTIEPRSSIEATRIDTPIIPETRPNYYVANSGPASSVRAVFYWSHSFTSQWFEYSSIIVKAGEDGILESPSNSLYYSKVVIYNDTDKRAFVTGYNKMNTTLINVSEKETLPVQTDIMLIAPYSVSTPDYEWDMSSLIKDAIIGGVGFIPVVGSAMSFLLGLFWPQQKDNTWEQILQKVEQMIENAQLNTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDTLVDRARNYIHTMYEREYDNAINTSTAASVTNNLLSVRGYCLLHGLECIETIEHLQNNSLNSGFYPKTISYSTVFDRPTNKTRIQALTEDDQMQEPFKPALIGGKYNKIKSLLGYVRRIGNAPRVGGIKVTFTNGSSYTLGTVTSETDSIELNESVIERLEVWGNGAVDEALFTLSDGRQLRIGERYAKKYRKYAVDGHYISGLYLASDEPSLAGQAAGIAVSYHMLADKK

SEQ ID NO: 25 - DNA, 408 bp, Xenorhabdus poinarii
  source 1..408: mol_type = other DNA
  misc_feature 1..408: note = A nucleic acid sequence obtained from Xenorhabdus poinarii strain86198 encoding a TIC7664 PirA pesticidal protein sequence.
atgatcacaatcaatatcagtggtggtaatgtaacaattaataacaatatcagttcagtaacggatatccaaaaaccccttgatgcagaacccctctcagtcacgaattataaggatctgacaatagagccgcactcatctattcaagcaaccagaacggacacccccattattcctgaaacacgccctaattattatgttgctaactcaggccctgcttcatccgttaaagcggtgttttattggtcgcattcgtttacatcggaatggttcgagtattcatctatcatagtaaaagcaggagaggatggaatattaaaatcaccgagtaatgccgtatattacagtaaagtagtaatttataatgatacagataagcgggcttttgtgactggatataacatgtaa

SEQ ID NO: 26 - protein, 135 aa, Xenorhabdus poinarii
  REGION 1..135: note = The amino acid sequence of the TIC7664 PirA protein.
MITINISGGNVTINNNISSVTDIQKPLDAEPLSVTNYKDLTIEPHSSIQATRTDTPIIPETRPNYYVANSGPASSVKAVFYWSHSFTSEWFEYSSIIVKAGEDGILKSPSNAVYYSKVVIYNDTDKRAFVTGYNM

SEQ ID NO: 27 - DNA, 1245 bp, Xenorhabdus poinarii
  source 1..1245: mol_type = other DNA
  misc_feature 1..1245: note = A nucleic acid sequence obtained from Xenorhabdus poinarii strain86198 encoding a TIC7665 PirB pesticidal protein sequence.
atgaataatagtccaatgaatgatcagttatcaatagcaccttattcaatttcgacacccaattatgaatgggatatgtcatcaatcataaaagatgccattatcggtggcataggatttattcccggaccaggctctgcaatctcttttttattagggctgttctggcctcaacagacagacaatacctgggatcaaatcctccaaaaaatcgaacagatgatagaagaagcgaatttaaaaaccattaaaggtatattaaatggagatatacaagaaattaaaggaaaaatggaccatgtgcaatatatgctagagaattctcctggcagccaggaaagccatgatgcttatatgtttttagcaaggtttttggtcagtattgatgaaaaattcaaatctttcgatgatagaacaaattatcaaattcttcccatgtatacgaacaccattatgttacaagcgccttattggaaaatgggcctcgaaaagaaagaggatatcggtttaagcgatattgaagttagcgaattaaaagaacttatcgataaattatatactaaatcatatgattatatccataacacgtataatcgtgaatatgataatgcaatcaatacgtcaaccgcagagagtatcaccaataatttattgtctgtcagaggatattgtttattacatggttgtgaatgtcttgaagttattgcgcatatacaaaacaatagccttgataaaggcttctaccctaaaacgatcagctattcgagtgttttcgatcgtcctacaaacaaaatgaggattcaggcgcttacagaagatgaccaaatgcaagaaccgttcaaaccttctttcgtcaatggtcaatataataaaataaaatcattggagggttatgtcacaaggatcggcaatgccccccgagtcggtggaattaaaatcacatttgaaaacaacgcatcttatactcttggtactgtgacttcagaaacaacctttattgaactcaatgagagtgttataaccagcatagaagtgtggggaaatggggccgttgatgaggcattctttacattgagtgacggtcgccaaatgcggcttggtcaacgctatgccagtcgctacagaaaatatgctgtcgatggtcattatatctcaggattgtacttagccagtgatgaaccatcccttgctggtcaagccgccggtattgccgtttcatatcatatgattgttgataaacaataa

SEQ ID NO: 28 - protein, 414 aa, Xenorhabdus poinarii
  REGION 1..414: note = The amino acid sequence of the TIC7665 PirB protein.
MNNSPMNDQLSIAPYSISTPNYEWDMSSIIKDAIIGGIGFIPGPGSAISFLLGLFWPQQTDNTWDQILQKIEQMIEEANLKTIKGILNGDIQEIKGKMDHVQYMLENSPGSQESHDAYMFLARFLVSIDEKFKSFDDRTNYQILPMYTNTIMLQAPYWKMGLEKKEDIGLSDIEVSELKELIDKLYTKSYDYIHNTYNREYDNAINTSTAESITNNLLSVRGYCLLHGCECLEVIAHIQNNSLDKGFYPKTISYSSVFDRPTNKMRIQALTEDDQMQEPFKPSFVNGQYNKIKSLEGYVTRIGNAPRVGGIKITFENNASYTLGTVTSETTFIELNESVITSIEVWGNGAVDEAFFTLSDGRQMRLGQRYASRYRKYAVDGHYISGLYLASDEPSLAGQAAGIAVSYHMIVDKQ

SEQ ID NO: 29 - DNA, 1650 bp, synthetic construct
  source 1..1650: mol_type = other DNA
  misc_feature 1..1650: note = A nucleic acid sequence encoding a PirAB fusion protein, TIC9319comprised of the TIC7664 and TIC7665 coding sequences in operablelinkage and in frame.
atgatcacaatcaatatcagtggtggtaatgtaacaattaataacaatatcagttcagtaacggatatccaaaaaccccttgatgcagaacccctctcagtcacgaattataaggatctgacaatagagccgcactcatctattcaagcaaccagaacggacacccccattattcctgaaacacgccctaattattatgttgctaactcaggccctgcttcatccgttaaagcggtgttttattggtcgcattcgtttacatcggaatggttcgagtattcatctatcatagtaaaagcaggagaggatggaatattaaaatcaccgagtaatgccgtatattacagtaaagtagtaatttataatgatacagataagcgggcttttgtgactggatataacatgatgaataatagtccaatgaatgatcagttatcaatagcaccttattcaatttcgacacccaattatgaatgggatatgtcatcaatcataaaagatgccattatcggtggcataggatttattcccggaccaggctctgcaatctcttttttattagggctgttctggcctcaacagacagacaatacctgggatcaaatcctccaaaaaatcgaacagatgatagaagaagcgaatttaaaaaccattaaaggtatattaaatggagatatacaagaaattaaaggaaaaatggaccatgtgcaatatatgctagagaattctcctggcagccaggaaagccatgatgcttatatgtttttagcaaggtttttggtcagtattgatgaaaaattcaaatctttcgatgatagaacaaattatcaaattcttcccatgtatacgaacaccattatgttacaagcgccttattggaaaatgggcctcgaaaagaaagaggatatcggtttaagcgatattgaagttagcgaattaaaagaacttatcgataaattatatactaaatcatatgattatatccataacacgtataatcgtgaatatgataatgcaatcaatacgtcaaccgcagagagtatcaccaataatttattgtctgtcagaggatattgtttattacatggttgtgaatgtcttgaagttattgcgcatatacaaaacaatagccttgataaaggcttctaccctaaaacgatcagctattcgagtgttttcgatcgtcctacaaacaaaatgaggattcaggcgcttacagaagatgaccaaatgcaagaaccgttcaaaccttctttcgtcaatggtcaatataataaaataaaatcattggagggttatgtcacaaggatcggcaatgccccccgagtcggtggaattaaaatcacatttgaaaacaacgcatcttatactcttggtactgtgacttcagaaacaacctttattgaactcaatgagagtgttataaccagcatagaagtgtggggaaatggggccgttgatgaggcattctttacattgagtgacggtcgccaaatgcggcttggtcaacgctatgccagtcgctacagaaaatatgctgtcgatggtcattatatctcaggattgtacttagccagtgatgaaccatcccttgctggtcaagccgccggtattgccgtttcatatcatatgattgttgataaacaataa

SEQ ID NO: 30 - protein, 549 aa, synthetic construct
  REGION 1..549: note = The amino acid sequence of the TIC9319 PirAB fusion protein.
MITINISGGNVTINNNISSVTDIQKPLDAEPLSVTNYKDLTIEPHSSIQATRTDTPIIPETRPNYYVANSGPASSVKAVFYWSHSFTSEWFEYSSIIVKAGEDGILKSPSNAVYYSKVVIYNDTDKRAFVTGYNMMNNSPMNDQLSIAPYSISTPNYEWDMSSIIKDAIIGGIGFIPGPGSAISFLLGLFWPQQTDNTWDQILQKIEQMIEEANLKTIKGILNGDIQEIKGKMDHVQYMLENSPGSQESHDAYMFLARFLVSIDEKFKSFDDRTNYQILPMYTNTIMLQAPYWKMGLEKKEDIGLSDIEVSELKELIDKLYTKSYDYIHNTYNREYDNAINTSTAESITNNLLSVRGYCLLHGCECLEVIAHIQNNSLDKGFYPKTISYSSVFDRPTNKMRIQALTEDDQMQEPFKPSFVNGQYNKIKSLEGYVTRIGNAPRVGGIKITFENNASYTLGTVTSETTFIELNESVITSIEVWGNGAVDEAFFTLSDGRQMRLGQRYASRYRKYAVDGHYISGLYLASDEPSLAGQAAGIAVSYHMIVDKQ

SEQ ID NO: 31 - DNA, 402 bp, Photorhabdus luminescens
  source 1..402: mol_type = other DNA
  misc_feature 1..402: note = A nucleic acid sequence obtained from Photorhabdus luminescensstrain 86197 encoding a TIC7666 PirA pesticidal protein sequence.
atgtctagaataaccattgttgttgattcagatgatcagaaagcagaattttattctaattctcctgttccagtatataaagatttaaatgcagttggtcctttgagtgatgtgactatatcacctcatgccagtgtggaagtgtttagaatagatacaccagtaattccagaatccagaagctctctgagagttgtaaatacagggctatcaaatagtgttacggctaaattttactggtctcatagttttacctctgaatggtttgagtctggttctatagatgtaggattaggagaagagaaggtgttaaacgtgcctagcaactctttttattatagtaaatttgttatctataataacacggacaaagttgcttatgtgacggcaaatttggtttaa

SEQ ID NO: 32 - protein, 133 aa, Photorhabdus luminescens
  REGION 1..133: note = The amino acid sequence of the TIC7666 PirA protein.
MSRITIVVDSDDQKAEFYSNSPVPVYKDLNAVGPLSDVTISPHASVEVFRIDTPVIPESRSSLRVVNTGLSNSVTAKFYWSHSFTSEWFESGSIDVGLGEEKVLNVPSNSFYYSKFVIYNNTDKVAYVTANLV

SEQ ID NO: 33 - DNA, 1260 bp, Photorhabdus luminescens
  source 1..1260: mol_type = other DNA
  misc_feature 1..1260: note = A nucleic acid sequence obtained from Photorhabdus luminescensstrain 86197 encoding a TIC7667 pesticidal PirB protein sequence.
atgcatacagaaaatgttttagacataagaaccattgtggctaatgaatatgctgtaaaaacgagtgcattagagtgggatgttactgatattgtaaaaaatgcaatcatagggggaatatcctttatcccttcggttggtcccgctatatcttttttagtcggtttattctggcctcaatcgaaagaaaatatatgggaagggattgtcaaacaaattgaaaggatgatagaggagtctgcgttaaagacgattaaaggtatccttgctggtgatattgcatatatacaagaacgaatggcaaccgttgctgatcttcttgataagcatccaggatcagaagaagcgaggagtgcttttaataacctggcagaaaatatagatggctatcacaaaaagtttagtaatttttcggatgatgttaattatcagatattacccatgttttctactacggttatgatgcagataacatattgggttgctggtttagagagaaaagatgaaattgggcttagtaatattgatgttgaaaaagtccgaggattaattaaaaagacggtagaacaggctaatagttatattaacaatatatatgatagagagcttaatgatgctcttaataactcgacggctgacactgttgcaaataatgttatgtctgttcatggtcactgtcgtttacatgggattgaatatatcagtatttgggataaattaagtgaagctgagtcggtaaataataaaatctatgttgatgttttaagttattctactttctttgaccgtcaaacagcaaaagccagaattcaggcattgactccagagaaagatatgactccacctctcaaaccggctcttaatggaggaaaaagaagaaagatagattcgttaacgggacatattgtgcgtattggaggggctgcgagggtaggagggctgacagttgtatttgatgatggtaatcgccatcaattaggtacaatatctggtgagacgtcatctatttctctgaatggtagtcgaattaccagtttggaagtatggggaaatggtgctgttgatcaagcggtctttactttaaatgatggtcgttcattgtcattgggctcgcctggaacatctcgatataggaagttttatgttggtgaaagccactatattgcagggatatatttgtccagtgattacaacccattagctggtcaggcagcaaatattgctgtatcttatcagttgataaatgatgatgaaaaataa

SEQ ID NO: 34 - protein, 419 aa, Photorhabdus luminescens
  REGION 1..419: note = The amino acid sequence of the TIC7667 PirB protein.
MHTENVLDIRTIVANEYAVKTSALEWDVTDIVKNAIIGGISFIPSVGPAISFLVGLFWPQSKENIWEGIVKQIERMIEESALKTIKGILAGDIAYIQERMATVADLLDKHPGSEEARSAFNNLAENIDGYHKKFSNFSDDVNYQILPMFSTTVMMQITYWVAGLERKDEIGLSNIDVEKVRGLIKKTVEQANSYINNIYDRELNDALNNSTADTVANNVMSVHGHCRLHGIEYISIWDKLSEAESVNNKIYVDVLSYSTFFDRQTAKARIQALTPEKDMTPPLKPALNGGKRRKIDSLTGHIVRIGGAARVGGLTVVFDDGNRHQLGTISGETSSISLNGSRITSLEVWGNGAVDQAVFTLNDGRSLSLGSPGTSRYRKFYVGESHYIAGIYLSSDYNPLAGQAANIAVSYQLINDDEK

SEQ ID NO: 35 - DNA, 1659 bp, synthetic construct
  source 1..1659: mol_type = other DNA
  misc_feature 1..1659: note = A nucleic acid sequence encoding a PirAB fusion protein, TIC9322comprised of the TIC7666 and TIC7667 coding sequences in operablelinkage and in frame.
atgtctagaataaccattgttgttgattcagatgatcagaaagcagaattttattctaattctcctgttccagtatataaagatttaaatgcagttggtcctttgagtgatgtgactatatcacctcatgccagtgtggaagtgtttagaatagatacaccagtaattccagaatccagaagctctctgagagttgtaaatacagggctatcaaatagtgttacggctaaattttactggtctcatagttttacctctgaatggtttgagtctggttctatagatgtaggattaggagaagagaaggtgttaaacgtgcctagcaactctttttattatagtaaatttgttatctataataacacggacaaagttgcttatgtgacggcaaatttggttatgcatacagaaaatgttttagacataagaaccattgtggctaatgaatatgctgtaaaaacgagtgcattagagtgggatgttactgatattgtaaaaaatgcaatcatagggggaatatcctttatcccttcggttggtcccgctatatcttttttagtcggtttattctggcctcaatcgaaagaaaatatatgggaagggattgtcaaacaaattgaaaggatgatagaggagtctgcgttaaagacgattaaaggtatccttgctggtgatattgcatatatacaagaacgaatggcaaccgttgctgatcttcttgataagcatccaggatcagaagaagcgaggagtgcttttaataacctggcagaaaatatagatggctatcacaaaaagtttagtaatttttcggatgatgttaattatcagatattacccatgttttctactacggttatgatgcagataacatattgggttgctggtttagagagaaaagatgaaattgggcttagtaatattgatgttgaaaaagtccgaggattaattaaaaagacggtagaacaggctaatagttatattaacaatatatatgatagagagcttaatgatgctcttaataactcgacggctgacactgttgcaaataatgttatgtctgttcatggtcactgtcgtttacatgggattgaatatatcagtatttgggataaattaagtgaagctgagtcggtaaataataaaatctatgttgatgttttaagttattctactttctttgaccgtcaaacagcaaaagccagaattcaggcattgactccagagaaagatatgactccacctctcaaaccggctcttaatggaggaaaaagaagaaagatagattcgttaacgggacatattgtgcgtattggaggggctgcgagggtaggagggctgacagttgtatttgatgatggtaatcgccatcaattaggtacaatatctggtgagacgtcatctatttctctgaatggtagtcgaattaccagtttggaagtatggggaaatggtgctgttgatcaagcggtctttactttaaatgatggtcgttcattgtcattgggctcgcctggaacatctcgatataggaagttttatgttggtgaaagccactatattgcagggatatatttgtccagtgattacaacccattagctggtcaggcagcaaatattgctgtatcttatcagttgataaatgatgatgaaaaataa

SEQ ID NO: 36 - protein, 552 aa, synthetic construct
  REGION 1..552: note = The amino acid sequence of the TIC9322 PirAB fusion protein.
MSRITIVVDSDDQKAEFYSNSPVPVYKDLNAVGPLSDVTISPHASVEVFRIDTPVIPESRSSLRVVNTGLSNSVTAKFYWSHSFTSEWFESGSIDVGLGEEKVLNVPSNSFYYSKFVIYNNTDKVAYVTANLVMHTENVLDIRTIVANEYAVKTSALEWDVTDIVKNAIIGGISFIPSVGPAISFLVGLFWPQSKENIWEGIVKQIERMIEESALKTIKGILAGDIAYIQERMATVADLLDKHPGSEEARSAFNNLAENIDGYHKKFSNFSDDVNYQILPMFSTTVMMQITYWVAGLERKDEIGLSNIDVEKVRGLIKKTVEQANSYINNIYDRELNDALNNSTADTVANNVMSVHGHCRLHGIEYISIWDKLSEAESVNNKIYVDVLSYSTFFDRQTAKARIQALTPEKDMTPPLKPALNGGKRRKIDSLTGHIVRIGGAARVGGLTVVFDDGNRHQLGTISGETSSISLNGSRITSLEVWGNGAVDQAVFTLNDGRSLSLGSPGTSRYRKFYVGESHYIAGIYLSSDYNPLAGQAANIAVSYQLINDDEK

SEQ ID NO: 37 - DNA, 402 bp, Photorhabdus luminescens
  source 1..402: mol_type = other DNA
  misc_feature 1..402: note = A nucleic acid sequence obtained from Photorhabdus luminescensstrain 86194 encoding a TIC7668 PirA pesticidal protein sequence.
atgtctagaataactattgttgttgattcagatgaacagaaagcagaagtttactctaattcccctgttccagtacataaagacttaaatgcagttggtcctttgagtgatgtgactatatcacctcatgctagtgtggaagtatttagaatagatacaccaataattccagaatccagaagatctctgagagttgtaaataccgggctagcaaatagtgtcacggctaaattttactggtctcatagttttacctctgaatggtttgagtctggttctatagatgtaggattaggagaagagaaggtgttaaacgtgcctaataactctttttattatagtaaatttgttatctataataatacggataaagttgcttatgtgacggcaaatttggtttaa

SEQ ID NO: 38 - protein, 133 aa, Photorhabdus luminescens
  REGION 1..133: note = The amino acid sequence of the TIC7668 PirA protein.
MSRITIVVDSDEQKAEVYSNSPVPVHKDLNAVGPLSDVTISPHASVEVFRIDTPIIPESRRSLRVVNTGLANSVTAKFYWSHSFTSEWFESGSIDVGLGEEKVLNVPNNSFYYSKFVIYNNTDKVAYVTANLV

SEQ ID NO: 39 - DNA, 1260 bp, Photorhabdus luminescens
  source 1..1260: mol_type = other DNA
  misc_feature 1..1260: note = A nucleic acid sequence obtained from Photorhabdus luminescensstrain 86194 encoding a TIC7669 PirB pesticidal protein sequence.
atgcatacagaaaatgttttagacataagaaccattgtggctaatgaatatgccgtaaaaacgagtgcagtagagtgggatgttactgatattgtaaaaaatgcaatcatcgggggaatatcttttataccttcagttggccctgctatatcttttttagtcggtttattctggcctcaatcaaaagaaaatatatgggaagggattgtcaaacaaattgaaaggatgatagaggagtctgcattaaagacgattaaaggtatccttgctggtgatattgcatatatacaagaacgaatggcaaccgttgctgatcttcttgataagcatccgggatctgaagaagcgaggagtgcttttaataacctggcagaaaatatagatggttatcacaaaaaatttaataatttttccgatgatgttaactatcagatattacccatgttttctactacggttatgatgcagataacatattgggtcgctggtttagagagaagaaatgaaatcgggcttagtgatattgatattgaaaaagtccgagggttaatcaaaaagacggtagaacaggcgaatagttatattaataatatatatgatagagagcttaatgatgctcttaataactcgacggctgacactgttgcaaataatgtgatgtctgttcatggtcactgtcgtttacatgggattgaatatatcagtatttgggataaattaagtgaagcagagtcagtaaataatagaatctatgttgatgttttaagttattctactttctttgaccgtcaaacagcaaaagccagaattcaggcattgactccagagaaagatatggctccacctctcaaaccggctcttaatgacgggaaaagaagaaagatcgattcgttaacgggacatattgtgcgtattggaggggctccgagagtcggagggctgacagttgtatttgatgatggtagtagccatcgattaggtacaatatctggtgagacggcatctatttctctgaatggcagtcgaattaccagtttggaagtatggggcaatggtgctgttgatcaagcggtctttactttgagtgatggtcgttcattatcatttggcgcacctggaacatctcgatataggaaattttatgttggcgaaagtcactatattgcaggggtatatttgtccagtgactacagcccattagcaggtcaggcagcaaatatcgctgtatcttatcagttgataaatgatgatgaaaaataa

SEQ ID NO: 40 - protein, 419 aa, Photorhabdus luminescens
  REGION 1..419: note = The amino acid sequence of the TIC7669 PirB protein.
MHTENVLDIRTIVANEYAVKTSAVEWDVTDIVKNAIIGGISFIPSVGPAISFLVGLFWPQSKENIWEGIVKQIERMIEESALKTIKGILAGDIAYIQERMATVADLLDKHPGSEEARSAFNNLAENIDGYHKKFNNFSDDVNYQILPMFSTTVMMQITYWVAGLERRNEIGLSDIDIEKVRGLIKKTVEQANSYINNIYDRELNDALNNSTADTVANNVMSVHGHCRLHGIEYISIWDKLSEAESVNNRIYVDVLSYSTFFDRQTAKARIQALTPEKDMAPPLKPALNDGKRRKIDSLTGHIVRIGGAPRVGGLTVVFDDGSSHRLGTISGETASISLNGSRITSLEVWGNGAVDQAVFTLSDGRSLSFGAPGTSRYRKFYVGESHYIAGVYLSSDYSPLAGQAANIAVSYQLINDDEK

SEQ ID NO: 41 - DNA, 1659 bp, synthetic construct
  source 1..1659: mol_type = other DNA
  misc_feature 1..1659: note = A nucleic acid sequence encoding a PirAB fusion protein, TIC9320comprised of the TIC7668 and TIC7669 coding sequences in operablelinkage and in frame.
atgtctagaataactattgttgttgattcagatgaacagaaagcagaagtttactctaattcccctgttccagtacataaagacttaaatgcagttggtcctttgagtgatgtgactatatcacctcatgctagtgtggaagtatttagaatagatacaccaataattccagaatccagaagatctctgagagttgtaaataccgggctagcaaatagtgtcacggctaaattttactggtctcatagttttacctctgaatggtttgagtctggttctatagatgtaggattaggagaagagaaggtgttaaacgtgcctaataactctttttattatagtaaatttgttatctataataatacggataaagttgcttatgtgacggcaaatttggttatgcatacagaaaatgttttagacataagaaccattgtggctaatgaatatgccgtaaaaacgagtgcagtagagtgggatgttactgatattgtaaaaaatgcaatcatcgggggaatatcttttataccttcagttggccctgctatatcttttttagtcggtttattctggcctcaatcaaaagaaaatatatgggaagggattgtcaaacaaattgaaaggatgatagaggagtctgcattaaagacgattaaaggtatccttgctggtgatattgcatatatacaagaacgaatggcaaccgttgctgatcttcttgataagcatccgggatctgaagaagcgaggagtgcttttaataacctggcagaaaatatagatggttatcacaaaaaatttaataatttttccgatgatgttaactatcagatattacccatgttttctactacggttatgatgcagataacatattgggtcgctggtttagagagaagaaatgaaatcgggcttagtgatattgatattgaaaaagtccgagggttaatcaaaaagacggtagaacaggcgaatagttatattaataatatatatgatagagagcttaatgatgctcttaataactcgacggctgacactgttgcaaataatgtgatgtctgttcatggtcactgtcgtttacatgggattgaatatatcagtatttgggataaattaagtgaagcagagtcagtaaataatagaatctatgttgatgttttaagttattctactttctttgaccgtcaaacagcaaaagccagaattcaggcattgactccagagaaagatatggctccacctctcaaaccggctcttaatgacgggaaaagaagaaagatcgattcgttaacgggacatattgtgcgtattggaggggctccgagagtcggagggctgacagttgtatttgatgatggtagtagccatcgattaggtacaatatctggtgagacggcatctatttctctgaatggcagtcgaattaccagtttggaagtatggggcaatggtgctgttgatcaagcggtctttactttgagtgatggtcgttcattatcatttggcgcacctggaacatctcgatataggaaattttatgttggcgaaagtcactatattgcaggggtatatttgtccagtgactacagcccattagcaggtcaggcagcaaatatcgctgtatcttatcagttgataaatgatgatgaaaaataa

SEQ ID NO: 42 - protein, 552 aa, synthetic construct
  REGION 1..552: note = The amino acid sequence of the TIC9320 PirAB fusion protein
MSRITIVVDSDEQKAEVYSNSPVPVHKDLNAVGPLSDVTISPHASVEVFRIDTPIIPESRRSLRVVNTGLANSVTAKFYWSHSFTSEWFESGSIDVGLGEEKVLNVPNNSFYYSKFVIYNNTDKVAYVTANLVMHTENVLDIRTIVANEYAVKTSAVEWDVTDIVKNAIIGGISFIPSVGPAISFLVGLFWPQSKENIWEGIVKQIERMIEESALKTIKGILAGDIAYIQERMATVADLLDKHPGSEEARSAFNNLAENIDGYHKKFNNFSDDVNYQILPMFSTTVMMQITYWVAGLERRNEIGLSDIDIEKVRGLIKKTVEQANSYINNIYDRELNDALNNSTADTVANNVMSVHGHCRLHGIEYISIWDKLSEAESVNNRIYVDVLSYSTFFDRQTAKARIQALTPEKDMAPPLKPALNDGKRRKIDSLTGHIVRIGGAPRVGGLTVVFDDGSSHRLGTISGETASISLNGSRITSLEVWGNGAVDQAVFTLSDGRSLSFGAPGTSRYRKFYVGESHYIAGVYLSSDYSPLAGQAANIAVSYQLINDDEK

SEQ ID NO: 43 - DNA, 420 bp, unidentified
  source 1..420: mol_type = other DNA
  misc_feature 1..420: note = A nucleic acid sequence obtained from an unknown bacterial straincomprised within a microbiome encoding a TIC7939 pesticidal PirAprotein sequence.
atgacgtgtgagattctgcatatgacaagcaaaggcgatgaaatgcagtcgattgcagcgacggatgctcaaacgttacaggaggcgcctaaagatgaagtgaattttaagcagacaaaaggggatatgatggtccctggggggcagtctgcacagggagcgcgctacgatactccgattattcctgaacttcatccgtcttactatgtatcaaattcaggacctgcagctacggtgaaagctgtcttctactggtcccactcatttacctcgaagtggtttgaatataattcagttacggttctcagggggactactgagcggcttagtgcgccaagcaactcactttattacagcaaggtcgttgtctttaataatgagaaagagcctgcttatgttactgtaacgaccattcggtaa

SEQ ID NO: 44 - protein, 139 aa, unidentified
  REGION 1..139: note = The amino acid sequence of the TIC7939 PirA protein.
MTCEILHMTSKGDEMQSIAATDAQTLQEAPKDEVNFKQTKGDMMVPGGQSAQGARYDTPIIPELHPSYYVSNSGPAATVKAVFYWSHSFTSKWFEYNSVTVLRGTTERLSAPSNSLYYSKVVVFNNEKEPAYVTVTTIR

SEQ ID NO: 45 - DNA, 1260 bp, unidentified
  source 1..1260: mol_type = other DNA
  misc_feature 1..1260: note = A nucleic acid sequence obtained from an unknown bacterial straincomprised within a microbiome encoding a TIC7940 PirB pesticidalprotein sequence.
atgaccacgaaccccattgttcatagagagtttgattatgcggacctttacggcgacgatagtcgatccagcccacaaaacaatgattgggactatttagttgtagcaaagattcttgttgttcaggggcttaagcatattcctgtcgtgggcggggctctttcgagtctaactaacgctctctggccgaaaaaaaaagataatgtctggacgcaagctcttggagagattgagcagtatattgatagtcagaatctgaaggtgattcagggcatactcaatggtgagatacttgagatccaagggaagatggagcacgttacagcgcttcttgagaagcatcctaataccaaggaatcttacaatgcgtataaggatttagctcagtatcttgatagtaagcagagaaagtttggagcatttgatgctgagcagaactatcatttgattcctatgtatgcatctatgattttgctgcaggcgacgtattggcgaaccggaattgacaggcgaaatgaaatcggcctgactgatatcgatgttgagtctctaaagaggcttattgctgatcttgttttcgaatcgagggaatatatcggtcgcgtctacgatgaggcggtcgaaagggtgtatgctgaagctaatcccagagatgtgacaaattatatgtactctgttaggggttatagcttgctgcacggtgttgagactgttgaaataatcgatcgcgttagaagattgggcgttgatagcggcttcaatgttggtgtggtaagttattctacggtggttgggacagttacaaaccgcgtaagaactcaagctctcactccagatgatgaaatgaaagagccattaaggccggagttcgttgacgatgaagttaatcagatcgcgtctataactgggtatattggtcctcttattgatagtaagcccacgattggcggcttgttcgtggtctttgaaaacggcaactgctacaaaatgggggcggagtcaggcacgtcttattctatagaccttcgtggaagtactatctcaaccgttgaagtttggtatcagggattactagaaggggttcggtttacattgagtgatgatagagatctgctgattggccagcggcaatccgagaggtccaaatatagacgatttgaagtggaagggcattacgtttcaggagtgtatttggatcgtgatgaaacgacctatcgcgggcgagctgccaatatttcagtgtcttaccatatcgcgtag

SEQ ID NO: 46 - protein, 419 aa, unidentified
  REGION 1..419: note = The amino acid sequence of the TIC7940 PirB protein.
MTTNPIVHREFDYADLYGDDSRSSPQNNDWDYLVVAKILVVQGLKHIPVVGGALSSLTNALWPKKKDNVWTQALGEIEQYIDSQNLKVIQGILNGEILEIQGKMEHVTALLEKHPNTKESYNAYKDLAQYLDSKQRKFGAFDAEQNYHLIPMYASMILLQATYWRTGIDRRNEIGLTDIDVESLKRLIADLVFESREYIGRVYDEAVERVYAEANPRDVTNYMYSVRGYSLLHGVETVEIIDRVRRLGVDSGFNVGVVSYSTVVGTVTNRVRTQALTPDDEMKEPLRPEFVDDEVNQIASITGYIGPLIDSKPTIGGLFVVFENGNCYKMGAESGTSYSIDLRGSTISTVEVWYQGLLEGVRFTLSDDRDLLIGQRQSERSKYRRFEVEGHYVSGVYLDRDETTYRGRAANISVSYHIA

SEQ ID NO: 47 - DNA, 1677 bp, synthetic construct
  source 1..1677: mol_type = other DNA
  misc_feature 1..1677: note = A nucleic acid sequence encoding a PirAB fusion protein, TIC9321comprised of the TIC7939 and TIC7940 coding sequences in operablelinkage and in frame.
atgacgtgtgagattctgcatatgacaagcaaaggcgatgaaatgcagtcgattgcagcgacggatgctcaaacgttacaggaggcgcctaaagatgaagtgaattttaagcagacaaaaggggatatgatggtccctggggggcagtctgcacagggagcgcgctacgatactccgattattcctgaacttcatccgtcttactatgtatcaaattcaggacctgcagctacggtgaaagctgtcttctactggtcccactcatttacctcgaagtggtttgaatataattcagttacggttctcagggggactactgagcggcttagtgcgccaagcaactcactttattacagcaaggtcgttgtctttaataatgagaaagagcctgcttatgttactgtaacgaccattcggatgaccacgaaccccattgttcatagagagtttgattatgcggacctttacggcgacgatagtcgatccagcccacaaaacaatgattgggactatttagttgtagcaaagattcttgttgttcaggggcttaagcatattcctgtcgtgggcggggctctttcgagtctaactaacgctctctggccgaaaaaaaaagataatgtctggacgcaagctcttggagagattgagcagtatattgatagtcagaatctgaaggtgattcagggcatactcaatggtgagatacttgagatccaagggaagatggagcacgttacagcgcttcttgagaagcatcctaataccaaggaatcttacaatgcgtataaggatttagctcagtatcttgatagtaagcagagaaagtttggagcatttgatgctgagcagaactatcatttgattcctatgtatgcatctatgattttgctgcaggcgacgtattggcgaaccggaattgacaggcgaaatgaaatcggcctgactgatatcgatgttgagtctctaaagaggcttattgctgatcttgttttcgaatcgagggaatatatcggtcgcgtctacgatgaggcggtcgaaagggtgtatgctgaagctaatcccagagatgtgacaaattatatgtactctgttaggggttatagcttgctgcacggtgttgagactgttgaaataatcgatcgcgttagaagattgggcgttgatagcggcttcaatgttggtgtggtaagttattctacggtggttgggacagttacaaaccgcgtaagaactcaagctctcactccagatgatgaaatgaaagagccattaaggccggagttcgttgacgatgaagttaatcagatcgcgtctataactgggtatattggtcctcttattgatagtaagcccacgattggcggcttgttcgtggtctttgaaaacggcaactgctacaaaatgggggcggagtcaggcacgtcttattctatagaccttcgtggaagtactatctcaaccgttgaagtttggtatcagggattactagaaggggttcggtttacattgagtgatgatagagatctgctgattggccagcggcaatccgagaggtccaaatatagacgatttgaagtggaagggcattacgtttcaggagtgtatttggatcgtgatgaaacgacctatcgcgggcgagctgccaatatttcagtgtcttaccatatcgcgtag

SEQ ID NO: 48 - protein, 558 aa, synthetic construct
  REGION 1..558: note = The amino acid sequence of the TIC9321 PirAB fusion protein.
MTCEILHMTSKGDEMQSIAATDAQTLQEAPKDEVNFKQTKGDMMVPGGQSAQGARYDTPIIPELHPSYYVSNSGPAATVKAVFYWSHSFTSKWFEYNSVTVLRGTTERLSAPSNSLYYSKVVVFNNEKEPAYVTVTTIRMTTNPIVHREFDYADLYGDDSRSSPQNNDWDYLVVAKILVVQGLKHIPVVGGALSSLTNALWPKKKDNVWTQALGEIEQYIDSQNLKVIQGILNGEILEIQGKMEHVTALLEKHPNTKESYNAYKDLAQYLDSKQRKFGAFDAEQNYHLIPMYASMILLQATYWRTGIDRRNEIGLTDIDVESLKRLIADLVFESREYIGRVYDEAVERVYAEANPRDVTNYMYSVRGYSLLHGVETVEIIDRVRRLGVDSGFNVGVVSYSTVVGTVTNRVRTQALTPDDEMKEPLRPEFVDDEVNQIASITGYIGPLIDSKPTIGGLFVVFENGNCYKMGAESGTSYSIDLRGSTISTVEVWYQGLLEGVRFTLSDDRDLLIGQRQSERSKYRRFEVEGHYVSGVYLDRDETTYRGRAANISVSYHIA

SEQ ID NO: 49 - DNA, 1695 bp, synthetic construct
  source 1..1695: mol_type = other DNA
  misc_feature 1..1695: note = A synthetic coding sequence used for expression in plant cellsencoding a TIC6880PL PirAB fusion protein with an additionalalanine codon inserted after the initiating methionine codon.
atggctatcacgataaacatctcgggcgggagcatcaagatttccaacaacattggctccgagacggacatccgcaacacgcccttctccgagccgctctccatctccaactacaaagacatgacgatagagcctcattcctccatccaggccacgcggaccgacacacccatcatccctgagacccggccgaactactacgtggccaactctggcccggcggcctccgtgcgcgccgtcttctactggtcgcacagtttcacatcggagtggtttgagcactcctccatcattgtcaaagctggcgaggacggcatccttaactcgccctccaacagcgtgtactacagcaaggtggtcatctacaatgacacggacaagcgcgccttcgtcacgggctacgacaagatgaacaacgaactcatgaacaccaacgagtcgcagccttccgagactctgagcctgattaacgagtccatcctcaccgcgccctacgcggtctccacgccgaactacgagtgggacatgtcgtcgattattaaggacgccataatcggcggtattggcttcatccctggccctgggagcgccatctccttcctccttggtttgttctggccgcagcaaaccgacaacacctgggagcagatactccagaaggtcgagcaaatgattgaggaagccaacctcaagaccatccagggcatcctgaacggcgacatccaggagatcaagggcaagatggagcacgtagagtacatgctggagacctcgccaggcactcaggagagtcacgacgcctacatgttcctggcgcgctacttagtttccatcgacgagaagttcaagagcttcgacaacaagacgaactaccagatcctgcccatgtacaccaacaccctgatgctccaggctccctactggaagatgggcatcgagaagaagaacgacatcctacttactgacattgaggtgaacgaactcaagcagctcatcgagagcctgtacgcgaaggccaacagctacatccacgaagtgtacacccgagagtacgacaacgcggtgaacaccagtaccgcgaccaccatcaccaacaacctcctgagcgtgcgaggctactgcctcttgcacgggctcgaatgcctggaggtgctcgaccacatccagaacaacaacctggaccagtcgttctacccgaagaccatctcctactccaccgtctttgaccggtccaccaacaagactcggctgcaagcgctgaccgaggacgagcagatggaggaacctctgaaaccatccttcatcaacggcgagtacaacaagatcaagagcctgatcgggtacgtgcagcggattggcaacgcgccgcgagtgggcggcatcaagataaccttcacgaacgggtccagccacaccttgggcaccgtcacgagcgagtccaactccatcgagctgaacgactccgtcatcacctccgtcgaggtgtggggcaatggtgcggtggacgaggcgttcttcactctctcggacggtcgccagttccgcctgggccagcgttacgcctccaactaccgcaagtacgcggtggacgggcactacatctccggcctgtacctggcctcggacgagccatccctggccgggcaagcggctggcattgccgtcagctaccacatcctggtggacaagaagtga

SEQ ID NO: 50 - protein, 564 aa, synthetic construct
  REGION 1..564: note = The amino acid sequence of the TIC6880PL PirAB fusion protein.
MAITINISGGSIKISNNIGSETDIRNTPFSEPLSISNYKDMTIEPHSSIQATRTDTPIIPETRPNYYVANSGPAASVRAVFYWSHSFTSEWFEHSSIIVKAGEDGILNSPSNSVYYSKVVIYNDTDKRAFVTGYDKMNNELMNTNESQPSETLSLINESILTAPYAVSTPNYEWDMSSIIKDAIIGGIGFIPGPGSAISFLLGLFWPQQTDNTWEQILQKVEQMIEEANLKTIQGILNGDIQEIKGKMEHVEYMLETSPGTQESHDAYMFLARYLVSIDEKFKSFDNKTNYQILPMYTNTLMLQAPYWKMGIEKKNDILLTDIEVNELKQLIESLYAKANSYIHEVYTREYDNAVNTSTATTITNNLLSVRGYCLLHGLECLEVLDHIQNNNLDQSFYPKTISYSTVFDRSTNKTRLQALTEDEQMEEPLKPSFINGEYNKIKSLIGYVQRIGNAPRVGGIKITFTNGSSHTLGTVTSESNSIELNDSVITSVEVWGNGAVDEAFFTLSDGRQFRLGQRYASNYRKYAVDGHYISGLYLASDEPSLAGQAAGIAVSYHILVDKK

SEQ ID NO: 51 - DNA, 1701 bp, synthetic construct
  source 1..1701: mol_type = other DNA
  misc_feature 1..1701: note = A synthetic coding sequence used for expression in plant cellsencoding a TIC9316 PirAB fusion protein.
atgaacaccatcaacatcaacatctccggctcgaccgtgacggtgatctcgaacaacgactccaacccggagccgctcacctacaacacgaacacaccagcgagcgatcctctgacggccagcccgtaccgggacatgaccatcgagccgcacagctccattgaggcgacgcgcacagacacgcccatcatcccggagacccggccgaactactacgtcgccaactccggcccggcctcctcagtccgcgctgtgttctactggtcgcacagcttcacgtcagaatggttcgagtacagctctatcatagtcaaggcgggcaaggacggcatcctccagtcgcccaacaacgccctgtactacagcaaggtcgtgatctacaacgacaccgacaagcgcgccttcgtcaccggctacaacaagatgaacatcagcccgatcaacgtctcggagaacgagacgctcccggagctgaccgacgtgatgctgatcgtcccatacacgaccagcacgccggattacgagtgggacatgtcgtcgatcattaaggatgcgatcatcggaggcgttggcttcatccctggcgcgggctcggccatgagcttcctgctcggcctgttctggccgcagcagaaggataacacttgggagcagatacttcagaaggtggaacagatgatcgagaacgcggtcctccaaacgatcaagggcatcctcaacggcgacatccaggagattaagggaaagatggagcacgttcagtacatgctcgaaaccagccctgggagccaggagagccacgacgcctacatgttcttggcacgttacctcgtctcgattgacgagaagttcaagtccttcgacaacaagacaaactaccagatcttgccaatgtacaccaatacggttatgttacagattccgtactggaagatgggcatcgagaagaagaatgacatcggcttgaccgacatcgaggtcaatgagcttaagcaacttatcgacaagctggtggacaaggccaagtcctacatccacacaatgtacaccaacgagtacaacgacgcgatcaacaccagcaccgcctcaagcgtcacaaacaacctcctgtccgtgcgcggttactgccttctgcacggcctggagtgcatcgaacttattgagcatctccagaacaacagcctggagtccggcttctacccgaagacgatcagctactccacggtcttcgaccggcagaccaacaagatgcggatacaagcgctcactgaggacgaccagatgcaagaacccttcaagccctcgctcatcaacgggaagtacaacaagatccagagcctcctcggctacgtccagcgcatcggcaacgcgccgcgcgtcggcgggatcaagatcacgttcgccaacgggtctagttacacgctgggcaccgtgaccagcgagacctccagtattgagcttaacgactcggtgatcgagcggctggaggtgtggggcaacggcgcggtggacgaggcgctgttcaccctctcggacgggcggcagctccgggtcggcgagcggtacgcgaccaagtaccgtaagtacgccgtggacggccactacatcgccggtctgtacctcgccagtgacgagcccagcctagcgggccaggcggctggcatcgccgtgtcgtaccacatgctcgacgacaagaagtga

SEQ ID NO: 52 - DNA, 1701 bp, synthetic construct
  source 1..1701: mol_type = other DNA
  misc_feature 1..1701: note = A synthetic coding sequence used for expression in plant cellsencoding a TIC9317 PirAB fusion protein.
atgattacgatcaacatcaacgtgaacggcaacgacgtgacgggcaccaacaacaatgagcccactccagtcagcacgacgtacggcccgaacactccggcctcggagccaccggtcgtctcgaactactccgacatcaccattgagccgcacagctcggtccaggccacgcggatcgacacgccggtgatcccggaggcccggccggactactacgtggcgaactcgggccctgcgccgtccgtgcgggccgtgttctactggtcgcactcgttcacctccgagtggttcgagtcgtccagcatcaccgtgaaggcgggcgaggacggaatcctcaaggctccagggaacagcctgtactacagcaaggtcgtcatctacaacgacacagacaagcgggccttcgttaccgggtacaacaagatgaacaccactccgattactgtaagcactaacgaaacatcgcctctcatgacggacgtgatgccgatggacctgtacgccatctcgacgccagactacgagtgggacatgagttccatcatcaaggacgccgtaattggcggcatcgggttcatccctgggcccggcccggccatctccttcctgctgggcctgttctggccgcagcagaaggacaacacatgggagcagatactccagaaggtcgagcaaatgattgagaatgccgtgttgcagacgatcaagggaatcctaaacggcgaagtacaggagatcaagggcaagatggagcacgtcgagtctatgctcaagaactcgccaggctctcaggagtcacacgacgcctacatgttcctggctcgttacctcgtttcaattgacgagaagttcaagagcttcgacaaccgcaccaactaccaactgttgccgatgtacaccaatacgattatgctccagataccttattggaagatgggcatcgagaagaagaaggacattggcctgaccgacattgaagtcaacgagcttaaggagctgatcgacaagctggtggacaaggccaagaactacatccacacaatgtacacgaacgagcacaacaacgccgtgaacaccagcactgccgagtccgtcacgaacaatctcctcagcgtgcgcggctactgcctgttacacgggctggagtgcattgagctaatcgagcacatccagaacaactccctggagagcgggttctacccgaagatcatcagctacagcaccgctttcgaccgcccgacaaacaagatgcgtatccaagcgctcacggaggacgacgcgatgcaagagccgtttaaaccgtcgctcattaacggcaagtacaacaagatccagagcctcacgggctacgtgcagcggatcggcaacgcgccgcgcgtcggcggcatccgcatcacgttcaccaacgggtcgtcctacacgctcgggacggtgacctccgagacgcacagcatcaagctgaacgactccgtgatcgagtcgttagaggtctggggaaacggtgccgtggacgaggccctgttcaagctgtccgacgggcggctcctccgcatcggcgagcggtacgccaagaagtaccgcaagtacgcggtggacaaccactacatcgcgggcatctacctagcgagcgacgagccgtccctggcgggtcaagccgccgggatcgccgtgagctatcacatgatggcggacaagaaatga

SEQ ID NO: 53 - DNA, 1701 bp, synthetic construct
  source 1..1701: mol_type = other DNA
  misc_feature 1..1701: note = A synthetic coding sequence used for expression in plant cellsencoding a TIC9318 PirAB fusion protein.
atgtccaccatcaacatcaacatctcctccagcacggtcactgtgattacaaacaatggcgagacgcccgtcccgctcacctacaacaccaacacgccggagtcggagccgctgacggtcaacccgtaccgcgacatgaccatcgagccgcgctcctccattgaggcgacccgcatcgacacgccgatcatcccggagacgagaccgaactactatgtggcgaactccggcccggccagcagcgtccgggcggtattctactggtcgcacagcttcacctcgcaatggttcgagtattcgtcgatcatcgtgaaggcgggtgaggacggcatactggagtcgccgtcgaacagcctctactacagcaaggtggtgatctacaacgacacggacaagagggcattcgtcacgggctacaacaagatgaacaccaccctcatcaacgtaagtgagaaggagacgctgccggtgcagaccgacatcatgctgatcgcgccgtactccgtgtccacgccggactacgagtgggacatgtcatcgctcatcaaggacgcgatcatcggcggcgtcggattcatccctgtcgtcggctcggccatgtccttcctcctcggcctgttctggccgcagcagaaggacaacacttgggaacaaatactgcagaaggtcgagcagatgatcgagaacgcgcagctcaacacgattaagggcattctgaacggcgacatccaggagattaagggcaagatggagcacgtgcagtacatgcttgagacaagtccagggagtcaggagtcacacgacgcctacatgttcttagcccgctacctagtgagcatcgacgagaagttcaagtcgttcgacaacaagacaaactaccaaatcttgccaatgtacacgaataccgtcatgctacagatcccatactggaagatgggaattgagaagaagaacgacattggcttgacggacatcgaagtcaacgagcttaaacagcttatcgacactctggtggaccgcgcgcgcaactacatccacaccatgtacgagcgagagtacgacaacgccatcaacacctcaaccgctgcctcggtgaccaacaacctgctctccgtgcgcgggtactgcctcctgcacgggctggagtgcatcgagactatcgagcaccttcagaacaacagcctcaacagtgggttctacccgaagaccatcagctacagcactgtcttcgaccggcccacgaacaagacccgcatccaggctctgacggaagacgaccaaatgcaagagccgttcaagcccgcgctgatcggcggcaagtacaacaagatcaagtccctgttgggctacgtgcgaaggatcggcaacgctccacgggtcggcggcatcaaggtgacgttcaccaatgggtcgagctacacgctcgggacggtcacgtcggaaaccgactccatcgaactgaacgagtcggtcatcgagcggctggaggtgtggggcaacggagccgtggacgaggccctctttaccctgagcgatggccgccagctccgcatcggcgagcggtacgccaagaaataccggaagtacgcggtggatgggcactacatcagcggcctctacctcgcgtcggacgagccctccctcgccggtcaagcagccgggatcgcggtgtcctaccacatgctcgcagacaagaagtga

SEQ ID NO: 54 - DNA, 1650 bp, synthetic construct
  source 1..1650: mol_type = other DNA
  misc_feature 1..1650: note = A synthetic coding sequence used for expression in plant cellsencoding a TIC9319 PirAB fusion protein.
atgattaccatcaacatctccggcgggaacgtgaccatcaacaacaacatctccagcgtaaccgacatccagaagccgctggacgccgagccgctgtccgtgacgaactacaaggacttgacgatagagccgcactcctcgatccaggcgacccgcacggacacgccgatcatcccggagacccgcccgaactactacgtggccaactcgggtccggcttccagcgtgaaggccgtgttctactggagccactccttcacgtcggagtggttcgagtacagctccatcattgtgaaggccggtgaggacggcatcctcaagagcccgtccaacgccgtgtactactcgaaggtcgtcatctacaacgacacggacaagcgggccttcgtgacgggctacaacatgatgaacaatagcccgatgaacgaccagctctccatcgcgccgtactccatctccacgcccaactacgagtgggacatgagcagtatcatcaaggacgccatcatcggcggcatcggcttcataccgggccctggcagcgccatctccttcctgctgggcctgttctggccgcagcaaacggataacacttgggaccaaatacttcagaagatcgagcaaatgattgaggaagctaaccttaagaccatcaagggaatactgaacggcgacatccaggagatcaagggcaagatggaccacgtgcagtacatgctggagaactctcccggaagccaggaatcccacgacgcctacatgttcctggcccgtttcctcgtgtccatcgacgagaagttcaagagcttcgacgaccggaccaactaccaaatcttgccaatgtacaccaacactatcatgttgcaagctccttactggaagatgggcctggagaagaaggaagacattggcctgtcggacattgaggtttcggaattgaaggagctaattgacaagctctacaccaagagctacgactacatccacaacacctacaaccgcgagtacgacaacgcgatcaacacatccaccgccgagtcaatcaccaacaatctgctcagcgtgcgcggctactgcctcctccacggctgtgaatgtctcgaagtcatcgcccacatccagaacaacagcctcgacaagggcttctacccgaagaccatcagctactcaagcgtgttcgaccgccctacgaacaagatgcgaatccaggcgctgaccgaagacgaccagatgcaagaacccttcaagccctcgttcgtaaacggccagtacaacaagatcaagagcctggagggctacgtgacccggatcggcaacgcgccgcgcgtcggcgggatcaagatcacgttcgagaacaacgccagctacacgctgggcaccgtcacctcggagaccacgttcatcgagctgaacgagtcggtcatcaccagcattgaggtctggggcaacggcgcggtggacgaggcgttcttcacgctgagcgacgggaggcagatgaggctcgggcagcggtacgcgagccgctaccgcaagtacgcggtggacgggcactacatctcgggcctgtacctcgccagcgacgagcctagcctcgcgggccaggcagccgggatcgccgtgagctaccacatgatcgtggacaagcagtga

SEQ ID NO: 55 - DNA, 1659 bp, synthetic construct
  source 1..1659: mol_type = other DNA
  misc_feature 1..1659: note = A synthetic coding sequence used for expression in plant cellsencoding a TIC9320 PirAB fusion protein.
atgagccgaatcacaattgtcgtggattcggacgaacagaaggccgaggtgtacagcaattctccggtcccggtccacaaggacttaaatgccgtgggcccgctttccgacgtcaccatctcgccgcacgcctcagtcgaggtcttccgcatcgacacgccgatcatcccggagtcccggcgctcgctgagggtggtgaacacgggcctggccaacagcgtgacggccaagttctactggagccactccttcacgagcgagtggttcgagtcgggctcaatcgacgtgggcctgggcgaggagaaggtgctaaatgtcccgaacaactcattctactactcgaagttcgtgatctacaacaacaccgacaaggtggcgtacgttaccgccaacctcgtaatgcacacggagaacgtgctcgacattcgtaccattgtggcaaacgagtacgccgtcaagacgtcggcggtcgagtgggacgtcaccgacattgttaagaacgcgatcatcggcggcatctcattcatccctagcgtgggcccggccatcagcttcctggtgggcctattctggccacagagcaaggagaacatctgggagggcatcgtcaagcagatagagcgcatgatcgaggagtccgccctcaagacgattaagggcatcctggcgggcgacatcgcctacatacaggagcggatggccacggtggccgacctcctggataaacaccctgggagtgaggaggcccgatctgcctttaacaacctcgccgagaacattgacggctaccacaagaaattcaacaacttctcagatgatgtcaattaccagatcctgccaatgttcagcactaccgtgatgatgcagatcacatactgggtcgctgggcttgagcgccgcaacgaaatcggattgtccgacatcgacatcgagaaggttcgcgggcttattaagaagaccgtggaacaagctaactcctacatcaataacatctacgaccgggagcttaacgatgccctgaacaattccaccgctgataccgttgccaacaacgttatgtctgtgcacgggcactgccgcctccacggcattgagtacatctccatctgggacaagctgagtgaggccgagtcggtcaacaaccgcatctatgtggacgtactgtcctactcaaccttcttcgaccgccagacggcgaaggcccgcatacaagctctgacaccggagaaggacatggctccgccgctgaaacccgcgctgaacgacgggaagcgccggaagatcgacagcctaacgggccacatcgtccgaatcggcggcgcaccacgagtcggcggcctgacggtggtgttcgacgacggcagttcgcatcgtctcggcacgataagtggcgaaaccgcttcaatcagcctcaacgggtcgcggatcacgtcgctggaggtgtggggaaatggtgccgtggaccaggcggtcttcacgctctcggacgggcggtccctcagcttcggagcgcctggcacctcgcgctaccgtaagttctacgtcggcgagtcgcattacatcgcgggcgtttatctgtcgtccgactactcacctctggcgggtcaagctgcgaacatcgcggtgtcctaccaactcatcaacgacgacgagaagtga

SEQ ID NO: 56 - DNA, 1659 bp, synthetic construct
  source 1..1659: mol_type = other DNA
  misc_feature 1..1659: note = A synthetic coding sequence used for expression in plant cellsencoding a TIC9322 PirAB fusion protein.
atgtcccgcatcacaatcgtagtggactcggacgaccagaaggccgagttctacagcaactcgccggtgcctgtgtacaaggacttgaacgccgtgggtccgctcagcgacgtcacaatctctcctcacgcatctgtggaggtcttccggatcgacacgccggtgatcccggagtctcggtcgtctctgcgcgtcgtcaacaccggcctctccaattcggtgacggctaagttctactggagccactcgttcacatcggagtggttcgagtccggctccatcgacgtgggcctcggcgaggagaaggtgcttaatgtgccgtccaactccttctactattccaagttcgtgatctacaacaacaccgacaaggtggcctacgtcaccgcgaacctcgtgatgcacaccgagaacgtcctagacatcaggaccatcgtggcgaacgagtacgcggtgaagacctccgccctggaatgggacgtgaccgacattgtcaagaatgcgatcatcggcgggatctcgttcatcccgagcgtgggaccggctatctcctttctcgtgggattgttctggccgcaatcgaaggagaacatctgggagggaatcgtgaagcagatcgagcggatgatcgaggaatctgctctcaagacgatcaagggcatcctcgcgggagacatcgcctacatccaggagcggatggccacggtggccgacctcctggacaagcaccctggatcggaggaggcccgaagcgcgttcaacaacctcgccgagaacatcgacggctaccacaagaaattctcaaactttagtgatgatgtcaactaccagatacttccgatgttcagcaccaccgtgatgatgcagattacatactgggtggcgggcctagagcgaaaggacgagatcggcctctcgaacatcgacgtggagaaggtgcgtgggcttattaagaagaccgtagaacaagcaaactcttacatcaacaacatctacgacagagagttaaacgacgcattgaacaactctaccgcagatactgtggcaaacaacgtcatgagcgtgcacgggcactgccgcctgcacgggattgagtacatcagtatttgggataagctgagtgaggccgaaagcgtgaacaacaaaatctacgttgacgtgctgagctactctacattctttgaccgccagacggcgaaggccaggatacaggcgttgacgccggagaaggacatgacgccgccgctcaagccagccctgaacggcggcaagcggcgcaagatcgactccctcaccggccacatcgtccgtatcggcggtgccgcgcgggtcggcggcctgaccgtggtgttcgacgacgggaacaggcaccagcttggaacgatcagcggcgagactagctcaatctcccttaatggctcccgcatcacctcgctggaggtgtggggaaacggcgcggtggatcaggccgtgttcacgctgaacgacggtcgttcgctcagcctgggctcgcccggcacctctcgctaccgcaagttctacgtgggcgagtcgcactacatcgcgggcatctacctcagcagtgactacaacccgctcgctgggcaagctgcgaacatcgccgtctcctaccagctcatcaacgacgacgagaagtag

SEQ ID NO: 57 - DNA, 345 bp, Shewanella violacea
  source 1..345: mol_type = other DNA
  misc_feature 1..345: note = A nucleic acid sequence obtained from Shewanella violacea strainDSS12 encoding a TIC10357 pesticidal PirA protein sequence.
atgagtgatttagaagtaaaaataggtgttaatcctgctgatgttcgttatacagctaattttaaagttgcaccaaacgacggatatgtgatgtatgaaaaaaatacgccaatcattccagaaattggtgtgaatattacggttataaatacaggtcgtgaagaaatggaagttcactatgaatgggctccaccatttggtggatggcaatgtgcatctacaacaataatcccacctgatggtaagcctgtttatattgctcatccgtcaaatgctttttattatcagcgaatcattgcttataacaaaaaagaatcaacagcgttcgggaattgcgaatactaa

SEQ ID NO: 58 - protein, 114 aa, Shewanella violacea
  REGION 1..114: note = The amino acid sequence of the TIC10357 PirA protein.
MSDLEVKIGVNPADVRYTANFKVAPNDGYVMYEKNTPIIPEIGVNITVINTGREEMEVHYEWAPPFGGWQCASTTIIPPDGKPVYIAHPSNAFYYQRIIAYNKKESTAFGNCEY

SEQ ID NO: 59 - DNA, 1284 bp, Shewanella violacea
  source 1..1284: mol_type = other DNA
  misc_feature 1..1284: note = A nucleic acid sequence obtained from Shewanella violacea strainDSS12 encoding a TIC10366 pesticidal PirB protein sequence.
atgaataatgaatatatcgtaacaatggaaaagaaaaacaacatagaattaaaaagtagtggtcgttatacattagatgatttttaccatgatcatgcttatgcatttaaagtcgctttgactattggacttaaaaaaataccttatgttggaagtattttatctacacttgttaaaatattatggcctactggagcatcaggtgaatctttacaaaacttatgggaaatggaaagaaatgaaattcaatcaatgattgatgaagctacacttcatactataaacgatatattaaacggaattgtaaattcactcggtgataaaatagccgatattaatagaactatagaaaattacgggtttgcagctgcaaaagatgattatataaacttaatttcaaattatataattggattggaagaacagtttaaatttgaaagtgaaggctctgaatttatagcttatgcaacaatgccactgttatctattactgttggtttgcaattatcatatttggcatttggtttagataataaagctaactttggacttgatagtgctgatatagataaatgtagtagaaacatagatgaaatttataaagatgttaaaaaatatatagaaaaatatgctaagtggtctgattctgactcttacagtaatgctaatagtgaaaacatatataatgaagttatgggatctcgtgctttttgtgctttaaatggatttgaacacattgaaatctggtctgaaatacaatcacgtaaatcacttgatttttcaattataagtacatctgtatcttattctgttgaggtcggtgttttaacgcctaacatgacaaggatggcaacagctgttgaagttggcccgcctttgttacctgttatggttgatggacatagaaacaagatagttaaaattgagggttgggatagtgtagaaattaatagttatcgtcgtgtcggttgccttaaaatcacttatgaaaatggtgatgtttacgatatgggtgttaaaacatctaatcctgttagcatttcacttgatggtgaatttgtagataccgtaaaagtcgttcaaggtgatacatatgcaattaattacatcaaattcacattaactgatggacgcacaatgtcagttggtgaacaaagcggtgatacacaactattaggttttgataatcatactattgctgcaatttttgttgatgaaggttcttcagataaaatttcatgtgttagcgtttcatgcattcctaagcagtacgaagaagaatag

SEQ ID NO: 60 - protein, 427 aa, Shewanella violacea
  REGION 1..427: note = The amino acid sequence of the TIC10366 PirB protein.
MNNEYIVTMEKKNNIELKSSGRYTLDDFYHDHAYAFKVALTIGLKKIPYVGSILSTLVKILWPTGASGESLQNLWEMERNEIQSMIDEATLHTINDILNGIVNSLGDKIADINRTIENYGFAAAKDDYINLISNYIIGLEEQFKFESEGSEFIAYATMPLLSITVGLQLSYLAFGLDNKANFGLDSADIDKCSRNIDEIYKDVKKYIEKYAKWSDSDSYSNANSENIYNEVMGSRAFCALNGFEHIEIWSEIQSRKSLDFSIISTSVSYSVEVGVLTPNMTRMATAVEVGPPLLPVMVDGHRNKIVKIEGWDSVEINSYRRVGCLKITYENGDVYDMGVKTSNPVSISLDGEFVDTVKVVQGDTYAINYIKFTLTDGRTMSVGEQSGDTQLLGFDNHTIAAIFVDEGSSDKISCVSVSCIPKQYEEE

SEQ ID NO: 61 - DNA, 1626 bp, synthetic construct
  source 1..1626: mol_type = other DNA
  misc_feature 1..1626: note = A nucleic acid sequence encoding a PirAB fusion protein, TIC10375comprised of the TIC10357 and TIC10366 coding sequences inoperable linkage and in frame.
atgagtgatttagaagtaaaaataggtgttaatcctgctgatgttcgttatacagctaattttaaagttgcaccaaacgacggatatgtgatgtatgaaaaaaatacgccaatcattccagaaattggtgtgaatattacggttataaatacaggtcgtgaagaaatggaagttcactatgaatgggctccaccatttggtggatggcaatgtgcatctacaacaataatcccacctgatggtaagcctgtttatattgctcatccgtcaaatgctttttattatcagcgaatcattgcttataacaaaaaagaatcaacagcgttcgggaattgcgaatacatgaataatgaatatatcgtaacaatggaaaagaaaaacaacatagaattaaaaagtagtggtcgttatacattagatgatttttaccatgatcatgcttatgcatttaaagtcgctttgactattggacttaaaaaaataccttatgttggaagtattttatctacacttgttaaaatattatggcctactggagcatcaggtgaatctttacaaaacttatgggaaatggaaagaaatgaaattcaatcaatgattgatgaagctacacttcatactataaacgatatattaaacggaattgtaaattcactcggtgataaaatagccgatattaatagaactatagaaaattacgggtttgcagctgcaaaagatgattatataaacttaatttcaaattatataattggattggaagaacagtttaaatttgaaagtgaaggctctgaatttatagcttatgcaacaatgccactgttatctattactgttggtttgcaattatcatatttggcatttggtttagataataaagctaactttggacttgatagtgctgatatagataaatgtagtagaaacatagatgaaatttataaagatgttaaaaaatatatagaaaaatatgctaagtggtctgattctgactcttacagtaatgctaatagtgaaaacatatataatgaagttatgggatctcgtgctttttgtgctttaaatggatttgaacacattgaaatctggtctgaaatacaatcacgtaaatcacttgatttttcaattataagtacatctgtatcttattctgttgaggtcggtgttttaacgcctaacatgacaaggatggcaacagctgttgaagttggcccgcctttgttacctgttatggttgatggacatagaaacaagatagttaaaattgagggttgggatagtgtagaaattaatagttatcgtcgtgtcggttgccttaaaatcacttatgaaaatggtgatgtttacgatatgggtgttaaaacatctaatcctgttagcatttcacttgatggtgaatttgtagataccgtaaaagtcgttcaaggtgatacatatgcaattaattacatcaaattcacattaactgatggacgcacaatgtcagttggtgaacaaagcggtgatacacaactattaggttttgataatcatactattgctgcaatttttgttgatgaaggttcttcagataaaatttcatgtgttagcgtttcatgcattcctaagcagtacgaagaagaatag

SEQ ID NO: 62 - protein, 541 aa, synthetic construct
  REGION 1..541: note = The amino acid sequence of the TIC10375 PirAB fusion protein.
MSDLEVKIGVNPADVRYTANFKVAPNDGYVMYEKNTPIIPEIGVNITVINTGREEMEVHYEWAPPFGGWQCASTTIIPPDGKPVYIAHPSNAFYYQRIIAYNKKESTAFGNCEYMNNEYIVTMEKKNNIELKSSGRYTLDDFYHDHAYAFKVALTIGLKKIPYVGSILSTLVKILWPTGASGESLQNLWEMERNEIQSMIDEATLHTINDILNGIVNSLGDKIADINRTIENYGFAAAKDDYINLISNYIIGLEEQFKFESEGSEFIAYATMPLLSITVGLQLSYLAFGLDNKANFGLDSADIDKCSRNIDEIYKDVKKYIEKYAKWSDSDSYSNANSENIYNEVMGSRAFCALNGFEHIEIWSEIQSRKSLDFSIISTSVSYSVEVGVLTPNMTRMATAVEVGPPLLPVMVDGHRNKIVKIEGWDSVEINSYRRVGCLKITYENGDVYDMGVKTSNPVSISLDGEFVDTVKVVQGDTYAINYIKFTLTDGRTMSVGEQSGDTQLLGFDNHTIAAIFVDEGSSDKISCVSVSCIPKQYEEE

SEQ ID NO: 63 - DNA, 435 bp, Photorhabdus luminescens
  source 1..435: mol_type = other DNA
  misc_feature 1..435: note = A nucleic acid sequence obtained from Photorhabdus luminescensstrain laumondii TTO1 encoding a TIC10358 pesticidal PirA proteinsequence.
atgccagtcaatcagattggcttacataatgaaaaggtgaaaaatatgagaaaaataacagttgataatgatgtggtaggacatgatactgaaatcaactcggttgtttcatcaactgcggagaaaattcgccaacagtttggagtaaaggtcgaccctaattcaagtcaggaaaagttctacattgcaacaccgattattcctgaatcccgaaagaatatcgttgtaaccaatgaaggtctcgccgatgttatcacggcgaaatattactggtcacattcttttacgtcagaatattttgaggataactcagttgatgtcaaggttggagagagcaaagtgttggttgccccttcaaacccgttgtattacagcaaagtagtcattttcaacaacactaaatccgtggcatttgtaacagtaagagaaaaataa

SEQ ID NO: 64 - protein, 144 aa, Photorhabdus luminescens
  REGION 1..144: note = The amino acid sequence of the TIC10358 PirA protein.
MPVNQIGLHNEKVKNMRKITVDNDVVGHDTEINSVVSSTAEKIRQQFGVKVDPNSSQEKFYIATPIIPESRKNIVVTNEGLADVITAKYYWSHSFTSEYFEDNSVDVKVGESKVLVAPSNPLYYSKVVIFNNTKSVAFVTVREK

SEQ ID NO: 65 - DNA, 1254 bp, Photorhabdus luminescens
  source 1..1254: mol_type = other DNA
  misc_feature 1..1254: note = A nucleic acid sequence obtained from Photorhabdus luminescensstrain laumondii TTO1 encoding a TIC10367 pesticidal PirB proteinsequence.
atgagcgatattgttaagtataacgatgtaagtgcaccgatcccttatgctgtttattcaaatgccgtatatgcatttgagtgggattcatctgctattctaaagcaagccgtcgtcaagggattgtcgtatgtaccacatgtagggaaatatctttcttacattgttggttttttttggaaagataaagagaaagacatttggcaggaggttgtaggaaaagttcaacaactggttgaagatagtatattaaaagcagttaaaggtatactctcaggtaacatcaatgaattaaaagaaaaaatgaatgaggtaatccgttctctggagaagaatttaggtacccaagaagcaagggatgactacatgcatcttgccaggagtatggttggaaaagaagctagcttgatttttcatgaaaataagacaaattttcacatattgccgatgtattccacacttgccctgatgcagattatgtattggactgttggcattgagcgtcgcaaggaaatcggattgagtgatattgaagtcgagaatctaaggtcatatatcaaaaagttagttagtgatgcagagcatcacgtgaatagagtttataagttagaacttgatagtgtagtgtcagactctgatgttaatcgcgtggctgataatatcatgtatgtccatggatattgtcaaatacatggtctggaatatatggacatcattaaaaatatccaatccagaggtaataatattactgggttttatccgagaactatcagctactctacattctttggttcgccaacaagtgatgcgcgtattttggcattaaggccagagaaggatatgccagaaccgttcaaacccaaatttttaaatgaacggtttaataaaattgcatcggtcaaagggtacatagtacgaattggtggcgctaaacgtgttggggggctggagataacatttgagaatggcagcaagtatcaacagggccaagcaacgaatgagcatgaaatcgtcaatctcaaaggtaatttgattaagacgttggaagtatgggggaatggggccattgatgaagcaaaatttacattaacgaatggagatgtgttgacaataggtcaacgtaattcctctaattaccgtaagttctctcttgatggtcattatatttgcggggtgttcatcgcaaatgatcgttctggactggctggtcaagcagctaatattgccgtttcttatcaccaattagttgagtaa

SEQ ID NO: 66 - protein, 417 aa, Photorhabdus luminescens
  REGION 1..417: note = The amino acid sequence of the TIC10367 PirB protein.
MSDIVKYNDVSAPIPYAVYSNAVYAFEWDSSAILKQAVVKGLSYVPHVGKYLSYIVGFFWKDKEKDIWQEVVGKVQQLVEDSILKAVKGILSGNINELKEKMNEVIRSLEKNLGTQEARDDYMHLARSMVGKEASLIFHENKTNFHILPMYSTLALMQIMYWTVGIERRKEIGLSDIEVENLRSYIKKLVSDAEHHVNRVYKLELDSVVSDSDVNRVADNIMYVHGYCQIHGLEYMDIIKNIQSRGNNITGFYPRTISYSTFFGSPTSDARILALRPEKDMPEPFKPKFLNERFNKIASVKGYIVRIGGAKRVGGLEITFENGSKYQQGQATNEHEIVNLKGNLIKTLEVWGNGAIDEAKFTLTNGDVLTIGQRNSSNYRKFSLDGHYICGVFIANDRSGLAGQAANIAVSYHQLVE

SEQ ID NO: 67 - DNA, 1686 bp, synthetic construct
  source 1..1686: mol_type = other DNA
  misc_feature 1..1686: note = A nucleic acid sequence encoding a PirAB fusion protein, TIC10376comprised of the TIC10358 and TIC10367 coding sequences inoperable linkage and in frame.
atgccagtcaatcagattggcttacataatgaaaaggtgaaaaatatgagaaaaataacagttgataatgatgtggtaggacatgatactgaaatcaactcggttgtttcatcaactgcggagaaaattcgccaacagtttggagtaaaggtcgaccctaattcaagtcaggaaaagttctacattgcaacaccgattattcctgaatcccgaaagaatatcgttgtaaccaatgaaggtctcgccgatgttatcacggcgaaatattactggtcacattcttttacgtcagaatattttgaggataactcagttgatgtcaaggttggagagagcaaagtgttggttgccccttcaaacccgttgtattacagcaaagtagtcattttcaacaacactaaatccgtggcatttgtaacagtaagagaaaaaatgagcgatattgttaagtataacgatgtaagtgcaccgatcccttatgctgtttattcaaatgccgtatatgcatttgagtgggattcatctgctattctaaagcaagccgtcgtcaagggattgtcgtatgtaccacatgtagggaaatatctttcttacattgttggttttttttggaaagataaagagaaagacatttggcaggaggttgtaggaaaagttcaacaactggttgaagatagtatattaaaagcagttaaaggtatactctcaggtaacatcaatgaattaaaagaaaaaatgaatgaggtaatccgttctctggagaagaatttaggtacccaagaagcaagggatgactacatgcatcttgccaggagtatggttggaaaagaagctagcttgatttttcatgaaaataagacaaattttcacatattgccgatgtattccacacttgccctgatgcagattatgtattggactgttggcattgagcgtcgcaaggaaatcggattgagtgatattgaagtcgagaatctaaggtcatatatcaaaaagttagttagtgatgcagagcatcacgtgaatagagtttataagttagaacttgatagtgtagtgtcagactctgatgttaatcgcgtggctgataatatcatgtatgtccatggatattgtcaaatacatggtctggaatatatggacatcattaaaaatatccaatccagaggtaataatattactgggttttatccgagaactatcagctactctacattctttggttcgccaacaagtgatgcgcgtattttggcattaaggccagagaaggatatgccagaaccgttcaaacccaaatttttaaatgaacggtttaataaaattgcatcggtcaaagggtacatagtacgaattggtggcgctaaacgtgttggggggctggagataacatttgagaatggcagcaagtatcaacagggccaagcaacgaatgagcatgaaatcgtcaatctcaaaggtaatttgattaagacgttggaagtatgggggaatggggccattgatgaagcaaaatttacattaacgaatggagatgtgttgacaataggtcaacgtaattcctctaattaccgtaagttctctcttgatggtcattatatttgcggggtgttcatcgcaaatgatcgttctggactggctggtcaagcagctaatattgccgtttcttatcaccaattagttgagtaa

SEQ ID NO: 68 - protein, 561 aa, synthetic construct
  REGION 1..561: note = The amino acid sequence of the TIC10376 PirAB fusion protein.
MPVNQIGLHNEKVKNMRKITVDNDVVGHDTEINSVVSSTAEKIRQQFGVKVDPNSSQEKFYIATPIIPESRKNIVVTNEGLADVITAKYYWSHSFTSEYFEDNSVDVKVGESKVLVAPSNPLYYSKVVIFNNTKSVAFVTVREKMSDIVKYNDVSAPIPYAVYSNAVYAFEWDSSAILKQAVVKGLSYVPHVGKYLSYIVGFFWKDKEKDIWQEVVGKVQQLVEDSILKAVKGILSGNINELKEKMNEVIRSLEKNLGTQEARDDYMHLARSMVGKEASLIFHENKTNFHILPMYSTLALMQIMYWTVGIERRKEIGLSDIEVENLRSYIKKLVSDAEHHVNRVYKLELDSVVSDSDVNRVADNIMYVHGYCQIHGLEYMDIIKNIQSRGNNITGFYPRTISYSTFFGSPTSDARILALRPEKDMPEPFKPKFLNERFNKIASVKGYIVRIGGAKRVGGLEITFENGSKYQQGQATNEHEIVNLKGNLIKTLEVWGNGAIDEAKFTLTNGDVLTIGQRNSSNYRKFSLDGHYICGVFIANDRSGLAGQAANIAVSYHQLVE

SEQ ID NO: 69 - DNA, 402 bp, Photorhabdus asymbiotica
  source 1..402: mol_type = other DNA
  misc_feature 1..402: note = A nucleic acid sequence obtained from Photorhabdus asymbioticaencoding a TIC10360 pesticidal PirA protein sequence.
atgtctagaataactatttttattgattcagatgaacaaaaatcagaatttaattctgattctcctgttccggtacgtaaagacttaaatacagttgttcctttgagtgatctgactatatcccctcgttctagtgtggaagtatttagaatagatacaccaataattccagaatccagaagatctctgagagttgtaaatacagggctggcaagtagtgttacggctaaattttactggtctcatagttttacctctgaatggtttgagtctggttctatcgatgtaggattaggagaagataaggtattaaacgtgcctaacaactctttttattatagtaaatttgttatctataataacacggataaagttgcttatattacggcaaatttggtttaa

SEQ ID NO: 70 - protein, 133 aa, Photorhabdus asymbiotica
  REGION 1..133: note = The amino acid sequence of the TIC10360 PirA protein.
MSRITIFIDSDEQKSEFNSDSPVPVRKDLNTVVPLSDLTISPRSSVEVFRIDTPIIPESRRSLRVVNTGLASSVTAKFYWSHSFTSEWFESGSIDVGLGEDKVLNVPNNSFYYSKFVIYNNTDKVAYITANLV

SEQ ID NO: 71 - DNA, 1260 bp, Photorhabdus asymbiotica
  source 1..1260: mol_type = other DNA
  misc_feature 1..1260: note = A nucleic acid sequence obtained from Photorhabdus asymbioticaencoding a TIC10369 pesticidal PirB protein sequence.
atgcagacagagaatgttttagacataagaaccattgtggctaatgaatatgctataaaaacgagtgcattagagtgggatgttactgatattgtaaaaaatgcaatcataggaggcatatcttttatacctacggttggtcctgctatatcttttttagtcggtttattttggcctcaatcaaaagaaaatatatgggaagggattgtcaaacaaattgagaggatgatagaggaatctgcattaaagacgattaaaggtatccttgctggtgatattgcctatatacaagagcgaatggcaactgttgctgatcttcttgagaaacatccaggatcggcagaagcgcggagtgcttttaataacctggcagaaaatatagatggttatcacaaaaaatttaataatttctcggatgatgtaaattatcagatattacccatgttttctactacagttatgatgcagataacatattgggttgctggtttagagagaaaagatgaaatagggcttagtgatattgatattgaaaaagtccgagggttaattaaaaagacagtagaacaggctaataattatattaataatatatatggtagagaacttaatgatgctcttaataattcgacggctgacactgttgcaaataatgttatgtctgttcatggtcattgtcgtttacatggaattgaatatatcagtatttgggatagattaagtgaaactgagtctgtaaataatagaatctatgttgatgttttaagttattctactttctttgaccgtcaaacagcaaaggccagaattcaggcattgacgccagagaaagatatggctccacctctcaaaccggctcttaatggaggaaagagaagaaagataaattcgttaatgggacatattgtacgtattggaggggcgccaagggtaggagggctgacagttatatttgatgatggtagtcgccatcaattagggacaatatctggtgagacggcatctatttctctggatggtaatcgaattactagtttggaagtatggggcaatggtgctgttgataaagctgtctttactttgagtgatggtcgttcgttgtcatttggcgcacctggaacatccagatataggaagttttatgttggtgaaagtcactacatttcagggatctatttgtccagtgattacagcccgttagcaggtcaggcagcaaatatagctgtatcttatcagctgataaatgatgatgaaaaataa

SEQ ID NO: 72 - protein, 419 aa, Photorhabdus asymbiotica
  REGION 1..419: note = The amino acid sequence of the TIC10369 PirB protein.
MQTENVLDIRTIVANEYAIKTSALEWDVTDIVKNAIIGGISFIPTVGPAISFLVGLFWPQSKENIWEGIVKQIERMIEESALKTIKGILAGDIAYIQERMATVADLLEKHPGSAEARSAFNNLAENIDGYHKKFNNFSDDVNYQILPMFSTTVMMQITYWVAGLERKDEIGLSDIDIEKVRGLIKKTVEQANNYINNIYGRELNDALNNSTADTVANNVMSVHGHCRLHGIEYISIWDRLSETESVNNRIYVDVLSYSTFFDRQTAKARIQALTPEKDMAPPLKPALNGGKRRKINSLMGHIVRIGGAPRVGGLTVIFDDGSRHQLGTISGETASISLDGNRITSLEVWGNGAVDKAVFTLSDGRSLSFGAPGTSRYRKFYVGESHYISGIYLSSDYSPLAGQAANIAVSYQLINDDEK

SEQ ID NO: 73 - DNA, 1659 bp, synthetic construct
  source 1..1659: mol_type = other DNA
  misc_feature 1..1659: note = A nucleic acid sequence encoding a PirAB fusion protein, TIC10377comprised of the TIC10360 and TIC10369 coding sequences inoperable linkage and in frame.
atgtctagaataactatttttattgattcagatgaacaaaaatcagaatttaattctgattctcctgttccggtacgtaaagacttaaatacagttgttcctttgagtgatctgactatatcccctcgttctagtgtggaagtatttagaatagatacaccaataattccagaatccagaagatctctgagagttgtaaatacagggctggcaagtagtgttacggctaaattttactggtctcatagttttacctctgaatggtttgagtctggttctatcgatgtaggattaggagaagataaggtattaaacgtgcctaacaactctttttattatagtaaatttgttatctataataacacggataaagttgcttatattacggcaaatttggttatgcagacagagaatgttttagacataagaaccattgtggctaatgaatatgctataaaaacgagtgcattagagtgggatgttactgatattgtaaaaaatgcaatcataggaggcatatcttttatacctacggttggtcctgctatatcttttttagtcggtttattttggcctcaatcaaaagaaaatatatgggaagggattgtcaaacaaattgagaggatgatagaggaatctgcattaaagacgattaaaggtatccttgctggtgatattgcctatatacaagagcgaatggcaactgttgctgatcttcttgagaaacatccaggatcggcagaagcgcggagtgcttttaataacctggcagaaaatatagatggttatcacaaaaaatttaataatttctcggatgatgtaaattatcagatattacccatgttttctactacagttatgatgcagataacatattgggttgctggtttagagagaaaagatgaaatagggcttagtgatattgatattgaaaaagtccgagggttaattaaaaagacagtagaacaggctaataattatattaataatatatatggtagagaacttaatgatgctcttaataattcgacggctgacactgttgcaaataatgttatgtctgttcatggtcattgtcgtttacatggaattgaatatatcagtatttgggatagattaagtgaaactgagtctgtaaataatagaatctatgttgatgttttaagttattctactttctttgaccgtcaaacagcaaaggccagaattcaggcattgacgccagagaaagatatggctccacctctcaaaccggctcttaatggaggaaagagaagaaagataaattcgttaatgggacatattgtacgtattggaggggcgccaagggtaggagggctgacagttatatttgatgatggtagtcgccatcaattagggacaatatctggtgagacggcatctatttctctggatggtaatcgaattactagtttggaagtatggggcaatggtgctgttgataaagctgtctttactttgagtgatggtcgttcgttgtcatttggcgcacctggaacatccagatataggaagttttatgttggtgaaagtcactacatttcagggatctatttgtccagtgattacagcccgttagcaggtcaggcagcaaatatagctgtatcttatcagctgataaatgatgatgaaaaataa

SEQ ID NO: 74 - protein, 552 aa, synthetic construct
  REGION 1..552: note = The amino acid sequence of the TIC10377 PirAB fusion protein.
MSRITIFIDSDEQKSEFNSDSPVPVRKDLNTVVPLSDLTISPRSSVEVFRIDTPIIPESRRSLRVVNTGLASSVTAKFYWSHSFTSEWFESGSIDVGLGEDKVLNVPNNSFYYSKFVIYNNTDKVAYITANLVMQTENVLDIRTIVANEYAIKTSALEWDVTDIVKNAIIGGISFIPTVGPAISFLVGLFWPQSKENIWEGIVKQIERMIEESALKTIKGILAGDIAYIQERMATVADLLEKHPGSAEARSAFNNLAENIDGYHKKFNNFSDDVNYQILPMFSTTVMMQITYWVAGLERKDEIGLSDIDIEKVRGLIKKTVEQANNYINNIYGRELNDALNNSTADTVANNVMSVHGHCRLHGIEYISIWDRLSETESVNNRIYVDVLSYSTFFDRQTAKARIQALTPEKDMAPPLKPALNGGKRRKINSLMGHIVRIGGAPRVGGLTVIFDDGSRHQLGTISGETASISLDGNRITSLEVWGNGAVDKAVFTLSDGRSLSFGAPGTSRYRKFYVGESHYISGIYLSSDYSPLAGQAANIAVSYQLINDDEK

SEQ ID NO: 75 - DNA, 432 bp, Xenorhabdus sp.
  source 1..432: mol_type = other DNA
  misc_feature 1..432: note = A nucleic acid sequence obtained from Xenorhabdus sp. strainNBAII XenSa04 encoding a TIC10361 pesticidal PirA proteinsequence.
atgatcacaataaatataaatacaaacggtgttaatggtattaccattacaaatagtaataatgaacctactccagtatcgacaacttacggtccaaatacaccagcatcagaaccccttacagtcagtaattatagtgatatcacaatagaaccacattcttctgtgcaggcaacaagaattgacacgcctattattcctgaaacacgtccagattactatgtagccaactccggccctgcaccaacagttagggctgttttttattggtctcattctttcacatcagaatggttcgaatcttcctctatcacagtgaaagcaggagaggatggaatattaaaagcacctggtaattctttatattacagcaaagtcgtcatttataatgacaccgataaacgggcttttgttactggatataataaataa

SEQ ID NO: 76 - protein, 143 aa, Xenorhabdus sp.
  REGION 1..143: note = The amino acid sequence of the TIC10361 PirA protein.
MITININTNGVNGITITNSNNEPTPVSTTYGPNTPASEPLTVSNYSDITIEPHSSVQATRIDTPIIPETRPDYYVANSGPAPTVRAVFYWSHSFTSEWFESSSITVKAGEDGILKAPGNSLYYSKVVIYNDTDKRAFVTGYNK

SEQ ID NO: 77 - DNA, 1278 bp, Xenorhabdus sp.
  source 1..1278: mol_type = other DNA
  misc_feature 1..1278: note = A nucleic acid sequence obtained from Xenorhabdus sp. strainNBAII XenSa04 encoding a TIC10370 pesticidal PirB proteinsequence.
atgaataccacacctattactgtatctgaaaatgaaacatcgcctttactgactgacgtaatgcctatggatctttatgcagtatccacccctgattatgaatgggatatgtcgtcaatcataaaggatgctattattggtggcataggatttattccaggtccgggtccggcattatccttcctgttaggactattttggcctcagcagaaagacaatacttgggagcaaattctccaaaaagtagagcagatgatagagaatgctgttctacaaaccattaaaggaatacttaatggagaaatacaagagatcaaagggaaaatggaacatgtagaatctatgctgaaaaactcgccgggtagtcaggaaagtcatgatgcatatatgttcctggcaagatatctggtgagtatagatgaaaaattcaaatcttttgataatagaacaaattaccagattctcccaatgtatactaatactattatgttacagatcccttattggaaaatgggaatagagaagaaaaaagatattgggctgacagatattgaagtcaatgaattaaaagaacttatcgacaaattagtaggtaaggccaagaactatattcatacgatgtatactaatgaatataacgatgctataaacacatcaacagcagggagtgtcactaataatttattatctgtaaggggatattgtttattacacggtttggagtgtattgagttaattgagcatatacagaataatagccttgaaagtggtttctatcctaaaactatcagttattcgacagtgtttgatcgtccgactaataaaatgagaattcaggctcttacagaagacgatgcaatgcaggagcctttcaagccatctttaattaatgggaaatacaataaaatacaatccataattggatatgtacaaagaattgggaatgcacctagagttggtggtattaaaattacatttaccaatggctcatcttatacacttggtacggtgacctcagaaacaaattcaattgaactaaatgatagtgttatcgagagcttggaagtatggggaaatggtgctgttgatgaggcattatttaagttgagtgatgggcgtttattgcgtattggtgagcgttacgcgaaaaaatacagaaaatatgctgttgatcatcactatattgcgggaatttacttggccagcgatgagccttcacttgctggtcaagccgcaggtattgccgtttcatatcatatgatggccgacaaaaaataa

SEQ ID NO: 78 - protein, 425 aa, Xenorhabdus sp.
  REGION 1..425: note = The amino acid sequence of the TIC10370 PirB protein.
MNTTPITVSENETSPLLTDVMPMDLYAVSTPDYEWDMSSIIKDAIIGGIGFIPGPGPALSFLLGLFWPQQKDNTWEQILQKVEQMIENAVLQTIKGILNGEIQEIKGKMEHVESMLKNSPGSQESHDAYMFLARYLVSIDEKFKSFDNRTNYQILPMYTNTIMLQIPYWKMGIEKKKDIGLTDIEVNELKELIDKLVGKAKNYIHTMYTNEYNDAINTSTAGSVTNNLLSVRGYCLLHGLECIELIEHIQNNSLESGFYPKTISYSTVFDRPTNKMRIQALTEDDAMQEPFKPSLINGKYNKIQSIIGYVQRIGNAPRVGGIKITFTNGSSYTLGTVTSETNSIELNDSVIESLEVWGNGAVDEALFKLSDGRLLRIGERYAKKYRKYAVDHHYIAGIYLASDEPSLAGQAAGIAVSYHMMADKK

SEQ ID NO: 79 - DNA, 1707 bp, synthetic construct
  source 1..1707: mol_type = other DNA
  misc_feature 1..1707: note = A nucleic acid sequence encoding a PirAB fusion protein, TIC10378comprised of the TIC10361 and TIC10370 coding sequences inoperable linkage and in frame.
atgatcacaataaatataaatacaaacggtgttaatggtattaccattacaaatagtaataatgaacctactccagtatcgacaacttacggtccaaatacaccagcatcagaaccccttacagtcagtaattatagtgatatcacaatagaaccacattcttctgtgcaggcaacaagaattgacacgcctattattcctgaaacacgtccagattactatgtagccaactccggccctgcaccaacagttagggctgttttttattggtctcattctttcacatcagaatggttcgaatcttcctctatcacagtgaaagcaggagaggatggaatattaaaagcacctggtaattctttatattacagcaaagtcgtcatttataatgacaccgataaacgggcttttgttactggatataataaaatgaataccacacctattactgtatctgaaaatgaaacatcgcctttactgactgacgtaatgcctatggatctttatgcagtatccacccctgattatgaatgggatatgtcgtcaatcataaaggatgctattattggtggcataggatttattccaggtccgggtccggcattatccttcctgttaggactattttggcctcagcagaaagacaatacttgggagcaaattctccaaaaagtagagcagatgatagagaatgctgttctacaaaccattaaaggaatacttaatggagaaatacaagagatcaaagggaaaatggaacatgtagaatctatgctgaaaaactcgccgggtagtcaggaaagtcatgatgcatatatgttcctggcaagatatctggtgagtatagatgaaaaattcaaatcttttgataatagaacaaattaccagattctcccaatgtatactaatactattatgttacagatcccttattggaaaatgggaatagagaagaaaaaagatattgggctgacagatattgaagtcaatgaattaaaagaacttatcgacaaattagtaggtaaggccaagaactatattcatacgatgtatactaatgaatataacgatgctataaacacatcaacagcagggagtgtcactaataatttattatctgtaaggggatattgtttattacacggtttggagtgtattgagttaattgagcatatacagaataatagccttgaaagtggtttctatcctaaaactatcagttattcgacagtgtttgatcgtccgactaataaaatgagaattcaggctcttacagaagacgatgcaatgcaggagcctttcaagccatctttaattaatgggaaatacaataaaatacaatccataattggatatgtacaaagaattgggaatgcacctagagttggtggtattaaaattacatttaccaatggctcatcttatacacttggtacggtgacctcagaaacaaattcaattgaactaaatgatagtgttatcgagagcttggaagtatggggaaatggtgctgttgatgaggcattatttaagttgagtgatgggcgtttattgcgtattggtgagcgttacgcgaaaaaatacagaaaatatgctgttgatcatcactatattgcgggaatttacttggccagcgatgagccttcacttgctggtcaagccgcaggtattgccgtttcatatcatatgatggccgacaaaaaataa

SEQ ID NO: 80 - protein, 568 aa, synthetic construct
  REGION 1..568: note = The amino acid sequence of the TIC10378 PirAB fusion protein.
MITININTNGVNGITITNSNNEPTPVSTTYGPNTPASEPLTVSNYSDITIEPHSSVQATRIDTPIIPETRPDYYVANSGPAPTVRAVFYWSHSFTSEWFESSSITVKAGEDGILKAPGNSLYYSKVVIYNDTDKRAFVTGYNKMNTTPITVSENETSPLLTDVMPMDLYAVSTPDYEWDMSSIIKDAIIGGIGFIPGPGPALSFLLGLFWPQQKDNTWEQILQKVEQMIENAVLQTIKGILNGEIQEIKGKMEHVESMLKNSPGSQESHDAYMFLARYLVSIDEKFKSFDNRTNYQILPMYTNTIMLQIPYWKMGIEKKKDIGLTDIEVNELKELIDKLVGKAKNYIHTMYTNEYNDAINTSTAGSVTNNLLSVRGYCLLHGLECIELIEHIQNNSLESGFYPKTISYSTVFDRPTNKMRIQALTEDDAMQEPFKPSLINGKYNKIQSIIGYVQRIGNAPRVGGIKITFTNGSSYTLGTVTSETNSIELNDSVIESLEVWGNGAVDEALFKLSDGRLLRIGERYAKKYRKYAVDHHYIAGIYLASDEPSLAGQAAGIAVSYHMMADKK

SEQ ID NO: 81 - DNA, 411 bp, Yersinia aldovae
  source 1..411: mol_type = other DNA
  misc_feature 1..411: note = A nucleic acid sequence obtained from Yersinia aldovae strain670-83 encoding a TIC10362 pesticidal PirA protein sequence.
atgagcaaggtaaccatcactattgatagctgttcaaatgaagtcgaagttaaaaatcaaactgaggttgataccgaaagtctagcgttgaccactgcacaagttagagccagggtgccaactgaggtcgcacctaattcaagcaccgaagttctgtatcggagtacaccgattattcctgaaagtcgtcgaaatgtaatgatcactaatgatggtgctgcaaatgtcattacagcccaatactactggtcgcatagtttcacgagtcaatggttcttatatacgtctattgacgtcaatgttggtgattctaagctattagtctcaccctccaactcattgtattacagtaaggttgttctgattaataacacaaaccgtaaagcatatgttactgccgaggaaaaatag

SEQ ID NO: 82 - protein, 136 aa, Yersinia aldovae
  REGION 1..136: note = The amino acid sequence of the TIC10362 PirA protein.
MSKVTITIDSCSNEVEVKNQTEVDTESLALTTAQVRARVPTEVAPNSSTEVLYRSTPIIPESRRNVMITNDGAANVITAQYYWSHSFTSQWFLYTSIDVNVGDSKLLVSPSNSLYYSKVVLINNTNRKAYVTAEEK

SEQ ID NO: 83 - DNA, 1251 bp, Yersinia aldovae
  source 1..1251: mol_type = other DNA
  misc_feature 1..1251: note = A nucleic acid sequence obtained from Yersinia aldovae strain670-83 encoding a TIC10371 pesticidal PirB protein sequence.
atgaataacattacagaatataacaatacagagaactttgtcccttataatgtatacgctacttcagcctttgaatttgactgggattcttcagccattcttaagcaagcagtgcttaaaggtatatcattcattccttatgtcggtgattatttatcctctattattggcttcttttggaaagaccaagagagagatatctggcaggaaattttgggccgggtacagcaacttatcgaagagaatgtgcttaaagctattaaaggcattttattgggcgatattgctgaacttaaagggaaggttgcatccgttgtcgcggccttgcaggaccatcctggtacaccggaagccaaaagtttatttatgagcgtatcggtacatttggatagcgtacaacgcaagtttactacttttgatcacaaaactaattaccatatcctgccgatgtattcagcaaccgcgttgatgcaaataatgtactggaccatgggcattgagcgtaaagacgatatcggattgaacagtaatgaagttgggcaacttcaacgaaatattaatctattggttacacatgtcgaggattatattcaagagatttacgatacagaattagagatccaatacaacgactcggcacctaatactgtagccaacaatgttatgtatgtacatggctactgtcgggtgcatggtttggagtataccgagatcattcaaagtattcagaagaatagaagcaatacacagggactgtattcaaaaatactgagctactcgactttctttggttggccaaccagtcaggcgcgaattctcgcattaaaagacgaaataaatatgccggagccatttaagccaaaattaataaatggccgtataaaccaagttaagtcagttaaaggctatatacgacgtatcggaggtgctttacgggtaggtggattagaaatcacctttgagaatggtagtaagtactcgcaaggaactgttactggtgaatttagttcaattgaccttaatgggagtgtcattgaaacaatggaaacttggggtagtggcgcaattgacgaagctaaatttaccttaagcgatggccgtacctttactgtcggtcaacgttattcaacaaattacagaaagtttgcacttgagggacactatatttctggcatttttattgccagcgatcgaagtgaacttgccggccaagctgcaaatatttgtgtttcttatcatcagaaacagtga

SEQ ID NO: 84 - protein, 416 aa, Yersinia aldovae
  REGION 1..416: note = The amino acid sequence of the TIC10371 PirB protein.
MNNITEYNNTENFVPYNVYATSAFEFDWDSSAILKQAVLKGISFIPYVGDYLSSIIGFFWKDQERDIWQEILGRVQQLIEENVLKAIKGILLGDIAELKGKVASVVAALQDHPGTPEAKSLFMSVSVHLDSVQRKFTTFDHKTNYHILPMYSATALMQIMYWTMGIERKDDIGLNSNEVGQLQRNINLLVTHVEDYIQEIYDTELEIQYNDSAPNTVANNVMYVHGYCRVHGLEYTEIIQSIQKNRSNTQGLYSKILSYSTFFGWPTSQARILALKDEINMPEPFKPKLINGRINQVKSVKGYIRRIGGALRVGGLEITFENGSKYSQGTVTGEFSSIDLNGSVIETMETWGSGAIDEAKFTLSDGRTFTVGQRYSTNYRKFALEGHYISGIFIASDRSELAGQAANICVSYHQKQ

SEQ ID NO: 85 - DNA, 1659 bp, synthetic construct
  source 1..1659: mol_type = other DNA
  misc_feature 1..1659: note = A nucleic acid sequence encoding a PirAB fusion protein, TIC10379comprised of the TIC10362 and TIC10371 coding sequences inoperable linkage and in frame.
atgagcaaggtaaccatcactattgatagctgttcaaatgaagtcgaagttaaaaatcaaactgaggttgataccgaaagtctagcgttgaccactgcacaagttagagccagggtgccaactgaggtcgcacctaattcaagcaccgaagttctgtatcggagtacaccgattattcctgaaagtcgtcgaaatgtaatgatcactaatgatggtgctgcaaatgtcattacagcccaatactactggtcgcatagtttcacgagtcaatggttcttatatacgtctattgacgtcaatgttggtgattctaagctattagtctcaccctccaactcattgtattacagtaaggttgttctgattaataacacaaaccgtaaagcatatgttactgccgaggaaaaaatgaataacattacagaatataacaatacagagaactttgtcccttataatgtatacgctacttcagcctttgaatttgactgggattcttcagccattcttaagcaagcagtgcttaaaggtatatcattcattccttatgtcggtgattatttatcctctattattggcttcttttggaaagaccaagagagagatatctggcaggaaattttgggccgggtacagcaacttatcgaagagaatgtgcttaaagctattaaaggcattttattgggcgatattgctgaacttaaagggaaggttgcatccgttgtcgcggccttgcaggaccatcctggtacaccggaagccaaaagtttatttatgagcgtatcggtacatttggatagcgtacaacgcaagtttactacttttgatcacaaaactaattaccatatcctgccgatgtattcagcaaccgcgttgatgcaaataatgtactggaccatgggcattgagcgtaaagacgatatcggattgaacagtaatgaagttgggcaacttcaacgaaatattaatctattggttacacatgtcgaggattatattcaagagatttacgatacagaattagagatccaatacaacgactcggcacctaatactgtagccaacaatgttatgtatgtacatggctactgtcgggtgcatggtttggagtataccgagatcattcaaagtattcagaagaatagaagcaatacacagggactgtattcaaaaatactgagctactcgactttctttggttggccaaccagtcaggcgcgaattctcgcattaaaagacgaaataaatatgccggagccatttaagccaaaattaataaatggccgtataaaccaagttaagtcagttaaaggctatatacgacgtatcggaggtgctttacgggtaggtggattagaaatcacctttgagaatggtagtaagtactcgcaaggaactgttactggtgaatttagttcaattgaccttaatgggagtgtcattgaaacaatggaaacttggggtagtggcgcaattgacgaagctaaatttaccttaagcgatggccgtacctttactgtcggtcaacgttattcaacaaattacagaaagtttgcacttgagggacactatatttctggcatttttattgccagcgatcgaagtgaacttgccggccaagctgcaaatatttgtgtttcttatcatcagaaacagtga

SEQ ID NO: 86 - protein, 552 aa, synthetic construct
  REGION 1..552: note = The amino acid sequence of the TIC10379 PirAB fusion protein.
MSKVTITIDSCSNEVEVKNQTEVDTESLALTTAQVRARVPTEVAPNSSTEVLYRSTPIIPESRRNVMITNDGAANVITAQYYWSHSFTSQWFLYTSIDVNVGDSKLLVSPSNSLYYSKVVLINNTNRKAYVTAEEKMNNITEYNNTENFVPYNVYATSAFEFDWDSSAILKQAVLKGISFIPYVGDYLSSIIGFFWKDQERDIWQEILGRVQQLIEENVLKAIKGILLGDIAELKGKVASVVAALQDHPGTPEAKSLFMSVSVHLDSVQRKFTTFDHKTNYHILPMYSATALMQIMYWTMGIERKDDIGLNSNEVGQLQRNINLLVTHVEDYIQEIYDTELEIQYNDSAPNTVANNVMYVHGYCRVHGLEYTEIIQSIQKNRSNTQGLYSKILSYSTFFGWPTSQARILALKDEINMPEPFKPKLINGRINQVKSVKGYIRRIGGALRVGGLEITFENGSKYSQGTVTGEFSSIDLNGSVIETMETWGSGAIDEAKFTLSDGRTFTVGQRYSTNYRKFALEGHYISGIFIASDRSELAGQAANICVSYHQKQ

SEQ ID NO: 87 - DNA, 414 bp, Xenorhabdus doucetiae
  source 1..414: mol_type = other DNA
  misc_feature 1..414: note = A nucleic acid sequence obtained from Xenorhabdus doucetiaestrain FRM16 encoding a TIC10363 pesticidal PirA proteinsequence.
atgattacaattaatataagtggtggttcagtaacaattaataacacttacaatatcacatcagaatctggcattcaaaatacccctgcctcagaacctctcaccgtcattccttatagagatatgacaatagaaccacactcttctattgaggcaacaagaactgatacgcctattattcctgaaacacgccccaattattatatcgccaattccggccctgcatcagaagttagagcagtgttttattggtcgcattctttcacatcacaatggttcgaatcttcctctatcatagtgaaagcaggggaagacggcatattacaatcaccaagcaactcgctatattacagcaaggttgtcatttataatgatacagataaacgcgcctttgtgactggatataataagtaa

SEQ ID NO: 88 - protein, 137 aa, Xenorhabdus doucetiae
  REGION 1..137: note = The amino acid sequence of the TIC10363 PirA protein.
MITINISGGSVTINNTYNITSESGIQNTPASEPLTVIPYRDMTIEPHSSIEATRTDTPIIPETRPNYYIANSGPASEVRAVFYWSHSFTSQWFESSSIIVKAGEDGILQSPSNSLYYSKVVIYNDTDKRAFVTGYNK

SEQ ID NO: 89 - DNA, 1293 bp, Xenorhabdus doucetiae
  source 1..1293: mol_type = other DNA
  misc_feature 1..1293: note = A nucleic acid sequence obtained from Xenorhabdus doucetiaestrain FRM16 encoding a TIC10372 pesticidal PirB proteinsequence.
atgaataacacatctataaatatcaatgagaatgaaacattacctttagaagttatcccttcaatgcctgaacccatgttaatcgttccttatgcaacttctactcctgattatgaatgggatgcctccggaataataaaagatgccattattggtggtataggatttattcctggcccaggtccggcaatatcttttctgttaggactcttttggcctcaacaggcagacaatacttgggaacaaattctgcaaaaagtcgagcagatgatagaggatgctgttctcaaaaccattcaaggtatattgaatggcgatatacaagaaattaaaggtaaaatggaacatgtccaatacatgttggaaacctcaccgggtagccaagaaagccgtgaagcttatatgtttctggcgagatatctggtgagtatagatgaaaaattcaagtcctttgataataaaacaaattaccaaattctcccgatgtataccaacactctcatgttacaggttccttattggaaaatgggcatagagaagcaaaaggatattggtttatccgatatagaagttaatgaattaaaacagcttatcgataaattatataccaaggctaacagctatattcatgaaacgtatacgcgtcaatataacgatgcgataaacacgtcaaccgcagcaaatatcaccaataatttattttctgtcagaggatattgtttgttacacggtttagagtgtcttgaaatgattgagcatctacaaaagaatagccttgaaagtggtttctatcccaaaaccatcagttattctaccgtattcgatcgtcagactcccaaaatgagaattcaggctctgacagaagacgatcaaatgcaggagccattaaagccatctttaatcaacggcaaatacaatcaaataaaatcattgactggatatgtccgtagaattggcaatgctcccagagtgggggggatgacgatcacatttgccaacggtgcatcttacacactgggtacagtaacatcagaaacgacgtcaattgagctcaatggcagtgtgatcgaaagcttggaagtctggggagatggcgcggttgatgaggcattatttacgttaagtgataaacgcctattccgtatcggtgagcgctacgccagaaaatacaaaaaatatgctgttgatagccactatattgcagggctttatttagccagtgatgagccttcacttgcaggtcaagccgcaggtattgccgtttcataccatatgctggatgacaaaaaataa

SEQ ID NO: 90 - protein, 430 aa, Xenorhabdus doucetiae
  REGION 1..430: note = The amino acid sequence of the TIC10372 PirB protein.
MNNTSININENETLPLEVIPSMPEPMLIVPYATSTPDYEWDASGIIKDAIIGGIGFIPGPGPAISFLLGLFWPQQADNTWEQILQKVEQMIEDAVLKTIQGILNGDIQEIKGKMEHVQYMLETSPGSQESREAYMFLARYLVSIDEKFKSFDNKTNYQILPMYTNTLMLQVPYWKMGIEKQKDIGLSDIEVNELKQLIDKLYTKANSYIHETYTRQYNDAINTSTAANITNNLFSVRGYCLLHGLECLEMIEHLQKNSLESGFYPKTISYSTVFDRQTPKMRIQALTEDDQMQEPLKPSLINGKYNQIKSLTGYVRRIGNAPRVGGMTITFANGASYTLGTVTSETTSIELNGSVIESLEVWGDGAVDEALFTLSDKRLFRIGERYARKYKKYAVDSHYIAGLYLASDEPSLAGQAAGIAVSYHMLDDKK

SEQ ID NO: 91 - DNA, 1704 bp, synthetic construct
  source 1..1704: mol_type = other DNA
  misc_feature 1..1704: note = A nucleic acid sequence encoding a PirAB fusion protein, TIC10380comprised of the TIC10363 and TIC10372 coding sequences inoperable linkage and in frame.
atgattacaattaatataagtggtggttcagtaacaattaataacacttacaatatcacatcagaatctggcattcaaaatacccctgcctcagaacctctcaccgtcattccttatagagatatgacaatagaaccacactcttctattgaggcaacaagaactgatacgcctattattcctgaaacacgccccaattattatatcgccaattccggccctgcatcagaagttagagcagtgttttattggtcgcattctttcacatcacaatggttcgaatcttcctctatcatagtgaaagcaggggaagacggcatattacaatcaccaagcaactcgctatattacagcaaggttgtcatttataatgatacagataaacgcgcctttgtgactggatataataagatgaataacacatctataaatatcaatgagaatgaaacattacctttagaagttatcccttcaatgcctgaacccatgttaatcgttccttatgcaacttctactcctgattatgaatgggatgcctccggaataataaaagatgccattattggtggtataggatttattcctggcccaggtccggcaatatcttttctgttaggactcttttggcctcaacaggcagacaatacttgggaacaaattctgcaaaaagtcgagcagatgatagaggatgctgttctcaaaaccattcaaggtatattgaatggcgatatacaagaaattaaaggtaaaatggaacatgtccaatacatgttggaaacctcaccgggtagccaagaaagccgtgaagcttatatgtttctggcgagatatctggtgagtatagatgaaaaattcaagtcctttgataataaaacaaattaccaaattctcccgatgtataccaacactctcatgttacaggttccttattggaaaatgggcatagagaagcaaaaggatattggtttatccgatatagaagttaatgaattaaaacagcttatcgataaattatataccaaggctaacagctatattcatgaaacgtatacgcgtcaatataacgatgcgataaacacgtcaaccgcagcaaatatcaccaataatttattttctgtcagaggatattgtttgttacacggtttagagtgtcttgaaatgattgagcatctacaaaagaatagccttgaaagtggtttctatcccaaaaccatcagttattctaccgtattcgatcgtcagactcccaaaatgagaattcaggctctgacagaagacgatcaaatgcaggagccattaaagccatctttaatcaacggcaaatacaatcaaataaaatcattgactggatatgtccgtagaattggcaatgctcccagagtgggggggatgacgatcacatttgccaacggtgcatcttacacactgggtacagtaacatcagaaacgacgtcaattgagctcaatggcagtgtgatcgaaagcttggaagtctggggagatggcgcggttgatgaggcattatttacgttaagtgataaacgcctattccgtatcggtgagcgctacgccagaaaatacaaaaaatatgctgttgatagccactatattgcagggctttatttagccagtgatgagccttcacttgcaggtcaagccgcaggtattgccgtttcataccatatgctggatgacaaaaaataa

SEQ ID NO: 92 - protein, 567 aa, synthetic construct
  REGION 1..567: note = The amino acid sequence of the TIC10380 PirAB fusion protein.
MITINISGGSVTINNTYNITSESGIQNTPASEPLTVIPYRDMTIEPHSSIEATRTDTPIIPETRPNYYIANSGPASEVRAVFYWSHSFTSQWFESSSIIVKAGEDGILQSPSNSLYYSKVVIYNDTDKRAFVTGYNKMNNTSININENETLPLEVIPSMPEPMLIVPYATSTPDYEWDASGIIKDAIIGGIGFIPGPGPAISFLLGLFWPQQADNTWEQILQKVEQMIEDAVLKTIQGILNGDIQEIKGKMEHVQYMLETSPGSQESREAYMFLARYLVSIDEKFKSFDNKTNYQILPMYTNTLMLQVPYWKMGIEKQKDIGLSDIEVNELKQLIDKLYTKANSYIHETYTRQYNDAINTSTAANITNNLFSVRGYCLLHGLECLEMIEHLQKNSLESGFYPKTISYSTVFDRQTPKMRIQALTEDDQMQEPLKPSLINGKYNQIKSLTGYVRRIGNAPRVGGMTITFANGASYTLGTVTSETTSIELNGSVIESLEVWGDGAVDEALFTLSDKRLFRIGERYARKYKKYAVDSHYIAGLYLASDEPSLAGQAAGIAVSYHMLDDKK

SEQ ID NO: 93 - DNA, 429 bp, Xenorhabdus griffiniae
  source 1..429: mol_type = other DNA
  misc_feature 1..429: note = A nucleic acid sequence obtained from Xenorhabdus griffiniaestrain BMMCB encoding a TIC10364 pesticidal PirA proteinsequence.
atgagcataatcaatataaatataagtggtagtagtgacattacaatcataaacaataccccatctaacccagaaccactcatttacaatacagacacacccgcatcagaacctcttaccgtcaatccttatagggatatgacaatagagccacactcttctattgaggcaataagaattgatacgccaattattcccgaaacccgccccaattattacgtagccaattctggccccgcatcatcagttagagccgttttttattggtctcactctttcacatcagaatggttcgaatattctgctatcacagtgaaagccggggaagacggcatattacaatcaccgagcaactctgtgtattacagcaaggtcgtcatttataacgaaaccgataaacgcgcctttgttactggatataataagtaa

SEQ ID NO: 94 - protein, 142 aa, Xenorhabdus griffiniae
  REGION 1..142: note = The amino acid sequence of the TIC10364 PirA protein.
MSIININISGSSDITIINNTPSNPEPLIYNTDTPASEPLTVNPYRDMTIEPHSSIEAIRIDTPIIPETRPNYYVANSGPASSVRAVFYWSHSFTSEWFEYSAITVKAGEDGILQSPSNSVYYSKVVIYNETDKRAFVTGYNK

SEQ ID NO: 95 - DNA, 1278 bp, Xenorhabdus griffiniae
  source 1..1278: mol_type = other DNA
  misc_feature 1..1278: note = A nucleic acid sequence obtained from Xenorhabdus griffiniaestrain BMMCB encoding a TIC10373 pesticidal PirB proteinsequence.
atgaataccacaccgattaatgtatctgaaaatgacacattgcctgtactcactgacgtcatgcttatcgtgccttataccacctctacccccgattatgaatgggatatgtcatcaataataaaggatgccattattggcggcgtagggtttattccaggagtaggttccgcaatgtctttcctgttagggctattttggccgcaacagaaagataatacctgggagcaaatcctccaaaaagtagagcaaatgatagagaatgctgctctacaaacgattaaaggaatacttaatggagatatacaagaaatcaaaggaaaaatggaacatgtgcaatacatgctggaaacctcgcctggcagccaggaaagccatgacgcctatatgttcctggctagatatctggtgagtatcgatgaaagattcaagtcttttgataataaaacaaactaccagatcctgccgatgtacactaacacggttatgttacagatcccttattggaaaatggggatagaaaagaaaaatgatattgggctgaccgatattgaagtcaatgagttaaaacaacttatcgacacattggttgacagagccagaaactatattcatacgatgtatactaatgaatataataatgccataaatacatcaacagcagagagtgtcactaataatttattgtctgtaagagggtattgtttattacacggtttagagtgtattgagttaattgaacatctacaaaataatagcctggaaagtggttttaatcctaaaactatcagttattcaaccgtatttgatcgtcctactaacaaaacgagaattcaggctctgacagaagacgatcaaatgcaggaacccttcaagccctctttaatcgacggtaaatacaataaaataaaatcattgcttggctatgtacgaagaatcggcaatgcccccagagtgggtggtattcaaatcacatttgccaacgattcatcctatacactcggcaccgtaacatcagaaacgagttctattgaactcaatgatagtgttatcgaaaggttggaagtatggggcaatggcgcggttgatgaggcgttatttacgttaagtgatgggcgtcaactcagagtcggtgaacgctacgcgacaaaatacagaaaatacgctgttgatggccactatattgcagggctgtacttagccagcgatgaaccttcacttgctggtcaggccgcaggtattgccgtttcataccatatgttagctgataaaaaataa

SEQ ID NO: 96 - protein, 425 aa, Xenorhabdus griffiniae
  REGION 1..425: note = The amino acid sequence of the TIC10373 PirB protein.
MNTTPINVSENDTLPVLTDVMLIVPYTTSTPDYEWDMSSIIKDAIIGGVGFIPGVGSAMSFLLGLFWPQQKDNTWEQILQKVEQMIENAALQTIKGILNGDIQEIKGKMEHVQYMLETSPGSQESHDAYMFLARYLVSIDERFKSFDNKTNYQILPMYTNTVMLQIPYWKMGIEKKNDIGLTDIEVNELKQLIDTLVDRARNYIHTMYTNEYNNAINTSTAESVTNNLLSVRGYCLLHGLECIELIEHLQNNSLESGFNPKTISYSTVFDRPTNKTRIQALTEDDQMQEPFKPSLIDGKYNKIKSLLGYVRRIGNAPRVGGIQITFANDSSYTLGTVTSETSSIELNDSVIERLEVWGNGAVDEALFTLSDGRQLRVGERYATKYRKYAVDGHYIAGLYLASDEPSLAGQAAGIAVSYHMLADKK

SEQ ID NO: 97 - DNA, 1704 bp, synthetic construct
  source 1..1704: mol_type = other DNA
  misc_feature 1..1704: note = A nucleic acid sequence encoding a PirAB fusion protein, TIC10381comprised of the TIC10364 and TIC10364 coding sequences inoperable linkage and in frame.
atgagcataatcaatataaatataagtggtagtagtgacattacaatcataaacaataccccatctaacccagaaccactcatttacaatacagacacacccgcatcagaacctcttaccgtcaatccttatagggatatgacaatagagccacactcttctattgaggcaataagaattgatacgccaattattcccgaaacccgccccaattattacgtagccaattctggccccgcatcatcagttagagccgttttttattggtctcactctttcacatcagaatggttcgaatattctgctatcacagtgaaagccggggaagacggcatattacaatcaccgagcaactctgtgtattacagcaaggtcgtcatttataacgaaaccgataaacgcgcctttgttactggatataataagatgaataccacaccgattaatgtatctgaaaatgacacattgcctgtactcactgacgtcatgcttatcgtgccttataccacctctacccccgattatgaatgggatatgtcatcaataataaaggatgccattattggcggcgtagggtttattccaggagtaggttccgcaatgtctttcctgttagggctattttggccgcaacagaaagataatacctgggagcaaatcctccaaaaagtagagcaaatgatagagaatgctgctctacaaacgattaaaggaatacttaatggagatatacaagaaatcaaaggaaaaatggaacatgtgcaatacatgctggaaacctcgcctggcagccaggaaagccatgacgcctatatgttcctggctagatatctggtgagtatcgatgaaagattcaagtcttttgataataaaacaaactaccagatcctgccgatgtacactaacacggttatgttacagatcccttattggaaaatggggatagaaaagaaaaatgatattgggctgaccgatattgaagtcaatgagttaaaacaacttatcgacacattggttgacagagccagaaactatattcatacgatgtatactaatgaatataataatgccataaatacatcaacagcagagagtgtcactaataatttattgtctgtaagagggtattgtttattacacggtttagagtgtattgagttaattgaacatctacaaaataatagcctggaaagtggttttaatcctaaaactatcagttattcaaccgtatttgatcgtcctactaacaaaacgagaattcaggctctgacagaagacgatcaaatgcaggaacccttcaagccctctttaatcgacggtaaatacaataaaataaaatcattgcttggctatgtacgaagaatcggcaatgcccccagagtgggtggtattcaaatcacatttgccaacgattcatcctatacactcggcaccgtaacatcagaaacgagttctattgaactcaatgatagtgttatcgaaaggttggaagtatggggcaatggcgcggttgatgaggcgttatttacgttaagtgatgggcgtcaactcagagtcggtgaacgctacgcgacaaaatacagaaaatacgctgttgatggccactatattgcagggctgtacttagccagcgatgaaccttcacttgctggtcaggccgcaggtattgccgtttcataccatatgttagctgataaaaaataa

SEQ ID NO: 98 - protein, 567 aa, synthetic construct
  REGION 1..567: note = The amino acid sequence of the TIC10381 PirAB fusion protein.
MSIININISGSSDITIINNTPSNPEPLIYNTDTPASEPLTVNPYRDMTIEPHSSIEAIRIDTPIIPETRPNYYVANSGPASSVRAVFYWSHSFTSEWFEYSAITVKAGEDGILQSPSNSVYYSKVVIYNETDKRAFVTGYNKMNTTPINVSENDTLPVLTDVMLIVPYTTSTPDYEWDMSSIIKDAIIGGVGFIPGVGSAMSFLLGLFWPQQKDNTWEQILQKVEQMIENAALQTIKGILNGDIQEIKGKMEHVQYMLETSPGSQESHDAYMFLARYLVSIDERFKSFDNKTNYQILPMYTNTVMLQIPYWKMGIEKKNDIGLTDIEVNELKQLIDTLVDRARNYIHTMYTNEYNNAINTSTAESVTNNLLSVRGYCLLHGLECIELIEHLQNNSLESGFNPKTISYSTVFDRPTNKTRIQALTEDDQMQEPFKPSLIDGKYNKIKSLLGYVRRIGNAPRVGGIQITFANDSSYTLGTVTSETSSIELNDSVIERLEVWGNGAVDEALFTLSDGRQLRVGERYATKYRKYAVDGHYIAGLYLASDEPSLAGQAAGIAVSYHMLADKK

SEQ ID NO: 99 - DNA, 408 bp, Xenorhabdus nematophila
  source 1..408: mol_type = other DNA
  misc_feature 1..408: note = A nucleic acid sequence obtained from Xenorhabdus nematophilaencoding a TIC10359 pesticidal PirA protein sequence.
atgattacaatcaatatcactggtgataatgtaagagttaataacaatatagcaacagaaaccgacctccaaaatacacctgcttcagcacccttatcaattattaattttagggatatgacaatagaacctcattcatctgttgaggcgataagaaccgatacaccgattattcctgaatcacgaccaaattactatgttgctaattctggcccggcctcatcagtcagagctgttttctattggtcccactcttttacatcagaatggtttgaatcttcctctattattgtaaaagcaggcgaagacggagtcttacattcaccgggtaattctttatattacagcaaggttgtaatttataacgatacagacaaacgtgcttttgttaccggctacaatctataa

SEQ ID NO: 100 - protein, 135 aa, Xenorhabdus nematophila
  REGION 1..135: note = The amino acid sequence of the TIC10359 PirA protein.
MITINITGDNVRVNNNIATETDLQNTPASAPLSIINFRDMTIEPHSSVEAIRTDTPIIPESRPNYYVANSGPASSVRAVFYWSHSFTSEWFESSSIIVKAGEDGVLHSPGNSLYYSKVVIYNDTDKRAFVTGYNL

SEQ ID NO: 101 - DNA, 1290 bp, Xenorhabdus nematophila
  source 1..1290: mol_type = other DNA
  misc_feature 1..1290: note = A nucleic acid sequence obtained from Xenorhabdus nematophilaencoding a TIC10368 pesticidal PirB protein sequence.
atgaataatgaaccgatgaatactaatgaatcacaagcttcagagatagtaccctcaatgaatgaatctatattagcagcaccttattcaatttctacacctaattatgaatgggatatgtcatcaataataaaagatgccattattggtggtataggctttattcctggtccgggctcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcacctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagataaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttaattaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctgttgatgaggcattctttacattaagtgacggacgtcaatttaggcttggccaacgctatgccagtaactatagaaaatatgctgtcgataaccactatatttcaggattgtacttagccagtgatgaaccttcattggcaggccaagcagcaggcattgcagtttcataccatatgatagctgataaaaaatcatag

SEQ ID NO: 102 - protein, 429 aa, Xenorhabdus nematophila
  REGION 1..429: note = The amino acid sequence of the TIC10368 PirB protein.
MNNEPMNTNESQASEIVPSMNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSPGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALINGKYNKIKSLIGYVQRIGNAPRVGGIKVTFANDASYTLGTVTSEVNSIELNDSVITSLEVWGNGAVDEAFFTLSDGRQFRLGQRYASNYRKYAVDNHYISGLYLASDEPSLAGQAAGIAVSYHMIADKKS

SEQ ID NO: 103 - DNA, 1766 bp, synthetic construct
  source 1..1766: mol_type = other DNA
  misc_feature 1..1766: note = A nucleic acid sequence encoding an operon comprised of thecoding sequences TIC10359 and TIC10368.
atgattacaatcaatatcactggtgataatgtaagagttaataacaatatagcaacagaaaccgacctccaaaatacacctgcttcagcacccttatcaattattaattttagggatatgacaatagaacctcattcatctgttgaggcgataagaaccgatacaccgattattcctgaatcacgaccaaattactatgttgctaattctggcccggcctcatcagtcagagctgttttctattggtcccactcttttacatcagaatggtttgaatcttcctctattattgtaaaagcaggcgaagacggagtcttacattcaccgggtaattctttatattacagcaaggttgtaatttataacgatacagacaaacgtgcttttgttaccggctacaatctataatgacgcagaaatacaatccatatttccaatgaatttcaaataacatccttaaggcaagaaacaaaatcatgaataatgaaccgatgaatactaatgaatcacaagcttcagagatagtaccctcaatgaatgaatctatattagcagcaccttattcaatttctacacctaattatgaatgggatatgtcatcaataataaaagatgccattattggtggtataggctttattcctggtccgggctcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcacctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagataaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttaattaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctgttgatgaggcattctttacattaagtgacggacgtcaatttaggcttggccaacgctatgccagtaactatagaaaatatgctgtcgataaccactatatttcaggattgtacttagccagtgatgaaccttcattggcaggccaagcagcaggcattgcagtttcataccatatgatagctgataaaaaatcatag

SEQ ID NO: 104 - DNA, 417 bp, Photorhabdus luminescens
  source 1..417: mol_type = other DNA
  misc_feature 1..417: note = A nucleic acid sequence obtained from Photorhabdus luminescensstrain Hm encoding a PirA_ABE68878 pesticidal PirA proteinsequence.
atgaggaaaataaatatgtctagaataaccattgttgttgattcagatacacaaaaagcagaagtttattctaattctcctgtgccggtacatagagatttaaatgcagttggtcctttgagtgatgtgactatatcacctcatgctagtgtggaagtatttagaatagacaccccaataattccagaatccagaagctctctgagagttgtaaatacagggttagcaaatagtgttacggctaaattttactggtctcatagttttacctctgaatggtttgaagctggatctatagatgtaggattaggagaagataaggtattaaacgtgcctagcagctctttttattatagtaaatttgttatctataataacacggatagagtggcttatgttacggcaaatttggtttaa

SEQ ID NO: 105 - protein, 138 aa, Photorhabdus luminescens
  REGION 1..138: note = The amino acid sequence of the PirA_ABE68878 PirA protein.
MRKINMSRITIVVDSDTQKAEVYSNSPVPVHRDLNAVGPLSDVTISPHASVEVFRIDTPIIPESRSSLRVVNTGLANSVTAKFYWSHSFTSEWFEAGSIDVGLGEDKVLNVPSSSFYYSKFVIYNNTDRVAYVTANLV

SEQ ID NO: 106 - DNA, 1260 bp, Photorhabdus luminescens
  source 1..1260: mol_type = other DNA
  misc_feature 1..1260: note = A nucleic acid sequence obtained from Photorhabdus luminescensstrain Hm encoding a PirB_ABE68879 pesticidal PirB proteinsequence.
atgcatacagaaaatgttttagacatcagaaccattgtggctaatgaatatgtggtaaaaacgagtgcattagagtgggatgttacggatattgtaaaaaatgcaatcatagggggtatctcttttataccttcggttggtcctgcgatatcttttttggtcggtttattctggcctcaatcgaaagaaaatatatgggaaggaattgtcaaacaaattgagaggatgatagaggagtctgcgttaaagacgattaaaggtatccttgcgggtgatattgcctatatacaagagcgcatggcaaccgttgctgatcttcttgataagcatccaggatctgacgaagcgaggagcgcctttaataacctggcagaaaatatagatggttatcacaaaaaatttaataatttttcggatgatgttaactatcaaatattacccatgttttctactacggttatgatgcagataacctattgggttgctggtttagagagaagagctgaaattgggcttagtgatattgatattgaaaaagtccgaggattaatcaaaaagacggtagaacaagctaatagttatattaatagtatctatgatagagagcttaatgatgctcttaataactcgacggcggacactgttgcaaataatgttatgtctgttcatggtcactgtcgtttacatgggattgaatatatcagtatttgggatagattaagtgaatctgagtctgtaaataatagaatctatgttgatgttttaagttattctactttctttgatcgtcaaacagcaaaagccagaattcaggcattgactccagagcaagatatggctccgcctctcaaaccagctcttaatggagggaagagaagaaagatagattctttaatgggacatattgtacgtattggaggagctccgagagtaggagggctgacagttgtatttgatgacggcagtagccatcgattaggtacaatatctggtgagacggcatctatttctctgaatggtagtcgaattaccagtttggaagtatggggcaatggtgctgttgatagagccgtctttactttgagtgatggtcggtttttgttatttggcgatcctggaacatctcgatataggaaattttatgttggtgatagtcactatatttcagggatatatttgtccagtgattacaacccattagcaggtcaggcagcaaatattgctgtatcttatcagttgataaatgatgatgaaaaatag

SEQ ID NO: 107 - protein, 419 aa, Photorhabdus luminescens
  REGION 1..419: note = The amino acid sequence of the PirB_ABE68879 PirB protein.
MHTENVLDIRTIVANEYVVKTSALEWDVTDIVKNAIIGGISFIPSVGPAISFLVGLFWPQSKENIWEGIVKQIERMIEESALKTIKGILAGDIAYIQERMATVADLLDKHPGSDEARSAFNNLAENIDGYHKKFNNFSDDVNYQILPMFSTTVMMQITYWVAGLERRAEIGLSDIDIEKVRGLIKKTVEQANSYINSIYDRELNDALNNSTADTVANNVMSVHGHCRLHGIEYISIWDRLSESESVNNRIYVDVLSYSTFFDRQTAKARIQALTPEQDMAPPLKPALNGGKRRKIDSLMGHIVRIGGAPRVGGLTVVFDDGSSHRLGTISGETASISLNGSRITSLEVWGNGAVDRAVFTLSDGRFLLFGDPGTSRYRKFYVGDSHYISGIYLSSDYNPLAGQAANIAVSYQLINDDEK

SEQ ID NO: 108 - DNA, 1674 bp, synthetic construct
  source 1..1674: mol_type = other DNA
  misc_feature 1..1674: note = A nucleic acid sequence encoding a PirAB fusion protein, TIC10434comprised of the PirA_ABE68878 and PirB_ABE68879 coding sequencesin operable linkage and in frame.
atgaggaaaataaatatgtctagaataaccattgttgttgattcagatacacaaaaagcagaagtttattctaattctcctgtgccggtacatagagatttaaatgcagttggtcctttgagtgatgtgactatatcacctcatgctagtgtggaagtatttagaatagacaccccaataattccagaatccagaagctctctgagagttgtaaatacagggttagcaaatagtgttacggctaaattttactggtctcatagttttacctctgaatggtttgaagctggatctatagatgtaggattaggagaagataaggtattaaacgtgcctagcagctctttttattatagtaaatttgttatctataataacacggatagagtggcttatgttacggcaaatttggttatgcatacagaaaatgttttagacatcagaaccattgtggctaatgaatatgtggtaaaaacgagtgcattagagtgggatgttacggatattgtaaaaaatgcaatcatagggggtatctcttttataccttcggttggtcctgcgatatcttttttggtcggtttattctggcctcaatcgaaagaaaatatatgggaaggaattgtcaaacaaattgagaggatgatagaggagtctgcgttaaagacgattaaaggtatccttgcgggtgatattgcctatatacaagagcgcatggcaaccgttgctgatcttcttgataagcatccaggatctgacgaagcgaggagcgcctttaataacctggcagaaaatatagatggttatcacaaaaaatttaataatttttcggatgatgttaactatcaaatattacccatgttttctactacggttatgatgcagataacctattgggttgctggtttagagagaagagctgaaattgggcttagtgatattgatattgaaaaagtccgaggattaatcaaaaagacggtagaacaagctaatagttatattaatagtatctatgatagagagcttaatgatgctcttaataactcgacggcggacactgttgcaaataatgttatgtctgttcatggtcactgtcgtttacatgggattgaatatatcagtatttgggatagattaagtgaatctgagtctgtaaataatagaatctatgttgatgttttaagttattctactttctttgatcgtcaaacagcaaaagccagaattcaggcattgactccagagcaagatatggctccgcctctcaaaccagctcttaatggagggaagagaagaaagatagattctttaatgggacatattgtacgtattggaggagctccgagagtaggagggctgacagttgtatttgatgacggcagtagccatcgattaggtacaatatctggtgagacggcatctatttctctgaatggtagtcgaattaccagtttggaagtatggggcaatggtgctgttgatagagccgtctttactttgagtgatggtcggtttttgttatttggcgatcctggaacatctcgatataggaaattttatgttggtgatagtcactatatttcagggatatatttgtccagtgattacaacccattagcaggtcaggcagcaaatattgctgtatcttatcagttgataaatgatgatgaaaaatag

SEQ ID NO: 109 - protein, 557 aa, synthetic construct
  REGION 1..557: note = The amino acid sequence of the TIC10434 PirAB fusion protein.
MRKINMSRITIVVDSDTQKAEVYSNSPVPVHRDLNAVGPLSDVTISPHASVEVFRIDTPIIPESRSSLRVVNTGLANSVTAKFYWSHSFTSEWFEAGSIDVGLGEDKVLNVPSSSFYYSKFVIYNNTDRVAYVTANLVMHTENVLDIRTIVANEYVVKTSALEWDVTDIVKNAIIGGISFIPSVGPAISFLVGLFWPQSKENIWEGIVKQIERMIEESALKTIKGILAGDIAYIQERMATVADLLDKHPGSDEARSAFNNLAENIDGYHKKFNNFSDDVNYQILPMFSTTVMMQITYWVAGLERRAEIGLSDIDIEKVRGLIKKTVEQANSYINSIYDRELNDALNNSTADTVANNVMSVHGHCRLHGIEYISIWDRLSESESVNNRIYVDVLSYSTFFDRQTAKARIQALTPEQDMAPPLKPALNGGKRRKIDSLMGHIVRIGGAPRVGGLTVVFDDGSSHRLGTISGETASISLNGSRITSLEVWGNGAVDRAVFTLSDGRFLLFGDPGTSRYRKFYVGDSHYISGIYLSSDYNPLAGQAANIAVSYQLINDDEK

SEQ ID NO: 110 - DNA, 1668 bp, synthetic construct
  source 1..1668: mol_type = other DNA
  misc_feature 1..1668: note = A nucleic acid sequence encoding a PirAB fusion protein, TIC11210comprised of the TIC7575 and TIC7665 coding sequences in operablelinkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgaataatagtccaatgaatgatcagttatcaatagcaccttattcaatttcgacacccaattatgaatgggatatgtcatcaatcataaaagatgccattatcggtggcataggatttattcccggaccaggctctgcaatctcttttttattagggctgttctggcctcaacagacagacaatacctgggatcaaatcctccaaaaaatcgaacagatgatagaagaagcgaatttaaaaaccattaaaggtatattaaatggagatatacaagaaattaaaggaaaaatggaccatgtgcaatatatgctagagaattctcctggcagccaggaaagccatgatgcttatatgtttttagcaaggtttttggtcagtattgatgaaaaattcaaatctttcgatgatagaacaaattatcaaattcttcccatgtatacgaacaccattatgttacaagcgccttattggaaaatgggcctcgaaaagaaagaggatatcggtttaagcgatattgaagttagcgaattaaaagaacttatcgataaattatatactaaatcatatgattatatccataacacgtataatcgtgaatatgataatgcaatcaatacgtcaaccgcagagagtatcaccaataatttattgtctgtcagaggatattgtttattacatggttgtgaatgtcttgaagttattgcgcatatacaaaacaatagccttgataaaggcttctaccctaaaacgatcagctattcgagtgttttcgatcgtcctacaaacaaaatgaggattcaggcgcttacagaagatgaccaaatgcaagaaccgttcaaaccttctttcgtcaatggtcaatataataaaataaaatcattggagggttatgtcacaaggatcggcaatgccccccgagtcggtggaattaaaatcacatttgaaaacaacgcatcttatactcttggtactgtgacttcagaaacaacctttattgaactcaatgagagtgttataaccagcatagaagtgtggggaaatggggccgttgatgaggcattctttacattgagtgacggtcgccaaatgcggcttggtcaacgctatgccagtcgctacagaaaatatgctgtcgatggtcattatatctcaggattgtacttagccagtgatgaaccatcccttgctggtcaagccgccggtattgccgtttcatatcatatgattgttgataaacaatag

SEQ ID NO: 111 - protein, 555 aa, synthetic construct
  REGION 1..555: note = The amino acid sequence of the TIC11210 PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMNNSPMNDQLSIAPYSISTPNYEWDMSSIIKDAIIGGIGFIPGPGSAISFLLGLFWPQQTDNTWDQILQKIEQMIEEANLKTIKGILNGDIQEIKGKMDHVQYMLENSPGSQESHDAYMFLARFLVSIDEKFKSFDDRTNYQILPMYTNTIMLQAPYWKMGLEKKEDIGLSDIEVSELKELIDKLYTKSYDYIHNTYNREYDNAINTSTAESITNNLLSVRGYCLLHGCECLEVIAHIQNNSLDKGFYPKTISYSSVFDRPTNKMRIQALTEDDQMQEPFKPSFVNGQYNKIKSLEGYVTRIGNAPRVGGIKITFENNASYTLGTVTSETTFIELNESVITSIEVWGNGAVDEAFFTLSDGRQMRLGQRYASRYRKYAVDGHYISGLYLASDEPSLAGQAAGIAVSYHMIVDKQ

SEQ ID NO: 112 - DNA, 1683 bp, synthetic construct
  source 1..1683: mol_type = other DNA
  misc_feature 1..1683: note = A nucleic acid sequence encoding a PirAB fusion protein, TIC11211comprised of the TIC7575 and TIC7667 coding sequences in operablelinkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgcatacagaaaatgttttagacataagaaccattgtggctaatgaatatgctgtaaaaacgagtgcattagagtgggatgttactgatattgtaaaaaatgcaatcatagggggaatatcctttatcccttcggttggtcccgctatatcttttttagtcggtttattctggcctcaatcgaaagaaaatatatgggaagggattgtcaaacaaattgaaaggatgatagaggagtctgcgttaaagacgattaaaggtatccttgctggtgatattgcatatatacaagaacgaatggcaaccgttgctgatcttcttgataagcatccaggatcagaagaagcgaggagtgcttttaataacctggcagaaaatatagatggctatcacaaaaagtttagtaatttttcggatgatgttaattatcagatattacccatgttttctactacggttatgatgcagataacatattgggttgctggtttagagagaaaagatgaaattgggcttagtaatattgatgttgaaaaagtccgaggattaattaaaaagacggtagaacaggctaatagttatattaacaatatatatgatagagagcttaatgatgctcttaataactcgacggctgacactgttgcaaataatgttatgtctgttcatggtcactgtcgtttacatgggattgaatatatcagtatttgggataaattaagtgaagctgagtcggtaaataataaaatctatgttgatgttttaagttattctactttctttgaccgtcaaacagcaaaagccagaattcaggcattgactccagagaaagatatgactccacctctcaaaccggctcttaatggaggaaaaagaagaaagatagattcgttaacgggacatattgtgcgtattggaggggctgcgagggtaggagggctgacagttgtatttgatgatggtaatcgccatcaattaggtacaatatctggtgagacgtcatctatttctctgaatggtagtcgaattaccagtttggaagtatggggaaatggtgctgttgatcaagcggtctttactttaaatgatggtcgttcattgtcattgggctcgcctggaacatctcgatataggaagttttatgttggtgaaagccactatattgcagggatatatttgtccagtgattacaacccattagctggtcaggcagcaaatattgctgtatcttatcagttgataaatgatgatgaaaaatag

SEQ ID NO: 113 - protein, 560 aa, synthetic construct
  REGION 1..560: note = The amino acid sequence of the TIC11211 PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMHTENVLDIRTIVANEYAVKTSALEWDVTDIVKNAIIGGISFIPSVGPAISFLVGLFWPQSKENIWEGIVKQIERMIEESALKTIKGILAGDIAYIQERMATVADLLDKHPGSEEARSAFNNLAENIDGYHKKFSNFSDDVNYQILPMFSTTVMMQITYWVAGLERKDEIGLSNIDVEKVRGLIKKTVEQANSYINNIYDRELNDALNNSTADTVANNVMSVHGHCRLHGIEYISIWDKLSEAESVNNKIYVDVLSYSTFFDRQTAKARIQALTPEKDMTPPLKPALNGGKRRKIDSLTGHIVRIGGAARVGGLTVVFDDGNRHQLGTISGETSSISLNGSRITSLEVWGNGAVDQAVFTLNDGRSLSLGSPGTSRYRKFYVGESHYIAGIYLSSDYNPLAGQAANIAVSYQLINDDEK

SEQ ID NO: 114 - DNA, 1668 bp, synthetic construct
  source 1..1668: mol_type = other DNA
  misc_feature 1..1668: note = A nucleic acid sequence encoding a PirAB fusion protein, TIC11212comprised of the TIC7662 and TIC7665 coding sequences in operablelinkage and in frame.
atgagtacaatcaatatcaatataagtagcagtaccgttaccgtcatcacgaataacggagaaacgccagtcccactcacttacaatacaaatacacctgaatcagaacctcttaccgtcaatccttatagggatatgacaatagagccacgctcttctattgaagcaacaaggattgatacaccgattattcccgaaacacgccctaattattatgtagccaattcaggcccggcttcatcagttagggccgttttttattggtcccattctttcacatcacaatggttcgaatattcctctatcatcgtcaaagccggggaagatggcatattagaatcaccaagcaattctttatattacagcaaagtcgtcatttataatgataccgataaacgcgcctttgtgacgggatataataagatgaataatagtccaatgaatgatcagttatcaatagcaccttattcaatttcgacacccaattatgaatgggatatgtcatcaatcataaaagatgccattatcggtggcataggatttattcccggaccaggctctgcaatctcttttttattagggctgttctggcctcaacagacagacaatacctgggatcaaatcctccaaaaaatcgaacagatgatagaagaagcgaatttaaaaaccattaaaggtatattaaatggagatatacaagaaattaaaggaaaaatggaccatgtgcaatatatgctagagaattctcctggcagccaggaaagccatgatgcttatatgtttttagcaaggtttttggtcagtattgatgaaaaattcaaatctttcgatgatagaacaaattatcaaattcttcccatgtatacgaacaccattatgttacaagcgccttattggaaaatgggcctcgaaaagaaagaggatatcggtttaagcgatattgaagttagcgaattaaaagaacttatcgataaattatatactaaatcatatgattatatccataacacgtataatcgtgaatatgataatgcaatcaatacgtcaaccgcagagagtatcaccaataatttattgtctgtcagaggatattgtttattacatggttgtgaatgtcttgaagttattgcgcatatacaaaacaatagccttgataaaggcttctaccctaaaacgatcagctattcgagtgttttcgatcgtcctacaaacaaaatgaggattcaggcgcttacagaagatgaccaaatgcaagaaccgttcaaaccttctttcgtcaatggtcaatataataaaataaaatcattggagggttatgtcacaaggatcggcaatgccccccgagtcggtggaattaaaatcacatttgaaaacaacgcatcttatactcttggtactgtgacttcagaaacaacctttattgaactcaatgagagtgttataaccagcatagaagtgtggggaaatggggccgttgatgaggcattctttacattgagtgacggtcgccaaatgcggcttggtcaacgctatgccagtcgctacagaaaatatgctgtcgatggtcattatatctcaggattgtacttagccagtgatgaaccatcccttgctggtcaagccgccggtattgccgtttcatatcatatgattgttgataaacaatag

SEQ ID NO: 115 - protein, 555 aa, synthetic construct
  REGION 1..555: note = The amino acid sequence of the TIC11212 PirAB fusion protein.
MSTININISSSTVTVITNNGETPVPLTYNTNTPESEPLTVNPYRDMTIEPRSSIEATRIDTPIIPETRPNYYVANSGPASSVRAVFYWSHSFTSQWFEYSSIIVKAGEDGILESPSNSLYYSKVVIYNDTDKRAFVTGYNKMNNSPMNDQLSIAPYSISTPNYEWDMSSIIKDAIIGGIGFIPGPGSAISFLLGLFWPQQTDNTWDQILQKIEQMIEEANLKTIKGILNGDIQEIKGKMDHVQYMLENSPGSQESHDAYMFLARFLVSIDEKFKSFDDRTNYQILPMYTNTIMLQAPYWKMGLEKKEDIGLSDIEVSELKELIDKLYTKSYDYIHNTYNREYDNAINTSTAESITNNLLSVRGYCLLHGCECLEVIAHIQNNSLDKGFYPKTISYSSVFDRPTNKMRIQALTEDDQMQEPFKPSFVNGQYNKIKSLEGYVTRIGNAPRVGGIKITFENNASYTLGTVTSETTFIELNESVITSIEVWGNGAVDEAFFTLSDGRQMRLGQRYASRYRKYAVDGHYISGLYLASDEPSLAGQAAGIAVSYHMIVDKQ

SEQ ID NO: 116 - DNA, 1701 bp, synthetic construct
  source 1..1701: mol_type = other DNA
  misc_feature 1..1701: note = A nucleic acid sequence encoding a PirAB fusion protein, TIC11301comprised of the TIC7575 and TIC7661 coding sequences in operablelinkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgaatactacacctattactgtatctacaaatgaaacatcgcctttaatgactgacgtaatgcccatggatctttatgcaatatccacacctgattatgaatgggacatgtcgtcaatcataaaggatgctgttattggtggcataggatttattccaggtccgggcccggcaatatccttcctgttagggctattttggcctcagcagaaagacaatacttgggagcaaattctccagaaagtagagcagatgatagagaatgctgttctacaaactattaaaggaatacttaatggagaagttcaagagatcaaagggaaaatggaacatgtagaatccatgctgaaaaactcgcctggcagtcaggaaagtcatgatgcatatatgttcctggcgagatatctggttagtatagatgaaaaattcaaatcttttgacaatagaacaaattaccagcttctcccaatgtatactaacactattatgttacagatcccttattggaaaatgggaatagagaagaaaaaagatattgggctgacagatattgaagttaatgaattaaaagaacttatcgataaattggttgataaggccaaaaactatattcatacgatgtatactaatgaacataataatgctgtaaacacatcaacagcagagagtgtcactaataatttattatctgtaagaggatattgtttattacacggtttagaatgtattgagttaatcgagcatatacagaataatagccttgagagtggtttctatcctaaaattatcagttattcgactgcgtttgatcgtcctactaacaaaatgagaattcaggctcttacagaagatgatgcaatgcaggagcctttcaaaccatctttaatcaatgggaaatataataaaatccaatccttgactggatatgtacaaagaattgggaatgcacctagagttggtggtatcagaatcacatttaccaacggctcatcttatacacttggtacagtgacctcagaaacgcattcaattaagctaaacgatagtgttatcgaaagcttggaagtatgggggaatggtgctgttgatgaggcgttatttaagttaagtgatgggcgtttattgcgtattggtgagcgctacgcgaaaaaatacagaaaatatgctgttgataatcactatattgcggggatttacttagccagcgatgagccttcacttgctggtcaagccgcaggtattgccgtttcatatcatatgatggctgacaaaaaataa

SEQ ID NO: 117 - protein, 566 aa, synthetic construct
  REGION 1..566: note = The amino acid sequence of the TIC11301 PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMNTTPITVSTNETSPLMTDVMPMDLYAISTPDYEWDMSSIIKDAVIGGIGFIPGPGPAISFLLGLFWPQQKDNTWEQILQKVEQMIENAVLQTIKGILNGEVQEIKGKMEHVESMLKNSPGSQESHDAYMFLARYLVSIDEKFKSFDNRTNYQLLPMYTNTIMLQIPYWKMGIEKKKDIGLTDIEVNELKELIDKLVDKAKNYIHTMYTNEHNNAVNTSTAESVTNNLLSVRGYCLLHGLECIELIEHIQNNSLESGFYPKIISYSTAFDRPTNKMRIQALTEDDAMQEPFKPSLINGKYNKIQSLTGYVQRIGNAPRVGGIRITFTNGSSYTLGTVTSETHSIKLNDSVIESLEVWGNGAVDEALFKLSDGRLLRIGERYAKKYRKYAVDNHYIAGIYLASDEPSLAGQAAGIAVSYHMMADKK

SEQ ID NO: 118 - DNA, 1701 bp, synthetic construct
  source 1..1701: mol_type = other DNA
  misc_feature 1..1701: note = A nucleic acid sequence encoding a f PirAB usion protein,TIC11302 comprised of the TIC7660 and TIC7576 coding sequences inoperable linkage and in frame.
atgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaaatgaatatctcaccgattaatgtatctgaaaatgaaacattacctgaactcactgatgttatgcttattgtgccttatacaacatctacccctgattatgaatgggatatgtcatcaattataaaggatgcgattattggcggcgtagggtttattccaggagcaggctctgcaatgtccttcctattgggactattttggcctcaacagaaagataatacatgggaacagatcctccaaaaagtagaacagatgatagagaatgccgttctgcaaactattaaaggaatacttaatggagatatacaagaaatcaaggggaaaatggaacatgtgcaatacatgctggaaacctcgcctggcagtcaggaaagtcatgacgcatatatgttcctggctagatacctggtgagtatagatgaaaaattcaagtcttttgataataaaacaaactaccagatcctgccgatgtacactaacacggttatgttacaaatcccttattggaaaatgggaatagagaagaaaaatgatattgggctgacagatattgaagtcaatgagttaaaacagcttatcgataaattggtcgacaaggccaagagttacatccatacgatgtatacgaatgaatataatgatgccataaatacatcaacagcatcgagtgtcactaataatttactctctgtaagaggatattgtttattacacggtttagagtgtattgagttaattgaacatctacaaaacaatagcctcgaaagtggtttttatcctaaaactatcagttattcaactgtatttgatcgtcagactaacaaaatgagaattcaggctcttacagaagacgatcaaatgcaggaaccctttaagccatctttaatcaacggcaaatacaataaaatacaatccttgcttggatatgtacaaagaattggaaatgcacctagagtggggggtattaaaatcacctttgccaacggttcatcctatacacttggcacagtaacatcagaaacgagttcaattgaactcaatgatagtgttatcgaaagattggaagtatggggcaatggcgctgttgatgaggcattatttacgttaagtgatgggcgtcaactcagagtcggtgagcgctacgcgacaaaatatagaaaatatgctgttgatggacactatattgcaggactgtacttagctagcgatgaaccttcacttgctggtcaagccgcaggtattgccgtttcataccatatgttggatgataaaaaataa

SEQ ID NO: 119 - protein, 566 aa, synthetic construct
  REGION 1..566: note = The amino acid sequence of the TIC11302 f PirAB fusion protein.
MITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNKMNISPINVSENETLPELTDVMLIVPYTTSTPDYEWDMSSIIKDAIIGGVGFIPGAGSAMSFLLGLFWPQQKDNTWEQILQKVEQMIENAVLQTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDKLVDKAKSYIHTMYTNEYNDAINTSTASSVTNNLLSVRGYCLLHGLECIELIEHLQNNSLESGFYPKTISYSTVFDRQTNKMRIQALTEDDQMQEPFKPSLINGKYNKIQSLLGYVQRIGNAPRVGGIKITFANGSSYTLGTVTSETSSIELNDSVIERLEVWGNGAVDEALFTLSDGRQLRVGERYATKYRKYAVDGHYIAGLYLASDEPSLAGQAAGIAVSYHMLDDKK

SEQ ID NO: 120 - DNA, 2097 bp, synthetic construct
  source 1..2097: mol_type = other DNA
  misc_feature 1..2097: note = A nucleic acid sequence encoding a PirAB fusion protein, TIC11440comprised of the TIC4771, TIC4771, and TIC4772 coding sequencesin operable linkage and in frame.
atgattacaataaatataagtggtggtagtataaaaattagtaacaacataggatcagaaactgatatcaaaaatacacctttttcagaacctctttcaattagtaattataaggatatgacaatagagccacattcgtctatccaagcaacaagaactgatacaccaattattcctgaaacacgaccaaattattatgtagctaattccggccctgccgcatcagtgagagctgttttttattggtctcattcttttacatcagaatggttcgaacattcatctatcattgtaaaagcaggagaagatggaatattgaactcacctagcaattctgtatattacagtaaggttgtcatttacaacgatacggataaacgggcctttgtcacaggttatgacaaaatgataaccattaacatctcaggaggtagcataaagattagtaataacattggctcggaaactgatatcaagaacacgccgttttctgaaccactttcaattagtaattataaagatatgactatagaaccacactcgtctattcaggctacaagaacagatacaccaattatacctgaaacacgacctaattactatgtagccaattccggacctgcggcatcagtaagagctgtcttttactggtcacattcttttacgtcagagtggtttgaacattcttcaatcatagtaaaagcaggagaggatggaattcttaactctcctagcaactctgtttattacagtaaagttgtgatatataatgataccgataagagagctttcgtgactggatacgataagatgaataacgaacttatgaacacaaatgaatcacaaccttcagagacattatctttaattaatgaatctatattaacagcaccttatgccgtttctacccctaattatgaatgggatatgtcatcaataataaaagatgccattattggaggtataggatttattcccgggccgggttcagcaatatcgtttttgctagggctattttggccgcaacaaacagacaatacctgggagcaaattctccaaaaagtagaacagatgatagaggaagcgaatttaaaaactattcaaggaatactgaacggagatatacaagaaataaaaggaaagatggaacatgtggaatatatgctagaaacctcaccaggcactcaagaaagccatgacgcatatatgttcttagcgagatatctggtaagtatagatgaaaaattcaaatcttttgataataaaacaaattatcaaattcttccaatgtacaccaatacgcttatgttacaggcaccttactggaaaatgggtatagagaagaaaaatgatattttgctaacagatatagaagttaatgaattaaaacagcttatcgaaaatctatatgccaaggccaatagctatattcatgaagtgtatacccgtgaatacgataatgcggtaaatacctcaacagcaacaacgattaccaataatttattgtctgtaagagggtattgtttattacatggattagagtgccttgaagtccttgatcatatacaaaataataatcttgatcagagcttctatccgaaaactatcagttattctactgtatttgatcgctcaacaaacaaaacaagactccaggctcttaccgaagacgagcaaatggaagaaccactcaaaccctcttttattaatggggaatataataaaataaaatcactgattggatatgtacagagaattggaaacgcccctagagttggaggtataaaaattacatttactaatggatcatctcatactctgggtacagtgacctcagaatcaaactcaattgaactaaatgatagtgttataaccagtgtggaagtatggggaaatggtgctgttgatgaggcattctttacattaagtgacggtcgtcaatttaggcttggtcaacgctatgccagtaactacagaaaatatgctgttgatggccactatatttcaggattgtacttagccagtgatgagccttcacttgctggtcaagccgcaggtattgcagtttcatatcatatattggttgataagaaataa

SEQ ID NO: 121 - protein, 698 aa, synthetic construct
  REGION 1..698: note = The amino acid sequence of the TIC11440 PirAB fusion protein.
MITINISGGSIKISNNIGSETDIKNTPFSEPLSISNYKDMTIEPHSSIQATRTDTPIIPETRPNYYVANSGPAASVRAVFYWSHSFTSEWFEHSSIIVKAGEDGILNSPSNSVYYSKVVIYNDTDKRAFVTGYDKMITINISGGSIKISNNIGSETDIKNTPFSEPLSISNYKDMTIEPHSSIQATRTDTPIIPETRPNYYVANSGPAASVRAVFYWSHSFTSEWFEHSSIIVKAGEDGILNSPSNSVYYSKVVIYNDTDKRAFVTGYDKMNNELMNTNESQPSETLSLINESILTAPYAVSTPNYEWDMSSIIKDAIIGGIGFIPGPGSAISFLLGLFWPQQTDNTWEQILQKVEQMIEEANLKTIQGILNGDIQEIKGKMEHVEYMLETSPGTQESHDAYMFLARYLVSIDEKFKSFDNKTNYQILPMYTNTLMLQAPYWKMGIEKKNDILLTDIEVNELKQLIENLYAKANSYIHEVYTREYDNAVNTSTATTITNNLLSVRGYCLLHGLECLEVLDHIQNNNLDQSFYPKTISYSTVFDRSTNKTRLQALTEDEQMEEPLKPSFINGEYNKIKSLIGYVQRIGNAPRVGGIKITFTNGSSHTLGTVTSESNSIELNDSVITSVEVWGNGAVDEAFFTLSDGRQFRLGQRYASNYRKYAVDGHYISGLYLASDEPSLAGQAAGIAVSYHILVDKK

SEQ ID NO: 122 - DNA, 2124 bp, synthetic construct
  source 1..2124: mol_type = other DNA
  misc_feature 1..2124: note = A nucleic acid sequence encoding a PirAB fusion protein, TIC11441comprised of the TIC7575, TIC7575, and TIC7576 coding sequencesin operable linkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgaacactataaacatcaacatttccggaagcacggtaacggtgatttctaacaatgattcaaaccctgagccattaacatataacactaatacgcctgcgagtgatccactaacagccagtccgtatagagatatgactatagagcctcattcttctattgaggcaacgagaacagatacaccaattattccagaaactcgtccgaattactatgtcgccaattctggtccagcatcatcagttagggctgtattctattggtctcattcattcacatcagagtggttcgaatattcatctatcatagtgaaagcaggcaaggacggtatactacaaagcccaaataacgcattatattactcgaaggttgtcatttacaatgatacggataagagagctttcgttactggatataacaaaatgaatatctcaccgattaatgtatctgaaaatgaaacattacctgaactcactgatgttatgcttattgtgccttatacaacatctacccctgattatgaatgggatatgtcatcaattataaaggatgcgattattggcggcgtagggtttattccaggagcaggctctgcaatgtccttcctattgggactattttggcctcaacagaaagataatacatgggaacagatcctccaaaaagtagaacagatgatagagaatgccgttctgcaaactattaaaggaatacttaatggagatatacaagaaatcaaggggaaaatggaacatgtgcaatacatgctggaaacctcgcctggcagtcaggaaagtcatgacgcatatatgttcctggctagatacctggtgagtatagatgaaaaattcaagtcttttgataataaaacaaactaccagatcctgccgatgtacactaacacggttatgttacaaatcccttattggaaaatgggaatagagaagaaaaatgatattgggctgacagatattgaagtcaatgagttaaaacagcttatcgataaattggtcgacaaggccaagagttacatccatacgatgtatacgaatgaatataatgatgccataaatacatcaacagcatcgagtgtcactaataatttactctctgtaagaggatattgtttattacacggtttagagtgtattgagttaattgaacatctacaaaacaatagcctcgaaagtggtttttatcctaaaactatcagttattcaactgtatttgatcgtcagactaacaaaatgagaattcaggctcttacagaagacgatcaaatgcaggaaccctttaagccatctttaatcaacggcaaatacaataaaatacaatccttgcttggatatgtacaaagaattggaaatgcacctagagtggggggtattaaaatcacctttgccaacggttcatcctatacacttggcacagtaacatcagaaacgagttcaattgaactcaatgatagtgttatcgaaagattggaagtatggggcaatggcgctgttgatgaggcattatttacgttaagtgatgggcgtcaactcagagtcggtgagcgctacgcgacaaaatatagaaaatatgctgttgatggacactatattgcaggactgtacttagctagcgatgaaccttcacttgctggtcaagccgcaggtattgccgtttcataccatatgttggatgataaaaaataa

SEQ ID NO: 123 - protein, 707 aa, synthetic construct
  REGION 1..707: note = The amino acid sequence of the TIC11441 f PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMNISPINVSENETLPELTDVMLIVPYTTSTPDYEWDMSSIIKDAIIGGVGFIPGAGSAMSFLLGLFWPQQKDNTWEQILQKVEQMIENAVLQTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDKLVDKAKSYIHTMYTNEYNDAINTSTASSVTNNLLSVRGYCLLHGLECIELIEHLQNNSLESGFYPKTISYSTVFDRQTNKMRIQALTEDDQMQEPFKPSLINGKYNKIQSLLGYVQRIGNAPRVGGIKITFANGSSYTLGTVTSETSSIELNDSVIERLEVWGNGAVDEALFTLSDGRQLRVGERYATKYRKYAVDGHYIAGLYLASDEPSLAGQAAGIAVSYHMLDDKK

SEQ ID NO: 124 - DNA, 2115 bp, synthetic construct
  source 1..2115: mol_type = other DNA
  misc_feature 1..2115: note = A nucleic acid sequence encoding a PirAB fusion protein, TIC11442comprised of the TIC7575, TIC4771, and TIC4472 coding sequencesin operable linkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgattacaataaatataagtggtggtagtataaaaattagtaacaacataggatcagaaactgatatcaaaaatacacctttttcagaacctctttcaattagtaattataaggatatgacaatagagccacattcgtctatccaagcaacaagaactgatacaccaattattcctgaaacacgaccaaattattatgtagctaattccggccctgccgcatcagtgagagctgttttttattggtctcattcttttacatcagaatggttcgaacattcatctatcattgtaaaagcaggagaagatggaatattgaactcacctagcaattctgtatattacagtaaggttgtcatttacaacgatacggataaacgggcctttgtcacaggttatgacaaaatgaataacgaacttatgaacacaaatgaatcacaaccttcagagacattatctttaattaatgaatctatattaacagcaccttatgccgtttctacccctaattatgaatgggatatgtcatcaataataaaagatgccattattggaggtataggatttattcccgggccgggttcagcaatatcgtttttgctagggctattttggccgcaacaaacagacaatacctgggagcaaattctccaaaaagtagaacagatgatagaggaagcgaatttaaaaactattcaaggaatactgaacggagatatacaagaaataaaaggaaagatggaacatgtggaatatatgctagaaacctcaccaggcactcaagaaagccatgacgcatatatgttcttagcgagatatctggtaagtatagatgaaaaattcaaatcttttgataataaaacaaattatcaaattcttccaatgtacaccaatacgcttatgttacaggcaccttactggaaaatgggtatagagaagaaaaatgatattttgctaacagatatagaagttaatgaattaaaacagcttatcgaaaatctatatgccaaggccaatagctatattcatgaagtgtatacccgtgaatacgataatgcggtaaatacctcaacagcaacaacgattaccaataatttattgtctgtaagagggtattgtttattacatggattagagtgccttgaagtccttgatcatatacaaaataataatcttgatcagagcttctatccgaaaactatcagttattctactgtatttgatcgctcaacaaacaaaacaagactccaggctcttaccgaagacgagcaaatggaagaaccactcaaaccctcttttattaatggggaatataataaaataaaatcactgattggatatgtacagagaattggaaacgcccctagagttggaggtataaaaattacatttactaatggatcatctcatactctgggtacagtgacctcagaatcaaactcaattgaactaaatgatagtgttataaccagtgtggaagtatggggaaatggtgctgttgatgaggcattctttacattaagtgacggtcgtcaatttaggcttggtcaacgctatgccagtaactacagaaaatatgctgttgatggccactatatttcaggattgtacttagccagtgatgagccttcacttgctggtcaagccgcaggtattgcagtttcatatcatatattggttgataagaaataa

SEQ ID NO: 125 - protein, 704 aa, synthetic construct
  REGION 1..704: note = The amino acid sequence of the TIC11442 PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMITINISGGSIKISNNIGSETDIKNTPFSEPLSISNYKDMTIEPHSSIQATRTDTPIIPETRPNYYVANSGPAASVRAVFYWSHSFTSEWFEHSSIIVKAGEDGILNSPSNSVYYSKVVIYNDTDKRAFVTGYDKMNNELMNTNESQPSETLSLINESILTAPYAVSTPNYEWDMSSIIKDAIIGGIGFIPGPGSAISFLLGLFWPQQTDNTWEQILQKVEQMIEEANLKTIQGILNGDIQEIKGKMEHVEYMLETSPGTQESHDAYMFLARYLVSIDEKFKSFDNKTNYQILPMYTNTLMLQAPYWKMGIEKKNDILLTDIEVNELKQLIENLYAKANSYIHEVYTREYDNAVNTSTATTITNNLLSVRGYCLLHGLECLEVLDHIQNNNLDQSFYPKTISYSTVFDRSTNKTRLQALTEDEQMEEPLKPSFINGEYNKIKSLIGYVQRIGNAPRVGGIKITFTNGSSHTLGTVTSESNSIELNDSVITSVEVWGNGAVDEAFFTLSDGRQFRLGQRYASNYRKYAVDGHYISGLYLASDEPSLAGQAAGIAVSYHILVDKK

SEQ ID NO: 126 - DNA, 2124 bp, synthetic construct
  source 1..2124: mol_type = other DNA
  misc_feature 1..2124: note = A nucleic acid sequence encoding a PirAB fusion protein, TIC11443comprised of the TIC7660, TIC7575, and TIC7576 coding sequencesin operable linkage and in frame.
atgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaaatgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgaatatctcaccgattaatgtatctgaaaatgaaacattacctgaactcactgatgttatgcttattgtgccttatacaacatctacccctgattatgaatgggatatgtcatcaattataaaggatgcgattattggcggcgtagggtttattccaggagcaggctctgcaatgtccttcctattgggactattttggcctcaacagaaagataatacatgggaacagatcctccaaaaagtagaacagatgatagagaatgccgttctgcaaactattaaaggaatacttaatggagatatacaagaaatcaaggggaaaatggaacatgtgcaatacatgctggaaacctcgcctggcagtcaggaaagtcatgacgcatatatgttcctggctagatacctggtgagtatagatgaaaaattcaagtcttttgataataaaacaaactaccagatcctgccgatgtacactaacacggttatgttacaaatcccttattggaaaatgggaatagagaagaaaaatgatattgggctgacagatattgaagtcaatgagttaaaacagcttatcgataaattggtcgacaaggccaagagttacatccatacgatgtatacgaatgaatataatgatgccataaatacatcaacagcatcgagtgtcactaataatttactctctgtaagaggatattgtttattacacggtttagagtgtattgagttaattgaacatctacaaaacaatagcctcgaaagtggtttttatcctaaaactatcagttattcaactgtatttgatcgtcagactaacaaaatgagaattcaggctcttacagaagacgatcaaatgcaggaaccctttaagccatctttaatcaacggcaaatacaataaaatacaatccttgcttggatatgtacaaagaattggaaatgcacctagagtggggggtattaaaatcacctttgccaacggttcatcctatacacttggcacagtaacatcagaaacgagttcaattgaactcaatgatagtgttatcgaaagattggaagtatggggcaatggcgctgttgatgaggcattatttacgttaagtgatgggcgtcaactcagagtcggtgagcgctacgcgacaaaatatagaaaatatgctgttgatggacactatattgcaggactgtacttagctagcgatgaaccttcacttgctggtcaagccgcaggtattgccgtttcataccatatgttggatgataaaaaataa

SEQ ID NO: 127 - protein, 707 aa, synthetic construct
  REGION 1..707: note = The amino acid sequence of the TIC11443 PirAB fusion protein.
MITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNKMNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMNISPINVSENETLPELTDVMLIVPYTTSTPDYEWDMSSIIKDAIIGGVGFIPGAGSAMSFLLGLFWPQQKDNTWEQILQKVEQMIENAVLQTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDKLVDKAKSYIHTMYTNEYNDAINTSTASSVTNNLLSVRGYCLLHGLECIELIEHLQNNSLESGFYPKTISYSTVFDRQTNKMRIQALTEDDQMQEPFKPSLINGKYNKIQSLLGYVQRIGNAPRVGGIKITFANGSSYTLGTVTSETSSIELNDSVIERLEVWGNGAVDEALFTLSDGRQLRVGERYATKYRKYAVDGHYIAGLYLASDEPSLAGQAAGIAVSYHMLDDKK

SEQ ID NO: 128 - DNA, 2124 bp, synthetic construct
  source 1..2124: mol_type = other DNA
  misc_feature 1..2124: note = A nucleic acid sequence encoding a PirAB fusion protein, TIC11444comprised of the TIC7660 and TIC7576 coding sequences in operablelinkage and in frame.
atgaatacaatcaatataaatataagtggcagtaccgttacagtcataagcaataacgattccaatccagaaccattaacttataatacaaacacaccagcatcagaccctcttacagccagtccttatagggatatgacaatagagccacactcttctattgaagcaacaagaaccgatacaccgattattcccgaaactcgtcccaattactatgtagccaattctggccccgcatcatcagttagggctgttttttattggtctcattctttcacatcagaatggttcgaatattcctctatcatagtgaaagccgggaaagacggaatattacaatcaccgaataacgctttatattacagtaaagttgtcatttataacgataccgataaacgtgcctttgttaccggatataataagatgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaaatgaatactacacctattactgtatctacaaatgaaacatcgcctttaatgactgacgtaatgcccatggatctttatgcaatatccacacctgattatgaatgggacatgtcgtcaatcataaaggatgctgttattggtggcataggatttattccaggtccgggcccggcaatatccttcctgttagggctattttggcctcagcagaaagacaatacttgggagcaaattctccagaaagtagagcagatgatagagaatgctgttctacaaactattaaaggaatacttaatggagaagttcaagagatcaaagggaaaatggaacatgtagaatccatgctgaaaaactcgcctggcagtcaggaaagtcatgatgcatatatgttcctggcgagatatctggttagtatagatgaaaaattcaaatcttttgacaatagaacaaattaccagcttctcccaatgtatactaacactattatgttacagatcccttattggaaaatgggaatagagaagaaaaaagatattgggctgacagatattgaagttaatgaattaaaagaacttatcgataaattggttgataaggccaaaaactatattcatacgatgtatactaatgaacataataatgctgtaaacacatcaacagcagagagtgtcactaataatttattatctgtaagaggatattgtttattacacggtttagaatgtattgagttaatcgagcatatacagaataatagccttgagagtggtttctatcctaaaattatcagttattcgactgcgtttgatcgtcctactaacaaaatgagaattcaggctcttacagaagatgatgcaatgcaggagcctttcaaaccatctttaatcaatgggaaatataataaaatccaatccttgactggatatgtacaaagaattgggaatgcacctagagttggtggtatcagaatcacatttaccaacggctcatcttatacacttggtacagtgacctcagaaacgcattcaattaagctaaacgatagtgttatcgaaagcttggaagtatgggggaatggtgctgttgatgaggcgttatttaagttaagtgatgggcgtttattgcgtattggtgagcgctacgcgaaaaaatacagaaaatatgctgttgataatcactatattgcggggatttacttagccagcgatgagccttcacttgctggtcaagccgcaggtattgccgtttcatatcatatgatggctgacaaaaaataa

SEQ ID NO: 129 - protein, 707 aa, synthetic construct
  REGION 1..707: note = The amino acid sequence of the TIC11444 PirAB fusion protein.
MNTININISGSTVTVISNNDSNPEPLTYNTNTPASDPLTASPYRDMTIEPHSSIEATRTDTPIIPETRPNYYVANSGPASSVRAVFYWSHSFTSEWFEYSSIIVKAGKDGILQSPNNALYYSKVVIYNDTDKRAFVTGYNKMITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNKMNTTPITVSTNETSPLMTDVMPMDLYAISTPDYEWDMSSIIKDAVIGGIGFIPGPGPAISFLLGLFWPQQKDNTWEQILQKVEQMIENAVLQTIKGILNGEVQEIKGKMEHVESMLKNSPGSQESHDAYMFLARYLVSIDEKFKSFDNRTNYQLLPMYTNTIMLQIPYWKMGIEKKKDIGLTDIEVNELKELIDKLVDKAKNYIHTMYTNEHNNAVNTSTAESVTNNLLSVRGYCLLHGLECIELIEHIQNNSLESGFYPKIISYSTAFDRPTNKMRIQALTEDDAMQEPFKPSLINGKYNKIQSLTGYVQRIGNAPRVGGIRITFTNGSSYTLGTVTSETHSIKLNDSVIESLEVWGNGAVDEALFKLSDGRLLRIGERYAKKYRKYAVDNHYIAGIYLASDEPSLAGQAAGIAVSYHMMADKK

SEQ ID NO: 130 - DNA, 2124 bp, synthetic construct
  source 1..2124: mol_type = other DNA
  misc_feature 1..2124: note = A nucleic acid sequence encoding a PirAB fusion protein, TIC11445comprised of the TIC7660, TIC7662, and TIC7663 coding sequencesin operable linkage and in frame.
atgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaaatgagtacaatcaatatcaatataagtagcagtaccgttaccgtcatcacgaataacggagaaacgccagtcccactcacttacaatacaaatacacctgaatcagaacctcttaccgtcaatccttatagggatatgacaatagagccacgctcttctattgaagcaacaaggattgatacaccgattattcccgaaacacgccctaattattatgtagccaattcaggcccggcttcatcagttagggccgttttttattggtcccattctttcacatcacaatggttcgaatattcctctatcatcgtcaaagccggggaagatggcatattagaatcaccaagcaattctttatattacagcaaagtcgtcatttataatgataccgataaacgcgcctttgtgacgggatataataagatgaataccactctgattaatgtatctgaaaaagaaacattgcctgtacaaactgatatcatgcttatcgcgccttattcagtatcgacccccgattatgaatgggatatgtcctcactcatcaaggatgccattattggtggcgtagggtttattcccgtcgtaggttccgcaatgtccttcctgctaggattattttggccccaacagaaagataatacttgggagcaaattctccaaaaagtcgagcagatgatcgagaatgcccagctaaatacgattaaaggaatacttaatggcgatatacaagagatcaaaggaaaaatggagcatgtacaatacatgttggaaacctcgccgggcagtcaagaaagtcatgatgcctatatgttcctggccagatatctggtgagtatcgatgagaaatttaagtcttttgataataaaacaaactatcaaattttgccgatgtatacgaacacggttatgttgcagatcccttattggaaaatggggatcgagaagaaaaatgatattgggctgaccgatattgaagtcaatgagttaaaacagcttatcgacacattggttgacagagccaggaactatattcatacgatgtatgaaagagaatatgataatgccatcaacacctcaaccgcggcgagcgtcactaataatttattgtccgtcagaggatattgcctgttacacggtttagagtgtattgaaaccattgaacatctgcaaaataatagccttaatagtggtttctatcctaaaaccattagttattcaacggtatttgatcgtcccacgaacaaaacgagaattcaggctctgaccgaagatgaccaaatgcaagagcctttcaagccagctttaattggcggtaagtacaataaaataaaatcattgcttggctatgtacgaagaattgggaatgcccccagagtggggggaattaaggtcacctttaccaacggatcatcttatacacttggcacagtcacatcagaaacggactcaattgagctaaatgagagtgttatcgaaagattagaagtatggggcaatggtgctgttgatgaggcattatttacgttaagcgatgggcgccaactcaggatcggcgagcgctacgcgaaaaaatacagaaaatatgctgttgatggacactatatttcagggctgtacttagccagcgatgaaccttcccttgctggtcaggccgcaggtattgccgtttcataccatatgcttgctgataaaaaataa

SEQ ID NO: 131 - protein, 707 aa, synthetic construct
  REGION 1..707: note = The amino acid sequence of the TIC11445 PirAB fusion protein.
MITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNKMSTININISSSTVTVITNNGETPVPLTYNTNTPESEPLTVNPYRDMTIEPRSSIEATRIDTPIIPETRPNYYVANSGPASSVRAVFYWSHSFTSQWFEYSSIIVKAGEDGILESPSNSLYYSKVVIYNDTDKRAFVTGYNKMNTTLINVSEKETLPVQTDIMLIAPYSVSTPDYEWDMSSLIKDAIIGGVGFIPVVGSAMSFLLGLFWPQQKDNTWEQILQKVEQMIENAQLNTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDTLVDRARNYIHTMYEREYDNAINTSTAASVTNNLLSVRGYCLLHGLECIETIEHLQNNSLNSGFYPKTISYSTVFDRPTNKTRIQALTEDDQMQEPFKPALIGGKYNKIKSLLGYVRRIGNAPRVGGIKVTFTNGSSYTLGTVTSETDSIELNESVIERLEVWGNGAVDEALFTLSDGRQLRIGERYAKKYRKYAVDGHYISGLYLASDEPSLAGQAAGIAVSYHMLADKK

SEQ ID NO: 132 - DNA, 2124 bp, synthetic construct
  source 1..2124: mol_type = other DNA
  misc_feature 1..2124: note = A nucleic acid sequence encoding a fusion protein, TIC11446comprised of the TIC7662, TIC7660, and TIC7661 coding sequencesin operable linkage and in frame.
atgagtacaatcaatatcaatataagtagcagtaccgttaccgtcatcacgaataacggagaaacgccagtcccactcacttacaatacaaatacacctgaatcagaacctcttaccgtcaatccttatagggatatgacaatagagccacgctcttctattgaagcaacaaggattgatacaccgattattcccgaaacacgccctaattattatgtagccaattcaggcccggcttcatcagttagggccgttttttattggtcccattctttcacatcacaatggttcgaatattcctctatcatcgtcaaagccggggaagatggcatattagaatcaccaagcaattctttatattacagcaaagtcgtcatttataatgataccgataaacgcgcctttgtgacgggatataataagatgatcacaataaatataaatgtaaacggcaatgatgttacaggtacaaataataatgaacctactccagtatcgacaacttacggtccaaatacaccagcatcagaaccccctgtagtcagtaattatagtgatataacaatagaaccgcattcttctgtgcaggcaacaagaattgatacgcctgttattcctgaagcacgccccgattactatgtagccaactccggccctgcaccatcagttagggctgttttttattggtctcattctttcacatcagaatggttcgaatcttcctctatcacagtgaaagcaggagaggacggaatattaaaagcacctggtaactctttatattacagcaaagtcgtcatttataatgatacggataaacgagcctttgttactggatataataaaatgaatactacacctattactgtatctacaaatgaaacatcgcctttaatgactgacgtaatgcccatggatctttatgcaatatccacacctgattatgaatgggacatgtcgtcaatcataaaggatgctgttattggtggcataggatttattccaggtccgggcccggcaatatccttcctgttagggctattttggcctcagcagaaagacaatacttgggagcaaattctccagaaagtagagcagatgatagagaatgctgttctacaaactattaaaggaatacttaatggagaagttcaagagatcaaagggaaaatggaacatgtagaatccatgctgaaaaactcgcctggcagtcaggaaagtcatgatgcatatatgttcctggcgagatatctggttagtatagatgaaaaattcaaatcttttgacaatagaacaaattaccagcttctcccaatgtatactaacactattatgttacagatcccttattggaaaatgggaatagagaagaaaaaagatattgggctgacagatattgaagttaatgaattaaaagaacttatcgataaattggttgataaggccaaaaactatattcatacgatgtatactaatgaacataataatgctgtaaacacatcaacagcagagagtgtcactaataatttattatctgtaagaggatattgtttattacacggtttagaatgtattgagttaatcgagcatatacagaataatagccttgagagtggtttctatcctaaaattatcagttattcgactgcgtttgatcgtcctactaacaaaatgagaattcaggctcttacagaagatgatgcaatgcaggagcctttcaaaccatctttaatcaatgggaaatataataaaatccaatccttgactggatatgtacaaagaattgggaatgcacctagagttggtggtatcagaatcacatttaccaacggctcatcttatacacttggtacagtgacctcagaaacgcattcaattaagctaaacgatagtgttatcgaaagcttggaagtatgggggaatggtgctgttgatgaggcgttatttaagttaagtgatgggcgtttattgcgtattggtgagcgctacgcgaaaaaatacagaaaatatgctgttgataatcactatattgcggggatttacttagccagcgatgagccttcacttgctggtcaagccgcaggtattgccgtttcatatcatatgatggctgacaaaaaataa

SEQ ID NO: 133 - protein, 707 aa, synthetic construct
  REGION 1..707: note = The amino acid sequence of the TIC11446 PirAB fusion protein.
MSTININISSSTVTVITNNGETPVPLTYNTNTPESEPLTVNPYRDMTIEPRSSIEATRIDTPIIPETRPNYYVANSGPASSVRAVFYWSHSFTSQWFEYSSIIVKAGEDGILESPSNSLYYSKVVIYNDTDKRAFVTGYNKMITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNKMNTTPITVSTNETSPLMTDVMPMDLYAISTPDYEWDMSSIIKDAVIGGIGFIPGPGPAISFLLGLFWPQQKDNTWEQILQKVEQMIENAVLQTIKGILNGEVQEIKGKMEHVESMLKNSPGSQESHDAYMFLARYLVSIDEKFKSFDNRTNYQLLPMYTNTIMLQIPYWKMGIEKKKDIGLTDIEVNELKELIDKLVDKAKNYIHTMYTNEHNNAVNTSTAESVTNNLLSVRGYCLLHGLECIELIEHIQNNSLESGFYPKIISYSTAFDRPTNKMRIQALTEDDAMQEPFKPSLINGKYNKIQSLTGYVQRIGNAPRVGGIRITFTNGSSYTLGTVTSETHSIKLNDSVIESLEVWGNGAVDEALFKLSDGRLLRIGERYAKKYRKYAVDNHYIAGIYLASDEPSLAGQAAGIAVSYHMMADKK

SEQ ID NO: 134 - DNA, 1305 bp, Xenorhabdus nematophila
  source 1..1305: mol_type = other DNA
  misc_feature 1..1305: note = A nucleic acid sequence obtained from Xenorhabdus nematophilastrain MDI-0035777 encoding a TIC11505 pesticidal PirB proteinsequence.
atgaataatgaaccgatgaatactaatgaatcacaagcttcagagatagtaccctcaatgaatgaatctatattaaatgaatctatattagcagcaccttattcaatttctacacctaattatgaatgggatatgtcatcaataataaaagatgccattattggtggtataggctttattcctggtccgggttcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcacctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagataaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttaattaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctgttgatgaggcattctttacattaagtgacggacgtcaatttaggcttggccaacgctatgccagtaactatagaaaatatgctgtcgataaccactatatttcaggattgtacttagccagtgatgaaccttcattggcaggccaagcagcaggcattgcagtttcataccatatgatagctgataaaaaatcatag

SEQ ID NO: 135 - protein, 434 aa, Xenorhabdus nematophila
  REGION 1..434: note = The amino acid sequence of the TIC11505 PirB protein.
MNNEPMNTNESQASEIVPSMNESILNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSPGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALINGKYNKIKSLIGYVQRIGNAPRVGGIKVTFANDASYTLGTVTSEVNSIELNDSVITSLEVWGNGAVDEAFFTLSDGRQFRLGQRYASNYRKYAVDNHYISGLYLASDEPSLAGQAAGIAVSYHMIADKKS

SEQ ID NO: 136 - DNA, 1731 bp, synthetic construct
  source 1..1731: mol_type = other DNA
  misc_feature 1..1731: note = A nucleic acid sequence encoding a PirAB fusion protein, TIC11506comprised of the TIC10364 and TIC11505 coding sequences inoperable linkage and in frame.
atgagcataatcaatataaatataagtggtagtagtgacattacaatcataaacaataccccatctaacccagaaccactcatttacaatacagacacacccgcatcagaacctcttaccgtcaatccttatagggatatgacaatagagccacactcttctattgaggcaataagaattgatacgccaattattcccgaaacccgccccaattattacgtagccaattctggccccgcatcatcagttagagccgttttttattggtctcactctttcacatcagaatggttcgaatattctgctatcacagtgaaagccggggaagacggcatattacaatcaccgagcaactctgtgtattacagcaaggtcgtcatttataacgaaaccgataaacgcgcctttgttactggatataataagatgaataatgaaccgatgaatactaatgaatcacaagcttcagagatagtaccctcaatgaatgaatctatattaaatgaatctatattagcagcaccttattcaatttctacacctaattatgaatgggatatgtcatcaataataaaagatgccattattggtggtataggctttattcctggtccgggttcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcacctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagataaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttaattaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctgttgatgaggcattctttacattaagtgacggacgtcaatttaggcttggccaacgctatgccagtaactatagaaaatatgctgtcgataaccactatatttcaggattgtacttagccagtgatgaaccttcattggcaggccaagcagcaggcattgcagtttcataccatatgatagctgataaaaaatcatag

SEQ ID NO: 137 - protein, 576 aa, synthetic construct
  REGION 1..576: note = The amino acid sequence of the TIC11506 PirAB fusion protein.
MSIININISGSSDITIINNTPSNPEPLIYNTDTPASEPLTVNPYRDMTIEPHSSIEAIRIDTPIIPETRPNYYVANSGPASSVRAVFYWSHSFTSEWFEYSAITVKAGEDGILQSPSNSVYYSKVVIYNETDKRAFVTGYNKMNNEPMNTNESQASEIVPSMNESILNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSPGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALINGKYNKIKSLIGYVQRIGNAPRVGGIKVTFANDASYTLGTVTSEVNSIELNDSVITSLEVWGNGAVDEAFFTLSDGRQFRLGQRYASNYRKYAVDNHYISGLYLASDEPSLAGQAAGIAVSYHMIADKKS

SEQ ID NO: 138 - DNA, 1290 bp, Xenorhabdus bovienii
  source 1..1290: mol_type = other DNA
  misc_feature 1..1290: note = A nucleic acid sequence obtained from Xenorhabdus bovienii strainMDI-0035808 encoding a TIC11510 pesticidal PirB protein sequence.
atgaataatgaaccgatgaatactaatgaatcacaagcttcagagatagtaccctcaatgaatgaatctatattagcagcaccttattcaatttctacacctaattatgaatgggatatgtcatcaataataaaagatgccattattggtggtataggctttattcctggtccgggctcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcatctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagatcaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttagttaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctattgatgaggcattctttacattaagtgacggacgtcaatttaggcttggccaacgctatgccagtaactatagaaaatatgctgtcgataaccactatatttcaggattgtacttagccagtgatgaaccttcattggcaggtcaagcagcaggcattgcagtttcataccatatgatagctgataaaaaatcatag

SEQ ID NO: 139 - protein, 429 aa, Xenorhabdus bovienii
  REGION 1..429: note = The amino acid sequence of the TIC11510 PirB protein.
MNNEPMNTNESQASEIVPSMNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSSGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALVNGKYNKIKSLIGYVQRIGNAPRVGGIKVTFANDASYTLGTVTSEVNSIELNDSVITSLEVWGNGAIDEAFFTLSDGRQFRLGQRYASNYRKYAVDNHYISGLYLASDEPSLAGQAAGIAVSYHMIADKKS

SEQ ID NO: 140 - DNA, 1716 bp, synthetic construct
  source 1..1716: mol_type = other DNA
  misc_feature 1..1716: note = A nucleic acid sequence encoding a PirAB fusion protein, TIC11512comprised of the TIC10364 and TIC11510 coding sequences inoperable linkage and in frame.
atgagcataatcaatataaatataagtggtagtagtgacattacaatcataaacaataccccatctaacccagaaccactcatttacaatacagacacacccgcatcagaacctcttaccgtcaatccttatagggatatgacaatagagccacactcttctattgaggcaataagaattgatacgccaattattcccgaaacccgccccaattattacgtagccaattctggccccgcatcatcagttagagccgttttttattggtctcactctttcacatcagaatggttcgaatattctgctatcacagtgaaagccggggaagacggcatattacaatcaccgagcaactctgtgtattacagcaaggtcgtcatttataacgaaaccgataaacgcgcctttgttactggatataataagatgaataatgaaccgatgaatactaatgaatcacaagcttcagagatagtaccctcaatgaatgaatctatattagcagcaccttattcaatttctacacctaattatgaatgggatatgtcatcaataataaaagatgccattattggtggtataggctttattcctggtccgggctcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcatctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagatcaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttagttaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctattgatgaggcattctttacattaagtgacggacgtcaatttaggcttggccaacgctatgccagtaactatagaaaatatgctgtcgataaccactatatttcaggattgtacttagccagtgatgaaccttcattggcaggtcaagcagcaggcattgcagtttcataccatatgatagctgataaaaaatcatag

SEQ ID NO: 141 - protein, 571 aa, synthetic construct
  REGION 1..571: note = The amino acid sequence of the TIC11512 PirAB fusion protein.
MSIININISGSSDITIINNTPSNPEPLIYNTDTPASEPLTVNPYRDMTIEPHSSIEAIRIDTPIIPETRPNYYVANSGPASSVRAVFYWSHSFTSEWFEYSAITVKAGEDGILQSPSNSVYYSKVVIYNETDKRAFVTGYNKMNNEPMNTNESQASEIVPSMNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSSGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALVNGKYNKIKSLIGYVQRIGNAPRVGGIKVTFANDASYTLGTVTSEVNSIELNDSVITSLEVWGNGAIDEAFFTLSDGRQFRLGQRYASNYRKYAVDNHYISGLYLASDEPSLAGQAAGIAVSYHMIADKKS

SEQ ID NO: 142 - DNA, 1290 bp, Xenorhabdus nematophila
  source 1..1290: mol_type = other DNA
  misc_feature 1..1290: note = A nucleic acid sequence obtained from Xenorhabdus nematophilastrain AN6/1 encoding a TIC11511 pesticidal PirB proteinsequence.
atgaataatgaaccgatgaatactaatgaatcacaagcttcagagatagtaccctcaatgaatgaatctatattagcagcaccttattcaatttctacacctaattatgaatgggatatgtcatcaataataaaagatgccattattggtggtataggctttattcctggtccgggctcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcacctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagataaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttaattaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctattgatgaggcattctttacattaagtgacggacgtcaatttaggcttggccaacgctatgccagtaactatagaaaatatgctgtcgataaccactatatttcaggattgtacttagccagtgatgaaccttcattggcaggccaagcagcaggcattgcagtttcataccatatgatagctgataaaaaatcatag

SEQ ID NO: 143 - protein, 429 aa, Xenorhabdus nematophila
  REGION 1..429: note = The amino acid sequence of the TIC11511 PirB protein.
MNNEPMNTNESQASEIVPSMNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSPGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALINGKYNKIKSLIGYVQRIGNAPRVGGIKVTFANDASYTLGTVTSEVNSIELNDSVITSLEVWGNGAIDEAFFTLSDGRQFRLGQRYASNYRKYAVDNHYISGLYLASDEPSLAGQAAGIAVSYHMIADKKS

SEQ ID NO: 144 - DNA, 1716 bp, synthetic construct
  source 1..1716: mol_type = other DNA
  misc_feature 1..1716: note = A nucleic acid sequence encoding a PirAB fusion protein, TIC11513comprised of the TIC10364 and TIC11511 coding sequences inoperable linkage and in frame.
atgagcataatcaatataaatataagtggtagtagtgacattacaatcataaacaataccccatctaacccagaaccactcatttacaatacagacacacccgcatcagaacctcttaccgtcaatccttatagggatatgacaatagagccacactcttctattgaggcaataagaattgatacgccaattattcccgaaacccgccccaattattacgtagccaattctggccccgcatcatcagttagagccgttttttattggtctcactctttcacatcagaatggttcgaatattctgctatcacagtgaaagccggggaagacggcatattacaatcaccgagcaactctgtgtattacagcaaggtcgtcatttataacgaaaccgataaacgcgcctttgttactggatataataagatgaataatgaaccgatgaatactaatgaatcacaagcttcagagatagtaccctcaatgaatgaatctatattagcagcaccttattcaatttctacacctaattatgaatgggatatgtcatcaataataaaagatgccattattggtggtataggctttattcctggtccgggctcagcaatatcatttttgttagggttattttggccacaacaaaccgacaatacttgggagcaaattctccaaaaagtagaacaaatgatcgagcaagccaatctcaaaactattcaaggaatattgaacggcgatatacaagaaattaaaggcaaaatggaacatgtgcaattcatgctagaatcctcacctggcactcaagaaagccatgacgcatacatgtttctggcgagatatctggtcagtatagacgaaaaattcaagtcttttgataacaaaacaaattatcaaattcttcccatgtataccaatacgattatgttacaagccccttattggaaaatgggtatagagagaaaagatgagataaaactaacagatatagaagttaatgaattaaaagagctgataggaaaattatctaccagcgccgataaatatattcatgatgtctatactcgtgaatatgataatgcgatgaacacttcaacagcagcaaatatcaccaataatttattatctgtaagaggctattgtttattacatggtttagaatgtctcgaagtcattaaccatatacaaaataatagccttgagcaaagtttttatcctaaaactatcagctactccaccgtattcgatcgccagacaaataaaacaagggttcaagccctgacagaagacgatcaaatgcaagagccattcaagcctgctttaattaatgggaagtacaacaaaataaaatcattgattgggtatgtacaaagaatcggaaacgcacccagagttggaggcattaaagtcacatttgcaaacgatgcatcttataccctcggtacagtaacttcagaagtaaactcaattgaactgaatgacagcgttataaccagcctggaagtatggggaaatggcgctattgatgaggcattctttacattaagtgacggacgtcaatttaggcttggccaacgctatgccagtaactatagaaaatatgctgtcgataaccactatatttcaggattgtacttagccagtgatgaaccttcattggcaggccaagcagcaggcattgcagtttcataccatatgatagctgataaaaaatcatag

SEQ ID NO: 145 - protein, 571 aa, synthetic construct
  REGION 1..571: note = The amino acid sequence of the TIC11513 PirAB fusion protein.
MSIININISGSSDITIINNTPSNPEPLIYNTDTPASEPLTVNPYRDMTIEPHSSIEAIRIDTPIIPETRPNYYVANSGPASSVRAVFYWSHSFTSEWFEYSAITVKAGEDGILQSPSNSVYYSKVVIYNETDKRAFVTGYNKMNNEPMNTNESQASEIVPSMNESILAAPYSISTPNYEWDMSSIIKDAIIGGIGFIPGPGSAISFLLGLFWPQQTDNTWEQILQKVEQMIEQANLKTIQGILNGDIQEIKGKMEHVQFMLESSPGTQESHDAYMFLARYLVSIDEKFKSFDNKTNYQILPMYTNTIMLQAPYWKMGIERKDEIKLTDIEVNELKELIGKLSTSADKYIHDVYTREYDNAMNTSTAANITNNLLSVRGYCLLHGLECLEVINHIQNNSLEQSFYPKTISYSTVFDRQTNKTRVQALTEDDQMQEPFKPALINGKYNKIKSLIGYVQRIGNAPRVGGIKVTFANDASYTLGTVTSEVNSIELNDSVITSLEVWGNGAIDEAFFTLSDGRQFRLGQRYASNYRKYAVDNHYISGLYLASDEPSLAGQAAGIAVSYHMIADKKS

SEQ ID NO: 146 - DNA, 1689 bp, synthetic construct
  source 1..1689: mol_type = other DNA
  misc_feature 1..1689: note = a synthetic coding sequence used for expression in plant cellsencoding a TIC10376PL PirAB fusion protein with an additionalalanine codon inserted after the initiating methionine codon.
atggctccggtgaaccagatcgggctccacaacgagaaggtcaagaacatgcggaagatcaccgtggacaatgacgtggtgggccacgacaccgagatcaacagcgtcgtgagcagcacggccgagaagatccggcagcagttcggcgtgaaagtcgatcccaattcgtcccaggagaagttctacatcgccacgcccatcatcccggagtcccgcaagaacatcgtggtcaccaacgagggcctggcggacgtcatcaccgccaagtactattggagccacagcttcaccagcgaatactttgaggacaactcggtggacgtcaaggtgggcgagagcaaagtgctggtcgctccgagcaaccctctctactacagcaaggtcgtcatcttcaacaacaccaagtcggtggccttcgtcacggtccgcgagaagatgagcgacatcgtgaagtacaacgacgtcagcgcgcccatcccgtacgcggtttacagtaacgccgtgtacgccttcgagtgggactcgtcggcgatcctcaagcaagcagtggtgaagggcttgtcctacgtgccgcacgtcgggaagtacctctcgtacatcgtcggattcttctggaaggacaaggagaaggacatctggcaagaggtggtgggcaaggtgcagcagttggtagaggactcgatcctcaaggcggtgaagggcatcctgtccggcaacatcaacgagctaaaggagaagatgaatgaggtgatccgctcccttgagaagaatttgggaacccaagaggcccgcgacgactacatgcacctggcccgcagcatggtgggcaaggaggcaagccttatcttccacgagaacaagaccaacttccacatcctgccgatgtactctacgctcgccctcatgcagatcatgtactggacggtgggcatcgaacgccgcaaggaaattggtctgagcgacatcgaggtcgagaacctccggagctacatcaagaagctcgtcagcgacgcggagcaccacgtgaaccgggtgtacaagctggagctggactccgtggtcagcgactcggacgtgaaccgggtggcggacaacatcatgtacgtccacggctactgccagattcacggcctggagtacatggacatcatcaagaacatacagtctcgtggcaacaacatcaccggcttctaccctaggactatctcgtactctacattctttggctcgccgaccagcgacgcgcggattctggccctgcggccggagaaggacatgcccgagcctttcaagcccaagttcctgaacgagcggttcaacaagatcgcgtcggtcaagggctacatcgtccggatcggaggtgccaagagggtcggcggcctggagatcacgttcgagaacgggtcgaagtaccagcaagggcaagcgaccaacgagcacgagatcgtgaacctcaagggcaaccttatcaagaccctggaggtgtggggaaacggtgccatcgacgaggccaagttcacgctgacgaacggcgacgtcctcaccatcgggcagcgcaacagcagcaactaccggaagttctccctggacggccactacatctgcggcgtgttcatcgccaacgaccgctcgggcctcgcgggacaagccgcgaacatcgccgtctcctaccatcagctcgtcgagtga

SEQ ID NO: 147 - protein, 562 aa, synthetic construct
  REGION 1..562: note = The amino acid sequence of the TIC10376PL PirAB fusion protein.
MAPVNQIGLHNEKVKNMRKITVDNDVVGHDTEINSVVSSTAEKIRQQFGVKVDPNSSQEKFYIATPIIPESRKNIVVTNEGLADVITAKYYWSHSFTSEYFEDNSVDVKVGESKVLVAPSNPLYYSKVVIFNNTKSVAFVTVREKMSDIVKYNDVSAPIPYAVYSNAVYAFEWDSSAILKQAVVKGLSYVPHVGKYLSYIVGFFWKDKEKDIWQEVVGKVQQLVEDSILKAVKGILSGNINELKEKMNEVIRSLEKNLGTQEARDDYMHLARSMVGKEASLIFHENKTNFHILPMYSTLALMQIMYWTVGIERRKEIGLSDIEVENLRSYIKKLVSDAEHHVNRVYKLELDSVVSDSDVNRVADNIMYVHGYCQIHGLEYMDIIKNIQSRGNNITGFYPRTISYSTFFGSPTSDARILALRPEKDMPEPFKPKFLNERFNKIASVKGYIVRIGGAKRVGGLEITFENGSKYQQGQATNEHEIVNLKGNLIKTLEVWGNGAIDEAKFTLTNGDVLTIGQRNSSNYRKFSLDGHYICGVFIANDRSGLAGQAANIAVSYHQLVE

SEQ ID NO: 148 - DNA, 1710 bp, synthetic construct
  source 1..1710: mol_type = other DNA
  misc_feature 1..1710: note = A synthetic coding sequence used for expression in plant cellsencoding a TIC10378PL PirAB fusion protein with an additionalalanine codon inserted after the initiating methionine codon.
atggctatcacgatcaacatcaacaccaacggcgtgaacggcatcaccatcaccaacagcaacaacgagccgacgcccgtaagcacgacctacggtcccaacactccggcgagcgagccgctcaccgtcagcaactactcggacatcaccatcgagccgcacagctccgtccaggccacgcgcatcgacacgccgatcatcccggagacccggccggactactacgtcgctaactccgggcccgcgccgaccgtccgcgccgtgttctactggtcgcacagcttcacgtccgagtggttcgagtccagctccataacggtgaaggcgggcgaggatggtatcctcaaagcgcccggaaactcgctctactacagcaaggtggtcatctacaatgacaccgacaagcgcgccttcgtgaccggctacaacaagatgaacacgacgccgatcaccgtgtcggagaacgagacgtcgcctctcctcaccgacgtcatgccgatggacctgtacgcggtgtccacacccgactacgagtgggacatgtcctccatcatcaaggacgccatcatcggcggcatcggcttcattcccggtcccggccctgctctgtcgttcttgctcggactgttctggccgcagcagaaggacaacacttgggagcaaatccttcagaaggtggagcagatgatcgagaacgcggtgctccagacgatcaagggcatcctgaacggcgagatccaggagatcaagggcaagatggagcacgtggagtccatgctcaagaatagccctggtagccaagagtcccacgacgcctacatgttcctagcccgttacctcgtttccatcgacgagaaattcaagtccttcgacaaccgcacgaactaccagatcctcccgatgtacaccaataccatcatgctccagattccctactggaagatgggcatcgagaagaagaaggacattggcctgacggacatcgaggtcaacgaacttaaagagctgatcgacaagctcgtcgggaaggccaagaattacatccacacaatgtacactaatgagtacaacgacgcgatcaacacgagcacggctgggtcggttaccaacaacctgctctccgtgcgcgggtactgcttgctgcacgggctagagtgcattgagctgatcgagcacatccagaacaatagcctggagagcgggttctaccctaagaccatctcgtactccacggtgttcgaccgccctacgaacaagatgcggatacaggcgctgaccgaggacgacgcgatgcaagaaccgttcaagccgagcctgattaacggcaagtacaacaagatccagtcgattatcgggtacgtccagcggatcgggaacgctccgcgcgtcggcgggattaagatcaccttcacaaacggtagcagttacacgctcgggactgtcacgtccgagacaaatagtatcgagttgaacgacagcgtgatcgagtccctggaggtgtggggcaacggcgcggtggacgaggcgctgtttaagctgtcggatggccgtctgctgcgaataggcgagcggtacgccaagaaataccgcaagtacgccgtggaccaccattacatcgccgggatctacctcgccagcgatgaaccctcgcttgcgggacaggcggccgggatcgcggtgtcgtatcacatgatggccgacaagaaatga

SEQ ID NO: 149 - protein, 569 aa, synthetic construct
  REGION 1..569: note = The amino acid sequence of the TIC10378PL PirAB fusion protein.
MAITININTNGVNGITITNSNNEPTPVSTTYGPNTPASEPLTVSNYSDITIEPHSSVQATRIDTPIIPETRPDYYVANSGPAPTVRAVFYWSHSFTSEWFESSSITVKAGEDGILKAPGNSLYYSKVVIYNDTDKRAFVTGYNKMNTTPITVSENETSPLLTDVMPMDLYAVSTPDYEWDMSSIIKDAIIGGIGFIPGPGPALSFLLGLFWPQQKDNTWEQILQKVEQMIENAVLQTIKGILNGEIQEIKGKMEHVESMLKNSPGSQESHDAYMFLARYLVSIDEKFKSFDNRTNYQILPMYTNTIMLQIPYWKMGIEKKKDIGLTDIEVNELKELIDKLVGKAKNYIHTMYTNEYNDAINTSTAGSVTNNLLSVRGYCLLHGLECIELIEHIQNNSLESGFYPKTISYSTVFDRPTNKMRIQALTEDDAMQEPFKPSLINGKYNKIQSIIGYVQRIGNAPRVGGIKITFTNGSSYTLGTVTSETNSIELNDSVIESLEVWGNGAVDEALFKLSDGRLLRIGERYAKKYRKYAVDHHYIAGIYLASDEPSLAGQAAGIAVSYHMMADKK

SEQ ID NO: 150 - DNA, 1707 bp, synthetic construct
  source 1..1707: mol_type = other DNA
  misc_feature 1..1707: note = A synthetic coding sequence used for expression in plant cellsencoding a TIC10380PL PirAB fusion protein with an additionalalanine codon inserted after the initiating methionine codon.
atggctatcaccatcaacatcagcggcgggtccgtgacgatcaacaacacctacaacatcacttccgagagcggcatccagaacacgccagcgtcggagcctctcaccgtcatcccgtaccgtgacatgaccatcgagcctcacagcagcattgaggccacccgcaccgacacgccgatcatcccggagacgcggcccaactactacatcgctaactcgggcccggcctccgaggtgcgcgcggtgttctactggtcgcactcgttcacgtcgcagtggttcgagtcgtccagcataatcgtcaaggcgggcgaggacggcatcctccagtcgccgtcgaacagtctgtactactcgaaggtggtcatctacaatgacaccgacaagcgcgcgttcgtcaccggctacaacaagatgaacaacacctccatcaacatcaatgagaacgagacgctgccgcttgaagtcatcccgtccatgcccgagcccatgctgatcgtcccgtatgccacgagcacgccggattacgagtgggacgccagcgggatcatcaaggatgccatcatcggcgggatcgggttcataccgggccctggcccagcgatctccttcctgctgggcttgttctggccgcaacaagctgacaacacctgggagcagatcctccagaaggtcgagcagatgatcgaggacgcagtgctaaagaccatccagggcatcctgaacggtgacatccaggagatcaagggcaagatggagcacgtgcagtacatgctggagacatcgcccggcagccaggagtcccgtgaggcgtacatgttcctcgcccgctacctcgtcagcattgacgagaagttcaagtccttcgacaacaagaccaattaccagatcctgcccatgtacacgaatactctcatgctccaagtgccatactggaagatgggcatcgagaagcagaaggacatcggcctctccgacatcgaagtcaacgagcttaaacagctaatcgacaagctctacaccaaagctaattcgtacatacacgaaacatacacacggcagtacaacgacgcgataaacaccagcacggcagccaacatcaccaacaacctgttcagcgtgcgcggctactgcttgctgcacgggctggagtgcctggagatgattgagcatcttcagaagaatagccttgaatcgggtttctaccctaagaccataagctacagcacagtctttgaccgtcagacgcccaagatgcggatacaagccctgaccgaggacgatcagatgcaagagccgctcaaaccctcgctgataaacggcaagtacaaccagatcaagagcctcaccggatacgtccggcggatcggcaacgctccgcgcgtcggcggaatgacgatcaccttcgccaatggcgcgtcctacaccctcgggacggtcacctccgagaccaccagtatcgagctgaacggctccgtgatcgagtccctggaggtgtggggcgacggcgcggtggacgaggcgctcttcaccttaagcgacaagcgcctgttccgtatcggcgagcgctatgcgcgcaagtacaagaagtatgccgtggacagccactacatcgctgggctctacctggcctcggacgagccatcgttggccgggcaagctgcgggcattgccgtcagctaccacatgctcgacgacaagaaatga

SEQ ID NO: 151 - protein, 568 aa, synthetic construct
  REGION 1..568: note = The amino acid sequence of the TIC10380PL PirAB fusion protein.
MAITINISGGSVTINNTYNITSESGIQNTPASEPLTVIPYRDMTIEPHSSIEATRTDTPIIPETRPNYYIANSGPASEVRAVFYWSHSFTSQWFESSSIIVKAGEDGILQSPSNSLYYSKVVIYNDTDKRAFVTGYNKMNNTSININENETLPLEVIPSMPEPMLIVPYATSTPDYEWDASGIIKDAIIGGIGFIPGPGPAISFLLGLFWPQQADNTWEQILQKVEQMIEDAVLKTIQGILNGDIQEIKGKMEHVQYMLETSPGSQESREAYMFLARYLVSIDEKFKSFDNKTNYQILPMYTNTLMLQVPYWKMGIEKQKDIGLSDIEVNELKQLIDKLYTKANSYIHETYTRQYNDAINTSTAANITNNLFSVRGYCLLHGLECLEMIEHLQKNSLESGFYPKTISYSTVFDRQTPKMRIQALTEDDQMQEPLKPSLINGKYNQIKSLTGYVRRIGNAPRVGGMTITFANGASYTLGTVTSETTSIELNGSVIESLEVWGDGAVDEALFTLSDKRLFRIGERYARKYKKYAVDSHYIAGLYLASDEPSLAGQAAGIAVSYHMLDDKK

SEQ ID NO: 152 - DNA, 1707 bp, synthetic construct
  source 1..1707: mol_type = other DNA
  misc_feature 1..1707: note = A synthetic coding sequence used for expression in plant cellsencoding a TIC10381PL PirAB fusion protein with an additionalalanine codon inserted after the initiating methionine codon.
atggcttcaatcatcaacatcaacattagcgggtcgtcggacattactatcattaacaacacgccgagcaacccggagccgctgatctacaacactgacactccggcctccgagccgctcaccgtcaacccttaccgggacatgaccatcgagccgcacagcagcatcgaggccattcgaatcgacacacccatcatcccggagacgcggcccaactattacgtggccaactcgggcccggcctcgtccgtccgggcggtgttctactggtcgcactccttcacgtccgagtggttcgagtacagcgccatcacggtcaaggcgggcgaagacggtatcctccaatcgccctcgaacagcgtgtactattcaaaggtcgtcatctacaacgagacggataagcgcgcgttcgtcaccggctacaacaagatgaacaccacgccgatcaacgtgagcgagaacgacaccctacccgtgctcaccgacgtgatgttgatcgtgccctacacaacctccacgccggactacgagtgggacatgagtagcatcatcaaggacgccatcatcggcggcgtgggcttcattcccggcgtcgggtcggcgatgtcgttcctgctgggcttgttctggccgcaacagaaggacaacacctgggagcagatccttcagaaggttgagcagatgatcgagaacgcggcactccagaccatcaagggcatcctgaacggcgacatccaggagatcaagggcaagatggagcacgtgcagtacatgctggaaacgtcgcccggctctcaggaatcccacgacgcctacatgttcctggccagatacctcgtctctatcgacgagcgcttcaagtccttcgacaacaagacgaattaccagatcctgccgatgtacacaaataccgtgatgctacaaattccctactggaagatgggcatcgagaagaagaatgacatcgggctgacggacatcgaggtgaacgagctaaagcagctaatcgacaccctggtggaccgcgcaaggaactacattcacacaatgtacactaacgagtacaacaacgccatcaacacttctactgccgaatccgtgaccaacaacctcctctccgtacgcggttactgcctcctgcacggcctagagtgcattgagctgatcgagcacctccagaacaattcgttagagtccggctttaacccgaagaccatcagctacagcaccgtcttcgaccggccgaccaacaagacccgcatccaggccctcacggaggacgaccaaatgcaagagccgttcaagccgagcctgatcgacgggaagtacaacaagatcaagtcgctgcttggctacgtccgcagaatcgggaacgcaccgcgcgtcggcggcatacagatcaccttcgccaacgactccagctacaccctgggcaccgtaacatccgagacgtcatccatcgagctgaacgacagcgttatcgagcggctggaggtgtggggcaacggcgcggtggatgaggccctgttcacgctgagcgacggccgccagctccgcgtcggcgagcggtacgccacgaagtatcggaagtacgcggtggatgggcactacatcgctggcttgtacctcgcgtccgacgagcccagcctcgcgggccaggcggctgggatcgccgtatcgtaccacatgctcgcggacaagaagtga

SEQ ID NO: 153 - protein, 568 aa, synthetic construct
  REGION 1..568: note = The amino acid sequence of the TIC10381PL PirAB fusion protein.
MASIININISGSSDITIINNTPSNPEPLIYNTDTPASEPLTVNPYRDMTIEPHSSIEAIRIDTPIIPETRPNYYVANSGPASSVRAVFYWSHSFTSEWFEYSAITVKAGEDGILQSPSNSVYYSKVVIYNETDKRAFVTGYNKMNTTPINVSENDTLPVLTDVMLIVPYTTSTPDYEWDMSSIIKDAIIGGVGFIPGVGSAMSFLLGLFWPQQKDNTWEQILQKVEQMIENAALQTIKGILNGDIQEIKGKMEHVQYMLETSPGSQESHDAYMFLARYLVSIDERFKSFDNKTNYQILPMYTNTVMLQIPYWKMGIEKKNDIGLTDIEVNELKQLIDTLVDRARNYIHTMYTNEYNNAINTSTAESVTNNLLSVRGYCLLHGLECIELIEHLQNNSLESGFNPKTISYSTVFDRPTNKTRIQALTEDDQMQEPFKPSLIDGKYNKIKSLLGYVRRIGNAPRVGGIQITFANDSSYTLGTVTSETSSIELNDSVIERLEVWGNGAVDEALFTLSDGRQLRVGERYATKYRKYAVDGHYIAGLYLASDEPSLAGQAAGIAVSYHMLADKK

SEQ ID NO: 154 - DNA, 1701 bp, synthetic construct
  source 1..1701: mol_type = other DNA
  misc_feature 1..1701: note = A synthetic coding sequence used for expression in plant cellsencoding a TIC11103 PirAB fusion protein comprised of the TIC7661and TIC7660 coding sequences operably linked.
atgaacaccactccgattactgtaagcactaacgaaacatcgcctctcatgacggacgtgatgccgatggacctgtacgccatctcgacgccagactacgagtgggacatgagttccatcatcaaggacgccgtaattggcggcatcgggttcatccctgggcccggcccggccatctccttcctgctgggcctgttctggccgcagcagaaggacaacacatgggagcagatactccagaaggtcgagcaaatgattgagaatgccgtgttgcagacgatcaagggaatcctaaacggcgaagtacaggagatcaagggcaagatggagcacgtcgagtctatgctcaagaactcgccaggctctcaggagtcacacgacgcctacatgttcctggctcgttacctcgtttcaattgacgagaagttcaagagcttcgacaaccgcaccaactaccaactgttgccgatgtacaccaatacgattatgctccagataccttattggaagatgggcatcgagaagaagaaggacattggcctgaccgacattgaagtcaacgagcttaaggagctgatcgacaagctggtggacaaggccaagaactacatccacacaatgtacacgaacgagcacaacaacgccgtgaacaccagcactgccgagtccgtcacgaacaatctcctcagcgtgcgcggctactgcctgttacacgggctggagtgcattgagctaatcgagcacatccagaacaactccctggagagcgggttctacccgaagatcatcagctacagcaccgctttcgaccgcccgacaaacaagatgcgtatccaagcgctcacggaggacgacgcgatgcaagagccgtttaaaccgtcgctcattaacggcaagtacaacaagatccagagcctcacgggctacgtgcagcggatcggcaacgcgccgcgcgtcggcggcatccgcatcacgttcaccaacgggtcgtcctacacgctcgggacggtgacctccgagacgcacagcatcaagctgaacgactccgtgatcgagtcgttagaggtctggggaaacggtgccgtggacgaggccctgttcaagctgtccgacgggcggctcctccgcatcggcgagcggtacgccaagaagtaccgcaagtacgcggtggacaaccactacatcgcgggcatctacctagcgagcgacgagccgtccctggcgggtcaagccgccgggatcgccgtgagctatcacatgatggcggacaagaaaatgattacgatcaacatcaacgtgaacggcaacgacgtgacgggcaccaacaacaatgagcccactccagtcagcacgacgtacggcccgaacactccggcctcggagccaccggtcgtctcgaactactccgacatcaccattgagccgcacagctcggtccaggccacgcggatcgacacgccggtgatcccggaggcccggccggactactacgtggcgaactcgggccctgcgccgtccgtgcgggccgtgttctactggtcgcactcgttcacctccgagtggttcgagtcgtccagcatcaccgtgaaggcgggcgaggacggaatcctcaaggctccagggaacagcctgtactacagcaaggtcgtcatctacaacgacacagacaagcgggccttcgttaccgggtacaacaagtga

SEQ ID NO: 155 - protein, 566 aa, synthetic construct
  REGION 1..566: note = The amino acid sequence of the TIC11103 PirAB fusion protein.
MNTTPITVSTNETSPLMTDVMPMDLYAISTPDYEWDMSSIIKDAVIGGIGFIPGPGPAISFLLGLFWPQQKDNTWEQILQKVEQMIENAVLQTIKGILNGEVQEIKGKMEHVESMLKNSPGSQESHDAYMFLARYLVSIDEKFKSFDNRTNYQLLPMYTNTIMLQIPYWKMGIEKKKDIGLTDIEVNELKELIDKLVDKAKNYIHTMYTNEHNNAVNTSTAESVTNNLLSVRGYCLLHGLECIELIEHIQNNSLESGFYPKIISYSTAFDRPTNKMRIQALTEDDAMQEPFKPSLINGKYNKIQSLTGYVQRIGNAPRVGGIRITFTNGSSYTLGTVTSETHSIKLNDSVIESLEVWGNGAVDEALFKLSDGRLLRIGERYAKKYRKYAVDNHYIAGIYLASDEPSLAGQAAGIAVSYHMMADKKMITININVNGNDVTGTNNNEPTPVSTTYGPNTPASEPPVVSNYSDITIEPHSSVQATRIDTPVIPEARPDYYVANSGPAPSVRAVFYWSHSFTSEWFESSSITVKAGEDGILKAPGNSLYYSKVVIYNDTDKRAFVTGYNK

SEQ ID NO: 156 - DNA, 1701 bp, synthetic construct
  source 1..1701: mol_type = other DNA
  misc_feature 1..1701: note = A synthetic coding sequence used for expression in plant cellsencoding a TIC11104 PirAB fusion protein comprised of the TIC7663and TIC7662 coding sequences operably linked.
atgaacaccaccctcatcaacgtaagtgagaaggagacgctgccggtgcagaccgacatcatgctgatcgcgccgtactccgtgtccacgccggactacgagtgggacatgtcatcgctcatcaaggacgcgatcatcggcggcgtcggattcatccctgtcgtcggctcggccatgtccttcctcctcggcctgttctggccgcagcagaaggacaacacttgggaacaaatactgcagaaggtcgagcagatgatcgagaacgcgcagctcaacacgattaagggcattctgaacggcgacatccaggagattaagggcaagatggagcacgtgcagtacatgcttgagacaagtccagggagtcaggagtcacacgacgcctacatgttcttagcccgctacctagtgagcatcgacgagaagttcaagtcgttcgacaacaagacaaactaccaaatcttgccaatgtacacgaataccgtcatgctacagatcccatactggaagatgggaattgagaagaagaacgacattggcttgacggacatcgaagtcaacgagcttaaacagcttatcgacactctggtggaccgcgcgcgcaactacatccacaccatgtacgagcgagagtacgacaacgccatcaacacctcaaccgctgcctcggtgaccaacaacctgctctccgtgcgcgggtactgcctcctgcacgggctggagtgcatcgagactatcgagcaccttcagaacaacagcctcaacagtgggttctacccgaagaccatcagctacagcactgtcttcgaccggcccacgaacaagacccgcatccaggctctgacggaagacgaccaaatgcaagagccgttcaagcccgcgctgatcggcggcaagtacaacaagatcaagtccctgttgggctacgtgcgaaggatcggcaacgctccacgggtcggcggcatcaaggtgacgttcaccaatgggtcgagctacacgctcgggacggtcacgtcggaaaccgactccatcgaactgaacgagtcggtcatcgagcggctggaggtgtggggcaacggagccgtggacgaggccctctttaccctgagcgatggccgccagctccgcatcggcgagcggtacgccaagaaataccggaagtacgcggtggatgggcactacatcagcggcctctacctcgcgtcggacgagccctccctcgccggtcaagcagccgggatcgcggtgtcctaccacatgctcgcagacaagaagatgtccaccatcaacatcaacatctcctccagcacggtcactgtgattacaaacaatggcgagacgcccgtcccgctcacctacaacaccaacacgccggagtcggagccgctgacggtcaacccgtaccgcgacatgaccatcgagccgcgctcctccattgaggcgacccgcatcgacacgccgatcatcccggagacgagaccgaactactatgtggcgaactccggcccggccagcagcgtccgggcggtattctactggtcgcacagcttcacctcgcaatggttcgagtattcgtcgatcatcgtgaaggcgggtgaggacggcatactggagtcgccgtcgaacagcctctactacagcaaggtggtgatctacaacgacacggacaagagggcattcgtcacgggctacaacaagtga

SEQ ID NO: 157 - protein, 566 aa, synthetic construct
  REGION 1..566: note = The amino acid sequence of the TIC11104 PirAB fusion protein.
MNTTLINVSEKETLPVQTDIMLIAPYSVSTPDYEWDMSSLIKDAIIGGVGFIPVVGSAMSFLLGLFWPQQKDNTWEQILQKVEQMIENAQLNTIKGILNGDIQEIKGKMEHVQYMLETSPGSQESHDAYMFLARYLVSIDEKFKSFDNKTNYQILPMYTNTVMLQIPYWKMGIEKKNDIGLTDIEVNELKQLIDTLVDRARNYIHTMYEREYDNAINTSTAASVTNNLLSVRGYCLLHGLECIETIEHLQNNSLNSGFYPKTISYSTVFDRPTNKTRIQALTEDDQMQEPFKPALIGGKYNKIKSLLGYVRRIGNAPRVGGIKVTFTNGSSYTLGTVTSETDSIELNESVIERLEVWGNGAVDEALFTLSDGRQLRIGERYAKKYRKYAVDGHYISGLYLASDEPSLAGQAAGIAVSYHMLADKKMSTININISSSTVTVITNNGETPVPLTYNTNTPESEPLTVNPYRDMTIEPRSSIEATRIDTPIIPETRPNYYVANSGPASSVRAVFYWSHSFTSQWFEYSSIIVKAGEDGILESPSNSLYYSKVVIYNDTDKRAFVTGYNK

SEQ ID NO: 158 - DNA, 1701 bp, synthetic construct
  source 1..1701: mol_type = other DNA
  misc_feature 1..1701: note = A synthetic coding sequence used for expression in plant cellsencoding a TIC11302 PirAB fusion protein.
atgattacgatcaacatcaacgtgaacggcaacgacgtgacgggcaccaacaacaatgagcccactccagtcagcacgacgtacggcccgaacactccggcctcggagccaccggtcgtctcgaactactccgacatcaccattgagccgcacagctcggtccaggccacgcggatcgacacgccggtgatcccggaggcccggccggactactacgtggcgaactcgggccctgcgccgtccgtgcgggccgtgttctactggtcgcactcgttcacctccgagtggttcgagtcgtccagcatcaccgtgaaggcgggcgaggacggaatcctcaaggctccagggaacagcctgtactacagcaaggtcgtcatctacaacgacacagacaagcgggccttcgttaccgggtacaacaagatgaacatcagcccgatcaacgtctcggagaacgagacgctcccggagctgaccgacgtgatgctgatcgtcccatacacgaccagcacgccggattacgagtgggacatgtcgtcgatcattaaggatgcgatcatcggaggcgttggcttcatccctggcgcgggctcggccatgagcttcctgctcggcctgttctggccgcagcagaaggataacacttgggagcagatacttcagaaggtggaacagatgatcgagaacgcggtcctccaaacgatcaagggcatcctcaacggcgacatccaggagattaagggaaagatggagcacgttcagtacatgctcgaaaccagccctgggagccaggagagccacgacgcctacatgttcttggcacgttacctcgtctcgattgacgagaagttcaagtccttcgacaacaagacaaactaccagatcttgccaatgtacaccaatacggttatgttacagattccgtactggaagatgggcatcgagaagaagaatgacatcggcttgaccgacatcgaggtcaatgagcttaagcaacttatcgacaagctggtggacaaggccaagtcctacatccacacaatgtacaccaacgagtacaacgacgcgatcaacaccagcaccgcctcaagcgtcacaaacaacctcctgtccgtgcgcggttactgccttctgcacggcctggagtgcatcgaacttattgagcatctccagaacaacagcctggagtccggcttctacccgaagacgatcagctactccacggtcttcgaccggcagaccaacaagatgcggatacaagcgctcactgaggacgaccagatgcaagaacccttcaagccctcgctcatcaacgggaagtacaacaagatccagagcctcctcggctacgtccagcgcatcggcaacgcgccgcgcgtcggcgggatcaagatcacgttcgccaacgggtctagttacacgctgggcaccgtgaccagcgagacctccagtattgagcttaacgactcggtgatcgagcggctggaggtgtggggcaacggcgcggtggacgaggcgctgttcaccctctcggacgggcggcagctccgggtcggcgagcggtacgcgaccaagtaccgtaagtacgccgtggacggccactacatcgccggtctgtacctcgccagtgacgagcccagcctagcgggccaggcggctggcatcgccgtgtcgtaccacatgctcgacgacaagaagtga

SEQ ID NO: 159 - DNA, 24 bp, synthetic construct
  source 1..24: mol_type = other DNA
  misc_feature 1..24: note = A synthetic coding sequence encoding a Histidine tag that isoperably linked to coding sequences expressed in Escherichia coliand used for protein purification.
caccaccatcacgctcaccatcac

SEQ ID NO: 160 - protein, 8 aa, synthetic construct
  REGION 1..8: note = The amino acid sequence of the Histidine tag.
HHHHAHHH